(19) United States
(12) Patent Application Publication
(10) Pub. No.: US 2019/0071474 A1
(43) Pub. Date: Mar. 7, 2019

(54) PRODUCTION OF GIBBERELLINS IN RECOMBINANT HOSTS

(71) Applicant: Evolva SA, Reinach (CH)

(72) Inventors: Esben Halkjaer Hansen, Frederiksberg (DK); Nina Nicoline Rasmussen, Hvidovre (DK); Michael Naesby, Basel (CH); Jane Dannow Dyekjaer, Copenhagen (DK); Simon Carlsen, Copenhagen (DK); Adam Matthew Takos, Valby (DK); Nicholas Ohler, Reinach (CH)

(21) Appl. No.: 16/080,741

(22) PCT Filed: Mar. 3, 2017
(86) PCT No.: PCT/EP2017/055083

§ 371 Date: Aug. 29, 2018

Related U.S. Application Data
(60) Provisional application No. 62/303,973, filed on Mar. 4, 2016.

(51) Int. Cl. C07K 14/415; C12N 9/02; C12P 27/00

(52) U.S. Cl. CPC C07K 14/415; C12N 9/0071; C12Y 114/13079; C12Y 114/14001; C12P 27/00

(57) ABSTRACT

The invention relates to recombinant microorganisms and methods for producing gibberellin compounds and gibberellin precursors.

### CROSS REFERENCE

This application is related to U.S. provisional patent application, Ser. No. 62/303,973, filed Mar. 4, 2016, the disclosure of which is incorporated by reference herein in its entirety.

### SEQUENCE LISTING

The sequence listing submitted herewith, entitled “15-1649-WO_SequenceListing_ST25.txt” and 713 kb in size, is incorporated by reference in its entirety.

### BACKGROUND OF THE INVENTION

### Field of the Invention

This disclosure relates to recombinant production of gibberellin compounds and gibberellin precursors in recombinant hosts. In particular, this disclosure relates to production of gibberellin A3 (i.e., GA₃) in recombinant hosts.

### Description of Related Art

Gibberellins are diterpene plant hormones that are biosynthesized through complex pathways and control diverse aspects of growth and development during a plant's life cycle, including, but not limited to, seed germination, stem elongation, sex expression, flowering, formation of fruits, and senescence. Gibberellin structure is shown in FIG. 1. Higher plants as well as some fungi and bacteria produce gibberellins, of which more than 130 are known. Only a small subset of these gibberellins, including gibberellin A1 (i.e., GA₁), GA₃, GA₄, and GA₇ are thought to exert an effect on plant growth and/or metabolism; the remainder are believed to be precursors for these gibberellins, or deactivated metabolites. GA₁, GA₃, GA₄, and GA₇ commonly have a hydroxyl group on C-3, a carboxylic acid group on C-6, and a lactone between C-4 and C-10. See, Yamaguchi, 2008, Annu. Rev. Plant Biol. 59:225-51; Bömke and Tudzynski, 2009, Phytochemistry 70:1876-93.

In plants, fungi, and bacteria, gibberellins are synthesized from kaurenoic acid in a stepwise fashion, wherein a series of functional group additions and oxidations are performed by cytochrome P450 monooxygenases (P450s) and 2-oxoglutarate-dependent dioxygenases (2-ODDs). See, FIG. 2. Although structurally identical gibberellins are synthesized biologically across plants, fungi, and bacteria, there are differences in the biosynthetic pathways and in the specific enzymes involved. For example, in plants, GA₄ can be synthesized from kaurenoic acid via a pathway that includes GA₁₂, GA₁₅, GA₂₄, and GA₉, while in fungi, GA₄ is synthesized from kaurenoic acid via a pathway that includes GA₁₄. In another example, conversion of GA₁₂ to GA₁₅ in plants is catalyzed by a P450 enzyme, while in bacteria conversion of GA₁₂ to GA₁₅ is catalyzed by a 2-ODD enzyme.

In plants, the P450 enzyme involved is kaurenoic acid oxidase (KAO) and the 2-ODD enzymes are GA oxidases (e.g., GA₂₀ₒₓ, GA₇ₒₓ, etc.). In fungi, the P450 enzymes P450-1, P450-2, and P450-3 are responsible for the majority of the gibberellin synthesis pathway, while GA₄ desaturase (DES) is the only 2-ODD enzyme involved. See, Yamaguchi, Annu. Rev. Plant Biol. 59:225-51 (2008); Bömke and Tudzynski, Phytochemistry 70:1876-93 (2009). In bacteria, P450 enzymes perform the majority of gibberellin biosynthesis. See, Bottini et al., 2004, Appl. Microbiol. Biotechnol. 65:497-503.

GA₃ (gibberellic acid), is used commercially for a variety of purposes, including inducing seed germination, inducing flowering, and increasing fruit size. Because plants produce only minute amounts of GA₃, the hormone is produced industrially by submerged fermentation using the fungus Gibberella fujikuroi (also known as Fusarium fujikuroi.) F. fujikuroi is not a preferred production host due to slow growth compared to other production hosts; an F. fujikuroi fermentation typically can last up 9 days, while a Saccharomyces cerevisiae fermentation usually is completed in 4-5 days. See Uthandi et al., 2009, Journal of Scientific & Industrial Research 69:211-4 and Rodrigues et al., 2009, Braz. Arch. Biol. Tech. 52(Special No.):181-8. As production, recovery, and purification of GA₃ and other gibberellins have proven to be costly, there remains a need for a recombinant production system that can accumulate high yields of desired gibberellins, such as GA₃, GA₄, GA₇, or GA₁, in a more cost-effective manner.

### SUMMARY OF THE INVENTION

It is against the above background that the present invention provides certain advantages and advancements over the prior art.

Although this invention as disclosed herein is not limited to specific advantages or functionalities, the invention provides a recombinant host cell, comprising:
(a) a recombinant gene encoding a first cytochrome P450 (P450) polypeptide; and/or
(b) a recombinant gene encoding a 2-oxoglutarate-dependent dioxygenase (2-ODD) polypeptide and/or a second cytochrome P450 (P450) polypeptide;

wherein the recombinant host cell is capable of producing a gibberellin precursor and/or a gibberellin compound.

In one aspect of the recombinant host cell disclosed herein, the gene encoding the first P450 polypeptide encodes a kaurenoic acid oxidase (KAO) polypeptide or a cytochrome P450 monooxygenase-1 (P450-1) polypeptide.

In one aspect of the recombinant host cell disclosed herein, the gene encoding the first P450 polypeptide comprises:
(a) a gene encoding a kaurenoic acid oxidase (KAO1) polypeptide;
(b) a gene encoding a kaurenoic acid oxidase (KAO2) polypeptide;
(c) a gene encoding a kaurenoic acid oxidase (KAO3) polypeptide;
(d) a gene encoding a kaurenoic acid oxidase (KAO4) polypeptide;
(e) a gene encoding a kaurenoic acid oxidase (KAO5) polypeptide;
(f) a gene encoding a kaurenoic acid oxidase (KAO6) polypeptide;
(g) a gene encoding a kaurenoic acid oxidase (KAO9) polypeptide;
(h) a gene encoding a kaurenoic acid oxidase (KAO10) polypeptide;
(i) a gene encoding a kaurenoic acid oxidase (KAO11) polypeptide;
(j) a gene encoding a cytochrome P450 monooxygenase-2 (P450-2) polypeptide;
(k) a gene encoding a cytochrome P450 monooxygenase-3 (P450-3) polypeptide;
(l) a gene encoding a cytochrome P-450 BJ-1 (CYP112) polypeptide; and/or
(m) a gene encoding a gibberellin A13-oxidase (GA₁₃ₒₓ) polypeptide.

In one aspect of the recombinant host cell disclosed herein,
(a) the KAO1 polypeptide comprises a KAO1 polypeptide having at least 50% sequence identity to the amino acid sequence set forth in SEQ ID NO:90;
(b) the KAO2 polypeptide comprises a KAO2 polypeptide having at least 50% sequence identity to the amino acid sequence set forth in SEQ ID NO:88;
(c) the KAO3 polypeptide comprises a KAO3 polypeptide having at least 50% sequence identity to the amino acid sequence set forth in SEQ ID NO:146;
(d) the KAO4 polypeptide comprises a KAO4 polypeptide having at least 50% sequence identity to the amino acid sequence set forth in SEQ ID NO:74;
(e) the KAO5 polypeptide comprises a KAO5 polypeptide having at least 60% sequence identity to the amino acid sequence set forth in SEQ ID NO:62;
(f) the KAO6 polypeptide comprises a KAO6 polypeptide having at least 60% sequence identity to the amino acid sequence set forth in SEQ ID NO:60;
(g) the KAO9 polypeptide comprises a KAO9 polypeptide having at least 70% sequence identity to the amino acid sequence set forth in SEQ ID NO:68;
(h) the KAO10 polypeptide comprises a KAO10 polypeptide having at least 70% sequence identity to the amino acid sequence set forth in SEQ ID NO:58;
(i) the KAO11 polypeptide comprises a KAO11 polypeptide having at least 65% sequence identity to the amino acid sequence set forth in SEQ ID NO:64;
(j) the P450-2 polypeptide comprises a P450-2 polypeptide having at least 50% sequence identity to the amino acid sequence set forth in SEQ ID NO:80;
(k) the P450-3 polypeptide comprises a P450-3 polypeptide having at least 50% sequence identity to the amino acid sequence set forth in SEQ ID NO:186;
(l) the CYP112 polypeptide comprises a CYP112 polypeptide having at least 70% sequence identity to the amino acid sequence set forth in SEQ ID NOs: 4, 6, 8, 10, 124, or 128; or
(m) the GA₁₃ₒₓ polypeptide comprises a GA₁₃ₒₓ polypeptide having at least 70% sequence identity to the amino acid sequence set forth in SEQ ID NO:98.

In one aspect of the recombinant host cell disclosed herein, the gene encoding the second P450 polypeptide comprises:
(a) a P450-2 polypeptide having at least 50% sequence identity to the amino acid sequence set forth in SEQ ID NO:80;
(b) a P450-2 polypeptide having at least 50% sequence identity to the amino acid sequence set forth in SEQ ID NO:233;
(c) a P450-2 polypeptide having at least 50% sequence identity to the amino acid sequence set forth in SEQ ID NO 235;
(d) a P450-2 polypeptide having at least 50% sequence identity to the amino acid sequence set forth in SEQ ID NO:237;
(e) a P450-2 polypeptide having at least 50% sequence identity to the amino acid sequence set forth in SEQ ID NO:18; or
(f) a CYP112 polypeptide having at least 70% sequence identity to the amino acid sequence set forth in SEQ ID NO:124.

In one aspect of the recombinant host cell disclosed herein, the gene encoding the 2-ODD polypeptide comprises:
(a) a gene encoding a desaturase (DES) polypeptide;
(b) a gene encoding a gibberellin A7-oxidase (GA₇ₒₓ) polypeptide;
(c) a gene encoding a gibberellin A3-oxidase (GA₃ₒₓ) polypeptide; or
(d) a gene encoding a gibberellin A20-oxidase (GA₂₀ₒₓ) polypeptide.

In one aspect of the recombinant host cell disclosed herein,
(a) the DES polypeptide comprises a DES polypeptide having at least 50% sequence identity to the amino acid sequence set forth in SEQ ID NO:26;
(b) the GA₇ₒₓ polypeptide comprises a GA₇ₒₓ polypeptide having 60% or greater sequence identity to the amino acid sequence set forth in SEQ ID NO:152;
(c) the GA₃ₒₓ polypeptide comprises a GA₃ₒₓ polypeptide having at least 50% sequence identity to the amino acid sequence set forth in SEQ ID NO:28, SEQ ID NO:30, SEQ ID NO:36, or SEQ ID NO:44; or
(d) the GA₂₀ₒₓ polypeptide comprises a GA₂₀ₒₓ polypeptide having at least 70% sequence identity to the amino acid sequence set forth in SEQ ID NO:40 or SEQ ID NO:42.

The invention further provides a recombinant host cell comprising:
(a) a gene encoding a kaurenoic acid oxidase (KAO4) polypeptide having at least 50% sequence identity to the amino acid sequence set forth in SEQ ID NO:74;
(b) a gene encoding a desaturase (DES) polypeptide having at least 50% sequence identity to the amino acid sequence set forth in SEQ ID NO:26;
(c) a gene encoding a cytochrome P450 monooxygenase-2 (P450-2) polypeptide having at least 50% sequence identity to the amino acid sequence set forth in SEQ ID NO:80; and
(d) a gene encoding a cytochrome P450 monooxygenase-3 (P450-3) polypeptide having at least 50% sequence identity to the amino acid sequence set forth in SEQ ID NO:186;

wherein the recombinant host cell is capable of producing a gibberellin precursor and/or a gibberellin compound.

The invention further provides a recombinant host cell, comprising:
(a) a gene encoding a kaurenoic acid oxidase (KAO4) polypeptide having at least 50% sequence identity to the amino acid sequence set forth in SEQ ID NO:74;
(b) a gene encoding a gibberellin A20-oxidase (GA₂₀ₒₓ) polypeptide having at least 50% sequence identity to the amino acid sequence set forth in SEQ ID NO:42;
(c) a gene encoding a cytochrome P450 monooxygenase-3 (P450-3) polypeptide having at least 50% sequence identity to the amino acid sequence set forth in SEQ ID NO: 186; and
(d) a gene encoding a desaturase (DES) polypeptide having at least 50% sequence identity to the amino acid sequence set forth in SEQ ID NO:26;

wherein the recombinant host cell is capable of producing a gibberellin precursor and/or a gibberellin compound.

The invention further provides a recombinant host cell comprising a gene encoding a kaurenoic acid oxidase (KAO) polypeptide having at least 60% sequence identity to the amino acid sequence set forth in SEQ ID NO:62, SEQ ID NO:60, or SEQ ID NO:152, at least 70% sequence identity to the amino acid sequence set forth in SEQ ID NO:58 or SEQ ID NO:68, at least 65% sequence identity to the amino acid sequence set forth in SEQ ID NO:64, or at least 50% sequence identity to the amino acid sequence set forth in SEQ ID NO:74;

wherein the recombinant host cell is capable of producing gibberellin precursor and/or a gibberellin compound.

In one aspect of the recombinant host cell disclosed herein, the recombinant host cell further comprises:
(a) a gene encoding a gibberellin A20-oxidase (GA₂₀ₒₓ) polypeptide having at least 70% sequence identity to the amino acid sequence set forth in SEQ ID NO:40; and
(b) a gene encoding a gibberellin A13-oxidase (GA₁₃ₒₓ) polypeptide having at least 70% sequence identity to the amino acid sequence set forth in SEQ ID NO:98.

The invention further provides a recombinant cell host, comprising:
(a) a gene encoding a kaurenoic acid oxidase (KAO11) polypeptide having at least 65% sequence identity to the amino acid sequence set forth in SEQ ID NO:64; and
(b) a gene encoding a cytochrome P-450 BJ-1 (CYP112) polypeptide having at least 70% sequence identity to the amino acid sequence set forth in SEQ ID NO:124;

wherein the recombinant host cell is capable of producing a gibberellin precursor and/or a gibberellin compound.

The invention further provides a recombinant host cell, comprising:
(a) a gene encoding a kaurenoic acid oxidase (KAO4) polypeptide having at least 50% sequence identity to the amino acid sequence set forth in SEQ ID NO:74; and
(b) a gene encoding a cytochrome P-450 BJ-1 (CYP112) polypeptide having at least 70% sequence identity to the amino acid sequence set forth in SEQ ID NO:124;

wherein the recombinant host cell is capable of producing a gibberellin precursor and/or a gibberellin compound.

In one aspect of the recombinant host cell disclosed herein, the recombinant host cell further comprises:
(a) a gene encoding a polypeptide capable of synthesizing geranylgeranyl pyrophosphate (GGPP) from farnesyl diphosphate (FPP) and isopentenyl diphosphate (IPP);
(b) a gene encoding a polypeptide capable of synthesizing ent-copalyl diphosphate from GGPP;
(c) a gene encoding a polypeptide capable of synthesizing ent-kaurene from ent-copalyl pyrophosphate;
(d) a gene encoding a bifunctional polypeptide capable of synthesizing ent-copalyl diphosphate from GGPP and synthesizing ent-kaurene from ent-copalyl pyrophosphate;
(e) a gene encoding a polypeptide capable of synthesizing ent-kaurenoic acid from ent-kaurene;
(f) a gene encoding a cytochrome B5 polypeptide;
(g) a gene encoding a polypeptide capable of reducing cytochrome B5 polypeptide;
(h) a gene encoding a polypeptide capable of reducing cytochrome P450 complex;
(i) a gene encoding a ferredoxin polypeptide;
(j) a gene encoding a ferredoxin reductase polypeptide; and/or
(k) an alcohol dehydrogenase (ADH) polypeptide capable of reducing a gibberellin intermediate.

In one aspect of the recombinant host cell disclosed herein,
(a) the polypeptide capable of synthesizing geranylgeranyl pyrophosphate (GGPP) from farnesyl diphosphate (FPP) and isopentenyl diphosphate (IPP) comprises a polypeptide having at least 70% sequence identity to the amino acid sequence set forth in SEQ ID NO:50, SEQ ID NO:134, or SEQ ID NO:178;
(b) the polypeptide capable of synthesizing ent-copalyl diphosphate from GGPP comprises a polypeptide having at least 70% sequence identity to the amino acid sequence set forth in SEQ ID NO:38, SEQ ID NO:102, SEQ ID NO:104, SEQ ID NO:106, SEQ ID NO:108, or SEQ ID NO:180;
(c) the polypeptide capable of synthesizing ent-kaurene from ent-copalyl pyrophosphate comprises a polypeptide having at least 70% sequence identity to the amino acid sequence set forth in SEQ ID NO:102 or SEQ ID NO:106;
(d) the bifunctional polypeptide capable of synthesizing ent-copalyl diphosphate from GGPP and synthesizing ent-kaurene from ent-copalyl pyrophosphate comprises a CDPS-KS polypeptide having at least 70% sequence identity to the amino acid sequence set forth in SEQ ID NO:104;
(e) the polypeptide capable of synthesizing ent-kaurenoic acid from ent-kaurene comprises a polypeptide having at least 70% sequence identity to the amino acid sequence set forth in SEQ ID NO:82, SEQ ID NO:164, SEQ ID NO:170, or SEQ ID NO:172;
(f) the cytochrome B5 polypeptide comprises a cytochrome B5 polypeptide having at least 60% sequence identity to the amino acid sequence set forth in SEQ ID NO:160 or SEQ ID NO:239;
(g) the cytochrome B5 reductase polypeptide comprises a cytochrome B5 reductase polypeptide having at least 80% sequence identity to the amino acid sequence set forth in SEQ ID NO:2 or SEQ ID NO:241;
(h) the polypeptide capable of reducing cytochrome P450 complex comprises a CPR reductase polypeptide having at least 50% sequence identity to the amino acid sequence set forth in SEQ ID NO:48, SEQ ID NO:100, SEQ ID NO:140, SEQ ID NO:158, SEQ ID NO:168, SEQ ID NO:192 or SEQ ID NO:194;
(i) the ferredoxin polypeptide comprises a ferredoxin polypeptide having at least 80% sequence identity to the amino acid sequence set forth in SEQ ID NO:148;
(j) the ferredoxin reductase polypeptide comprises a ferredoxin reductase polypeptide having at least 70% sequence identity to the amino acid sequence set forth in SEQ ID NO:150; and/or
(k) the ADH polypeptide comprises an ADH polypeptide having at least 70% sequence identity to the amino acid sequence set forth in SEQ ID NO:116.

In one aspect of the recombinant host cell disclosed herein, the recombinant host cell further comprises:
(a) a gene encoding an open reading frame (ORF) polypeptide;
(b) a gene encoding an aldehyde dehydrogenase (ALDH) polypeptide;
(c) a gene encoding a myo-inositol transport protein ITR1 (smt) polypeptide;
(d) a gene encoding an endoplasmic reticulum (ER) membrane polypeptide; and/or
(e) a gene encoding a damage resistance protein 1 (DAP) polypeptide.

In one aspect of the recombinant host cell disclosed herein,
(a) the ORF polypeptide comprises an ORF polypeptide having at least 50% sequence identity to the amino acid sequence set forth in SEQ ID NO:154 or SEQ ID NO:156;
(b) the AIdDH polypeptide comprises an AIdDH polypeptide having at least 60% sequence identity to the amino acid sequence set forth in SEQ ID NO:202;
(c) the smt polypeptide comprises an smt polypeptide having at least 90% sequence identity to the amino acid sequence set forth in SEQ ID NO:209;
(d) the ER membrane polypeptide comprises an inheritance of cortical ER protein 2 (ICE2) polypeptide having at least 60% sequence identity to the amino acid sequence set forth in SEQ ID NO:206; and/or
(e) the DAP polypeptide comprises a DAP polypeptide having at least 70% sequence identity to the amino acid sequence set forth in SEQ ID NO:213, SEQ ID NO:215, SEQ ID NO:217, or SEQ ID NO:224.

In one aspect of the recombinant host cell disclosed herein, expression of the recited genes increases the portion of the gibberellin precursor and/or the gibberellin compound produced by the recombinant host cell by at least about 10%, 25%, 50%, 75%, 80%, 90%, 95%, 100% or more.

In one aspect of the recombinant host cells disclosed herein, the gibberellin compound comprises GA₁, GA₃, GA₄, GA₅, GA₇, GA₉, GA₁₂, GA₁₃, GA₁₄, GA₁₅, GA₁₉, GA₂₀, GA₂₄, GA₂₅, GA₃₆, GA₃₇, GA₄₄, GA₅₃, and/or GA₁₁₀.

In one aspect of the recombinant host cells disclosed herein, the recombinant host comprises a plant cell, a mammalian cell, an insect cell, a fungal cell, an algal cell or a bacterial cell.

The invention further provides a method of producing a gibberellin precursor and/or a gibberellin compound in a cell culture, comprising growing the recombinant host cell disclosed herein in a cell culture, under conditions in which the genes are expressed;

wherein the gibberellin precursor and/or the gibberellin compound is produced by the recombinant host cell.

In one aspect, the method disclosed herein further comprises isolating the gibberellin precursor and/or the gibberellin compound from the cell culture.

In one aspect of the method of producing a gibberellin precursor and/or gibberellin compound in a cell culture, the isolating step comprises:
(a) contacting the cell culture comprising the gibberellin precursor and/or the gibberellin compound with:
   (i) one or more adsorbent resins in order to bind at least a portion of the gibberellin precursor and/or the gibberellin compound to the resin, thereby isolating the gibberellin precursor and/or the gibberellin compound; or
   (ii) one or more ion exchange or reversed-phase chromatography columns in order to bind at least a portion of the gibberellin precursor and/or the gibberellin compound in the column, thereby isolating the gibberellin precursor and/or the gibberellin compound; or
(b) crystallizing and/or extracting the gibberellin precursor and/or the gibberellin compound from the cell culture, thereby isolating the gibberellin precursor and/or the gibberellin compound; or
(c) separating the cell culture into a solid phase and a liquid phase, wherein the liquid phase comprises the gibberellin precursor and/or the gibberellin compound; and
   (i) contacting the liquid phase with one or more adsorbent resins in order to bind at least a portion of the gibberellin precursor and/or the gibberellin compound to the resin, thereby isolating the gibberellin precursor and/or the gibberellin compound;
   (ii) contacting the liquid phase with one or more ion exchange or reversed-phase chromatography columns in order to bind at least a portion of the gibberellin precursor and/or the gibberellin compound in the column, thereby isolating the gibberellin precursor and/or the gibberellin compound; or
   (iii) crystallizing and/or extracting the gibberellin precursor and/or the gibberellin compound from the liquid phase, thereby isolating the gibberellin precursor and/or the gibberellin compound.

In one aspect, the method disclosed herein further comprises recovering the gibberellin precursor and/or the gibberellin compound.

In one aspect, the method disclosed herein further comprises
(a) one or more steps of converting kaurenoic acid to GA₁₂ and GA₁₄ catalyzed by a first P450 polypeptide; and
(b) a step of converting GA₁₄ to GA₄ catalyzed by a second P450 polypeptide.

In one aspect of the methods disclosed herein:
(a) the first P450 polypeptide comprises:
   (i) a KAO4 polypeptide having at least 50% sequence identity to the amino acid sequence set forth in SEQ ID NO:74;
   (ii) a KAO1 polypeptide having at least 50% sequence identity to the amino acid sequence set for in SEQ ID NO:90; or
   (iii) a KAO3 polypeptide having at least 50% sequence identity to the amino acid sequence set for in SEQ ID NO:146; and
(b) the second P450 polypeptide comprises:
   (i) a P450-2 polypeptide having at least 50% sequence identity to the amino acid sequence set forth in SEQ ID NO:80;
   (ii) a P450-2 polypeptide having at least 50% sequence identity to the amino acid sequence set forth in SEQ ID NO:233;
   (iii) a P450-2 polypeptide having at least 50% sequence identity to the amino acid sequence set forth in SEQ ID NO 235;
   (iv) a P450-2 polypeptide having at least 50% sequence identity to the amino acid sequence set forth in SEQ ID NO:237;
   (v) a P450-2 polypeptide having at least 50% sequence identity to the amino acid sequence set forth in SEQ ID NO:18; or
   (vi) a CYP112 polypeptide having at least 70% sequence identity to the amino acid sequence set forth in SEQ ID NO:124.

In one aspect, the method disclosed herein further comprises a step of converting GA₄ to GA₁ catalyzed by a third P450 polypeptide.

In one aspect of the method disclosed herein, the third P450 polypeptide comprises a P450-3 polypeptide having at least 50% sequence identity to the amino acid sequence set forth in SEQ ID NO:186; or a GA₁₃ₒₓ₋₁ polypeptide having at least 50% sequence identity to the amino acid sequence set forth in SEQ ID NO:98.

In one aspect, the method disclosed herein further comprises:
(a) a step of converting GA₄ to GA₇ catalyzed by a 2-ODD polypeptide; and
(b) a step of converting GA₇ to GA₃ catalyzed by a fourth P450 polypeptide.

In one aspect of the method disclosed herein:
(a) the 2-ODD polypeptide comprises a DES polypeptide having at least 50% sequence identity to the amino acid sequence set forth in SEQ IN NO:26; and
(b) the fourth P450 polypeptide comprises a P450-3 polypeptide having at least 50% sequence identity to the amino acid sequence set forth in SEQ ID NO:186; or a GA₁₃ₒₓ₋₁ polypeptide having at least 50% sequence identity to the amino acid sequence set forth in SEQ ID NO:98.

In one aspect, the method disclosed herein further comprises:
(a) one or more steps of converting kaurenoic acid to GA₁₂ and/or GA₁₄ catalyzed by a first P450 polypeptide; and
(b) a step of converting GA₁₄ to GA₄ catalyzed by a 2-ODD polypeptide.

In one aspect of the method disclosed herein:
(a) the first P450 polypeptide comprises a KAO4 polypeptide having at least 50% sequence identity to the amino acid sequence set forth in SEQ ID NO:74; and
(b) the 2-ODD polypeptide comprises a GA₂₀ₒₓ polypeptide having at least 70% sequence identity to the amino acid sequence set forth in SEQ ID NO:40 or SEQ ID NO:42.

In one aspect, the method disclosed herein further comprises a step of converting GA₄ to GA₁ catalyzed by a second P450 polypeptide.

In one aspect of the method disclosed herein, the second P450 polypeptide comprises a P450-3 polypeptide having at least 50% sequence identity to the amino acid sequence set forth in SEQ ID NO:186; or a GA₁₃ₒₓ₋₁ polypeptide having at least 50% sequence identity to the amino acid sequence set forth in SEQ ID NO:98.

In one aspect, the method disclosed herein further comprises:
(a) a step of converting GA₄ to GA₇ catalyzed by a second 2-ODD polypeptide; and
(b) a step of converting GA₇ to GA₃ catalyzed by a second P450 polypeptide.

In one aspect of the method disclosed herein:
(a) the second 2-ODD polypeptide comprises a DES polypeptide having at least 50% sequence identity to the amino acid sequence set forth in SEQ ID NO:26; and
(b) the second P450 polypeptide comprises a P450-3 polypeptide having at least 50% sequence identity to the amino acid sequence set forth in SEQ ID NO:186.

In one aspect of the method disclosed herein the recombinant host cell is grown in a fermentor at a temperature for a period of time, wherein the temperature and period of time facilitate the production of the gibberellin precursor and/or the gibberellin compound.

In one aspect of the methods disclosed herein, the gibberellin compound comprises GA₃ and its precursors, metabolites, or related compounds, including: GA₁, GA₄, GA₅, GA₇, GA₉, GA₁₂, GA₁₃, GA₁₄, GA₁₅, GA₁₉, GA₂₀, GA₂₄, GA₂₅, GA₃₆, GA₃₇, GA₄₄, GA₅₃, and/or GA₁₁₀.

In one aspect of the methods disclosed herein, the recombinant host comprises a plant cell, a mammalian cell, an insect cell, a fungal cell, an algal cell or a bacterial cell.

The invention further provides a cell culture, comprising the recombinant host cell disclosed herein, the cell culture further comprising:
(a) the gibberellin precursor and/or the gibberellin compound produced by the recombinant host cell;
(b) a carbon source; and
(c) supplemental nutrients comprising trace metals, vitamins, salts, YNB, a nitrogen source, and/or amino acids;

wherein one or more gibberellin precursors and/or the gibberellin compounds are present at a concentration of at least 100 mg/liter of the cell culture.

The invention further provides a cell lysate from the recombinant host cell disclosed herein and grown in the cell culture, comprising:
(a) the gibberellin precursor and/or the gibberellin compound produced by the recombinant host cell;
(b) a carbon source; and
(c) supplemental nutrients comprising trace metals, vitamins, salts, YNB, and/or amino acids;

wherein one or more gibberellin precursors and/or the gibberellin compounds are present at a concentration of at least 100 mg/liter of the cell culture.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a general chemical structure for a gibberellin, with carbon atoms numbered according to IUPAC nomenclature.

FIG. 2A shows a schematic of gibberellin biosynthesis pathways. The starting material for gibberellin biosynthesis, ent-kaurenoic acid, is formed by successive conversions of geranylgeranyl diphosphate (GGPP) to ent-copalyl diphosphate (ent-copalyl-PP), to ent-Kaurene, and finally to ent-kaurenoic acid, catalyzed by a copalyl diphosphate synthase (CDPS) enzyme, a kaurene synthase (KS) enzyme, and a kaurene oxidase (KO) enzyme, respectively.

FIG. 2B shows a schematic of gibberellin biosynthesis in fungi, plants, and/or bacteria.

FIG. 3 shows a biosynthetic route from kaurenoic acid to GA₃ in an S. cerevisiae strain comprising genes encoding a G. fujikuroi P450-2-1 polypeptide (SEQ ID NO:79, SEQ ID NO:80), a G. fujikuroi P450-3-4 polypeptide (SEQ ID NO:185, SEQ ID NO:186), a Sphaceloma manihoticola KAO4 polypeptide (SEQ ID NO:73, SEQ ID NO:74), a G. fujikuroi DES-1 polypeptide (SEQ ID NO:25, SEQ ID NO:26), and an A. niger cytochrome P450 reductase-16 (CPR16) polypeptide (SEQ ID NO:157, SEQ ID NO:158), as described in Example 2.

FIG. 4A shows gibberellin accumulation by an S. cerevisiae strain comprising genes encoding a truncated CDPS polypeptide (SEQ ID NO:179, SEQ ID NO:180), a KS polypeptide (SEQ ID NO:181, SEQ ID NO:182), a first KO polypeptide (SEQ ID NO:171, SEQ ID NO:172), a second KO polypeptide (SEQ ID NO:169, SEQ ID NO:170), a CPR polypeptide (SEQ ID NO:167, SEQ ID NO:168), an ERG20-GGPPS7 polypeptide (SEQ ID NO:195, SEQ ID NO:196), and either i) genes encoding a G. fujikuroi P450-2-1 polypeptide (SEQ ID NO:79, SEQ ID NO:80), G. fujikuroi P450-3-4 polypeptide (SEQ ID NO:185, SEQ ID NO:186), S. manihoticola KAO4 polypeptide (SEQ ID NO:73, SEQ ID NO:74), G. fujikuroi DES-1 polypeptide (SEQ ID NO:25, SEQ ID NO:26), G. fujikuroi cytochrome B5 polypeptide (SEQ ID NO:159, SEQ ID NO:160), and G. fujikuroi cytochrome B5 reductase polypeptide (SEQ ID NO:1, SEQ ID NO:2) (Strain “N”), ii) genes encoding a G. fujikuroi P450-2-1 polypeptide (SEQ ID NO:79, SEQ ID NO:80), G. fujikuroi P450-3-4 polypeptide (SEQ ID NO:185, SEQ ID NO:186), S. manihoticola KAO4 polypeptide (SEQ ID NO:73, SEQ ID NO:74), and G. fujikuroi DES-1 polypeptide (SEQ ID NO:25, SEQ ID NO:26) (Strain “I”), or iii) genes encoding a Cucurbita maxima GA₂₀ₒₓ₋₄ polypeptide (SEQ ID NO:39, SEQ ID NO:40), G. fujikuroi P450-3-4 polypeptide (SEQ ID NO:185, SEQ ID NO:186), S. manihoticola KAO4 polypeptide (SEQ ID NO:73, SEQ ID NO:74), G. fujikuroi DES-1 polypeptide (SEQ ID NO:25, SEQ ID NO:26), and A. niger CPR16 (SEQ ID NO:157, SEQ ID NO:158) (Strain “F”).

FIG. 4B shows a Liquid Chromatography-Mass Spectrometry (LC-MS) chromatogram analyzing accumulation of gibberellins and gibberellin precursors, including GA₃, GA₄, GA₁₂, GA₁₄, and kaurenoic acid by an S. cerevisiae strain (strain “A”) comprising genes encoding a truncated CDPS polypeptide (SEQ ID NO:179, SEQ ID NO:180), a KS polypeptide (SEQ ID NO:181, SEQ ID NO:182), a first KO polypeptide (SEQ ID NO:171, SEQ ID NO:172), a second KO polypeptide (SEQ ID NO:169, SEQ ID NO:170), a CPR polypeptide (SEQ ID NO:167, SEQ ID NO:168), an ERG20-GGPPS7 polypeptide (SEQ ID NO:195, SEQ ID NO:196), a G. fujikuroi P450-2-1 polypeptide (SEQ ID NO:79, SEQ ID NO:80), a G. fujikuroi P450-3-1 polypeptide (SEQ ID NO:185, SEQ ID NO:186), an S. manihoticola KAO4 polypeptide (SEQ ID NO:73, SEQ ID NO:74), a G. fujikuroi DES-1 polypeptide (SEQ ID NO:25, SEQ ID NO:26), and an A. niger CPR16 polypeptide (SEQ ID NO:157, SEQ ID NO:158), as described in Example 2.

FIG. 5 shows a biosynthetic route from ent-kaurenoic acid to GA₃ in an S. cerevisiae strain comprising S. manihoticola KAO4 polypeptide (SEQ ID NO:73, SEQ ID NO:74), G. fujkuroi P450-3-4 (SEQ ID NO:185, SEQ ID NO:186), A. niger CPR16 (SEQ ID NO:157, SEQ ID NO:158), G. fujikuroi DES-1 (SEQ ID NO:25, SEQ ID NO:26) and either Arabidopsis thaliana GA₂₀ₒₓ₋₁ (SEQ ID NO:41, SEQ ID NO:42) or C. maxima GA₂₀ₒₓ₋₄ (SEQ ID NO:39, SEQ ID NO:40), as described in Example 2.

FIG. 6A shows a Liquid Chromatography Time of Flight (LC-TOF) mass spectrum of the peak corresponding to GA₃ from a kaurenoic acid-producing S. cerevisiae strain comprising G. fujikuroi P450-2-1 (SEQ ID NO:79, SEQ ID NO:80), G. fujikuroi P450-3-4 (SEQ ID NO:185, SEQ ID NO:186), S. manihoticola KAO4 polypeptide (SEQ ID NO:73, SEQ ID NO:74), and G. fujikuroi DES-1 (SEQ ID NO:25, SEQ ID NO:26), as described in Example 2.

FIG. 6B shows an LC-TOF mass spectrum of the peak corresponding to GA₃ from an S. cerevisiae strain comprising C. maxima GA₂₀ₒₓ₋₄ (SEQ ID NO:39, SEQ ID NO:40).

FIG. 7 shows a biosynthetic route from ent-kaurenoic acid to GA₁₂ in an ent-kaurenoic acid-producing S. cerevisiae strain comprising a KAO, as described in Example 6.

FIG. 8 shows accumulation of GA₁₂ (as measured by area-under-the-curve) for S. cerevisiae strains comprising KAO4 (SEQ ID NO:73, SEQ ID NO:74), KAO5 (SEQ ID NO:61, SEQ ID NO:62), KAO6 (SEQ ID NO:59, SEQ ID NO:60), KAO9 (SEQ ID NO:67, SEQ ID NO:68), KAO10 (SEQ ID NO:57, SEQ ID NO:58), or KAO11 (SEQ ID NO:63, SEQ ID NO:64) as well as C. maxima Ga₇ₒₓ₋₁ (SEQ ID NO:151, SEQ ID NO:152), as described in Example 6.

FIG. 9A shows a biosynthetic route from ent-kaurenoic acid to GA₉ and GA₂₀, as described in Example 6.

FIG. 9B shows GA₉ and GA₂₀ accumulation in an ent-kaurenoic acid-producing S. cerevisiae strain comprising GA₂₀ₒₓ (SEQ ID NO:39, SEQ ID NO:40) and Oryza sativa GA₁₃ₒₓ (SEQ ID NO:97, SEQ ID NO:98).

FIG. 10 shows an exemplary biosynthetic route from ent-kaurenoic acid to GA₉ by an S. cerevisae strain comprising Pisum sativum KAO11 (SEQ ID NO:63, SEQ ID NO:64), C. maxima (SEQ ID NO:151, SEQ ID NO:152), Bradyrhizobium diazoefficiens alcohol dehydrogenase (ADH) (SEQ ID NO:115, SEQ ID NO:116), and B. diazoefficiens CYP112 (SEQ ID NO:123, SEQ ID NO:124), as described in Example 7.

FIG. 11A shows a Liquid Chromatography Mass Spectrometry (LC-MS) Total Ion Current (TIC) chromatogram of a GA₉ standard.

FIG. 11B shows an LC-MS Selected Ion Recording (SIR) chromatogram, wherein the peak having an m/z 315.16 corresponds to GA₉ accumulated by an S. cerevisiae strain comprising P. sativum KAO11 (SEQ ID NO:63, SEQ ID NO:64), C. maxima (SEQ ID NO:151, SEQ ID NO:152), B. diazoefficiens ADH (SEQ ID NO:115, SEQ ID NO:116), B. diazoefficiens CYP112 (SEQ ID NO:123, SEQ ID NO:124), Pseudomonas putida ferredoxin (SEQ ID NO:147, SEQ ID NO:148), and P. putida ferredoxin reductase (SEQ ID NO:149, SEQ ID NO:150).

FIG. 11C shows an LC-MS TIC chromatogram of GA₉ accumulation by the S. cerevisiae strain described for FIG. 11B. See Example 7.

FIG. 12 shows a biosynthetic route for production of GA₄ from ent-kaurenoic acid by S. cerevisae strain comprising S. manihoticola KAO4 polypeptide (SEQ ID NO:73, SEQ ID NO:74), KO (SEQ ID NO:169, SEQ ID NO:170), B. diazoefficiens ADH (SEQ ID NO:115, SEQ ID NO:116), and B. diazoefficiens CYP112 (SEQ ID NO:123, SEQ ID NO:124), as described in Example 7.

FIG. 13A shows an LC-MS TIC chromatogram of a GA₄ standard.

FIG. 13B shows a LC-MS SIR chromatogram, wherein the peak having an m/z 331.16 corresponds to GA₄ accumulated by an S. cerevisiae strain comprising S. manihoticola KAO4 polypeptide (SEQ ID NO:73, SEQ ID NO:74), KO (SEQ ID NO:169, SEQ ID NO:170), B. diazoefficiens ADH (SEQ ID NO:115, SEQ ID NO:116), and B. diazoefficiens CYP112 (SEQ ID NO:123, SEQ ID NO:124), P. putida ferredoxin (SEQ ID NO:147, SEQ ID NO:148), and P. putida ferredoxin reductase (SEQ ID NO:149, SEQ ID NO:150).

FIG. 13C shows an LC-MS TIC chromatogram of GA4 accumulation by the S. cerevisiae strain described for FIG. 13B.

FIG. 14A shows kaurenoic acid levels in an S. cerevisiae strain comprising a gene encoding a truncated CDPS polypeptide (SEQ ID NO:179, SEQ ID NO:180), a KS polypeptide (SEQ ID NO:181, SEQ ID NO:182), a KO polypeptide (SEQ ID NO:171, SEQ ID NO:172), a CPR polypeptide (SEQ ID NO:167, SEQ ID NO:168), and an ERG20-GGPPS7 polypeptide (SEQ ID NO:195, SEQ ID NO:196), and either S. manihoticola KAO4 polypeptide (SEQ ID NO:73, SEQ ID NO:74) or G. fujikuroi KAO1 polypeptide (SEQ ID NO:89, SEQ ID NO:90).

FIG. 14B shows GA₁₄ accumulation in an S. cerevisiae strain comprising a gene encoding a truncated CDPS polypeptide (SEQ ID NO:179, SEQ ID NO:180), a KS polypeptide (SEQ ID NO:181, SEQ ID NO:182), a KO polypeptide (SEQ ID NO:171, SEQ ID NO:172), a CPR polypeptide (SEQ ID NO:167, SEQ ID NO:168), and an ERG20-GGPPS7 polypeptide (SEQ ID NO:195, SEQ ID NO:196), and either S. manihoticola KAO4 polypeptide (SEQ ID NO:73, SEQ ID NO:74) or G. fujikuroi KAO1 polypeptide (SEQ ID NO:89, SEQ ID NO:90).

FIG. 15 shows gibberellin accumulation in an S. cerevisiae strain comprising a gene encoding a truncated CDPS polypeptide (SEQ ID NO:179, SEQ ID NO:180), a KS polypeptide (SEQ ID NO:181, SEQ ID NO:182), a KO polypeptide (SEQ ID NO:171, SEQ ID NO:172), a CPR polypeptide (SEQ ID NO:167, SEQ ID NO:168), and an ERG20-GGPPS7 polypeptide (SEQ ID NO:195, SEQ ID NO:196), and either A. niger CPR16 (SEQ ID NO:157, SEQ ID NO:158), Phaeosphaeria sp. CPR14 (SEQ ID NO:99, SEQ ID NO:100), or Candida apicola CPR15 (SEQ ID NO:139, SEQ ID NO:140).

FIG. 16 shows gibberellin accumulation in an S. cerevisiae strain comprising a gene encoding a truncated DAP1-2 polypeptide (SEQ ID NO:212, SEQ ID NO:213), a ICE2-2 polypeptide (SEQ ID NO:205, SEQ ID NO:206), a CDPS-KS6 polypeptide (SEQ ID NO:101, SEQ ID NO:102), a KS5 polypeptide (SEQ ID NO:181, SEQ ID NO:182), a FfCytB5-1 polypeptide (SEQ ID NO:159, SEQ ID NO:160), a KAO3 polypeptide (SEQ ID NO:145, SEQ ID NO:146), a CPR19 polypeptide (SEQ ID NO: 193; SEQ ID NO:194), a CPR12 polypeptide (SEQ ID NO:167, SEQ ID NO:168), a RsKO polypeptide (SEQ ID NO:169, SEQ ID NO:170), a GGPPS-7 polypeptide (SEQ ID NO:177, SEQ ID NO:178), a KO1 polypeptide (SEQ ID NO:171, SEQ ID NO:172) and a P450-2-1 polypeptide (SEQ ID NO:79, SEQ ID NO:80).

FIG. 17 shows chromatograms of sample B1 (top panel) and a GA₃ standard (bottom panel) from Example 11. The peak maxima of the EICs exhibit the same retention time.

FIG. 18A shows mass spectra from sample B1 of Example 11. Both sample B1 and GA₃ standard (FIG. 18B) show the [M-H]⁻ ion at 345.1336 corresponding to GA₃. MRM analysis lead to the formation of the fragments at m/z 143 and 221, which are the most abundant fragment ions of GA₃.

FIG. 18B shows mass spectra from a GA₃ standard from Example 11. Both GA₃ standard and B1 sample (FIG. 18A) show the [M-H]⁻ ion at 345.1336 corresponding to GA₃. MRM analysis lead to the formation of the fragments at m/z 143 and 221, which are the most abundant fragment ions of GA₃.

### DETAILED DESCRIPTION OF THE INVENTION

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. The term “transformant(s)” is intended to refer a host to which at least one DNA sequence has been introduced. Such DNA sequences for “recombinant host” and “transformant(s)” include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms, for example bacteria, fungi or yeast.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in Saccharomyces cerevisiae (S. cerevisiae).

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to S. cerevisiae, including, but not limited to S. cerevisiae strain S288C. In some embodiments, an endogenous yeast gene is overexpressed. As used herein, the term “overexpress” is used to refer to the expression of a gene in an organism at levels higher than the level of gene expression in a wild type organism. In some embodiments, an endogenous yeast gene, for example ADH, is deleted. As used herein, the terms “deletion,” “deleted,” “knockout,” and “knocked out” can be used interchangeably to refer to an endogenous gene that has been manipulated to no longer be expressed in an organism, including, but not limited to, S. cerevisiae.

As used herein, the terms “heterologous sequence” and “heterologous coding sequence” are used to describe a sequence derived from a species other than the recombinant host. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

A “selectable marker” can be one of any number of genes that complement host cell auxotrophy, provide antibiotic resistance, or result in a color change. Non-limiting examples of a selectable marker can include a URA3 marker and a NatMx maker. Linearized DNA fragments of the gene replacement vector then are introduced into the cells using methods well known in the art. Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see e.g., U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

As used herein, the term “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of a gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene with inactivation thereof.

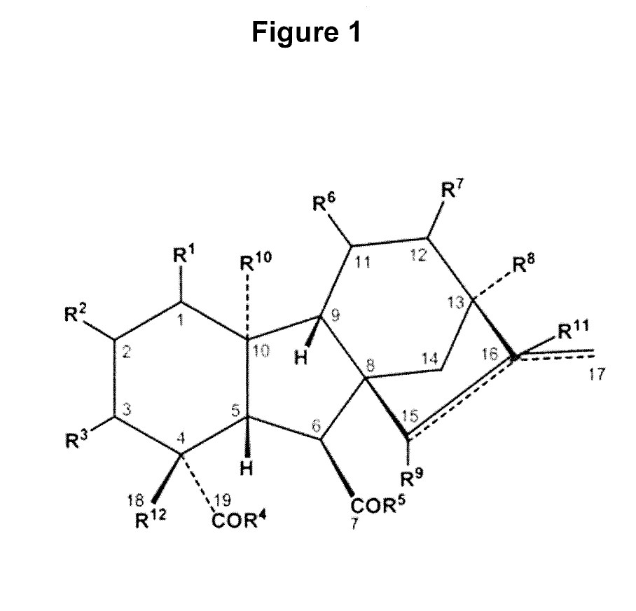

As used herein, the term “gibberellin” refers to a diterpene plant hormone having the structure of the molecule shown in Formula I and FIG. 1. Gibberellins include, but are not limited to, gibberellin A1 (GA₁), gibberellin A3 (GA₃), epoxide gibberellin A3 (epoxide GA₃), gibberellin A4 (GA₄), gibberellin A5 (GA₅), gibberellin A7 (GA₇), gibberellin A9 (GA₉), gibberellin A12 (GA₁₂), gibberellin A13 (GA₁₃), gibberellin A14 (GA₁₄), gibberellin A15 (GA₁₅), gibberellin A19 (GA₁₉), gibberellin A20 (GA₂₀), gibberellin A24 (GA₂₄), gibberellin A25 (GA₂₅), gibberellin A36 (GA₃₆), gibberellin A37 (GA₃₇), gibberellin A44 (GA₄₄), gibberellin A53 (GA₅₃), and gibberellin A110 (GA₁₁₀). In particular, the gibberellin can be a gibberellin described in Table 1, Formula I, and FIG. 1.

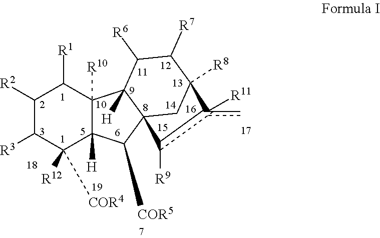

TABLE 1
Gibberellin structure.

| | R¹ | R² | R³ | R⁴ | R¹⁰ | R⁸ |
|---|---|---|---|---|---|---|
| GA₁ | | | β-OH | —O—C10 | —O—C19 | —OH |
| GA₃ | ═C2 | ═C1 | β-OH | —O—C10 | —O—C19 | —OH |
| GA₄ | | | β-OH | —O—C10 | —O—C19 | |
| GA₅ | | ═C3 | ═C2 | —O—C10 | —O—C19 | —OH |
| GA₇ | ═C2 | ═C1 | β-OH | —O—C10 | —O—C19 | |
| GA₉ | | | | —O—C10 | —O—C19 | |
| GA₁₂ | | | | —OH | —CH₃ | |
| GA₁₄ | | | β-OH | —OH | —CH₃ | |
| GA₁₅ | | | | —OH | —CH₂OH | |
| open lactone | | | | | | |
| GA₁₅ | | | | —O—CH₂—C10 | —CH₂—O—C19 | |
| GA₁₉ | | | | —OH | —CHO | —OH |
| GA₂₀ | | | | —O—C10 | —O—C19 | —OH |
| GA₂₄ | | | | —OH | —CHO | |
| GA₄₄ | | | | —O—CH₂—C10 | —CH₂—O—C19 | —OH |
| GA₅₃ | | | | —OH | —CH₃ | —OH |

R⁵ = —OH,
R¹² = —CH₃,
all other R = —H

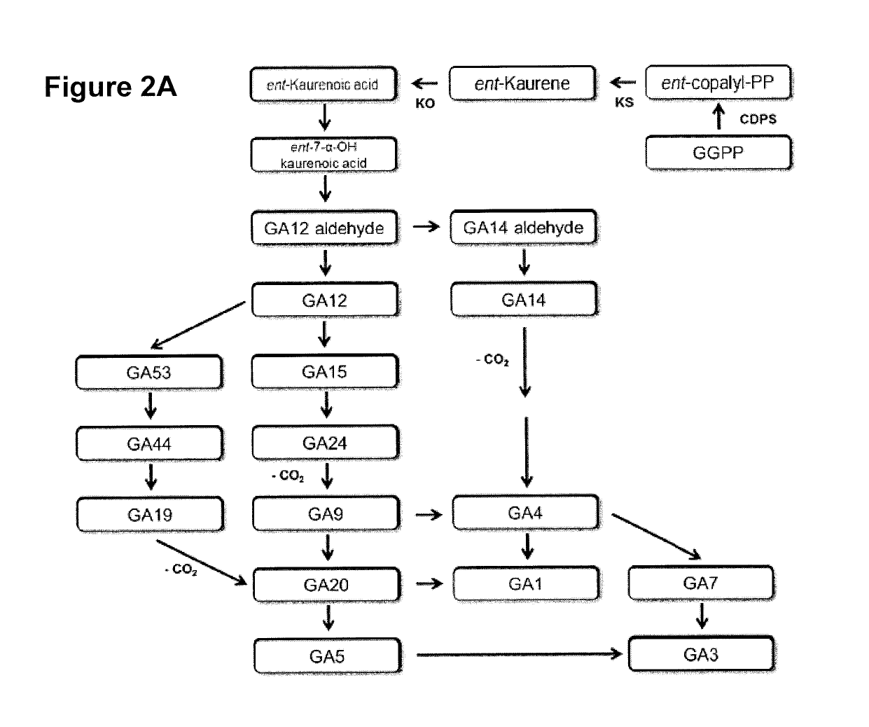

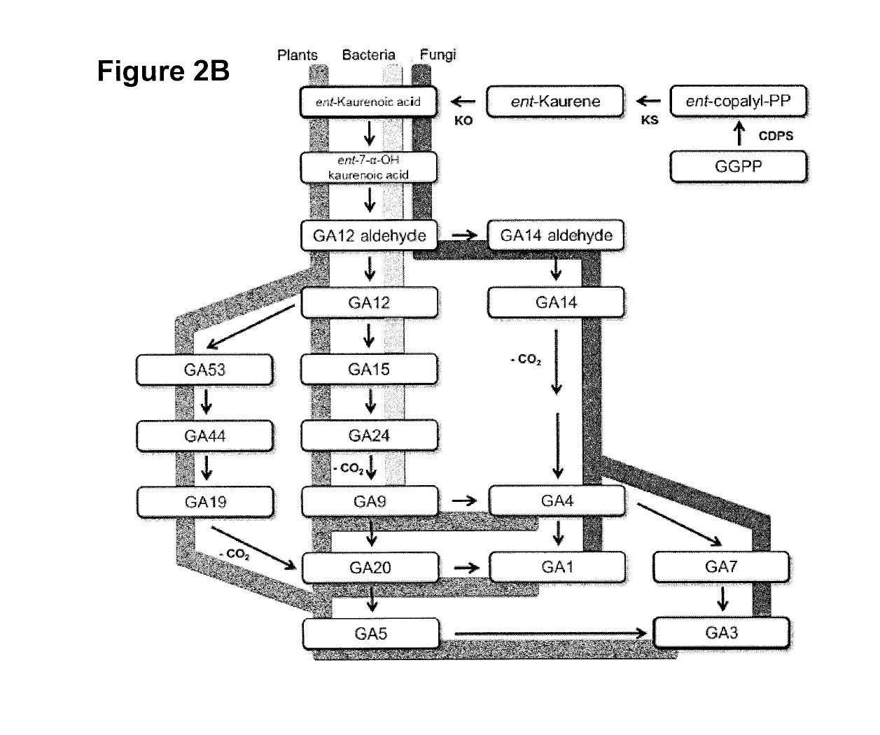

As used herein, the term “gibberellin precursor” refers to intermediate compounds in a gibberellin biosynthetic pathway. Gibberellin precursors include, but are not limited to, GGPP, ent-copalyl-diphosphate, ent-kaurene, ent-kaurenoic acid, and ent-kaurenoic acid-7-α-OH kaurenoic acid. See, e.g., FIG. 2. In some embodiments, gibberellin precursors are gibberellin aldehydes, such as GA₁₂ aldehyde or GA₁₄ aldehyde. In some embodiments, gibberellin precursors are themselves gibberellin compounds. For example, GA₇ and GA₅ are gibberellin precursors to GA₃.

In some aspects, gibberellins and gibberellin precursors are accumulated in an ent-kaurenoic acid-producing host. Recombinant ent-kaurenoic acid-producing and terpene-producing Saccharomyces cerevisiae (S. cerevisiae) strains are described in WO 2011/153378, WO 2013/022989, WO 2014/122227, and WO 2014/122328, each of which has been incorporated by reference herein in its entirety. Methods of producing terpenes in recombinant hosts, by whole cell bio-conversion, and in vitro are also described in WO 2011/153378, WO 2013/022989, WO 2014/122227, and WO 2014/122328.

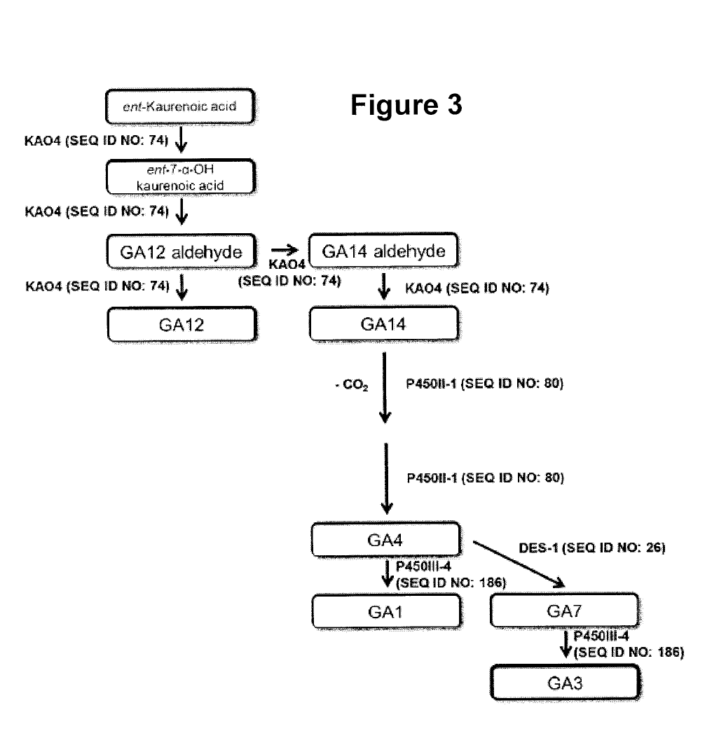

In some embodiments, gibberellins and/or gibberellin precursors are produced in vivo through expression of one or more enzymes involved in a gibberellin biosynthetic pathway in a recombinant host. For example, an ent-kaurenoic acid-producing recombinant host expressing one or more of a gene encoding a cytochrome P450 (P450) monooxygenase polypeptide, a gene encoding a cytochrome P450 reductase (CPR) polypeptide, and a gene a 2-ODD polypeptide can accumulate a gibberellin or gibberellin precursor in vivo. See, e.g., FIGS. 3, 5, 7, 9A, 10, and 12. The skilled worker will appreciate that one or more of these genes can be endogenous to the host provided that at least one (and in some embodiments, all) of these genes is a recombinant gene introduced into the recombinant host.

In some embodiments, gibberellins and/or gibberellin precursors are produced through contact of a gibberellin precursor with one or more enzymes involved in the gibberellin pathway in vitro. For example, contacting GA₇ with a cytochrome P450 polypeptide can result in production of GA₃ in vitro. In some embodiments, a gibberellin is produced through contact of a gibberellin precursor with one or more enzymes involved in the gibberellin pathway in vitro. For example, contacting ent-kaurene with a KO enzyme can result in production of ent-kaurenoic acid in vitro.

In some embodiments, a gibberellin or gibberellin precursor is produced by whole cell bioconversion. For whole cell bioconversion to occur, a host cell expressing one or more enzymes involved in the gibberellin pathway takes up and modifies a gibberellin precursor in the cell; following modification (e.g., addition of a double bond or oxidation) in vivo, a gibberellin remains in the cell and/or diffuses or is excreted into the culture medium. For example, a host cell expressing a gene encoding a cytochrome P450 monooxygenase polypeptide can take up GA₇ and oxidize C13 of GA₇ in the cell; following such a modification in vivo, GA₃ can be excreted into the culture medium. In some embodiments, the cell can be permeabilized to take up a substrate to be modified or to excrete a modified product.

In some embodiments, one or more gibberellin precursors and/or one or more gibberellins are produced by co-culturing of two or more hosts. In some embodiments, one or more hosts, each expressing one or more enzymes involved in the gibberellin pathway, produce one or more gibberellin precursors and/or one or more gibberellins. For example, a host comprising a GGPPS, an CDPS, and/or a KO and a host comprising a cytochrome P450 monooxygenase, a cytochrome P450 reductase, and/or a 2-ODD produce one or more gibberellins.

In some aspects, a host comprises a heterologous gene encoding a GGPPS polypeptide. In some embodiments, the GGPPS polypeptide is a GGPPS polypeptide having the amino acid sequence set forth in SEQ ID NO:50, SEQ ID NO:134, or SEQ ID NO:178. The GGPPS polypeptide can catalyze conversion of farnesyl diphosphate (FPP) to GGPP.

In some aspects, a host comprises a heterologous gene encoding a CDPS polypeptide. In some embodiments, the CDPS polypeptide is a CDPS polypeptide having the amino acid sequence set forth in SEQ ID NO:102, SEQ ID NO:106, SEQ ID NO:108, or SEQ ID NO:180 or a bi-functional a CDPS polypeptide having the amino acid sequence set forth in SEQ ID NO:104, SEQ ID NO:227 or SEQ ID NO:229. The CDPS polypeptide can catalyze conversion of GGPP to ent-copalyl pyrophosphate. In some embodiments, the bi-functional CDPS polypeptide of SEQ ID NO:104 further comprises a P571S and/or L654P substitution. In some embodiments, a host comprising the mutant CDPS polypeptide accumulates greater levels of gibberellins, as compared to a host that does not comprise a gene encoding a mutant CDPS polypeptide.

In some aspects, a host comprises a heterologous gene encoding a KS polypeptide. In some embodiments, the KS polypeptide is a KS polypeptide having the amino acid sequence set forth in SEQ ID NO:102 or SEQ ID NO:106. The KS polypeptide can catalyze conversion of ent-copalyl pyrophosphate to ent-kaurene.

In some aspects, a host comprises a heterologous gene encoding a KO polypeptide. In some embodiments, the KO polypeptide is a KO polypeptide having the amino acid sequence set forth in SEQ ID NO:82, SEQ ID NO:164, SEQ ID NO:170, or SEQ ID NO:172. The KO polypeptide can catalyze conversion of ent-kaurene to ent-kaurenoic acid.

In some aspects, a host comprises a gene encoding a KAO polypeptide. The KAO polypeptide can be a plant-derived KAO polypeptide. In some embodiments, the KAO polypeptide is a KAO polypeptide having the amino acid sequence set forth in SEQ ID NO:56, SEQ ID NO:58, SEQ ID NO:60, SEQ ID NO:62, SEQ ID NO:64, SEQ ID NO:66, SEQ ID NO:68, SEQ ID NO:74, SEQ ID NO:88, SEQ ID NO:90, or SEQ ID NO:146. The KAO polypeptide can catalyze, for example, conversion of ent-kaurenoic acid to ent-7α-OH kaurenoic acid, ent-7α-OH kaurenoic acid to GA₁₂ aldehyde, GA₁₂ aldehyde to GA₁₂, and GA₁₂ aldehyde to GA₁₄ aldehyde. See, e.g., FIGS. 3, 5, 7, 9A, 10, and 12 and Example 6.

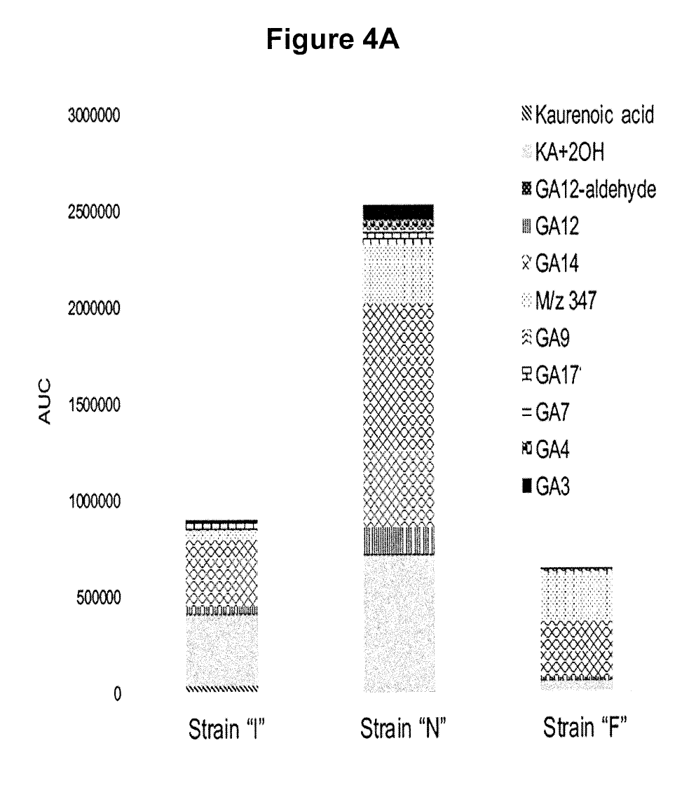

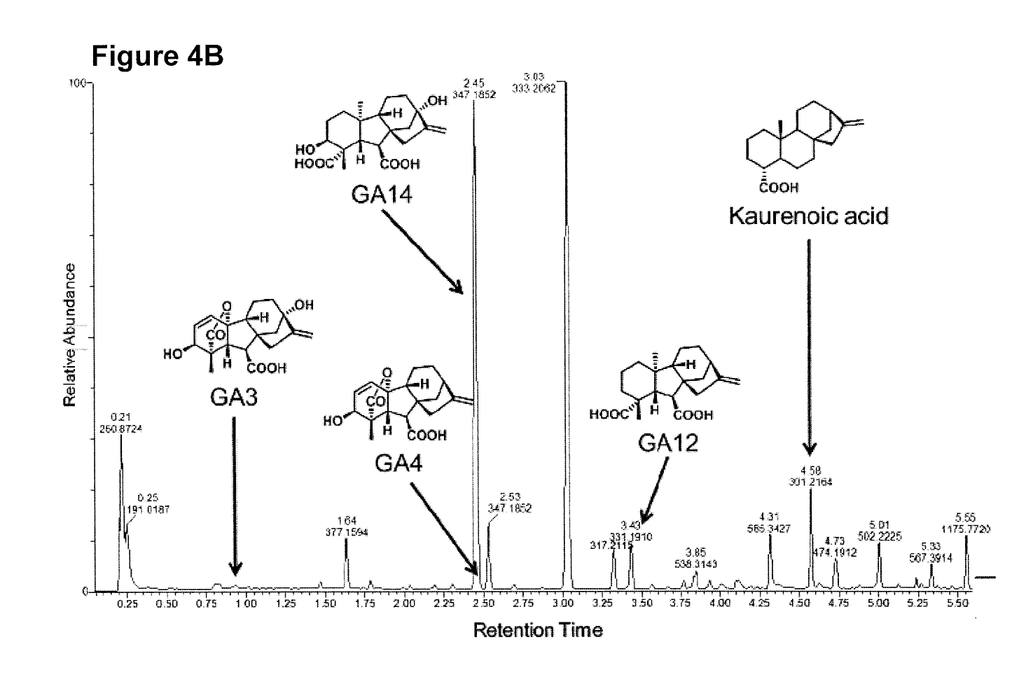

In some embodiments, a cytochrome B5 polypeptide (i.e., a cytochrome B5 polypeptide of SEQ ID NO:160) and/or a cytochrome B5 reductase polypeptide (i.e., a cytochrome B5 reductase polypeptide of SEQ ID NO:2) increases activity of a KAO polypeptide and/or a cytochrome P450 polypeptide. In some aspects, increased activity of a KAO polypeptide is evidenced by increased levels of GA₁₄ and GA₃ in an S. cerevisiae strain comprising a gene encoding a cytochrome B5 polypeptide and a gene encoding a cytochrome b5 reductase polypeptide. See Example 2 and FIG. 4A.

In some aspects, a host comprises a gene encoding a P450-1 polypeptide. The P450-1 polypeptide can be a fungus-derived P450-1 polypeptide. In some embodiments, the P450-1 polypeptide is a P450-1 polypeptide having the amino acid sequence set forth in SEQ ID NO:74, SEQ ID NO:88, SEQ ID NO:90, or SEQ ID NO:146. The P450-1 polypeptide can catalyze conversion of ent-kaurenoic acid to ent-7α-OH kaurenoic acid, ent-7α-OH kaurenoic acid to GA₁₂ aldehyde, and GA₁₂ aldehyde to GA₁₄ aldehyde. In some aspects, a P450-1 polypeptide can have KAO and GA₃ₒₓ activity. See Example 8. The fungal KAO enzymes (e.g., S. manihoticola KAO4 polypeptide (SEQ ID NO:73, SEQ ID NO:74) and G. fujikuroi KAO1 polypeptide (SEQ ID NO:89, SEQ ID NO:90) also have GA₃ₒₓ activity.

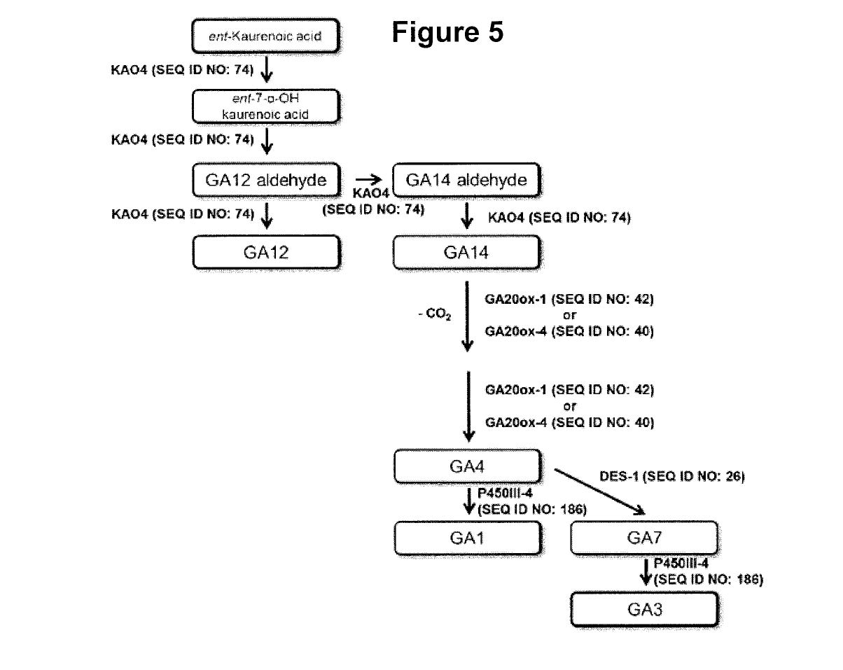

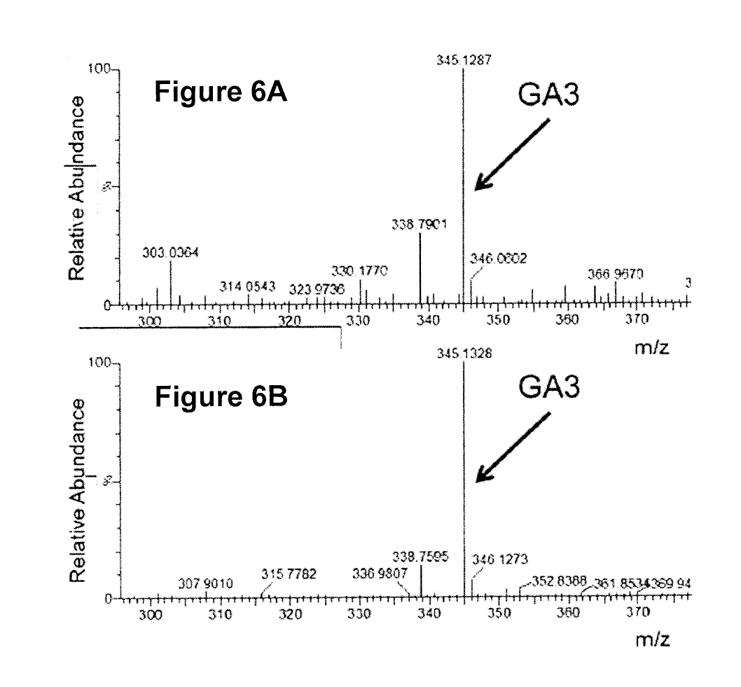

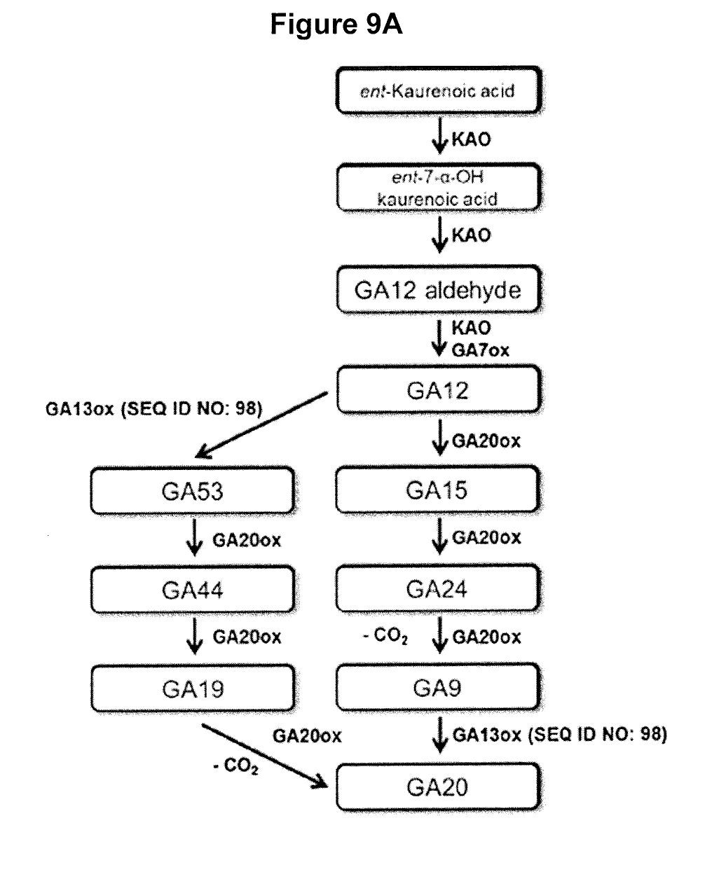

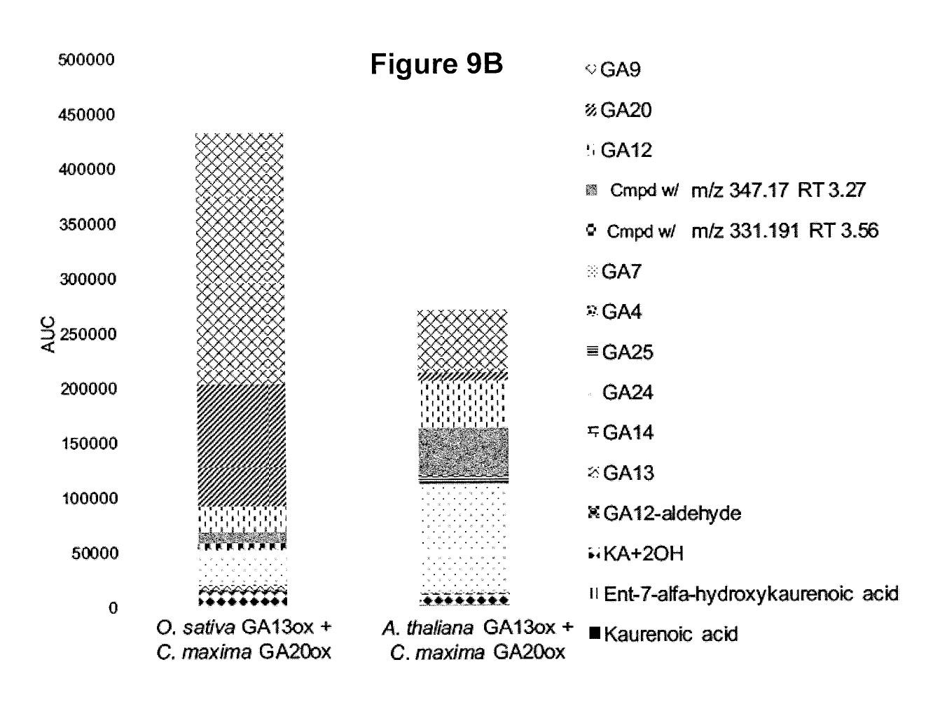

In some aspects, a host comprises a gene encoding a GA 20-oxidase (GA₂₀ₒₓ) polypeptide. The GA₂₀ₒₓ polypeptide can be a plant-derived GA₂₀ₒₓ polypeptide. In some embodiments, the GA₂₀ₒₓ polypeptide comprises a GA₂₀ₒₓ polypeptide having the amino acid sequence set forth in SEQ ID NO:32, SEQ ID NO:34, SEQ ID NO:40, or SEQ ID NO:42. The GA₂₀ₒₓ polypeptide is a 2-ODD polypeptide and can catalyze conversion of GA₁₄ to GA₄, GA₁₂ to GA₁₅, GA₂₄ to GA₉, GA₅₃ to GA₄₄, and GA₄₄ to GA₁₉. See FIGS. 5 and 9A.

In other embodiments, a host comprises a GA 7-oxidase (GA₇ₒₓ) and/or a GA 3-oxidase (GA₃ₒₓ). GA₇ₒₓ and GA₃ₒₓ polypeptides can be plant-derived 2-ODD polypeptides. In some embodiments, the GA₇ₒₓ polypeptide comprises a GA₇ₒₓ polypeptide having the amino acid sequence set forth in SEQ ID NO:16 or SEQ ID NO:162. In some embodiments, the GA₃ₒₓ polypeptide comprises a GA₃ₒₓ polypeptide having the amino acid sequence set forth in SEQ ID NO:28, SEQ ID NO:30, SEQ ID NO:36, or SEQ ID NO:44.

In some embodiments, a host comprises a GA 13-oxidase (GA₁₃ₒₓ). A GA₁₃ₒₓ polypeptide can be a plant-derived GA₁₃ₒₓ polypeptide. In some embodiments, the GA₁₃ₒₓ polypeptide comprises a GA₁₃ₒₓ polypeptide having the amino acid sequence set forth in SEQ ID NO:72, SEQ ID NO:78, or SEQ ID NO:98. In some embodiments, a cytochrome B5 polypeptide (i.e., a cytochrome B5 polypeptide of SEQ ID NO:160) and/or a cytochrome B5 reductase polypeptide (i.e., a cytochrome B5 reductase polypeptide of SEQ ID NO:2) increases activity of a GA₁₃ₒₓ polypeptide. In some embodiments, the GA₁₃ₒₓ polypeptide can catalyze conversion of GA₉ to GA₂₀. See FIG. 9A.

In some aspects, a host comprises a gene encoding a P450-2 polypeptide. The P450-2 polypeptide can be a fungus-derived P450-2 polypeptide. In some embodiments, the P450-2 polypeptide comprises a P450-2 polypeptide having the amino acid sequence set forth in SEQ ID NO:14, SEQ ID NO:18, SEQ ID NO:70, SEQ ID NO:80, SEQ ID NO:94, SEQ ID NO:142, SEQ ID NO:233, SEQ ID NO:235, or SEQ ID NO:237. The P450-2 polypeptide can catalyze conversion of GA₁₄ to GA₄ and conversion of GA₁₂ to GA₉. See FIG. 3.

In some aspects, a host comprises a gene encoding a P450-3 polypeptide. The P450-3 polypeptide can be a fungus-derived P450-3 polypeptide. In some embodiments, the P450-3 polypeptide comprises a P450-3 polypeptide having the amino acid sequence set forth in SEQ ID NO:46, SEQ ID NO:144, SEQ ID NO:184, or SEQ ID NO:186. The P450-3 polypeptide can catalyze conversion of GA₄ to GA₁ or GA₇ to GA₃. See FIGS. 3 and 5.

In some embodiments, a host comprises a gene encoding a GA₄ desaturase (DES) polypeptide. The DES polypeptide can be a fungus-derived DES polypeptide. In some embodiments, the DES polypeptide comprises a DES polypeptide having the amino acid sequence set forth in SEQ ID NO:20, SEQ ID NO:22, SEQ ID NO:24, or SEQ ID NO:26. In some aspects, the DES polypeptide of SEQ ID NO:22 and/or the DES polypeptide of SEQ ID NO:26 comprises an L233P substitution. The DES polypeptide is a 2-ODD polypeptide and can catalyze conversion of GA₄ to GA₇. See FIGS. 3 and 5.

In some embodiments, a host comprises a gene encoding a cytochrome B5 polypeptide and/or a gene encoding a cytochrome B5 reductase polypeptide. In some aspects, a cytochrome B5 reductase provides electrons to a P450 monooxygenase through cytochrome B5. In some aspects, the cytochrome B5 electron transport system assists a cytochrome P450 reductase by supplying an electron of the catalytic cycle or by acting as an allosteric activator. See, e.g., Troncoso et al., 2008, Phytochemistry 69(3):672-83. In some embodiments, the cytochrome B5 polypeptide comprises a cytochrome B5 polypeptide having the amino acid sequence set forth in SEQ ID NO:160. In some embodiments, the cytochrome B5 reductase polypeptide comprises a cytochrome B5 polypeptide having the amino acid sequence set forth in SEQ ID NO:2. See Example 2.

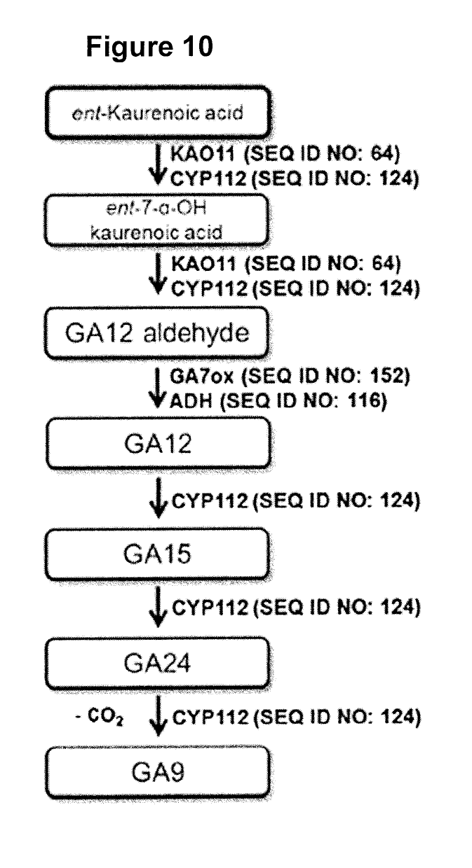

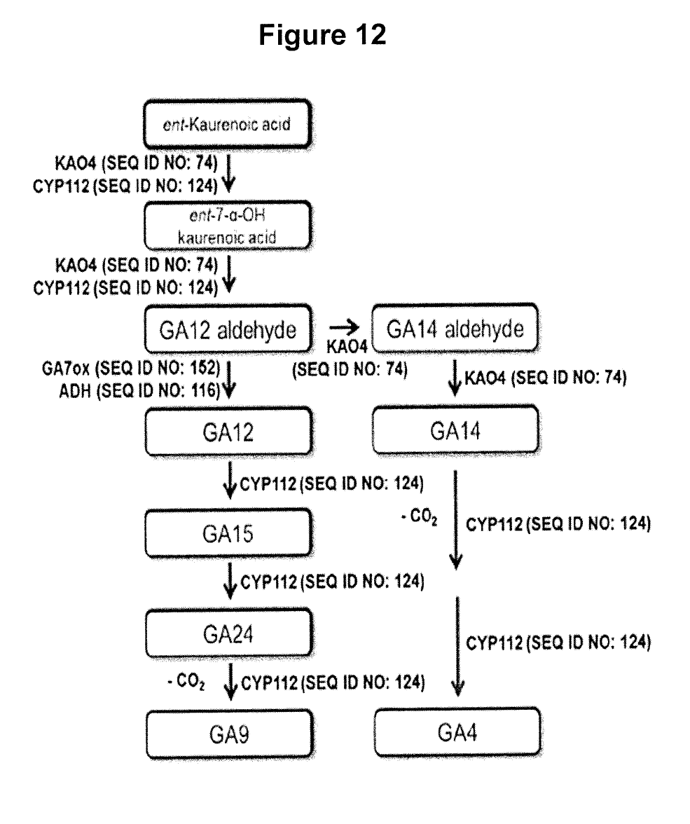

In some embodiments, a host comprises a CYP112 polypeptide. In some embodiments, the CYP112 polypeptide comprises a CYP112 polypeptide having the amino acid sequence set forth in SEQ ID NO:4, SEQ ID NO:6, SEQ ID NO:8, SEQ ID NO:10, SEQ ID NO:124, or SEQ ID NO:128. The CYP112 polypeptide can catalyze conversion of GA₁₂ to GA₁₅, GA₁₅ to GA₂₄, GA₂₄ to GA₉, and GA₁₄ to GA₄. See FIGS. 10 and 12.

In some embodiments, a host comprises one or more heterologous genes encoding one or more alcohol dehydrogenase (ADH) polypeptides. The ADH polypeptide can be an ADH polypeptide having the amino acid sequence set forth in SEQ ID NO:112, SEQ ID NO:116, or SEQ ID NO:118. See FIG. 10. In some aspects, the ADH polypeptide converts GA₁₂ aldehyde or GA₁₄ aldehyde to GA₁₂ or GA₁₄, respectively. In some aspects, the ADH polypeptide converts kaurenal to kaurenoic acid.

In some embodiments, a host comprising CDPS-KS bifunctional polypeptides can be comparatively tested in a host inserted with CytB5-1 and CytB5red-1. The host may then be transformed with CPR12 (SEQ ID NO:167 which encodes SEQ ID NO:168), RsKO_GA (SEQ ID NO:169 which encodes SEQ ID NO:170), GGPPS7 (SEQ ID NO:176 and SEQ ID NO:178), KO1 (SEQ ID NO:171 which encodes SEQ ID NO:172), and either CDPS-KS6+KS5 (SEQ ID NO:101 which encodes SEQ ID NO:102; and SEQ ID NO:181 which encodes SEQ ID NO:182), CDPS-KS6 (SEQ ID NO:101 which encodes SEQ ID NO:102), CDPS-KS4 (SEQ ID NO:226 which encodes SEQ ID NO:227), or CDPS-KS9 (SEQ ID NO 228 which encodes SEQ ID NO:229). See Example 3 and Table 6. In some aspects, the CDPS-KS activity converts GGPPS to kaurenoic acid.

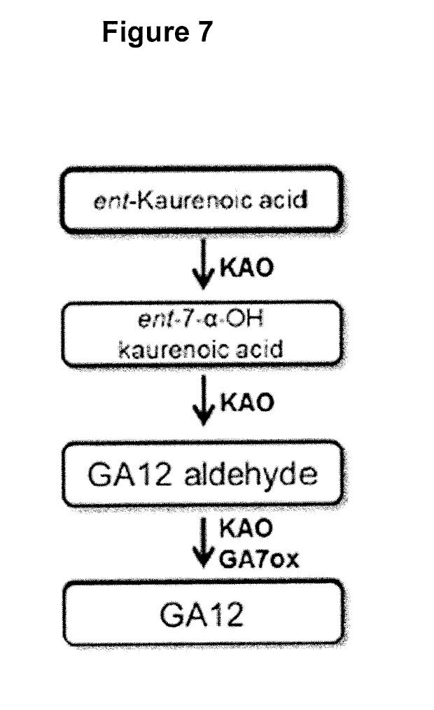

In some embodiments, a host may comprise KO1 (SEQ ID NO:171 (nt) and SEQ ID NO:172 (aa) and CPR19 (SEQ ID NO:193 (nt) and SEQ ID NO:194 (aa)) and may be transformed with CDPS-KS6 (SEQ ID NO:101), KS5 (SEQ ID NO:181), GGPPS7 (SEQ ID NO:177), KO1 (SEQ ID NO:171), KAO and CPR genes using USER™ based DNA assembler vectors and NatMx marker. The host may co-express KAO-3/CPR19 polypeptides (SEQ ID NO:230 and SEQ ID NO:193), KAO-4/CPR17 (SEQ ID NO:73 and SEQ ID NO:187) or CPR19 (SEQ ID NO:193) polypeptides, or KAO-5/CPR12 (SEQ ID NO:61 and SEQ ID NO:167) or CPR19 polypeptides (for example, SEQ ID NO:193). See Example 4, FIG. 7, and Table 7. In some aspects, the KAO polypeptide converts GA₁₂ aldehyde or GA₁₄ aldehyde to GA₁₂ or GA₁₄, respectively.

In some embodiments, a host may comprise FfCytB5-1 (SEQ ID NO:159 (nt) and SEQ ID NO:160 (aa)), FfCytB5red-1 (SEQ ID NO:01 (nt) and SEQ ID NO:02 (aa)), CPR19 (SEQ ID NO:193 (nt) and SEQ ID NO:194 (aa)), RsKO-GA (SEQ ID NO:169 (nt) and SEQ ID NO:170 (aa)), KS5 (SEQ ID NO:181 (nt) and SEQ ID NO:182 (aa)), tCDPS5 (SEQ ID NO:179 (nt) and SEQ ID NO:180 (aa)), GGPPS-7 (SEQ ID NO:177 (nt) and SEQ ID NO:178 (aa)), and KO1 (SEQ ID NO:171 (nt) and SEQ ID NO:172 (aa)) and be transformed with P450-3-1 (SEQ ID NO:45), P450-2-4 (SEQ ID NO:141), P450-3-4 (SEQ ID NO:185), DES-1 (SEQ ID NO:25), and either KAO1 (SEQ ID NO:89), KAO3 (SEQ ID NO:145), KAO4 (SEQ ID NO:73) or KAO5 (SEQ ID NO:61). See Example 4, FIG. 7, and Table 8. In some aspects, the KAO activity leads to the production of GA₁, GA₃, GA₄, GA₇, and epoxide GA₃.

In some embodiments, a host may be inserted with P450-3-4 (SEQ ID NO:141(nt) and SEQ ID NO:142 (aa)), KO1 (SEQ ID NO:170 (nt) and SEQ ID NO:171 (aa)), GGPPS-7 (SEQ ID NO:177 (nt) and SEQ ID NO:178 (aa)), CDPS-KS6 (SEQ ID NO:101 (nt) and SEQ ID NO:102 (aa)), KAO4 (SEQ ID NO:73 (nt) and SEQ ID NO:74 (aa)), FfCytB5-1 (SEQ ID NO:159 (nt) and SEQ ID NO:160 (aa)), CPR1 (SEQ ID NO:165 (nt) and SEQ ID NO:166 (aa)), CPR19 (SEQ ID NO:193 (nt) and SEQ ID NO:194 (aa)), and various P450-2 genes: P450-2-1 (SEQ ID NO:79 (nt) and SEQ ID NO:80 (aa)), P450-2-8 (SEQ ID NO:232 (nt) and SEQ ID NO:233 (aa)), P450-2-9 (SEQ ID NO:234 (nt) and SEQ ID NO:235 (aa)), and P450-2-10 (SEQ ID NO:236 (nt) and SEQ ID NO:237 (aa)). See Example 5, Table 9, and FIG. 3. In some aspects, the P450-2 activity can convert GA₁₄ to GA₄.

In some embodiments, P450-2 genes may be introduced by integration into a host using a USER™ cloning based vector system using the URA3 selection marker. P450-2 genes integrated may be selected from SEQ ID NO:13, SEQ ID NO:17, SEQ ID NO:80, and SEQ ID NO:141. See Example 5, Table 10, and FIG. 3. In some aspects, the P450-2 activity can convert GA₁₄ to GA₄.

In some embodiments, an S. cerevisiae strain (strain “N”) comprising a gene encoding a truncated CDPS polypeptide (SEQ ID NO:179, SEQ ID NO:180), a gene encoding a KS polypeptide (SEQ ID NO:181, SEQ ID NO:182), a first gene encoding a KO polypeptide (SEQ ID NO:171, SEQ ID NO:172), a second gene encoding a KO polypeptide (SEQ ID NO:169, SEQ ID NO:170), a gene encoding a CPR polypeptide (SEQ ID NO:167, SEQ ID NO:168), a gene encoding an ERG20-GGPPS7 polypeptide (SEQ ID NO:195, SEQ ID NO:196), a gene encoding a Gibberellin fujikuroi P450-2-1 polypeptide (SEQ ID NO:79, SEQ ID NO:80), a gene encoding a Gibberellin fujikuroi P450-3-4 polypeptide (SEQ ID NO:185, SEQ ID NO:186), a gene encoding an S. manihoticola KAO4 polypeptide (SEQ ID NO:73, SEQ ID NO:74), a gene encoding a G. fujikuroi DES-1 polypeptide (SEQ ID NO:25, SEQ ID NO:26), a gene encoding a G. fujikuroi cytochrome B5 polypeptide (SEQ ID NO:159, SEQ ID NO:160), and a gene encoding a G. fujikuroi cytochrome B5 reductase polypeptide (SEQ ID NO:1, SEQ ID NO:2) accumulate gibberellins, including, but not limited to, GA₃, GA₄, GA₁₂, GA₁₄, and GA₁₇. See Example 2; Tables 2 and 4; and FIGS. 3 and 4A.

In some embodiments, an S. cerevisiae strain (strain “A”) comprising a gene encoding a truncated CDPS polypeptide (SEQ ID NO:179, SEQ ID NO:180), a gene encoding a KS polypeptide (SEQ ID NO:181, SEQ ID NO:182), a first gene encoding a KO polypeptide (SEQ ID NO:171, SEQ ID NO:172), a second gene encoding a KO polypeptide (SEQ ID NO:169, SEQ ID NO:170), a gene encoding a CPR polypeptide (SEQ ID NO:167, SEQ ID NO:168), a gene encoding an ERG20-GGPPS7 polypeptide (SEQ ID NO:195, SEQ ID NO:196), a gene encoding a Gibberellin fujikuroi P450-2-1 polypeptide (SEQ ID NO:79, SEQ ID NO:80), a gene encoding a Gibberellin fujikuroi P450-3-4 polypeptide (SEQ ID NO:185, SEQ ID NO:186), a gene encoding an S. manihoticola KAO4 polypeptide (SEQ ID NO:73, SEQ ID NO:74), a gene encoding a G. fujikuroi DES-1 polypeptide (SEQ ID NO:25, SEQ ID NO:26), and a gene encoding an A. niger CPR12 polypeptide (SEQ ID NO:157, SEQ ID NO:158) accumulates gibberellins, including, but not limited to, GA₃, GA₄, GA₁₂, GA₁₃, GA₁₄, GA₂₅. See Example 2; Tables 3 and 4; and FIGS. 3 and 4B.

In some embodiments, expression of ORF1 (SEQ ID NO:153, SEQ ID NO:154), ORF2 (SEQ ID NO:155, SEQ ID NO:156), AIdDH (SEQ ID NO:201, SEQ ID NO:202), ADH (SEQ ID NO:109, SEQ ID NO:110), ANK (SEQ ID NO:210, SEQ ID NO:225) and/or smt (SEQ ID NO:222, SEQ ID NO:209), which are clustered with various gibberellin pathway genes in G. fujikuroi, can improve turnover of gibberellin-producing S. cerevisiae strains described herein. See e.g., Bömke et al., 2009, Phytochemistry, 70(15-16):1876-93.

In some embodiments, an S. cerevisiae strain (strain “F”) comprising a gene encoding a truncated CDPS polypeptide (SEQ ID NO:179, SEQ ID NO:180), a gene encoding a KS polypeptide (SEQ ID NO:181, SEQ ID NO:182), a first gene encoding a KO polypeptide (SEQ ID NO:171, SEQ ID NO:172), a second gene encoding a KO polypeptide (SEQ ID NO:169, SEQ ID NO:170), a gene encoding a CPR polypeptide (SEQ ID NO:167, SEQ ID NO:168), a gene encoding an ERG20-GGPPS7 polypeptide (SEQ ID NO:195, SEQ ID NO:196), a gene encoding an A. thaliana GA₂₀ₒₓ₋₄ polypeptide (SEQ ID NO:39, SEQ ID NO:40), a gene encoding a G. fujikuroi P450-3-4 polypeptide (SEQ ID NO:185, SEQ ID NO:186), a gene encoding an S. manihoticola KAO4 polypeptide (SEQ ID NO:73, SEQ ID NO:74), a gene encoding a G. fujikuroi DES-1 polypeptide (SEQ ID NO:25, SEQ ID NO:26), and a gene encoding an A. niger CPR16 polypeptide (SEQ ID NO:157, SEQ ID NO:158) accumulates gibberellins, including, but not limited to, GA₃, GA₄, GA₁₂, and GA₁₄. See Example 2, FIGS. 4A and 5, and Table 4.

In some embodiments, an S. cerevisiae strain comprising a gene encoding a truncated CDPS polypeptide (SEQ ID NO:179, SEQ ID NO:180), a gene encoding a KS polypeptide (SEQ ID NO:181, SEQ ID NO:182), a first gene encoding a KO polypeptide (SEQ ID NO:171, SEQ ID NO:172), a second gene encoding a KO polypeptide (SEQ ID NO:169, SEQ ID NO:170), a gene encoding a CPR polypeptide (SEQ ID NO:167, SEQ ID NO:168), a gene encoding an ERG20-GGPPS7 polypeptide (SEQ ID NO:195, SEQ ID NO:196), a gene encoding a C. maxima GA₂₀ₒₓ₋₁ polypeptide (SEQ ID NO:39, SEQ ID NO:40), a gene encoding a G. fujikuroi P450-3-4 polypeptide (SEQ ID NO:185, SEQ ID NO:186), a gene encoding a G. fujikuroi DES-1 polypeptide (SEQ ID NO:25, SEQ ID NO:26), and a gene encoding an A. niger CPR16 polypeptide (SEQ ID NO:157, SEQ ID NO:158) accumulates gibberellins. See FIG. 5.

In some embodiments, expression of a gene encoding a KAO polypeptide (such as, but not limited to, a KAO11 polypeptide having the amino acid sequence SEQ ID NO:64) in an ent-kaurenoic acid-producing S. cerevisiae strain that further coexpresses C. maxima GA₂₀ₒₓ (SEQ ID NO:39, SEQ ID NO:40) and Oryza sativa GA₁₃ₒₓ (SEQ ID NO:97, SEQ ID NO:98) results in accumulation of GA₉ and GA₂₀. See FIGS. 9A and 9B. In some aspects, further expression of a gene encoding a GA₃ₒₓ polypeptide (SEQ ID NO:27, SEQ ID NO:28, SEQ ID NO:29, SEQ ID NO:30, SEQ ID NO:41, SEQ ID NO:42, SEQ ID NO:43, SEQ ID NO:44), a gene encoding a P450-3 polypeptide (SEQ ID NO:183, SEQ ID NO:184, SEQ ID NO:185, SEQ ID NO:186), and a gene encoding a DES polypeptide (SEQ ID NO:25, SEQ ID NO:26, SEQ ID NO:21, SEQ ID NO:22, SEQ ID NO:19, SEQ ID NO:20) results in accumulation of GA₁₂, GA₇, GA₄, GA₂₅, GA₂₄, and GA₁₃.

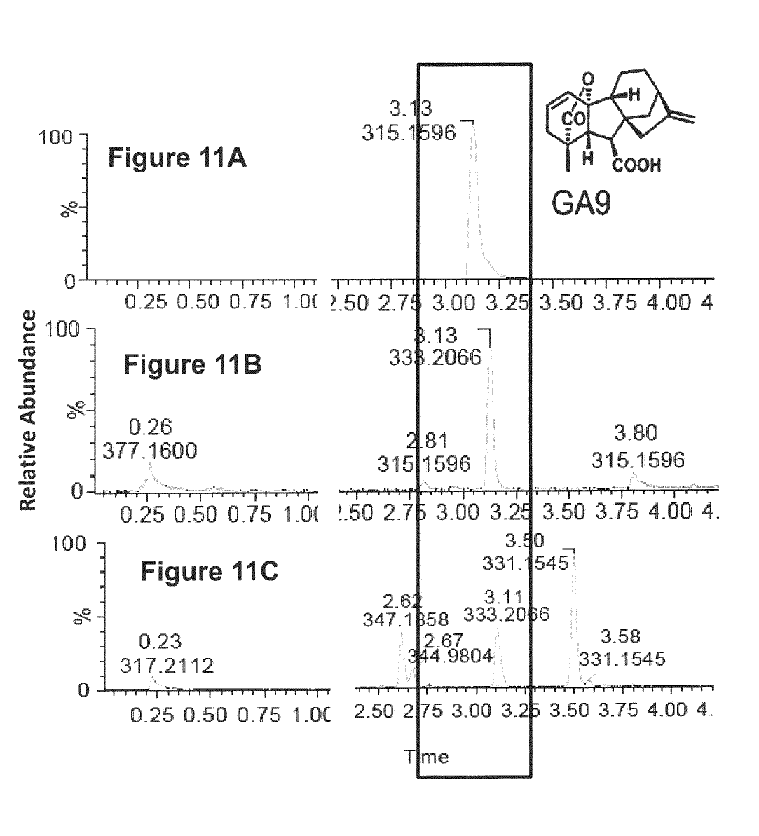

In some embodiments, an S. cerevisiae strain (strain “P”) comprising a gene encoding a truncated CDPS polypeptide (SEQ ID NO:179, SEQ ID NO:180), a gene encoding a KS polypeptide (SEQ ID NO:181, SEQ ID NO:182), a first gene encoding a KO polypeptide (SEQ ID NO:171, SEQ ID NO:172), a second gene encoding a KO polypeptide (SEQ ID NO:169, SEQ ID NO:170), a gene encoding a CPR polypeptide (SEQ ID NO:167, SEQ ID NO:168), a gene encoding an ERG20-GGPPS7 polypeptide (SEQ ID NO:195, SEQ ID NO:196), a gene encoding a P. sativum KAO11 polypeptide (SEQ ID NO:63, SEQ ID NO:64), a gene encoding a C. maxima GA₇ₒₓ polypeptide (SEQ ID NO:151, SEQ ID NO:152), a gene encoding a B. diazoefficiens ADH polypeptide (SEQ ID NO:115, SEQ ID NO:116), a gene encoding a B. diazoefficiens CYP112 polypeptide (SEQ ID NO:123, SEQ ID NO:124), a gene encoding a P. putida ferredoxin polypeptide (SEQ ID NO:147, SEQ ID NO:148), and a gene encoding a P. putida ferredoxin reductase polypeptide (SEQ ID NO:149, SEQ ID NO:150) accumulates GA₉. See Example 7, FIGS. 10 and 11, and Table 12. In some embodiments, a ferredoxin reductase polypeptide or a cytochrome P450 reductase reduce CYP112.

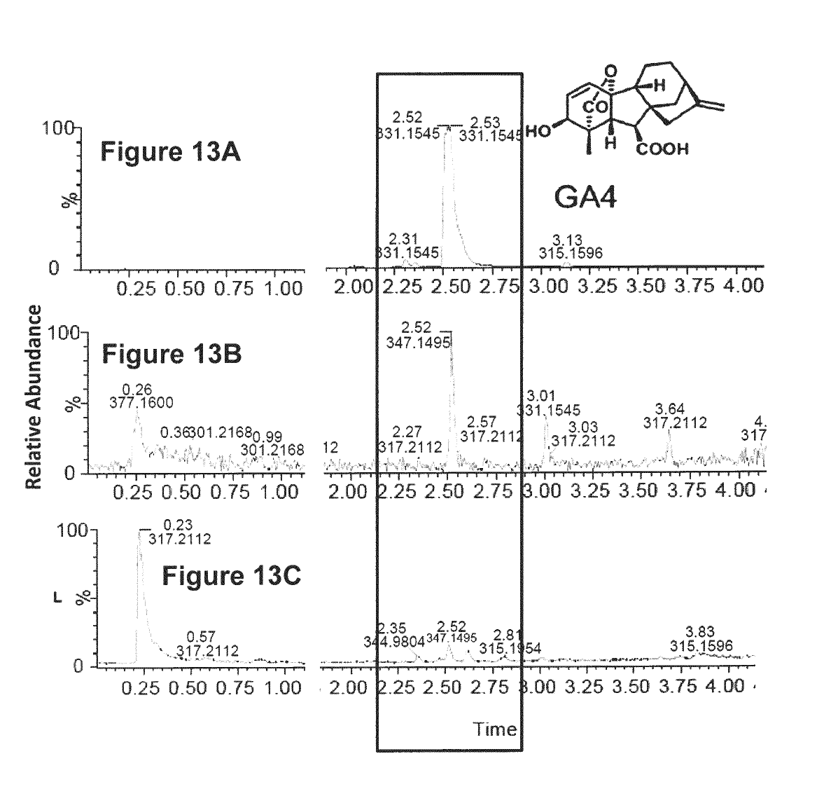

In some embodiments, an S. cerevisiae strain (strain “U”) comprising a gene encoding a truncated CDPS polypeptide (SEQ ID NO:179, SEQ ID NO:180), a gene encoding a KS polypeptide (SEQ ID NO:181, SEQ ID NO:182), a first gene encoding a KO polypeptide (SEQ ID NO:171, SEQ ID NO:172), a second gene encoding a KO polypeptide (SEQ ID NO:169, SEQ ID NO:170), a gene encoding a CPR polypeptide (SEQ ID NO:167, SEQ ID NO:168), a gene encoding an ERG20-GGPPS7 polypeptide (SEQ ID NO:195, SEQ ID NO:196), a gene encoding an S. manihoticola KAO4 polypeptide (SEQ ID NO:73, SEQ ID NO:74), a gene encoding a KO polypeptide (SEQ ID NO:169, SEQ ID NO:170), a gene encoding a B. diazoefficiens ADH polypeptide (SEQ ID NO:115, SEQ ID NO:116), a gene encoding a B. diazoefficiens CYP112 polypeptide (SEQ ID NO:123, SEQ ID NO:124), a gene encoding a P. putida ferredoxin polypeptide (SEQ ID NO:147, SEQ ID NO:148), and a gene encoding a P. putida ferredoxin reductase polypeptide (SEQ ID NO:149, SEQ ID NO:150) accumulates GA₄. See Example 7, FIGS. 12 and 13, and Table 13.

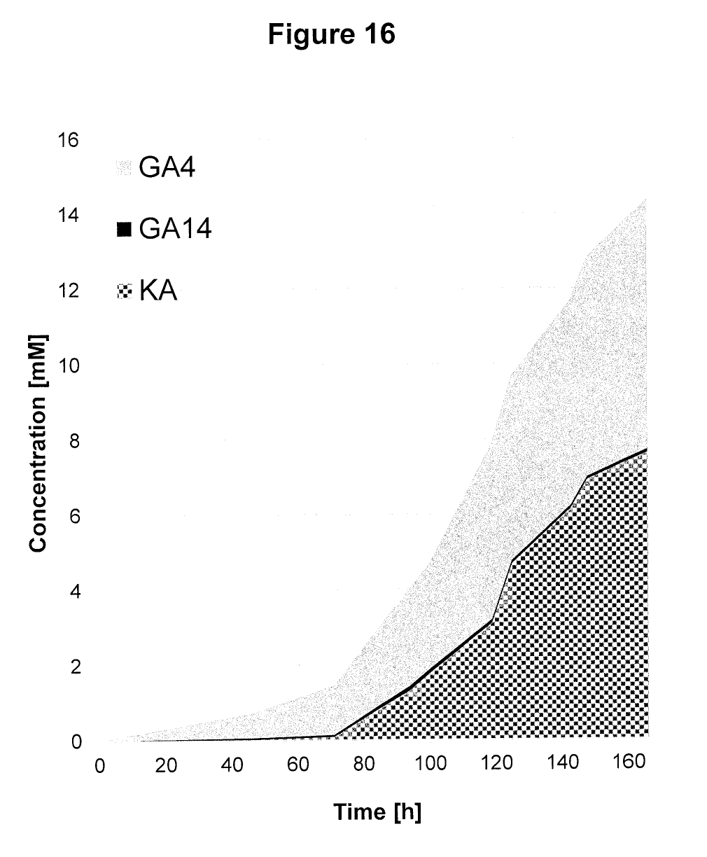

In some embodiments, an S. cerevisiae strain comprising a gene encoding a DAP1-2 polypeptide (SEQ ID NO:212, SEQ ID NO:213), a gene encoding a CytB5-2 polypeptide (SEQ ID NO:238, SEQ ID NO:239), a gene encoding a CytB5red-4 polypeptide (SEQ ID NO:240, SEQ ID NO:241), a gene encoding a FfCytB5-1 polypeptide (SEQ ID NO:159, SEQ ID NO:160), a gene encoding a FfCytB5red-1 polypeptide (SEQ ID NO:01, SEQ ID NO:02), a gene encoding an KAO11 polypeptide (SEQ ID NO:63, SEQ ID NO:64), a gene encoding CPR12 polypeptide (SEQ ID NO:167, SEQ ID NO:168), a gene encoding a CDPS-KS6 polypeptide (SEQ ID NO:101, SEQ ID NO:102), a gene encoding a KS5 polypeptide (SEQ ID NO:181, SEQ ID NO:182), a gene encoding a GGPPS-7 polypeptide (SEQ ID NO:177, SEQ ID NO:178), a gene encoding a KO1 polypeptide (SEQ ID NO:171, SEQ ID NO:172), a gene encoding a O. sativa GA₁₃ₒₓ₋₁ polypeptide (SEQ ID NO:97, SEQ ID NO:98) a gene encoding a C. maxima GA₂₀ₒₓ₋₄ polypeptide (SEQ ID NO:39, SEQ ID NO:40), and a gene encoding a M. macrocarpus GA₃ₒₓ₋₁ polypeptide (SEQ ID NO:27, SEQ ID NO:28). The strain produces GA₄ and other gibberellin intermediates. See Example 12, FIG. 16, and Tables 21 and 22.

In some embodiments, an S. cerevisiae strain comprising a gene encoding a DAP1-2 polypeptide (SEQ ID NO:212, SEQ ID NO:213), a gene encoding an ICE2-2 polypeptide (SEQ ID NO:206, SEQ ID NO:206), a gene encoding a CDPS-KS6 polypeptide (SEQ ID NO:101, SEQ ID NO:102), a gene encoding a KS5 polypeptide (SEQ ID NO:181, SEQ ID NO:182), a gene encoding a FfCytB5-1 polypeptide (SEQ ID NO:159, SEQ ID NO:160) a gene encoding a FfCytB5red-1 polypeptide (SEQ ID NO:01, SEQ ID NO:02), a gene encoding an KAO3 polypeptide (SEQ ID NO:145, SEQ ID NO:146), a gene encoding a CPR19 polypeptide (SEQ ID NO:193, SEQ ID NO:194), a gene encoding CPR12 polypeptide (SEQ ID NO:167, SEQ ID NO:168), a gene encoding a RsKO polypeptide (SEQ ID NO:169, SEQ ID NO:170), a gene encoding a GGPPS-7 polypeptide (SEQ ID NO:177, SEQ ID NO:178), a gene encoding a KO1 polypeptide (SEQ ID NO:171, SEQ ID NO:172), a gene encoding a P450-2-1 polypeptide (SEQ ID NO:79, SEQ ID NO:80) a gene encoding a KAO4 polypeptide (SEQ ID NO:73, SEQ ID NO:74), and a gene encoding a DES-1 polypeptide (SEQ ID NO:25, SEQ ID NO:26). The strain produces GA₃ and other gibberellin intermediates. See Example 11 and Tables 19 and 20.

In some aspects, a gibberellin-producing host or gibberellin precursor-producing host comprises a damage resistance protein 1 (DAP1) polypeptide. In some embodiments, the DAP1 polypeptide is a DAP1 polypeptide as set forth in GenBank Accession No. YPL170W (SEQ ID NO:223, SEQ ID NO:224). In some aspects, the DAP1 enzyme is a G. fujikuroi DAP1 polypeptide is a polypeptide having the amino acid sequence set forth in SEQ ID NO:215, SEQ ID NO:217, or SEQ ID NO:219 (encoded by a nucleotide sequence set forth in SEQ ID NO:214, SEQ ID NO:216, or SEQ ID NO:217, respectively). In some aspects, expression of a DAP polypeptide increases cytochrome P450 activity.

In some aspects, a gibberellin-producing host or gibberellin precursor-producing host comprises inheritance of cortical ER protein 2 (ICE2) polypeptide. In some aspects, the ICE2 polypeptide can be a G. fujikuroi ICE2 (SEQ ID NO:205, SEQ ID NO:206). In some aspects, ICE2 is overexpressed.

In some embodiments, one or more endogenous genes encoding one or more alcohol dehydrogenase polypeptides are disrupted in a host. In some aspects, an alcohol dehydrogenase is knocked out or disrupted individually or in combination with one or more additional alcohol dehydrogenases. In some aspects, disruption of an endogenous alcohol dehydrogenase prevents reduction of aldehyde pathway intermediates to their corresponding alcohols. For example, disruption of one or more alcohol dehydrogeases can prevent reduction of GA₁₂-aldehyde, GA₁₄-aldehyde, kaurenal, GA₂₄, and/or GA₃₆. In some aspects, disruption of an endogenous alcohol dehydrogenase results in an increased accumulation of gibberellins.

Gibberellin production can be detected and/or analyzed by techniques generally available to one skilled in the art, for example, but not limited to, LC-MS, thin layer chromatography (TLC), high-performance liquid chromatography (HPLC), ultraviolet visible spectroscopy/spectrophotometry (UV-Vis), mass spectrometry (MS), and nuclear magnetic resonance spectroscopy (NMR). In some aspects, GA₃ accumulates at least 100 mg/liter in fed batch fermentation methods.

### Functional Homologs

Functional homologs of the polypeptides described above are also suitable for use in producing gibberellins in a recombinant host. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of gibberellin biosynthesis polypeptides. Sequence analysis can involve BLAST, Reciprocal BLAST, or PSI-BLAST analysis of non-redundant databases using a cytochrome P450 monooxygenase, cytochrome P450 reductase, and/or 2-ODD amino acid sequence as the reference sequence. Amino acid sequence is, in some instances, deduced from the nucleotide sequence. Those polypeptides in the database that have greater than 40% sequence identity are candidates for further evaluation for suitability as a gibberellin biosynthesis polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in gibberellin biosynthesis polypeptides, e.g., conserved functional domains. In some embodiments, nucleic acids and polypeptides are identified from transcriptome data based on expression levels rather than by using BLAST analysis.

Conserved regions can be identified by locating a region within the primary amino acid sequence of a gibberellin biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing gibberellins in a recombinant host include functional homologs of cytochrome P450 monooxygenase, cytochrome P450 reductase, and/or 2-ODD. Methods to modify the substrate specificity of, for example, cytochrome P450 monooxygenase, cytochrome P450 reductase, and/or 2-ODD, are known to those skilled in the art, and include without limitation site-directed/rational mutagenesis approaches, random directed evolution approaches and combinations in which random mutagenesis/saturation techniques are performed near the active site of the enzyme. For example, see Osmani et al., 2009, Phytochemistry 70: 325-47.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A percent (%) identity for any candidate nucleic acid or polypeptide relative to a reference nucleic acid or polypeptide can be determined as follows. A reference sequence (e.g., a nucleic acid sequence or the amino acid sequence described herein) is aligned to one or more candidate sequences using the computer program ClustalW (version 1.83, default parameters), which allows alignments of nucleic acid or polypeptide sequences to be carried out across their entire length (global alignment).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: % age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gin, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent (%) identity of a candidate nucleic acid or amino acid sequence to a reference sequence, the sequences are aligned using ClustalW, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “% identity” as used herein about amino acid sequences means the degree of identity in percent between two amino acid sequences obtained when using the Needleman-Wunsch algorithm as implemented in the Needle program of the EMBOSS package (EMBOSS: The European Molecular Biology Open Software Suite), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

[(identical amino acid residues)/(Length of alignment−total number of gaps in alignment)]×100

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information. BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins

Furthermore the degree of local identity between the amino acid sequence query or nucleic acid sequence query and the retrieved homologous sequences is determined by the BLAST program. However only those sequence segments are compared that give a match above a certain threshold. Accordingly the program calculates the identity only for these matching segments. Therefore the identity calculated in this way is referred to as local identity.

It will be appreciated that functional cytochrome P450, cytochrome P450 monooxygenase, cytochrome P450 reductase, and/or 2-ODD proteins can include additional amino acids that are not involved in the enzymatic activities carried out by the enzymes. In some embodiments, cytochrome P450, cytochrome P450 monooxygenase, cytochrome P450 reductase, and/or 2-ODD proteins are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to polypeptides engineered through the joining of two or more genes that code for different polypeptides (i.e., a polypeptide operatively-linked to a different polypeptide). For example, a polypeptide encoded by a nucleic acid sequence containing a coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more, but typically fewer than 10, 9, 8, 7, or 6 amino acids. The protein product encoded by a fusion construct is referred to as a fusion polypeptide. A chimeric or fusion protein provided herein can include one or more For example, a non-limiting example of a fusion protein can include a CDPS gene fused to a KS gene to generate a CDPS-KS fusion protein when expressed. In some embodiments, a nucleic acid sequence encoding a cytochrome P450, cytochrome P450 monooxygenase, cytochrome P450 reductase, and/or 2-ODD polypeptide can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, Conn.). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein.

In some embodiments, a protein is a protein altered by circular permutation, which consists in the covalent attachment of the ends of a protein that would be opened elsewhere afterwards. Thus, the order of the sequence is altered without causing changes in the amino acids of the protein. In some embodiments, a targeted circular permutation can be produced, for example but not limited to, by designing a spacer to join the ends of the original protein. Once the spacer has been defined, there are several possibilities to generate permutations through generally accepted molecular biology techniques, for example but not limited to, by producing concatemers by means of PCR and subsequent amplification of specific permutations inside the concatemer or by amplifying discrete fragments of the protein to exchange to join them in a different order. The step of generating permutations can be followed by creating a circular gene by binding the fragment ends and cutting back at random, thus forming collections of permutations from a unique construct. In some embodiments, a polypeptide disclosed herein is altered by circular permutation.

### Gibberellin Biosynthesis Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids may be introduced at positions other than the position where the native sequence is found or kept extrachromosomally in episomes.

As used herein, the term “regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

One or more genes can be combined in a recombinant nucleic acid construct in “modules” useful for a discrete aspect of gibberellin precursor and/or gibberellin production. Combining a plurality of genes in a module, particularly a polycistronic module, facilitates the use of the module in a variety of species. For example, a gibberellin biosynthesis gene cluster, or a UGT gene cluster, can be combined in a polycistronic module such that, after insertion of a suitable regulatory region, the module can be introduced into a wide variety of species. As another example, a UGT gene cluster can be combined such that each UGT coding sequence is operably linked to a separate regulatory region, to form a UGT module. Such a module can be used in those species for which monocistronic expression is necessary or desirable. In addition to genes useful for gibberellin precursor or gibberellin production, a recombinant construct typically also contains an origin of replication, and one or more selectable markers for maintenance of the construct in appropriate species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

In some cases, it is desirable to inhibit one or more functions of an endogenous polypeptide in order to divert metabolic intermediates towards gibberellin precursor or gibberellin biosynthesis. For example, it may be desirable to downregulate synthesis of sterols in a yeast strain in order to further increase gibberellin precursor or gibberellin production, e.g., by downregulating squalene epoxidase. As another example, it may be desirable to inhibit degradative functions of certain endogenous gene products, e.g., glycohydrolases that remove glucose moieties from secondary metabolites or phosphatases as discussed herein. In such cases, a nucleic acid that overexpresses the polypeptide or gene product may be included in a recombinant construct that is transformed into the strain. Alternatively, mutagenesis can be used to generate mutants in genes for which it is desired to increase or enhance function.

### Host Microorganisms

Recombinant hosts can be used to express polypeptides for the producing gibberellins, including mammalian, insect, plant, and algal cells. A number of prokaryotes and eukaryotes are also suitable for use in constructing the recombinant microorganisms described herein, e.g., gram-negative bacteria, yeast, and fungi. A species and strain selected for use as a gibberellin production strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

In some embodiments, the bacterial cell comprises Escherichia cells, Lactobacillus cells, Lactococcus cells, Corynebacterium cells, Acetobacter cells, Acinetobacter cells, Pseudomonas cells, or Streptomyces cells.

In some embodiments, the fungal cell comprises a yeast cell. For example, the yeast cell can be a Saccharomycete. The yeast cell can comprise a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species. In an embodiment, the yeast cell is a cell from the Saccharomyces cerevisiae species. In another embodiment, the fungal cell of the fungal cell comprises a filamentous fungal cell.

Typically, the recombinant microorganism is grown in a fermenter at a temperature(s) for a period of time, wherein the temperature and period of time facilitate the production of a gibberellin precursor and/or gibberellin compound. For example, the period of time can be approximately 120 hours. Growth in a fermenter can be performed with agitation. The constructed and genetically engineered microorganisms provided by the invention can be cultivated using conventional fermentation processes, including, inter alia, chemostat, batch, fed-batch cultivations, semi-continuous fermentations such as draw and fill, continuous perfusion fermentation, and continuous perfusion cell culture. Depending on the particular microorganism used in the method, other recombinant genes such as isopentenyl biosynthesis genes and terpene synthase and cyclase genes may also be present and expressed. Levels of substrates and intermediates, e.g., isopentenyl diphosphate, dimethylallyl diphosphate, GGPP, ent-kaurene and ent-kaurenoic acid, can be determined by extracting samples from culture media for analysis according to published methods.

As used herein “a carbon source” or “carbon sources” can include any molecule that can be metabolized by a recombinant host cell to facilitate growth and/or production of the gibberellins. Examples of suitable carbon sources include, but are not limited to, sucrose (e.g., as found in molasses), fructose, xylose, ethanol, glycerol, glucose, cellulose, starch, cellobiose, maltodextrin, mannitol, other sugars or other glucose-comprising polymer. In embodiments employing yeast as a host, for example, carbons sources such as sucrose, fructose, xylose, ethanol, glycerol, and glucose are suitable. The carbon source can be provided to the host organism throughout the cultivation period or alternatively, the organism can be grown for a period of time in the presence of another energy source, e.g., protein, and then provided with a source of carbon only during the fed-batch phase.

After the recombinant microorganism has been grown in culture for the period of time, wherein the temperature and period of time facilitate the production of a gibberellin precursor and/or gibberellin compound, the gibberellin precursor and/or gibberellin compound can then be recovered from the culture using various techniques known in the art. In some embodiments, a permeabilizing agent can be added to aid the feedstock entering into the host and product getting out. For example, a crude lysate of the cultured microorganism can be centrifuged to obtain a supernatant. The resulting supernatant can then be applied to a chromatography column, e.g., a C-18 column, and washed with water to remove hydrophilic compounds, followed by elution of the compound(s) of interest with a solvent such as methanol. The compound(s) can then be further purified by preparative HPLC. See for example, WO 2009/140394.

It will be appreciated that the various genes and modules discussed herein can be present in two or more recombinant hosts rather than a single host. When a plurality of recombinant hosts is used, they can be grown in a mixed culture to accumulate gibberellin precursors and/or gibberellins.

Alternatively, the two or more hosts each can be grown in a separate culture medium and the product of the first culture medium, e.g., ent-kaurenoic acid, can be introduced into second culture medium to be converted into a subsequent intermediate, or into an end product such as, for example, GA₃. The product produced by the second, or final host is then recovered. It will also be appreciated that in some embodiments, a recombinant host is grown using nutrient sources other than a culture medium and utilizing a system other than a fermenter.

Exemplary prokaryotic and eukaryotic species are described in more detail below. However, it will be appreciated that other species can be suitable. For example, suitable species can be in a genus such as Agaricus, Bacillus, Candida, Corynebacterium, Eremothecium, Escherichia, Bradyrhizobium, Rhizobium, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Cyberlindnera jadinii, Physcomitrella patens, Rhodoturula glutinis, Rhodoturula mucilaginosa, Phaffia rhodozyma, Bradyrhizobium japonicum, Xanthophyllomyces dendrorhous, F. fujikuroi/G. fujikuroi, Candida utilis, Candida glabrata, Candida albicans, and Yarrowia lipolytica.

In some embodiments, a microorganism can be a prokaryote such as Escherichia bacteria cells, for example, Escherichia coli cells; Lactobacillus bacteria cells; Lactococcus bacteria cells; Corynebacterium bacteria cells; Acetobacter bacteria cells; Acinetobacter bacteria cells; or Pseudomonas bacterial cells.

In some embodiments, a microorganism can be an Ascomycete such as G. fujikuroi, Kluyveromyces lactis, Schizosaccharomyces pombe, A. niger, Yarrowia lipolytica, Ashbya gossypii, or S. cerevisiae.

In some embodiments, a microorganism can be an algal cell such as Blakeslea trispora, Dunaliella salina, Haematococcus pluvialis, Chlorella sp., Undaria pinnatifida, Sargassum, Laminaria japonica, Scenedesmus almeriensis species.

In some embodiments, a microorganism can be a cyanobacterial cell such as Blakeslea trispora, Dunaliella salina, Haematococcus pluvialis, Chlorella sp., Undaria pinnatifida, Sargassum, Laminaria japonica, Scenedesmus almeriensis.

### Saccharomyces spp.

Saccharomyces is a widely used organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

### Aspergillus spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for producing gibberellins. E. coli

E. coli, another widely-used organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Agaricus, Gibberella, and Phanerochaete spp.

Agaricus, Gibberella, and Phanerochaete spp. can be useful because they are known to produce large amounts of isoprenoids in culture. Thus, the terpene precursors for producing large amounts of gibberellins are already produced by endogenous genes. Thus, modules comprising recombinant genes for gibberellin biosynthesis polypeptides can be introduced into species from such genera without the necessity of introducing mevalonate or MEP pathway genes.

Arxula adeninivorans (Blastobotrys adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications and is an oleaginous microorganism. Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biochimie 91(6):692-6; Bankar et al., 2009, Appl Microbiol Biotechnol. 84(5):847-65.

### Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

Rhodosporidium toruloides

Rhodosporidium toruloides is oleaginous yeast and useful for engineering lipid-production pathways. See, e.g., Zhu et al., 2013, Nature Commun. 3:1112; Ageitos et al., 2011, Applied Microbiology and Biotechnology 90(4):1219-27).

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

Kluyveromyces lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et al., 2014, N Biotechnol. 31(6):532-7.

### Physcomitrella spp.

Physcomitrella mosses, when grown in suspension culture, have characteristics similar to yeast or other fungal cultures. This genera can be used for producing plant secondary metabolites, which can be difficult to produce in other types of cells.

It can be appreciated that the recombinant host cell disclosed herein can comprise a plant cell, a mammalian cell, an insect cell, a fungal cell, comprising a yeast cell, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species or is a Saccharomycete or is a Saccharomyces cerevisiae cell, an algal cell or a bacterial cell, comprising Escherichia cells, Lactobacillus cells, Lactococcus cells, Cornebacterium cells, Acetobacter cells, Acinetobacter cells, or Pseudomonas cells.

### Plants

Various plants can be used as recombinant host cells (e.g., plant cells, both monocotyledenous and dicotyledenous). In an embodiment, the plants or host cells used in the methods can be derived from monocots, particularly the members of the taxonomic family known as the Gramineae. This includes all members of the grass family of which the edible varieties are known as cereals. The cereals include a wide variety of species such as wheat (Triticum sps.), rice (Oryza sps.) barley (Hordeum sps.) oats, (Avena sps.) rye (Secale sps.), corn (maize) [Zea sps.) and millet (Pennisettum sps.). In another embodiment, the plants or host cells used can be derived from dicots (e.g., soybean (Glycine spp.)). In order to produce transgenic plants that produce gibberellins, plant cells or tissues derived from them are transformed or integrated with genes coding for various enzymes the result in the production of gibberellins. The transgenic plant cells are cultured in medium containing the appropriate selection agent to identify and select for plant cells which express the heterologous nucleic acid sequence. After plant cells that express the heterologous nucleic acid sequence are selected, whole plants can be regenerated from the selected transgenic plant cells. Techniques for regenerating whole plants from transformed plant cells are generally known in the art.

Plant cells or tissues can be transformed with expression constructs (i.e., heterologous nucleic acid constructs) using a variety of standard techniques. In some embodiments, the heterologous nucleic acid sequences can be stably integrated into the host cell genome so that the integrated nucleic acid sequences are passed onto successive plant generations. The skilled artisan will recognize that a wide variety of transformation techniques exist in the art. Any technique that is suitable for the target host plant may be employed. For example, the nucleic acid sequences can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. When Agrobacterium is used for plant cell transformation, a vector is introduced into the Agrobacterium host for homologous recombination with T-DNA or the Ti- or Ri-plasmid present in the Agrobacterium host. The Ti- or Ri-plasmid containing the T-DNA for recombination may be armed (capable of causing gall formation) or disarmed (incapable of causing gall formation), the latter being permissible, so long as the vir genes are present in the transformed Agrobacterium host. The armed plasmid can give a mixture of normal plant cells and gall. In some embodiments, Agrobacterium can be used as the vehicle for transforming host plant cells. The expression or transcription construct bordered by the T-DNA border region(s) is inserted into a broad host range vector capable of replication in E. coli and Agrobacterium, for example pRK2 or derivatives thereof. Alternatively, one may insert the sequences to be expressed in plant cells into a vector containing separate replication sequences, one of which stabilizes the vector in E. coli, and the other in Agrobacterium. A number of markers have been developed for use with plant cells, such as resistance to chloramphenicol, kanamycin, the aminoglycoside G418, hygromycin, or the like.

It will be appreciated that the various genes and modules discussed herein can be present in two or more recombinant microorganisms rather than a single microorganism. When a plurality of recombinant microorganisms is used, they can be grown in a mixed culture to produce gibberellin precursors and/or gibberellins. For example, a first microorganism can comprise one or more biosynthesis genes for producing a gibberellin precursor, while a second microorganism comprises gibberellin biosynthesis genes. The product produced by the second, or final microorganism is then recovered. It will also be appreciated that in some embodiments, a recombinant microorganism is grown using nutrient sources other than a culture medium and utilizing a system other than a fermenter.

Alternatively, the two or more microorganisms each can be grown in a separate culture medium and the product of the first culture medium, e.g., ent-kaurenoic acid, can be introduced into second culture medium to be converted into a subsequent intermediate, or into an end product such as GA₃. The product produced by the second, or final microorganism is then recovered.

### Down Stream Processing

A number of different methods can be used to isolate and purify the gibberellin precursors and/or gibberellin compounds produced by the methods and host cells disclosed herein. For example, the isolating steps may comprise: (a) contacting the cell culture comprising the gibberellin precursor and/or the gibberellin compound with: (i) one or more adsorbent resins in a packed column in order to bind at least a portion of the gibberellin precursor and/or the gibberellin compound to the resin, thereby isolating the gibberellin precursor and/or the gibberellin compound; or (ii) one or more ion exchange or reversed-phase chromatography columns in order to bind at least a portion of the gibberellin precursor and/or the gibberellin compound in the column, thereby isolating the gibberellin precursor and/or the gibberellin compound; or (b) crystallizing and/or extracting the gibberellin precursor and/or the gibberellin compound from the cell culture, thereby isolating the gibberellin precursor and/or the gibberellin compound; or (c) separating the cell culture into a solid phase and a liquid phase, wherein the liquid phase comprises the gibberellin precursor and/or the gibberellin compound; and (i) contacting the liquid phase with one or more adsorbent resins in order to bind at least a portion of the gibberellin precursor and/or the gibberellin compound to the resin, thereby isolating the gibberellin precursor and/or the gibberellin compound; (ii) contacting the liquid phase with one or more ion exchange or reversed-phase chromatography columns in order to bind at least a portion of the gibberellin precursor and/or the gibberellin compound in the column, thereby isolating the gibberellin precursor and/or the gibberellin compound; or (iii) crystallizing and/or extracting the gibberellin precursor and/or the gibberellin compound from the liquid phase, thereby isolating the gibberellin precursor and/or the gibberellin compound.

In an embodiment, the isolating step can comprise, separating the solid phase from the liquid phase using a process comprising tangential flow filtration with diafiltration membranes to generate a permeate stream comprising the gibberellin precursor and/or the gibberellin compound, wherein the membranes used in the tangential flow filtration are ultrafiltration or nanofiltration membranes. In an embodiment, the permeate stream is extracted by an organic solvent which phase-separates from the aqueous phase to generate an extracted gibberellin product in the organic solvent

Optionally the permeate stream containing the gibberellin product could be concentrated by some combination of reverse osmosis, nanofiltration, and evaporation to produce a crystallized gibberellin precursor and/or the gibberellin compound.

The aqueous gibberellin-containing permeate or the concentrate can be extracted by an organic solvent which phase-separates from the aqueous phase. The pH of the aqueous phase is adjusted to less than 4.0, or less than 3.0, in order to protonate the gibberellin molecules and ensure they partition into the organic phase to a high degree. The solvent extraction could be performed in a counter-current extraction centrifuge such as a Podbelniak extractor, or in a counter-current extraction column such as a Karr or Scheibel column. This yields the gibberellin product in an organic solvent suitable for subsequent purification processing.

It will be understood that organic solvent extraction can be replaced with a series of process operations which yield a similar organic solution of gibberellins. The series of process operations would include (a) precipitation of gibberellins from the aqueous concentrate produced by addition of acid until pH is less than 4.0 or less than 3.0; (b) filtration and optionally water-washing of the resulting gibberellins-containing solids; and (c) dissolution of the filtered gibberellins-containing solids into an organic solvent suitable for purification processing.

Optionally the organic extract can be contacted with carbon to adsorb impurities and color bodies. Optionally the carbon contacting can be done by mixing carbon in the organic extract and filtering the carbon out of the resulting suspension, or by feeding the organic extract to a column or filter containing a fixed bed of carbon and collecting a purified effluent stream. The organic extract can be crystallized by concentrating the solution evaporatively. The resulting gibberellins product crystals can be filtered, washed, and dried to yield a high-purity gibberellins product.

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1. LC-MS Analytical Procedures

Liquid chromatography-mass spectrometry (LC-MS) analyses were performed on Waters ACQUITY UPLC® (Waters Corporation) with a Waters ACQUITY UPLC® BEH C18 column (2.1×50 mm, 1.7 μm particles, 130 Å pore size) equipped with a pre-column (2.1×5 mm, 1.7 μm particles, 130 Å pore size) coupled to a Waters ACQUITY TQD triple quadropole mass spectrometer with electrospray ionization (ESI) operated in negative ionization mode. Compound separation was achieved using a gradient of the two mobile phases: phase A (water with 0.1% formic acid) and phase B (MeCN with 0.1% formic acid) were separated by increasing from 20% to 50% B between 0.3 to 2.0 minutes, increasing to 100% B at 2.01 minutes and holding 100% B for 0.6 minutes, and re-equilibrating for 0.6 minutes. The flow rate was 0.6 mL/min, and the column temperature was set at 55° C. Gibberellins were monitored using SIM (Single Ion Monitoring) and quantified by comparing against authentic standards.

### Example 2. Engineering of Gibberellin-Producing S. Cerevisiae Strain

An ent-kaurenoic acid-producing S. cerevisiae strain comprising genes encoding a truncated copalyl diphosphate synthase (CDPS) polypeptide (SEQ ID NO:179 (nt), SEQ ID NO:180 (aa)), a kaurene synthase (KS) polypeptide (SEQ ID NO:181 (nt), SEQ ID NO:182 (aa)), a first KO polypeptide (SEQ ID NO:171 (nt), SEQ ID NO:172 (aa)), a second KO polypeptide (SEQ ID NO:169 (nt), SEQ ID NO:170 (aa)), a CPR polypeptide (SEQ ID NO:167 (nt), SEQ ID NO:168 (aa)), and an ERG20-GGPPS7 polypeptide (SEQ ID NO:195 (nt), SEQ ID NO:196 (aa)) was engineered to accumulate gibberellins. Strains “A,” “N,” and “F” were transformed into this ent-kaurenoic acid-producing strain background; the genes of Table 2 or Table 3 were introduced into the strain using the USER™ based yeast integration vector system. See, e.g., Mikkelsen et al., 2012, Metabolic Engineering 14:104-11. See also, the pathway described in FIG. 3.

TABLE 2
Genes expressed in S. cerevisiae strain “N.”

| | Gene 1 | | Gene 2 |
|---|---|---|---|
| Gene 1 | SEQ ID NOs | Gene 2 | SEQ ID NOs |
| G. fujikuroi | SEQ ID NO: | G. fujikuroi | SEQ ID NO: |
| P450-2-1 | 79 (nt) | P450-3-4 | 185 (nt) |
| | SEQ ID NO: | | SEQ ID NO: |
| | 80 (aa) | | 186 (aa) |
| S. manihoticola | SEQ ID NO: | G. fujikuroi | SEQ ID NO: |
| KAO4 | 73 (nt) | DES-1 | 25 (nt) |
| | SEQ ID NO: | | SEQ ID NO: |
| | 74 (aa) | | 26 (aa) |
| G. fujikuroi | SEQ ID NO: | G. fujikuroi | SEQ ID NO: |
| Cytochrome B5 | 159 (nt) | Cytochrome B5 | 1 (nt) |
| | SEQ ID NO: | reductase | SEQ ID NO: |
| | 160 (aa) | | 2 (aa) |

TABLE 3
Genes expressed in S. cerevisiae strain “A.”

| | Gene 1 | | Gene 2 |
|---|---|---|---|
| Gene 1 | SEQ ID NOs | Gene 2 | SEQ ID NOs |
| G. fujikuroi | SEQ ID NO: | G. fujikuroi | SEQ ID NO: |
| P450-2-1 | 79 (nt) | P450-3-4 | 185 (nt) |
| | SEQ ID NO: | | SEQ ID NO: |
| | 80 (aa) | | 186 (aa) |
| S. manihoticola | SEQ ID NO: | G. fujikuroi | SEQ ID NO: |
| KAO4 | 73 (nt) | DES-1 | 25 (nt) |
| | SEQ ID NO: | | SEQ ID NO: |
| | 74 (aa) | | 26 (aa) |
| A. niger | SEQ ID NO: | | |
| CPR16 | 157 (nt) | | |
| | SEQ ID NO: | | |
| | 158 (aa) | | |

Furthermore, the ent-kaurenoic acid-producing S. cerevisiae strain described above was also transformed with the genes of Table 4 using the USER™ cloning based yeast integration system to engineer strain “F.” See the pathway described in FIG. 5. As with S. cerevisiae strains comprising a gene encoding a G. fujikuroi P450-2-1 polypeptide (strains “N,” “A,” and “I”), ent-kaurenoic acid-producing S. cerevisiae strains comprising a gene encoding a C. maxima GA₂₀ₒₓ₋₄ polypeptide (SEQ ID NO:39 (nt), SEQ ID NO:40 (aa)) accumulated gibberellins. See FIG. 4A. Thus, S. cerevisiae strains comprising both fungal pathway genes and a plant gene (i.e. GA₂₀ₒₓ) are capable of producing gibberellins such as GA₃.

TABLE 4
Genes expressed in S. cerevisiae strain “F.”

| | Gene 1 | | Gene 2 |
|---|---|---|---|
| Gene 1 | SEQ ID NOs | Gene 2 | SEQ ID NOs |
| C. maxima | SEQ ID NO: | G. fujikuroi | SEQ ID NO: |
| GA₂₀ₒₓ₋₄ | 39 (nt) | P450-3-4 | 185 (nt) |
| | SEQ ID NO: | | SEQ ID NO: |
| | 40 (aa) | | 186 (aa) |
| S. manihoticola | SEQ ID NO: | G. fujikuroi | SEQ ID NO: |
| KAO4 | 73 (nt) | DES-1 | 25 (nt) |
| | SEQ ID NO: | | SEQ ID NO: |
| | 74 (aa) | | 26 (aa) |
| A. niger | SEQ ID NO: | | |
| CPR16 | 157 (nt) | | |
| | SEQ ID NO: | | |
| | 158 (aa) | | |

Gibberellin accumulation was observed with these recombinant S. cerevisiae strains and was measured using one of two LC-MS methods. In the first method, LC-MS analysis was performed using a Waters ACQUITY I-class UPLC system fitted with a Waters ACQUITY UPLC® BEH shield RP18 column (2.1×50 mm, 1.7 μm particles, 130 Å pore size) equipped with an ACQUITY UPLC® BEH C18 VanGuard pre-column (130 Å, 1.7 μm, 2.1 mm×5 mm) connected to a Waters Xevo SQ Detector 2 single quadrupole mass spectrometer equipped with an electrospray ionization (ESI) source. Compound separation was carried out using mobile phase of eluent B (ACN with 0.1% formic acid) and eluent A (water with 0.1% formic acid) using gradient separation. Quantification of gibberellins was performed by comparing obtained signals with authentic standards. Gibberellin accumulation was detected using single ion reaction (SIR) in negative ionization mode using the traces described in Table 5. In the second method, LC-MS analysis was performed using a Waters ACQUITY I-class UPLC system fitted with a Waters ACQUITY UPLC® BEH shield RP18 column (2.1×50 mm, 1.7 μm particles, 130 Å pore size) equipped with an ACQUITY UPLC® BEH C18 VanGuard pre-column (130 Å, 1.7 μm, 2.1 mm×5 mm) connected to a Waters XEVO® G2-S quadrupole time-of-flight (QTOF) mass spectrometer equipped with an electrospray ionization (ESI) source operated in negative ionization mode. Compound separation was carried out using the gradient of the first LC-MS method. Gibberellin accumulation was detected by investigating extracted ion chromatograms (EICs) corresponding to their theoretical accurate mass.

TABLE 5
LC-MS analytical characterization.

| | | | | Typical |
|---|---|---|---|---|
| | | | | retention |
| Descrip- | Molecular | Monoisotopic | m/z trace | time (t_{R}) |
| tion | formula | mass | (SIR) | [min] |
| GA₃ | C₁₉H₂₂O₆ | [M] 346.1416 | 344.98 ± 0.5 | 1.02 |
| | | [M − H]⁻ 345.1338 | | |
| GA₄ | C₁₉H₂₄O₅ | [M] 332.1545 | 331.16 ± 0.5 | 2.53 |
| | | [M − H]⁻ 331.1545 | | |
| GA₇ | C₁₉H₂₂O₅ | [M] 330.1467 | 329.15 ± 0.5 | 2.47 |
| | | [M − H]⁻ 329.1389 | | |

As shown in FIG. 4A, gibberellins, including, but not limited to, GA₃, GA₄, GA₁₂, GA₁₄, and GA₁₇, accumulated upon expression of the genes of Table 2 (strain “N”). Surprisingly, gibberellin accumulation for strain “N” was approximately 3-fold higher than that of strain “1,” which is identical to strain “N” except for that it does not comprise G. fujikuroi cytochrome B5 (SEQ ID NO:159 (nt), SEQ ID NO:160 (aa)) or G. fujikuroi cytochrome B5 reductase (SEQ ID NO:01 (nt), SEQ ID NO:02 (aa)). Thus, cytochrome B5 and cytochrome B5 reductase significantly improve gibberellin accumulation and this result was unexpected and novel.

As shown in FIG. 4B, gibberellins, including, but not limited to, GA₃, GA₄, GA₁₂, GA₁₃, GA₁₄, GA₂₅, accumulated upon expression of the genes of Table 3 (strain “A”) in the ent-kaurenoic acid-producing S. cerevisiae strain. GA₃ accumulated at approximately 2-10 mg/L in the culture medium of strain “A”. These surprising and unexpected results are thereby the first demonstration of biosynthesis of gibberellins in a heterologous host, S. cerevisiae, which is suitable for efficient large scale commercial production of secondary metabolites.

### Example 3. Analysis of Bifunctional CDPS-KS Homologs

The expression of GGPPS producing genes alone has been shown to cause cell toxicity, therefore, GGPPS was removed by the expression of CDPS and KS genes. CDPS-KS bifunctional genes were constructed to determine the efficiency of each CDPS/KS combination for removing GGPPS by converting GGPPS to kaurenoic acid. CDPS-KS bifunctional fusion genes were comparatively tested in a yeast strain inserted with CytB5-1 and CytB5red-1. The strain was then transformed with CPR12 (SEQ ID NO:167 (nt) and SEQ ID NO:168 (aa)), RsKO_GA (SEQ ID NO:169 (nt) and SEQ ID NO:170 (aa)), GGPPS7 (SEQ ID NO:176 (aa) and SEQ ID NO:178 (aa)), KO1 (SEQ ID NO:171 (nt) and SEQ ID NO:172 (aa)), and either CDPS-KS6+KS5 (SEQ ID NO:101 (nt) and SEQ ID NO:102 (aa), and SEQ ID NO:181 (nt) and SEQ ID NO:182 (aa)), CDPS-KS6 (SEQ ID NO:101 (aa) and SEQ ID NO:102 (nt)), CDPS-KS4 (SEQ ID NO:226 (nt) and SEQ ID NO:227 (aa)), or CDPS-KS9 (SEQ ID NO:228 (nt) and SEQ ID NO:229 (aa)). The expression of the giberellin pathway genes along with CDPS-KS bifunctional genes were tested to determine the production level of kaurenoic acid. Greater levels of production of kaurenoic acid by a bifunctional CDPS-KS gene alone were produced by the expression of the CDPS-KS6 gene (115.14 μM) and this was enhanced by the co-expression of KS5 (CDPS-KS6+KS5) (182.70 μM). The bifunctional CDPS-K4 was less effective in the removal of GGPP as evidenced by the smaller amount of production of kaurenoic acid (8.80 μM) when compared to bifunctional CDPS-KS6 (see Table 6).

TABLE 6
Conversion of GGPPS to Kaurenoic Acid
by bifunctional CDPS-KS homologs.

| | Kaurenoic | |
|---|---|---|
| Bi-Functional CDPS-KS gene | Acid (μM) | Stddev |
| CDPS-KS6 + KS5 | 182.70 | 17.8 |
| CDPS-KS6 | 115.14 | 14.10024 |
| CDPS-KS4 | 8.80 | 2.975735 |

### Example 4. Analysis of KAO Homologs

The production level of gibberellins and gibberellin metabolites can vary depending on the expression of a KAO gene. To determine the amount of GA₁₂ and GA₁₄ produced by KAO activity, a yeast strain containing KO1 (SEQ ID NO:171 (nt) and SEQ ID NO:172 (aa)) and CPR19 (SEQ ID NO:193 (nt) and SEQ ID NO:194 (aa)) was transformed with CDPS-KS6 (SEQ ID NO:101), KS5 (SEQ ID NO:181), GGPPS7 (SEQ ID NO:177), KO1 (SEQ ID NO:171), KAO and CPR genes using USER™ based DNA assembler vectors and NatMx marker. Transformants were then grown and metabolites were analyzed using LC-MS. Yeast strains co-expressed KAO3/CPR19 genes (SEQ ID NO:230 and SEQ ID NO:193), KAO4/CPR17 (SEQ ID NO:73 and SEQ ID NO:187) or CPR19 (SEQ ID NO:193) genes, or KAO5/CPR12 (SEQ ID NO:61 and SEQ ID NO:167) or CPR19 genes (SEQ ID NO:193). The KAO3 and KAO5 genes used were obtained from Integrated DNA Technologies (IDT), and the KAO4 gene used was obtained from GeneArt™ (Invitrogen). Expression of KAO3 resulted in the production 1205 (AUC) of GA₁₂ and 25055 (AUC) of GA₁₄. Expression of KAO4 resulted in the production 4175 (AUC) GA₁₂ and 127115 (AUC) GA₁₄. Lastly, expression of KAO5 resulted in the production of 1605 (AUC) GA₁₄.

TABLE 7
Production of GA₁₂ and GA₁₄ by KAO homologs.

| KAO homolog gene | GA₁₂ | GA₁₄ |
|---|---|---|
| KAO3 (Fusarium fujikuroi) (IDT) | 1205 | 25055 |
| KAO4 (Spaceloma manihoticola) (GeneArt ™) | 4175 | 127115 |
| KAO5 (Ustilaginoidea virens) (IDT) | | 1605 |

Additional yeast studies were conducted to determine the production of gibberellins by various codon-optimized versions of KAO. KAO1 and KAO3 were both codon-optimized versions of F. fujikuroi while KAO2 and KAO4 were codon-optimized versions of F. proliferatum and S. manihoticola, respectively. A yeast train containing FfCytB5-1 (SEQ ID NO:159 (nt) and SEQ ID NO:160 (aa)), FfCytB5red-1 (SEQ ID NO:01 (nt) and SEQ ID NO:02 (aa)), CPR19 (SEQ ID NO:193 (nt) and SEQ ID NO:194 (aa)), RsKO-GA (SEQ ID NO:169 (nt) and SEQ ID NO:170 (aa)), KS5 (SEQ ID NO:181 (nt) and SEQ ID NO:182 (aa)), tCDPS5 (SEQ ID NO:179 (nt) and SEQ ID NO:180 (aa)), GGPPS7 (SEQ ID NO:177 (nt) and SEQ ID NO:178 (aa)), and KO1 (SEQ ID NO:171 (nt) and SEQ ID NO:172 (aa)) was transformed with P450-3-1 (SEQ ID NO:45), P450-2-4 (SEQ ID NO:141), P450-3-4 (SEQ ID NO:185), DES-1 (SEQ ID NO:25), and either KAO1 (SEQ ID NO:89), KAO3 (SEQ ID NO:145), KAO4 (SEQ ID NO:73) or KAO5 (SEQ ID NO:61). USER™ based DNA assembler vectors and URA3 markers were used. Transformants were then grown and metabolites were analyzed using LC-MS. Various amounts of metabolites from GA₁₄ and further downstream the gibberellin pathway were produced (see Table 8). All numerical values in Tables 7 and 8 are area under curve (AUC).

TABLE 8
Production of Gibberellins and Gibberellin
Metabolites by KAO homologs

| KAO homolog | | | | | | |
|---|---|---|---|---|---|---|
| gene | GA₁ | GA₁₂ | GA₁₄ | GA₃ | GA₄ | GA₇ |
| KAO1 | 1100 | 19150 | 15745 | 15645 | 5235 | 935 |
| (F. fujikuroi) | | | | | | |
| KAO2 | — | 3175 | 1615 | 1895 | 795 | 995 |
| (F. proliferatum) | | | | | | |
| KAO3 | 1290 | 16385 | 16635 | 14715 | 7050 | 870 |
| (F. fujikuroi) | | | | | | |
| KAO4 | 5065 | 30895 | 43295 | 24305 | 15065 | 2675 |
| (S. manihoticola) | | | | | | |

### Example 5. Analysis of P450-2 Homologs

Gibberellin acid 14 (GA₁₄) is converted to GA₄ and GA₁ by P450 enzymes. A comparative study of P450-2 homologs was conducted to determine the production level of gibberellins. A yeast strain inserted with P450-3-4 (SEQ ID NO:141 (nt) and SEQ ID NO:142 (aa)), KO1 (SEQ ID NO:170 (nt) and SEQ ID NO:171 (aa)), GGPPS7 (SEQ ID NO:177 (nt) and SEQ ID NO:178 (aa)), CDPS-KS6 (SEQ ID NO:101 (nt) and SEQ ID NO:102 (aa)), KAO4 (SEQ ID NO:73 (nt) and SEQ ID NO:74 (aa)), FfCytB5-1 (SEQ ID NO:159 (nt) and SEQ ID NO:160 (aa)), CPR1 (SEQ ID NO:165 (nt) and SEQ ID NO:166 (aa)), CPR19 (SEQ ID NO:193 (nt) and SEQ ID NO:194 (aa)), and various P450-2 genes. To identify which P450-2 gene was more efficient at the production of GA₁, P450-2-1 (SEQ ID NO:79 (nt) and SEQ ID NO:80 (aa)), P450-2-8 (SEQ ID NO:232 (nt) and SEQ ID NO:233 (aa)), P450-2-9 (SEQ ID NO:234 (nt) and SEQ ID NO:235 (aa)), and P450-2-10 (SEQ ID NO:236 (nt) and SEQ ID NO:237 (aa)) were tested. The combination of genes resulted in the production of GA₁. P450-2-1 produced greater levels of both GA₁ (30309 AUC) and GA₄ (34370 AUC) when compared to the other P450-2 enzymes tested, while P450-2-10 produced a smaller amount of GA₁ (13611 AUC) and P450-2-8 produced a smaller amount of GA₄ (17854 AUC) when compared to the other P450-2 enzymes tested (see Table 9).

TABLE 9
Productionof GA₁ and GA₄ by the
expression of P450-2 homologs.

| P450-2 Homolog | GA₁ | GA₄ |
|---|---|---|
| P450-2-1 F. fujikuroi | 30309 | 34370 |
| P450-2-8 F. fujikuroi | 16472 | 17854 |
| codon optimized (IDT) | | |
| P450-2-9 F. Fujikuroi | 18618 | 30038 |
| P450-2-10 Phaeosphaeria sp. L487 | 13611 | 20440 |

P450-2 enzymes use GA₁₄ as a substrate to produce GA₄. To determine the production level of GA₄ by P450-2 activity, P450-2 genes were introduced into a GA₁₄ producing strain by integration into the yeast genome using a USER™ cloning based vector system. Each P450 gene was introduced using the URA3 selection marker. P450-2-1 and P450-2-6 (SEQ ID NO:17, SEQ ID NO:18) produced surprising levels of GA₄ that were greater levels of GA₄ (581,138 AUC and 279,002 AUC, respectively) when compared to the other P450-2 enzymes tested, while P450-2-4 produced a smaller amount of GA₄ (3456.88 AUC) (see Table 10). All numerical values in Tables 9 and 10 are area under the curve (AUC).

TABLE 10
Production of GA₄ by
the expression of P450-2 genes.

| P450-2 Homolog | GA₄ |
|---|---|
| Fusarium fujikuroi P450-2-1 | 581,138 |
| Fusarium fujikuroi P450-2-4 | 3,457 |
| Ustilaginoidea virens P450-2-5 | 24,058 |
| Fusarium oxysporum P450-2-6 | 279,002 |

### Example 6. Activity of KAO Genes in GA₁₂-Producing S. Cerevisiae Strains

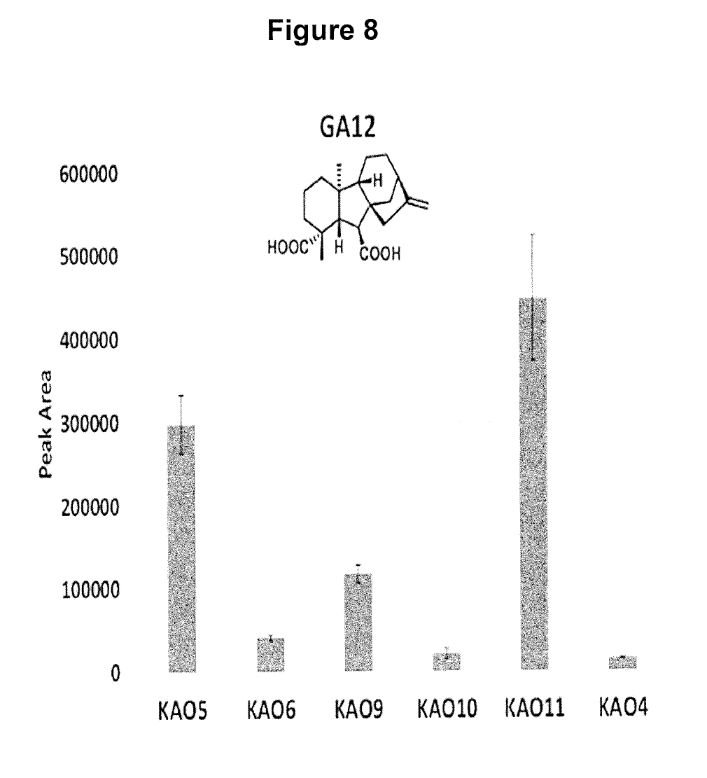

Using the USER™ cloning based yeast integration system, the genes in Table 11 were individually introduced into an S. cerevisiae strain that further comprised a gene encoding a G. fujikuroi CPR5 polypeptide (SEQ ID NO:47 (nt), SEQ ID NO:48 (aa)), a gene encoding a CPR12 polypeptide (SEQ ID NO:167 (nt), SEQ ID NO:168 (aa)), a gene encoding an A. thaliana KS5 polypeptide (SEQ ID NO:181 (nt), SEQ ID NO:182 (aa)), a gene encoding a truncated Zea mays CDPS polypeptide (SEQ ID NO:179 (nt), SEQ ID NO:180 (aa)), a gene encoding an ERG20-GGPPS7 polypeptide (SEQ ID NO:220 (nt), SEQ ID NO:221 (aa)), and a gene encoding a Stevia rebaudiana KO1 polypeptide (SEQ ID NO:171 (nt), SEQ ID NO:172 (aa)). See the pathway described in FIG. 7. GA₁₂ was accumulated upon expression of each of the KAO genes of Table 11 as well as C. maxima GA₇ₒₓ₋₁. See FIG. 8.

TABLE 11
KAO genes tested for production of gibberellins.

| KAO | SEQ ID NO |
|---|---|
| A. thaliana KAO5 | SEQ ID NO: 61 (nt) |
| | SEQ ID NO: 62 (aa) |
| A. thaliana KAO6 | SEQ ID NO: 59 (nt) |
| | SEQ ID NO: 60 (aa) |
| H. vulgare KAO9 | SEQ ID NO: 67 (nt) |
| | SEQ ID NO: 68 (aa) |
| P. sativum KAO10 | SEQ ID NO: 57 (nt) |
| | SEQ ID NO: 58 (aa) |
| P. sativum KAO11 | SEQ ID NO: 63 (nt) |
| | SEQ ID NO: 64 (aa) |
| S. manihoticola KAO4 | SEQ ID NO: 73 (nt) |
| | SEQ ID NO: 74 (aa) |

Co-expression of C. maxima GA₂₀ₒₓ₋₄ (SEQ ID NO:39 (nt), SEQ ID NO:40 (aa)), Oryza sativa GA₁₃ₒₓ (SEQ ID NO:97, SEQ ID NO:98), P. sativum KAO11 (SEQ ID NO:63 (nt), SEQ ID NO:64 (aa)), and C. maxima Ga₇ₒₓ₋₁ (SEQ ID NO:151 (nt), SEQ ID NO:152 (aa)) in the kaurenoic acid-producing S. cerevisiae strain further resulted in accumulation of GA₉ and GA₂₀. See the pathway described in FIG. 9A and graph in FIG. 9B. Additional gibberellins accumulated, including GA₁₂, GA₇, GA₄, GA₂₅, GA₂₄, and GA₁₃, as shown in FIG. 9B.

### Example 7. Activity of CYP117, CYP114, and CYP112 in GA₄- and GA₉-Producing S. Cerevisiae Strains

Using the USER™ cloning based yeast integration system, the genes in Table 12 or Table 13 were introduced into an S. cerevisiae strain that further comprised a gene encoding a G. fujikuroi CPR5 polypeptide (SEQ ID NO:47 (nt), SEQ ID NO:48 (aa)), a gene encoding a CPR12 polypeptide (SEQ ID NO:167 (nt), SEQ ID NO:168 (aa)), a gene encoding an A. thaliana KS5 polypeptide (SEQ ID NO:181 (nt), SEQ ID NO:182 (aa)), a gene encoding a truncated Z. mays CDPS polypeptide (SEQ ID NO:179 (nt), SEQ ID NO:180 (aa)), a gene encoding an ERG20-GGPPS7 polypeptide (SEQ ID NO:220 (nt), SEQ ID NO:221 (aa)), and a gene encoding a Stevia rebaudiana KO1 polypeptide (SEQ ID NO:171 (nt), SEQ ID NO:172 (aa)). See the pathways described in FIGS. 10 and 12. CYP112 (SEQ ID NO:123 (nt), SEQ ID NO:124 (aa)) was active in the presence of the KO of encoded by the nucleotide sequence set forth in SEQ ID NO:169. GA₉ was accumulated by the S. cerevisiae strain comprising KAO-11 (SEQ ID NO:63 (nt), SEQ ID NO:64 (aa)) and CYP112-KO anchor (SEQ ID NO:123 (nt), SEQ ID NO:124 (aa)). See FIG. 11. GA₄ was accumulated by the S. cerevisiae strain comprising KAO4 (SEQ ID NO:73 (nt), SEQ ID NO:74 (aa)) and the CYP112-KO anchor (SEQ ID NO:123 (nt), SEQ ID NO:124 (aa)). See FIG. 13.

TABLE 12
Genes expressed in S. cerevisiae strain “P.”

| | Gene 1 SEQ | | Gene 2 SEQ |
|---|---|---|---|
| Gene 1 | ID NOs | Gene 2 | ID NOs |
| P. sativum | SEQ ID NO: | C. maxima | SEQ ID NO: |
| KAO11 | 63 (nt) | GA₇ₒₓ | 151 (nt) |
| | SEQ ID NO: | | SEQ ID NO: |
| | 64 (aa) | | 152 (aa) |
| B. diazoefficiens | SEQ ID NO: | B. diazoefficiens | SEQ ID NO: |
| ADH | 115 (nt) | CYP112 | 123 (nt) |
| | SEQ ID NO: | | SEQ ID NO: |
| | 116 (aa) | | 124 (aa) |
| P. putida | SEQ ID NO: | P. putida | SEQ ID NO: |
| ferredoxin | 147 (nt) | ferredoxin | 149 (nt) |
| | SEQ ID NO: | reductase | SEQ ID NO: |
| | 148 (aa) | | 150 (aa) |

TABLE 13
Genes expressed in S. cerevisiae strain “U.”

| | Gene 1 SEQ | | Gene 2 SEQ |
|---|---|---|---|
| Gene 1 | ID NOs | Gene 2 | ID NOs |
| S. manihoticola | SEQ ID NO: | KO | SEQ ID NO: |
| KAO4 | 73 (nt) | | 169 (nt) |
| | SEQ ID NO: | | SEQ ID NO: |
| | 74 (aa) | | 170 (aa) |
| B. diazoefficiens | SEQ ID NO: | B. diazoefficiens | SEQ ID NO: |
| ADH | 115 (nt) | CYP112 | 123 (nt) |
| | SEQ ID NO: | | SEQ ID NO: |
| | 116 (aa) | | 124 (aa) |
| P. putida | SEQ ID NO: | P. putida | SEQ ID NO: |
| ferredoxin | 147 (nt) | ferredoxin | 149 (nt) |
| | SEQ ID NO: | reductase | SEQ ID NO: |
| | 148 (aa) | | 150 (aa) |

### Example 8. Expression of P450-1 Genes for Production of GA₁₄

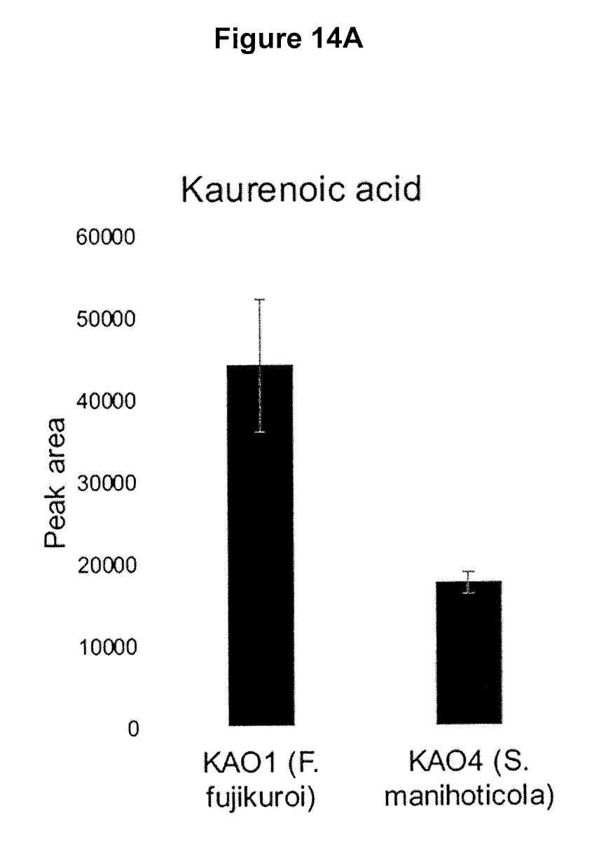

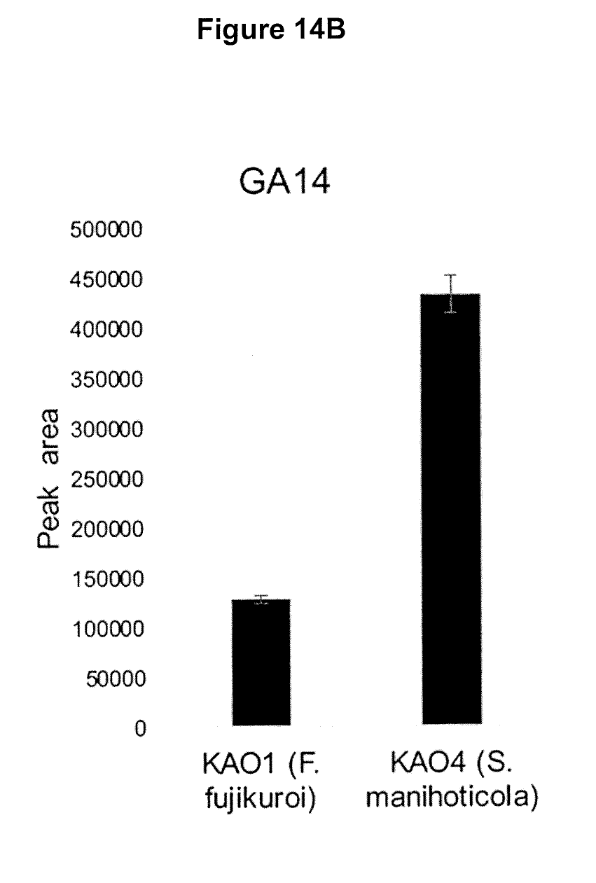

An S. cerevisiae strain comprising a gene encoding a P450-1 polypeptide (SEQ ID NO:87 (nt), SEQ ID NO:88 (aa)) or a P450-1 polypeptide (SEQ ID NO:145 (nt), SEQ ID NO:146 (aa)), a KAO4 polypeptide (SEQ ID NO:73 (nt), SEQ ID NO:74 (aa)), or a KAO1 polypeptide (SEQ ID NO:89 (nt), SEQ ID NO:90 (aa)) was engineered to accumulate kaurenoic acid, as described in Example 2. Using the USER™ based yeast integration vector system, S. manihoticola KAO4 polypeptide (SEQ ID NO:73 (nt), SEQ ID NO:74 (aa)) or G. fujikuroi KAO1 polypeptide (SEQ ID NO:89 (nt), SEQ ID NO:90 (aa)) individually introduced into the S. cerevisiae strain. As shown in FIGS. 14A and 14B, greater levels of kaurenoic were converted to GA₁₄ in the strain comprising KAO4 (SEQ ID NO:73, SEQ ID NO:74 (aa)), as compared to the strain comprising KAO1 (SEQ ID NO:89 (nt), SEQ ID NO:90 (aa)).

### Example 9. Engineering of S. Cerevisiae Strain Comprising Cytochrome B5 and Cytochrome B5 Reductase with CPR14, CPR15, or CPR16

Using the USER™ based yeast integration vector system, the genes in Table 14, Table 15, or Table 16 were introduced into an S. cerevisiae strain that further comprised a gene encoding a truncated CDPS polypeptide (SEQ ID NO:179 (nt), SEQ ID NO:180 (aa)), a KS polypeptide (SEQ ID NO:181 (nt), SEQ ID NO:182 (aa)), a KO polypeptide (SEQ ID NO:171 (nt), SEQ ID NO:172 (aa)), a CPR polypeptide (SEQ ID NO:167 (nt), SEQ ID NO:168 (aa)), and an ERG20-GGPPS7 polypeptide (SEQ ID NO:195 (nt), SEQ ID NO:196 (aa)). The strains described in Tables 14-16 were identical, except that they comprised either CPR14, CPR15, or CPR16.

TABLE 14
Genes expressed in S. cerevisiae strain “CPR16.”

| | Gene 1 SEQ | | Gene 2 SEQ |
|---|---|---|---|
| Gene 1 | ID NOs | Gene 2 | ID NOs |
| G. fujikuroi | SEQ ID NO: | G. fujikuroi | SEQ ID NO: |
| P450-2-1 | 79 (nt) | P450-3-4 | 185 (nt) |
| | SEQ ID NO: | | SEQ ID NO: |
| | 80 (aa) | | 186 (aa) |
| A. niger | SEQ ID NO: | G. fujikuroi | SEQ ID NO: |
| CPR16 | 157 (nt) | DES-1 | 25 (nt) |
| | SEQ ID NO: | | SEQ ID NO: |
| | 158 (aa) | | 26 (aa) |
| G. fujikuroi | SEQ ID NO: | G. fujikuroi | SEQ ID NO: |
| Cytochrome B5 | 159 (nt) | Cytochrome B5 | 01 (nt) |
| | SEQ ID NO: | reductase | SEQ ID NO: |
| | 160 (aa) | | 02 (aa) |

TABLE 15
Genes expressed in S. cerevisiae strain “CPR14.”

| | Gene 1 SEQ | | Gene 2 SEQ |
|---|---|---|---|
| Gene 1 | ID NOs | Gene 2 | ID NOs |
| G. fujikuroi | SEQ ID NO: | G. fujikuroi | SEQ ID NO: |
| P450-2-1 | 79 (nt) | P450-3-4 | 185 (nt) |
| | SEQ ID NO: | | SEQ ID NO: |
| | 80 (aa) | | 186 (aa) |
| Phaeosphaeria sp. | SEQ ID NO: | G. fujikuroi | SEQ ID NO: |
| CPR14 | 99 (nt) | DES-1 | 25 (nt) |
| | SEQ ID NO: | | SEQ ID NO: |
| | 100 (aa) | | 26 (aa) |
| G. fujikuroi | SEQ ID NO: | G. fujikuroi | SEQ ID NO: |
| Cytochrome B5 | 159 (nt) | Cytochrome B5 | 01 (nt) |
| | SEQ ID NO: | reductase | SEQ ID NO: |
| | 160 (aa) | | 02 (aa) |

TABLE 16
Genes expressed in S. cerevisiae strain “CPR15.”

| | Gene 1 SEQ | | Gene 2 SEQ |
|---|---|---|---|
| Gene 1 | ID NOs | Gene 2 | ID NOs |
| G. fujikuroi | SEQ ID NO: | G. fujikuroi | SEQ ID NO: |
| P450-2-1 | 79 (nt) | P450-3-4 | 185 (nt) |
| | SEQ ID NO: | | SEQ ID NO: |
| | 80 (aa) | | 186 (aa) |
| Candida | SEQ ID NO: | G. fujikuroi | SEQ ID NO: |
| apicola | 139 (nt) | DES-1 | 25 (nt) |
| CPR15 | SEQ ID NO: | | SEQ ID NO: |
| | 140 (aa) | | 26 (aa) |
| G. fujikuroi | SEQ ID NO: | G. fujikuroi | SEQ ID NO: |
| Cytochrome | 159 (nt) | Cytochrome | 01 (nt) |
| B5 | SEQ ID NO: | B5 reductase | SEQ ID NO: |
| | 160 (aa) | | 02 (aa) |

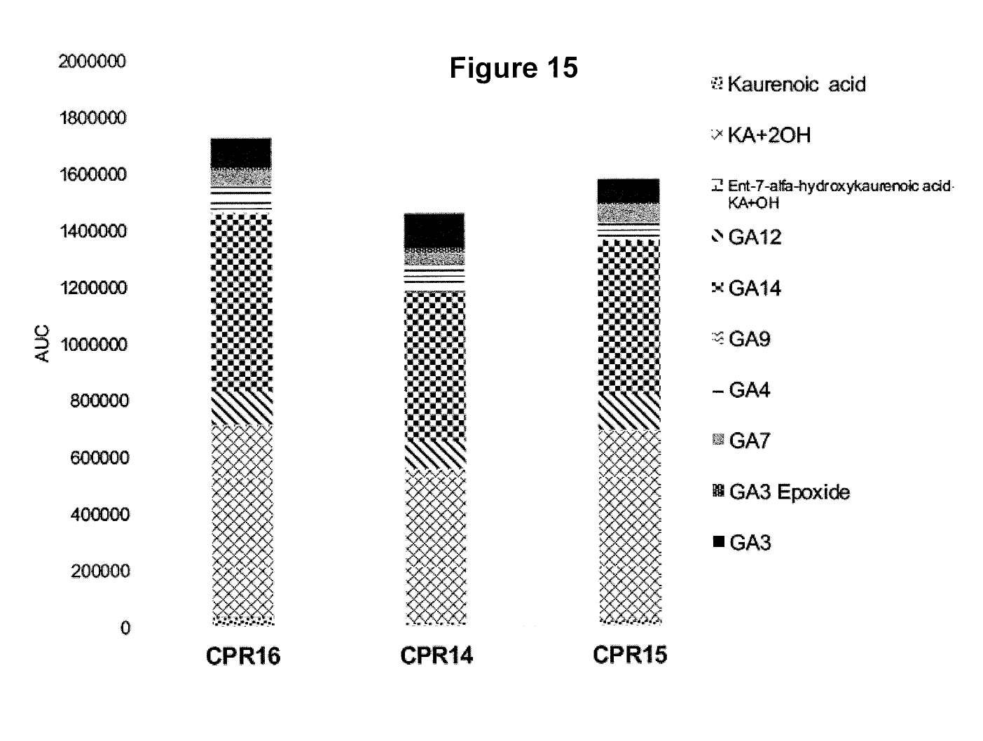

As shown in FIG. 15, each of the strains accumulated gibberellins, including, but not limited to, GA₃, GA₄, GA₇, GA₁₂, and GA₁₄ (see also, FIG. 4B). Thus, expression of G. fujikuroi cytochrome B5 and G. fujikuroi cytochrome B5 reductase boosts production of gibberellins.

### Example 10. Engineering of S. Cerevisiae Strain for Production of Gibberellin A4 (GA₄)

Using the USER™ based yeast integration vector system, the genes in Table 17 were stably integrated into an S. cerevisiae strain. The strain was grown in a 2 L Sartorius fermentor using a fed batch process. Temperature, pH, agitation, and aeration rate were controlled throughout the cultivation. The temperature was maintained at 30° C. Air was used for sparging the bioreactor at 1 vvm (L gas/(L liquid×min)). pH was controlled at pH 5.0 by automatic addition of NH₄OH. An 8% NH₄OH solution was used for the first 45 hours of the process; a 16% solution was used for the final part. The stirrer speed was initially set to 800 rpm and increased to up to 1600 rpm during the process. The basis for the medium used for the batch phase is 0.5 L minimal medium containing glucose, salts, vitamins and trace metals. The feed solution was either a high density glucose solution with salts, trace metals and vitamins (glucose feed) or 96% ethanol (ethanol feed). Antifoam was included in the batch medium and feed medium. The fermentation was inoculated using a seed train in shake flasks grown at 30° C. using a minimal medium with similar content as the medium used for the batch phase in the fermentation. The batch fermentation lasted for 16 hours. During the carbon-limited fed batch phase, feed was added following an exponential feed profile feeding with glucose feed from 16-70 hours and ethanol feed from 70-160 hours. Since the ethanol feed only contained the carbon source, concentrated feed components (salts, vitamins, trace metals and antifoam) were combined, sterile filtered and added to the fermentation broth once or twice per day during feeding with ethanol feed.

TABLE 17
Genes integrated in S. cerevisiae strain
for production of gibberellin A4 (GA₄).

| Gene | SEQ ID NOs |
|---|---|
| DAP1-2 | SEQ ID NO: 212 (nt) |
| F. fujikuroi | SEQ ID NO: 213 (aa) |
| ICE2-2 | SEQ ID NO: 205 (nt) |
| F. fujikuroi | SEQ ID NO: 206 (aa) |
| CDPS-K56 | SEQ ID NO: 101 (nt) |
| F. fujikuroi | SEQ ID NO: 102 (aa) |
| KS5 | SEQ ID NO: 181 (nt) |
| A. thaliana | SEQ ID NO: 182 (aa) |
| FfCytB5-1 | SEQ ID NO: 159 (nt) |
| (codon optimized) | SEQ ID NO: 160 (aa) |
| F. fujikuroi | |
| KAO3 | SEQ ID NO: 145 (nt) |
| G. fujikuroi | SEQ ID NO: 146 (aa) |
| CPR19 | SEQ ID NO: 193 (nt) |
| G. fujikuroi | SEQ ID NO: 194 (aa) |
| CPR12 | SEQ ID NO: 167 (nt) |
| R. suavissimus | SEQ ID NO: 168 (aa) |
| RsKO | SEQ ID NO: 169 (nt) |
| R. suavissimus | SEQ ID NO: 170 (aa) |
| GGPPS-7 | SEQ ID NO: 177 (nt) |
| Synecococcus sp. | SEQ ID NO: 178 (aa) |
| KO1 | SEQ ID NO: 171 (nt) |
| S. rebaudiana | SEQ ID NO: 172 (aa) |
| P450-2-1 | SEQ ID NO: 79 (nt) |
| G. fujikuroi | SEQ ID NO: 80 (aa) |

As shown in FIG. 16, the strain accumulated gibberellins, including, but not limited to GA₄ and GA₁₄. After approximately 160 hours of fermentation, the titer in growth medium was 2.2 g/L of GA₄, 55 mg/L of GA₁₄ and 2.3 g/L of KA; 1.04 mM of kaurenol, 4.65 mM of kaurenal and 1.12 mM ent-kaurene. The production of additional gibberellins and intermediate molecules at various time points during growth in the culture medium is shown in Table 18.

TABLE 18
Production of additional
gibberellins and intermediates.

| Time point | GA₄ | GA₁₄ | KA |
|---|---|---|---|
| (hrs) | (μM) | (μM) | (μM) |
| 0 | 0 | 0 | 0 |
| 47 | 642 | 44 | 15 |
| 71 | 1313 | 54 | 79 |
| 95 | 2600 | 120 | 1308 |
| 100 | 2800 | 118 | 1754 |
| 119 | 4659 | 96 | 3090 |
| 125 | 4900 | 88 | 4666 |
| 143 | 5462 | 80 | 6138 |
| 149 | 5845 | 73 | 6898 |
| 167 | 6644 | 88 | 7608 |

### Example 11. Production of GA₃ Using Fungal Gibberellin Pathway Genes

Using the USER™ based yeast integration vector system, the genes in Table 19 were stably integrated into an S. cerevisiae strain. The strain was grown in a 2 L Sartorius fermentor using a fed batch process. Temperature, pH, agitation, and aeration rate were controlled throughout the cultivation. The temperature was maintained at 30° C. Air was used for sparging the bioreactor at 1 vvm (L gas/(L liquid×min)). pH was controlled at pH 5.0 by automatic addition of NH₄OH. An 8% NH₄OH solution was used for the first 45 hours of the process; a 16% solution was used for the final part. The stirrer speed was initially set to 800 rpm and increased to up to 1600 rpm during the process. The basis for the medium used for the batch phase is 0.5 L minimal medium containing glucose, salts, vitamins and trace metals. The feed solution was either a high density glucose solution with salts, trace metals and vitamins (glucose feed) or 96% ethanol (ethanol feed). Antifoam was included in the batch medium and feed medium. The fermentation was inoculated using a seed train in shake flasks grown at 30° C. using a minimal medium with similar content as the medium used for the batch phase in the fermentation. The batch fermentation lasted for 16 hours. During the carbon-limited fed batch phase, feed was added following an exponential feed profile feeding with glucose feed from 16-70 hours and ethanol feed from 70-148 hours. Since the ethanol feed only contained the carbon source, concentrated feed components (salts, vitamins, trace metals and antifoam) were combined, sterile filtered and added to the fermentation broth once or twice per day during feeding with ethanol feed. After 148 hours of fermentation, the titer in growth medium was measured to be 491 mg/L (1.42 mM) of GA₃ and 2.15 mM of kaurenol, 4.26 mM of kaurenal and 1.28 mM ent-kaurene. The production of GA₃, GA₄ and additional gibberellins and intermediate molecules at various time points during growth in the culture medium is shown in Table 20. The results demonstrate that a yeast strain comprising fungal gibberellin genes can produce gibberellins.

TABLE 19
Genes integrated in S. cerevisiae strain
for production of gibberellins, including GA₃

| Gene | SEQ ID NOs |
|---|---|
| DAP1-2 | SEQ ID NO: 212 (nt) |
| F. fujikuroi | SEQ ID NO: 213 (aa) |
| ICE2-2 | SEQ ID NO: 205 (nt) |
| F. fujikuroi | SEQ ID NO: 206 (aa) |
| CDPS-KS-6 | SEQ ID NO: 101 (nt) |
| F. fujikuroi | SEQ ID NO: 102 (aa) |
| KS5 | SEQ ID NO: 181 (nt) |
| A. thaliana | SEQ ID NO: 182 (aa) |
| FfCytB5-1 | SEQ ID NO: 159 (nt) |
| (codon optimized) | SEQ ID NO: 160 (aa) |
| F. fujikuroi | |
| FfCytB5red-1 | SEQ ID NO: 01 (nt) |
| (codon optimized) | SEQ ID NO: 02 (aa) |
| F. fujikuroi | |
| KAO3 | SEQ ID NO: 145 (nt) |
| G. fujikuroi | SEQ ID NO: 146 (aa) |
| CPR19 | SEQ ID NO: 193 (nt) |
| G. fujikuroi | SEQ ID NO: 194 (aa) |
| CPR12 | SEQ ID NO: 167 (nt) |
| R. suavissimus | SEQ ID NO: 168 (aa) |
| RsKO | SEQ ID NO: 169 (nt) |
| R. suavissimus | SEQ ID NO: 170 (aa) |
| GGPPS-7 | SEQ ID NO: 177 (nt) |
| Synecococcus sp. | SEQ ID NO: 178 (aa) |
| KO1 | SEQ ID NO: 171 (nt) |
| S. rebaudiana | SEQ ID NO: 172 (aa) |
| P450-2-1 | SEQ ID NO: 79 (nt) |
| G. fujikuroi | SEQ ID NO: 80 (aa) |
| KAO4 | SEQ ID NO: 73 (nt) |
| S. manihoticola | SEQ ID NO: 74 (aa) |
| DES-1 | SEQ ID NO: 25 (nt) |
| F. fujikuroi | SEQ ID NO: 26 (aa) |

TABLE 20
Gibberellin production in samples.

| | GA₁ | GA₃ | GA₄ | GA₁₃ | GA₁₄ | GA₂₅ | KA |
|---|---|---|---|---|---|---|---|
| Hours | (mM) | (mM) | (mM) | (mM) | (mM) | (mM) | (mM) |
| 0 | 0 | 0 | 0 | 0 | 0 | 0 | 0 |
| 46 | 0 | 0.261 | 0.0219 | 0.025 | 0.0504 | 0.00166 | 0.1723 |
| 71 | 0.0475 | 0.7185 | 0.0479 | 0.087 | 0.111 | 0.0482 | 1.472 |
| 94 | 0.2074 | 0.8399 | 0.1604 | 0.119 | 0.2169 | 0.114 | 1.486 |
| 118 | 0.4897 | 1.1385 | 0.1726 | 0.187 | 0.3098 | 0.187 | 2.836 |
| 124 | 0.5221 | 1.3315 | 0.1693 | 0.215 | 0.3197 | 0.203 | 2.825 |
| 142 | 0.547 | 1.3452 | 0.1438 | 0.224 | 0.1983 | 0.187 | 2.6623 |
| 148 | 0.681 | 1.4241 | 0.1684 | 0.257 | 0.1985 | 0.209 | 3.833 |

### Example 12. Engineering of S. Cerevisiae Strain for Production of Gibberellin Å₃ (GA₃) Comprising Plant GA₃ₒₓ Genes

Using the USER™ based yeast integration vector system, the genes in Table 19 were stably integrated into an S. cerevisiae strain. The strain was grown in DELFT culture medium supplemented with uracil to complement uracil auxotrophy of the strain for 96 hours. Samples were extracted with acetonitrile (80% final) and cultures were analysed using LC-MS. The production of GA₄ and additional gibberellins and intermediate molecules at various time points during growth in the culture medium is shown in Table 22.

TABLE 21
Genes integrated in S. cerevisiae strain
for production of Gibberellins, including GA₃.

| Gene | SEQ ID NOs |
|---|---|
| DAP1-2 | SEQ ID NO: 212 (nt) |
| F. fujikuroi | SEQ ID NO: 213 (aa) |
| CytB5-2 | SEQ ID NO: 238 (nt) |
| | SEQ ID NO: 239 (aa) |
| CytB5red-4 | SEQ ID NO: 240 (nt) |
| | SEQ ID NO: 241 (aa) |
| FfCytB5-1 | SEQ ID NO: 159 (nt) |
| (codon optimized) | SEQ ID NO: 160 (aa) |
| F. fujikuroi | |
| FfCytB5red-1 | SEQ ID NO: 01 (nt) |
| (codon optimized) | SEQ ID NO: 02 (aa) |
| F. fujikuroi | |
| KAO11 | SEQ ID NO: 63 (nt) |
| P. sativum | SEQ ID NO: 64 (aa) |
| CPR12 | SEQ ID NO: 167 (nt) |
| R. suavissimus | SEQ ID NO: 168 (aa) |
| CDPS-K56 | SEQ ID NO: 101 (nt) |
| F. fujikuroi | SEQ ID NO: 102 (aa) |
| KS5 | SEQ ID NO: 181 (nt) |
| A. thaliana | SEQ ID NO: 182 (aa) |
| GGPPS-7 | SEQ ID NO: 177 (nt) |
| Synecococcus sp. | SEQ ID NO: 178 (aa) |
| KO1 | SEQ ID NO: 171 (nt) |
| S. rebaudiana | SEQ ID NO: 172 (aa) |
| GA13ox-1 | SEQ ID NO: 97 (nt) |
| O. sativa | SEQ ID NO: 98 (aa) |
| GA20ox-4 | SEQ ID NO: 39 (nt) |
| C. maxima | SEQ ID NO: 40 (aa) |
| GA3ox-1 | SEQ ID NO: 27 (nt) |
| M. macrocarpus | SEQ ID NO: 28 (aa) |

TABLE 22
Gibberellin production in samples.

| Sample | | | | | GA₄ | | | Kaurenoic |
|---|---|---|---|---|---|---|---|---|
| Name | Genes added to strain | GA₁₂ | GA₂₀ | GA₄ | (μM) | GA₅₃ | GA₉ | acid (μM) |
| A8 | GA13-1 + GA20-4 + | 131195 | | 12295 | 1.7 | 2650 | 9515 | 142 |
| | GA3-1 + CPR12 | | | | | | | |
| B1 | GA13-1 + GA20-4 + | 49045 | 1655 | 20950 | 2.8 | 56745 | — | 267 |
| | GA3-1 | | | | | | | |
| C5 | GA13-1 + GA20-4 + | 52565 | 12895 | 1000 | 0.4 | 25460 | 48505 | 248 |
| | GA3-2 + CPR12 | | | | | | | |
| D12 | GA13-1 + GA20-4 + | 37435 | 8295 | 1810 | 0.5 | 36520 | 39465 | 222 |
| | GA3-2 | | | | | | | |
| E11 | GA13-1 + GA20-4 + | 44830 | 9910 | — | 0.0 | 21450 | 46010 | 214 |
| | GA3-3 + CPR12 | | | | | | | |
| F7 | GA13-1 + GA20-4 + | 33515 | 14010 | — | 0.0 | 26235 | 33335 | 220 |
| | GA3-3 | | | | | | | |
| G4 | GA13-1 + GA20-4 + | 28910 | 8860 | 26495 | 3.6 | 7845 | 25180 | 108 |
| | GA3-4 + CPR12 | | | | | | | |
| H9 | GA13-1 + GA20-4 + | 34550 | 9990 | 16545 | 2.2 | 31830 | 4160 | 144 |
| | GA3-4 | | | | | | | |
| | Values are AUC | | | | | | | |
| | values, except for | | | | | | | |
| | GA(uM) and Kaurenoic | | | | | | | |
| | acid (uM) | | | | | | | |

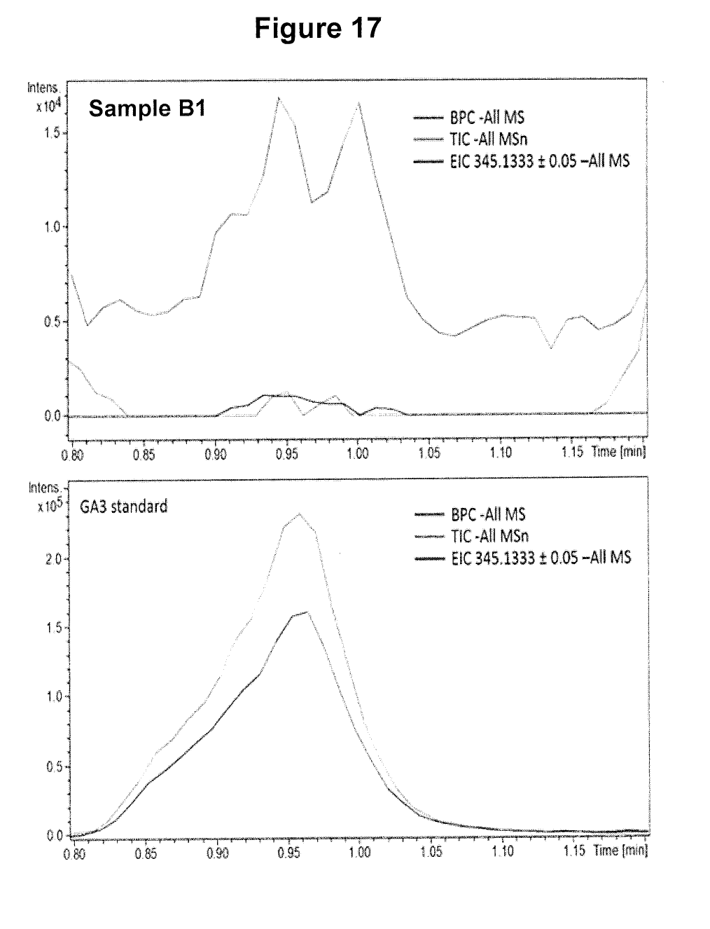

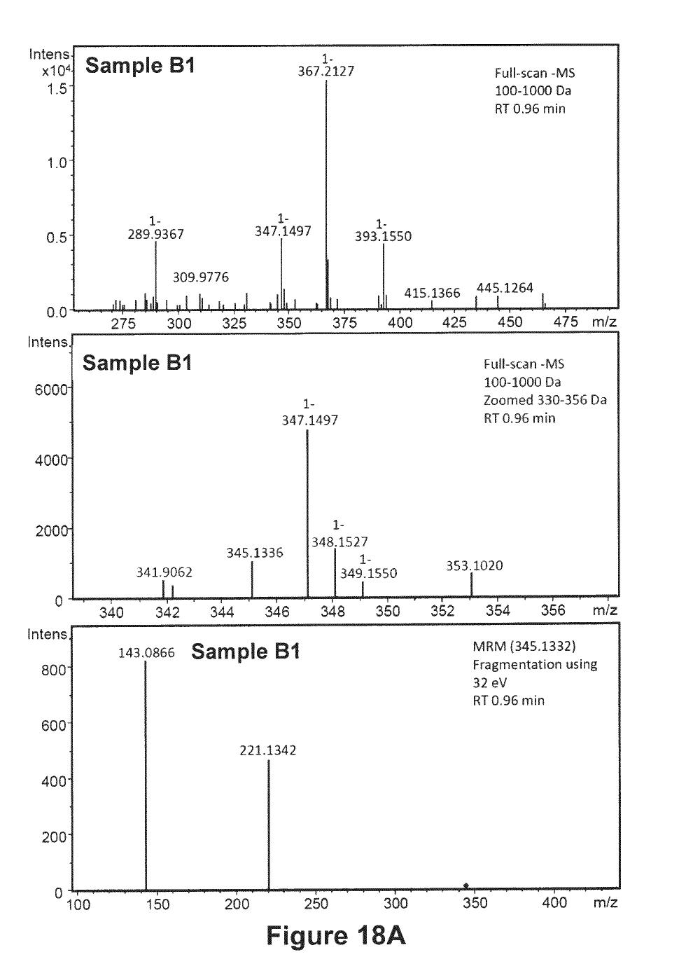

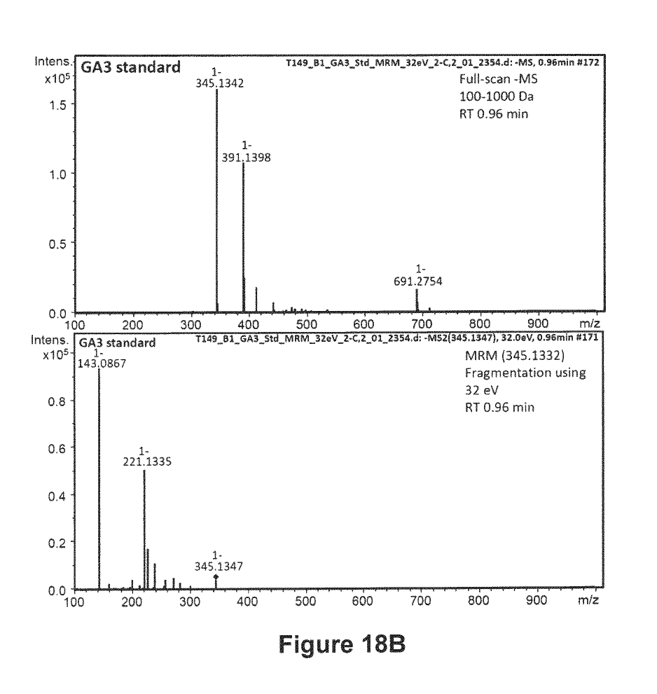

These results demonstrated that plant GA13 ox, GA20 ox and GA3 ox genes were all active in yeast and that when combined they can catalyse the reactions from GA₁₂ to GA₅₃ (GA13 ox reaction) to GA₉ (GA20 ox reaction) to GA₂₀ (GA13 ox+GA20 ox reactions via either GA₅₃ or GA₉) and then further GA₉ to GA₄ reaction catalyzed by GA₃ₒₓ genes. Further analysis revealed that sample B1 and sample C5 also contained small amounts of GA₃, which thereby demonstrated a fully functional GA₃ pathway from ent-kaurene based on plant derived genes (see FIG. 17 and FIG. 18). Mass spectra corresponding to the peaks with RT 0.96 were extracted as seen in FIG. 17. The signal detected at m/z 345.1336 fit with the mass of GA₃ (2.1 ppm error). To further investigate, samples were analyzed using MRM to investigate fragment formation. Using a collision energy of 32 eV, ions with m/z were isolated and fragmented. MS/MS spectra can be seen in FIG. 18.

### Example 13. Production of Gibberellin Å₃ (GA₃) and Other Gibberellins Using a S. Cerevisiae Strain Comprising Plant GA20 Oxidase, GA3 Oxidase and GA13 Oxidase Genes

The “B1” strain from Example 12 was grown in a 2 L Sartorius fermentor using a fed batch process. Temperature, pH, agitation, and aeration rate were controlled throughout the cultivation. The temperature was maintained at 30° C. Air was used for sparging the bioreactor at 1 vvm (L gas/(L liquid×min)). pH was controlled at pH 5.0 by automatic addition of NH₄OH. An 8% NH₄OH solution was used for the first 45 hours of the process; a 16% solution was used for the final part. The stirrer speed was initially set to 800 rpm and increased to up to 1600 rpm during the process. The basis for the medium used for the batch phase is 0.5 L minimal medium containing glucose, salts, vitamins and trace metals. The feed solution was either a high density glucose solution with salts, trace metals and vitamins (glucose feed) or 96% ethanol (ethanol feed) supplemented with uracil to complement uracil auxotrophy of the strain. Antifoam was included in the batch medium and feed medium. The fermentation was inoculated using a seed train in shake flasks grown at 30° C. using a minimal medium with similar content as the medium used for the batch phase in the fermentation. The batch fermentation lasted for 16 hours. During the carbon-limited fed batch phase, feed was added following an exponential feed profile feeding with glucose feed from 16-70 hours and ethanol feed from 70-138 hours. Since the ethanol feed only contained the carbon source, concentrated feed components (salts, vitamins, trace metals and antifoam) were combined, sterile filtered and added to the fermentation broth once or twice per day during feeding with ethanol feed.

As shown in Table 23, the strain accumulated gibberellins, including, but not limited to GA₃, GA₄ and GA₁₄. After approximately 138 hours of fermentation, the titer in growth medium was 1.7 μM of GA₃, 73 μM of GA₁, 82 μM of GA₄, 1.8 μM GA₇, 2400 μM of KA as well as estimated amounts of 214 μM of GA₂₀, 1.5 μM of GA₉, 134 μM of GA₂₄, 128 μM of GA₅₃ and 142 μM of GA₁₂. The production of additional gibberellins and intermediate molecules at various time points during growth in the culture medium is shown in Table 23.

TABLE 23
Production of additional gibberellins and intermediates.

| Time | | | | | | | | | | |
|---|---|---|---|---|---|---|---|---|---|---|
| point | GA₃ | GA₁ | GA₇ | GA₄ | KA | GA₂₀ | GA₉ | GA₂₄ | GA₅₃ | GA₁₂ |
| (hrs) | (μM) | (μM) | (μM) | (μM) | (μM) | (μM) | (μM) | (μM) | (μM) | (μM) |
| 116 h | 1.4 | 47 | 0 | 48 | 388 | 4.7 | 2.5 | 32 | 38 | 39 |
| 138 h | 1.7 | 73 | 1.8 | 482 | 2400 | 214 | 1.5 | 134 | 128 | 142 |

### Example 14. Engineering of S. Cerevisiae Strain for Production of Gibberellin A₃ (GA₃) Comprising Plant GA₁₃ₒₓ Genes

Using the USER™ based yeast integration vector system, the genes in Table 24 and Table 25 were stably integrated into an S. cerevisiae strain comprising the genes as shown in Table 17. The strain was grown in DELFT culture medium for 96 hours. Samples were extracted with acetonitrile (80% final) and cultures were analyzed using LC-MS. By testing plant GA₁₃ oxidase in a GA₄ producing strain, the results demonstrate that the plant GA₁₃ oxidase can replace the fungal P450-3 enzyme, which is demonstrated by the formation of GA₁ and GA₃. See Table 26.

TABLE 24
Genes integrated in S. cerevisiae
strain for production of Gibberellins
(Transformants H1, H2 and H3)

| Gene | SEQ ID No. |
|---|---|
| GA13ₒₓ₋₁ | SEQ ID NO: 97 (nt) |
| Oryza sativa | SEQ ID NO: 98 (aa) |
| DES-1 | SEQ ID NO: 25 (nt) |
| Fusarium fujikuroi | SEQ ID NO: 26 (aa) |

TABLE 25
Genes integrated in S. cerevisiae strain
for production of Gibberellins
(Transformants I1, I2 and I3)

| Gene | SEQ ID No. |
|---|---|
| P450-3-4 | SEQ ID NO: 185 (nt) |
| Fusarium fujikuroi | SEQ ID NO: 186 (aa) |
| DES-1 | SEQ ID NO: 25 (nt) |
| Fusarium fujikuroi | SEQ ID NO: 26 (aa) |

TABLE 26
Production of additional gibberellins and intermediates.

| | Unit: | | | | | | | | | | | | |
|---|---|---|---|---|---|---|---|---|---|---|---|---|---|
| | AUC | | μM | | | | | μM | | | | | AUC |
| | Epoxide | AUC | GA1 | AUC | AUC | AUC | AUC | GA3 | AUC | AUC | AUC | AUC | Kaurenoic |
| Sample | GA3 | GA1 | (μM) | GA12 | GA13 | GA14 | GA3 | (μM) | GA4 | GA53 | GA7 | GA9 | acid |
| H1 | | 37360 | 13 | 30740 | 67050 | 205185 | 3180 | 0.40 | 144395 | 29615 | 235460 | 6705 | 895 |
| H2 | | 58995 | 20 | 19280 | 71720 | 106075 | 1955 | 0.10 | 248700 | 43880 | 205265 | 8160 | |
| H3 | | 60405 | 21 | 22755 | 52950 | 82190 | 800 | 0.04 | 345500 | 42085 | 91190 | 6575 | |
| I1 | 10760 | 85885 | 30 | 45290 | 85105 | 103710 | 87870 | 22.10 | 114315 | 0 | 6495 | 9955 | |
| I2 | 8265 | 62160 | 21 | 28320 | 79895 | 73755 | 63840 | 15.90 | 145540 | 0 | 7630 | 8395 | |
| I3 | 11700 | 91795 | 32 | 56205 | 92035 | 111315 | 90000 | 22.70 | 119980 | 0 | 6840 | 11305 | |

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

TABLE 27
Sequences disclosed herein.

SEQ ID NO: 1

| | |
|---|---|
| atgtcctcta acggtgataa ccattctttg ttcgccagac attacatcga ttatgtttat | 60 |
| gctccaggtt tgttgttggt tgtcggtact ttgatcgtta agaaagaatg ggctccatgg | 120 |
| gctttgttag ttgctgttgt ttttggtatc tacaacttca tggccttcca agttaagact | 180 |
| actttgaagc cagatgtttt ccaagaattt gaattggaag aaaagaccat cgtcagtcat | 240 |
| aacgttgcta tctacagatt caaattgcca tccccaaaac acattttggg tttgccaatt | 300 |
| ggtcaacaca tttctattgg tgctccatgt ccacaaccag atggtactac aaaagaaatc | 360 |
| gttagatcct acaccccaat ctctggtgat catcaaccag gtcatgttga tttgttgatc | 420 |
| aagtcttacc cacaaggtaa catctccaaa catatggcat ctttgactgt tggtcaaacc | 480 |
| attaaggtta gaggtccaaa aggtgctttt gtctacactc caaatatggt tagacacttc | 540 |
| ggtatgattg ctggtggtac tggtattact ccaatgttgc aagttattag agccatcgtt | 600 |
| agaggtagag ctgctggtga taagactgaa gttgatttga ttttcgctaa cgttaccgcc | 660 |
| caagacatct tgttgaaaga agatttggac gctttggcca agcaagattc tggtattaga | 720 |
| gttcattacg tcttggacaa acctgaagaa ggttggactg gtggtgttgg ttatgttact | 780 |
| gctgatatga tcgataagta cttgccaaaa ccagccgatg atgttaagat tttgttgtgt | 840 |
| ggtccaccac caatgatttc tggtttgaaa aaagctaccg aatccttggg ttttaagaag | 900 |
| gctagaccag tttctaagtt ggttgaccaa gttttcgctt tttaa | 945 |

SEQ ID NO: 2

| | |
|---|---|
| MSSNGDNHSL FARHYIDYVY APGLLLVVGT LIVKKEWAPW ALLVAVVFGI YNFMAFQVKT | 60 |
| TLKPDVFQEF ELEEKTIVSH NVAIYRFKLP SPKHILGLPI GQHISIGAPC PQPDGTTKEI | 120 |
| VRSYTPISGD HQPGHVDLLI KSYPQGNISK HMASLTVGQT IKVRGPKGAF VYTPNMVRHF | 180 |
| GMIAGGTGIT PMLQVIRAIV RGRAAGDKTE VDLIFANVTA QDILLKEDLD ALAKQDSGIR | 240 |
| VHYVLDKPEE GWTGGVGYVT ADMIDKYLPK PADDVKILLC GPPPMISGLK KATESLGFKK | 300 |
| ARPVSKLVDQ VFAF | 314 |

SEQ ID NO: 3

| | |
|---|---|
| atgtcagggc aatctctgcc aacactacct atgtggcgtg ttgatcatat agaaccgagt | 60 |
| cccgaaatgt tggcactgag ggctaatggt ccaatccata gggtaaggtt tccgtctggg | 120 |
| cacgagggtt ggtgggtgac aggttacgaa gaggccaagg cagtgttgag cgacgccgct | 180 |
| tttagaccat ccggtatgcc gccagcagca ttcacacccg caacagtcat acttggttcc | 240 |
| ccaggttggt tgggaagtca tgaaggttct gaacatgcaa gattgagaac aattgtagct | 300 |
| cccgcatttt caaatagacg tgtgaagcta ctagcacaac agatcgaagc aattgctgca | 360 |
| caattgtttg aaacgctagc agcacaacct cagcccgctg atctgagaca ttacttatcc | 420 |
| tttcctcttc ctgctatggt gattagtgcc ttgatgggtg taccatatga agatcacgct | 480 |
| ttttttgcag aacttagtga cgaagttatg acccaccaac atgagtccgg tcctagaagc | 540 |
| gctgcgctac tggcatgggg agagttaagg acctacatca gaggcaaaat gagggggaaa | 600 |
| agacaagacc caggagataa tctacttact gacttacttg ctgccgttga tcagggcaag | 660 |
| gcaactgagg aagaagccat aggtcttgct gcaggaattc ttgttgcagg ccacgaatca | 720 |
| actgttgcac aaatagaatt tggtttactg gctatgtcca gacaccctca tcagcgtgag | 780 |
| agattagttg gagatccatc tttagtcgac aaggcagtgg aggaaatttt acgtatgtac | 840 |
| cctccaggcg ccggatggga tggtattatg agatatccta gaactgatgt gacaatagcg | 900 |
| ggggttcata ttccagctga aagcaaagtg ttagttggct tgcctgccac aagttttgat | 960 |
| ccccatcact tcgacgatcc tgagaacttt gatataggaa gagcagaaaa gcctcactta | 1020 |
| gctttttcat atggtcctca ttattgcatt ggtgaagcct tggcacgttt agaacttaag | 1080 |
| gtagtctttg gttccatctt tcaaagattc ccgacgttgc gtttggctgt cgcacccgaa | 1140 |
| gagttaaagt taagaaagga tataatcaca ggaggattcg aagaattccc cgtattatgg | 1200 |
| taa | 1203 |

SEQ ID NO: 4

| | |
|---|---|
| MSGQSLPTLP MWRVDHIEPS PEMLALRANG PIHRVRFPSG HEGWWVTGYE EAKAVLSDAA | 60 |
| FRPSGMPPAA FTPATVILGS PGWLGSHEGS EHARLRTIVA PAFSNRRVKL LAQQIEAIAA | 120 |
| QLFETLAAQP QPADLRHYLS FPLPAMVISA LMGVPYEDHA FFAELSDEVM THQHESGPRS | 180 |
| AALLAWGELR TYIRGKMRGK RQDPGDNLLT DLLAAVDQGK ATEEEAIGLA AGILVAGHES | 240 |
| TVAQIEFGLL AMSRHPHQRE RLVGDPSLVD KAVEEILRMY PPGAGWDGIM RYPRTDVTIA | 300 |
| GVHIPAESKV LVGLPATSFD PHHFDDPENF DIGRAEKPHL AFSYGPHYCI GEALARLELK | 360 |
| VVFGSIFQRF PTLRLAVAPE ELKLRKDIIT GGFEEFPVLW | 400 |

SEQ ID NO: 5

| | |
|---|---|
| atgttcgaac agcctttgcc gaccttgccg atgtggagag ttgatcacat cgaaccttct | 60 |
| cctgagatgt tagctctaag ggctaaaggt ccaatccata gagtgcgttt tccttcaggg | 120 |
| catgagggat ggtgggttac tggttacgac gaagctcaag cagttttatc agatgctgcc | 180 |
| tttagaccag ccggtatgcc tccagaaaca tttacaccgg attcagttat tttgggtagt | 240 |
| ccaggttggc ttgtatctca cgaaggaggt aaacacgctt ggctaagaat gattgttgcc | 300 |
| ccagcattct caaataggag ggtgaaattg ttagcccaac aagtcgaggc catagctgct | 360 |
| caattgttcg aaacactggc tgctcaacca caaccagccg atttaagaag acacttatca | 420 |
| tttccattgc cagctatggt gatttcagca ctaatgggcg ttttatatga agatcatata | 480 |
| ttcttcgccg gtttatcaga cgaagtcatg acccaccaac atgagtccgg cccgagatct | 540 |
| gccagcagag tcgcttggga agagcttaga acctacattt gcagaaagat gagaggtaag | 600 |
| agggaagagc caggtgacaa tttacttacc gatttgttgg cggctgtgga tcatggcaaa | 660 |
| gcaactgaag aagaggcagt tggtttggct gccggtgttc ttgtagcagg ccatgaaagt | 720 |
| actgtagctc aaattgaatt tggcctgtta gctatgttca ggcaccccca acaaagggag | 780 |
| agattggtta gagacccatt cctagccgat aaagctgtag aggaaatttt aagaatgtac | 840 |
| agccccggcg ctggttggga tggcattatg agatacccta gaactgatgt cactatagct | 900 |
| ggtatggaca ttcccgccga atcaaaagtc ttagtgggtt tacctgccac ttcattcgac | 960 |
| ccaaggcact tcgaagatcc ggaagtattt gatataggta gggatccaaa cccacaccta | 1020 |
| gcgttttcct atggcccaca caattgcatc ggtgcagcat tggctagact tgaattaaaa | 1080 |
| gtggtatttg gttccatatt ccagagattc ccggccctaa ggctagctgt agctccagaa | 1140 |
| gaactgaagt tgagaaaaga aataattacg ggcgggtttg aagaatttcc agtcctatgg | 1200 |

SEQ ID NO: 6

| | |
|---|---|
| MFEQPLPTLP MWRVDHIEPS PEMLALRAKG PIHRVRFPSG HEGWWVTGYD EAQAVLSDAA | 60 |
| FRPAGMPPET FTPDSVILGS PGWLVSHEGG KHAWLRMIVA PAFSNRRVKL LAQQVEAIAA | 120 |
| QLFETLAAQP QPADLRRHLS FPLPAMVISA LMGVLYEDHI FFAGLSDEVM THQHESGPRS | 180 |
| ASRVAWEELR TYICRKMRGK REEPGDNLLT DLLAAVDHGK ATEEEAVGLA AGVLVAGHES | 240 |
| TVAQIEFGLL AMFRHPQQRE RLVRDPFLAD KAVEEILRMY SPGAGWDGIM RYPRTDVTIA | 300 |
| GMDIPAESKV LVGLPATSFD PRHFEDPEVF DIGRDPNPHL AFSYGPHNCI GAALARLELK | 360 |
| VVFGSIFQRF PALRLAVAPE ELKLRKEIIT GGFEEFPVLW | 400 |

SEQ ID NO: 7

| | |
|---|---|
| atgtctgaac aaccactacc gacccttcca atgtggagag tagaccacat tgaaccgagt | 60 |
| cccgaaatgt tggcccttag agctaatgga cccatccata gagtgagatt cccatccgga | 120 |
| cacgaaggct ggtgggtcac tggatatgat gaagccaagg ctgtcttaag tgatactgcg | 180 |
| ttcagaccag ccggaatgcc accagctgct tttactccgg atagcgttat ccttggcagt | 240 |
| ccgggttggt tagtttcaca cgaaggaggt gagcatacaa gattaaggac catagtcgcc | 300 |
| cctgcgtttg gtgattcaag aatcaaattg TTagcacagc aagtcgaggc cattgcagca | 360 |
| caacttttta aaactttatc cacacagcct caaccagctg acttaagacg tcatctttcc | 420 |
| tttcctttac cagccatggt tatatcagcc ttgatgggtg ttcgttacga agatcatgct | 480 |
| tttttcgcag gtctgtcaga tgaagtaatg actcaccagc atgaatccgg acccaggagc | 540 |
| gccagtcgtc ttgcatggga agaattgaga gcatatataa gagatcgtat gcgtgaaaag | 600 |
| agacaggatc caggtgataa cctgctgact gatttattgg cggcggtgga tcaaggtaaa | 660 |
| gcaagtgaag aagaagctat tggactggca gctggcatgt tagttgctgg gcatgagagc | 720 |
| acagcagctc aaatagaatg tggtctatta gcgatgttta gacatccaca gcaaagagaa | 780 |
| aggcttgttg ctgacccaag tttattagat aaaaccgtcg aggaaatttt aagaatgtac | 840 |
| ccacctgggg ctggttggga tgggattatg agatacccta gaacagatgt gactatcgct | 900 |
| ggtgtacaca tccctgctga atctaaagtc cttgtgggat tacctgctac ctcttttgat | 960 |
| ccgaggcagt ttgatgatcc tgagatattt gacatcggta gagacgagaa acctcatctg | 1020 |
| gctttttcct acggtccgca ctattgcatc ggcggtgcat tggctagatt ggaattgaag | 1080 |
| gcagttttcg gatctatttt ccaaagattt cctggtttaa gattagcagt tgctccagaa | 1140 |
| gaattacgtc tgagaaaaga gattattaca ggcggatttg aggagatgcc agtgctgtgg | 1200 |
| taa | 1203 |

SEQ ID NO: 8

| | |
|---|---|
| MSEQPLPTLP MWRVDHIEPS PEMLALRANG PIHRVRFPSG HEGWWVTGYD EAKAVLSDTA | 60 |
| FRPAGMPPAA FTPDSVILGS PGWLVSHEGG EHTRLRTIVA PAFGDSRIKL LAQQVEAIAA | 120 |
| QLFKTLSTQP QPADLRRHLS FPLPAMVISA LMGVRYEDHA FFAGLSDEVM THQHESGPRS | 180 |
| ASRLAWEELR AYIRDRMREK RQDPGDNLLT DLLAAVDQGK ASEEEAIGLA AGMLVAGHES | 240 |
| TAAQIECGLL AMFRHPQQRE RLVADPSLLD KTVEEILRMY PPGAGWDGIM RYPRTDVTIA | 300 |
| GVHIPAESKV LVGLPATSFD PRQFDDPEIF DIGRDEKPHL AFSYGPHYCI GGALARLELK | 360 |
| AVFGSIFQRF PGLRLAVAPE ELRLRKEIIT GGFEEMPVLW | 400 |

SEQ ID NO: 9

| | |
|---|---|
| atgagcgaac agcctttacc tatgttgccc atgtggagag tagatcacat cgagccatca | 60 |
| cccgaaatgt tagcactgag agcaaaaggg cctatacacc gtgttagatt tccgtctggt | 120 |
| gatgaaggtt ggtgggtgac cggttacgac gaagcaaaag cggtgttatc agatgctgcg | 180 |
| tttaggccca gcggtatgcc ccctgcagct gtgactagtg ctacagtcat attgggttca | 240 |
| ccgggctggt tggggagcca tgagggttct gaacacgcta gactgagaac catcgtagcc | 300 |
| cctgcctttt cttcaggtag agtcaaattg ttagcacaac aagtggaagc cattgcagct | 360 |
| gagttattcg aaaccttggc ggcccaacca cagccagcag acctgagaag acacttgagt | 420 |
| tttccgcttc ccgctatggt gatttctgcc ttaatgggcg tgctgtatga agaccatgcc | 480 |
| tttttcgccc gtttgagtga taaagtaatg acccatcaat atgaaagtgg tcctcgttca | 540 |
| gcggcacgtt tggcgtggga ggagttaaga gcatatatta gaggcaagat gcgtgataag | 600 |
| agacaagacc ccggagacaa cttgctaacc gatttgcttg cagcagtgga tcaaggtaaa | 660 |
| gcaacggaag aggaagcaat aggattggca gcaggtatgt tggtcgcagg acatgaaacc | 720 |
| acagtggcgc agattgaatt cggtctattg gctatgttta ggcatccaca gcaaagagag | 780 |
| agattagttg gcgacccgag tttggtcgat aaggcagtag aggagatttt gagaatgtat | 840 |
| cctcctggtg ccggatggga tggtattatg aggtatccaa gaacagacgt cactattgca | 900 |
| ggagtacata tcccagccga gagcaaggtc ctggttggtt tgccggctac atcctttgat | 960 |
| cccagacatt ttgacgatcc agaaattttt gatgtgggaa gagaggaaaa acctcatcta | 1020 |
| gccttctcat atggaccaca ttactgcatc ggagtggagt tggcacgttt ggaattgaga | 1080 |
| gttgtctttg gttcaatatt ccagagattt ccagcgctta gactggcggt ggccccagag | 1140 |
| gaattgaaat tgagaaaggc catcattact ggcggttttg aagcttttcc cgttttatgg | 1200 |
| tga | 1203 |

SEQ ID NO: 10

| | |
|---|---|
| MSEQPLPMLP MWRVDHIEPS PEMLALRAKG PIHRVRFPSG DEGWWVTGYD EAKAVLSDAA | 60 |
| FRPSGMPPAA VISATVILGS PGWLGSHEGS EHARLRTIVA PAFSSGRVKL LAQQVEAIAA | 120 |
| ELFETLAAQP QPADLRRHLS FPLPAMVISA LMGVLYEDHA FFARLSDKVM THQYESGPRS | 180 |
| AARLAWEELR AYIRGKMRDK RQDPGDNLLT DLLAAVDQGK ATEEEAIGLA AGMLVAGHET | 240 |
| TVAQIEFGLL AMFRHPQQRE RLVGDPSLVD KAVEEILRMY PPGAGWDGIM RYPRTDVTIA | 300 |
| GVHIPAESKV LVGLPATSFD PRHFDDPEIF DVGREEKPHL AFSYGPHYCI GVELARLELR | 360 |
| VVFGSIFQRF PALRLAVAPE ELKLRKAIIT GGFEAFPVLW | 400 |

SEQ ID NO: 11

| | |
|---|---|
| atggcggaat tagatacgtt agatatcgtt gttttaggcg ctttattgtt gggcacatta | 60 |
| gcgtatttta cgaagggcac attatggggt gtcactaagg atccttatgc aaacgctttc | 120 |
| gcaaatgcta acggagctaa agccggcaga tcaagaaata tcgttgaaaa aatggatgaa | 180 |
| tctggtaaaa actgcgtcat attctacggt tctcaaactg gaacggcaga ggattacgca | 240 |
| tcaagattag cgaaagaagg aaagtcaaga ttcgggttag ggactatggt tgcagattta | 300 |
| gaagaatatg attatgataa ccttgataca atgagcggcg ataaggttgc catgtttgtt | 360 |
| cttgctacct atggcgaggg cgaaccaact gacaacgcag tagagtttta tgaatttatt | 420 |
| actggtgaag gggttgcttt tagtgaagga aacgatcccc ccttaggcaa tctgaactac | 480 |
| gtggcctttg gactggggaa caatacttat gaacactaca attcaatggt cagaaatgtc | 540 |
| gataaagccc ttaggaatct gggtgctcat aggatcggag aggctggtga aggcgatgac | 600 |
| ggtgctggca caatggaaga agattttcta gcatggaagg aaccaatgtg ggccgcctta | 660 |
| gctgacaaaa tgggtttgga ggaaagggaa gcagtatatg accctgtgtt cagtatcgtt | 720 |
| gatcgtgata atttgactcc tgaaagccca gaagtctatt tgggtgaacc taataaaatg | 780 |
| catttagagg atgcggtcaa gggcccattt aattctcata atccatatat agcaccaata | 840 |
| gctgaatcta gagaattgtt tagtgttaaa gacaggaatt gcatccatat ggaaattgac | 900 |
| atagacggtt caaatttgag ctatcaaact ggggatcatg tggctatttg gcctaccaac | 960 |
| ccaggagatg aagtggatag atttttagac atcattgatt taaaggataa acgtgacaag | 1020 |
| gttataggag tgaaagcact tgaaccaact gcaaaggtcc cttttccaac accaacaaca | 1080 |
| tatgacgtta tcgccaggta tcatttagaa atctgtgcac cggtctctag acagtttgtg | 1140 |
| tccactctag cagcattctc cccaaatgat gaggtaaaag cagaaatgac tagattgggt | 1200 |
| aacgataagg attattttca tgataagacg ggcccacatt attataatat cgcccgtttt | 1260 |
| ctagctgcgg ttggtaaggg cgagaaatgg tcaaatatcc ctttttctgt ttttgtcgaa | 1320 |
| ggtttaacga aattacaacc aagatattat tcaatctcct cttcaagcct agtacaacca | 1380 |
| aaaaaaatat caataacggc agtaattgag tcacaggtta tacctgccag gcaagatcca | 1440 |
| tttagaggtg tagctacgaa ctacttattt gcattgaaac agaagcaaaa cggtgatcca | 1500 |
| aatccctccc catttggaca tacttatgca ttaaacggcc ctagaaataa atttgacggt | 1560 |
| atacacgtcc ccgtccacgt aaggcactcc aatttcaaac taccgagcga tccagcaaaa | 1620 |
| ccagttatta tggttggtcc aggaactgga gtggctccgt ttagaggttt catccaagag | 1680 |
| agagctaaac aggcccagga tggggccaca gtaggccgta ctatcttgtt cttcggttgc | 1740 |
| caacgtaggt ccgaagattt tttgtacgaa agtgaatgga aagaatacaa ggaagttcta | 1800 |
| ggagataccc ttgagatagt cactgccttc tccagggaaa catcaaagaa agtttatgtg | 1860 |
| cagcacaggt tgaaagagag atccaaagaa atcggagaac tattatcaca gaaagcatac | 1920 |
| ttttatgtgt gtggcgatgc tgctcatatg gctagagaag ttaatactgt attggctcaa | 1980 |
| attatcgctg aatctagggg tgtaagtgaa gccaagggtg aagagattgt taaaaatatg | 2040 |
| agggctgcta atcagtacca agttaggagg gggaacaatg tctttttttg ggctataagt | 2100 |
| ggttctattg atatgacggc caataccgcc aacttacaag aagatgtgtg gagctga | 2157 |
| SEQ ID NO: 12 | |
| MAELDTLDIV VLGALLLGTL AYFTKGTLWG VTKDPYANAF ANANGAKAGR SRNIVEKMDE | 60 |
| SGKNCVIFYG SQTGTAEDYA SRLAKEGKSR FGLGTMVADL EEYDYDNLDT MSGDKVAMFV | 120 |
| LATYGEGEPT DNAVEFYEFI TGEGVAFSEG NDPPLGNLNY VAFGLGNNTY EHYNSMVRNV | 180 |
| DKALRNLGAH RIGEAGEGDD GAGTMEEDFL AWKEPMWAAL ADKMGLEERE AVYDPVFSIV | 240 |
| DRDNLTPESP EVYLGEPNKM HLEDAVKGPF NSHNPYIAPI AESRELFSVK DRNCIHMEID | 300 |
| IDGSNLSYQT GDHVAIWPTN PGDEVDRFLD IIDLKDKRDK VIGVKALEPT AKVPFPTPTT | 360 |
| YDVIARYHLE ICAPVSRQFV STLAAFSPND EVKAEMTRLG NDKDYFHDKT GPHYYNIARF | 420 |
| LAAVGKGEKW SNIPFSVFVE GLTKLQPRYY SISSSSLVQP KKISITAVIE SQVIPARQDP | 480 |
| FRGVATNYLF ALKQKQNGDP NPSPFGHTYA LNGPRNKFDG IHVPVHVRHS NFKLPSDPAK | 540 |
| PVIMVGPGTG VAPFRGFIQE RAKQAQDGAT VGRTILFFGC QRRSEDFLYE SEWKEYKEVL | 600 |
| GDTLEIVTAF SRETSKKVYV QHRLKERSKE IGELLSQKAY FYVCGDAAHM AREVNTVLAQ | 660 |
| IIAESRGVSE AKGEEIVKNM RAANQYQVRR GNNVFFWAIS GSIDMTANTA NLQEDVWS | 718 |

SEQ ID NO: 13

| | |
|---|---|
| atggcaacct tggttcacgt gggtcacttt ggtagaccct tgtgttcagg acaagctttg | 60 |
| cctttgcttc tagccggaat cttggcggca gccttagcaa tcaaagctgc ggcgtggtgc | 120 |
| gctcgtaaac gtcatctagc agaaattcca ttggccaacc caccttcatg gttatttttc | 180 |
| tctagacctg ctgaaagagt agctttggtt aggagtgctg ctgaagcatt gctgcgtgct | 240 |
| agagacgatt tcccacatgg accttttcgt tttctaagtg actggggtga acttctaatc | 300 |
| ttgcctcctg agttcgccga agaaataaga aatgaaccta aactatcttt tgggctagct | 360 |
| gcaatgagag ataatcatgc gaatatacct ggttttgaaa ctgttagaat tgtcggtaga | 420 |
| gatgatcaac ttttacaagc tgttgctaga aaacatctaa caaaacactt ggccaaggcc | 480 |
| atcgaaccat tgtgcgcgga agcaagcctg gctctagcag ttaatctagg tgagtcacca | 540 |
| gactggcaaa cagttagatt gcaacctgcc gtgctggata ttattgcaag gctatccagt | 600 |
| agagtgtatc tgggtgagca attgtgtaga tctcaggact ggttggctgt tacaaagact | 660 |
| tatgccacag cgttttatgc tgcatcatcc aaattgagaa tgtttccaag agctctacgt | 720 |
| ccattggtac attggtggat gccagagtgt cgtagactaa gagctcaacg tagggcagcc | 780 |
| gaagccatta tccgtccttt ggttaggcgt agacaacaag ctaaacaagc ggcagcagcc | 840 |
| gccgggcatc cagcccccgt gtttcatgat gcccttgagt gggctgaaca ggaggctgca | 900 |
| acagctgccg ctgcggccgc agctgggagg tctagatctt gtgatccagt tgtatttcaa | 960 |
| ttggcactgt ccttgctagc aattcacaca acatatgatc ttctgcagca agcaatgact | 1020 |
| gatctagctt ctaatccaca atacataggt cctttaagag atgaagtcgc aagagttgtt | 1080 |
| gggcaagacg ggtggagtaa agcttctttg tataagatga agcttttgga tagtgccctt | 1140 |
| aaggaaactc aaaggttaaa acccggttct attgttacca tgaggcgtgt tgctactgat | 1200 |
| gatgttgctt tgtcatccgg tcttgtgttg aaaaaaggta ccagggttaa cgtcgataat | 1260 |
| aggagaatga ctgacgcggc agtttatgcc gatcctagag tttacaaccc ctggagattt | 1320 |
| tatcaaatga ggctgcaacc cggtaaagaa catgtagctc aattggtttc tacctcccca | 1380 |
| gatcacttgg gatttggcca cggcttgcat tcatgtcctg gtaggttctt cgctgcgaat | 1440 |
| gaagttaagg tagctttggg tcatatgttg ttaaagtatg actggaagct tgctcctgcg | 1500 |
| acggacaaga caccagattg tagaggaatg ttggcaaaag ctagcccaac tactgatgtg | 1560 |
| atgatcagga ggagacatga cgaggctgat acaggcgctg cagcaagaga atag | 1614 |

SEQ ID NO: 14

| | |
|---|---|
| MATLVHVGHF GRPLCSGQAL PLLLAGILAA ALAIKAAAWC ARKRHLAEIP LANPPSWLFF | 60 |
| SRPAERVALV RSAAEALLRA RDDFPHGPFR FLSDWGELLI LPPEFAEEIR NEPKLSFGLA | 120 |
| AMRDNHANIP GFETVRIVGR DDQLLQAVAR KHLTKHLAKA IEPLCAEASL ALAVNLGESP | 180 |
| DWQTVRLQPA VLDIIARLSS RVYLGEQLCR SQDWLAVTKT YATAFYAASS KLRMFPRALR | 240 |
| PLVHWWMPEC RRLRAQRRAA EAIIRPLVRR RQQAKQAAAA AGHPAPVFHD ALEWAEQEAA | 300 |
| TAAAAAAAGR SRSCDPVVFQ LALSLLAIHT TYDLLQQAMT DLASNPQYIG PLRDEVARVV | 360 |
| GQDGWSKASL YKMKLLDSAL KETQRLKPGS IVTMRRVATD DVALSSGLVL KKGTRVNVDN | 420 |
| RRMTDAAVYA DPRVYNPWRF YQMRLQPGKE HVAQLVSTSP DHLGFGHGLH SCPGRFFAAN | 480 |
| EVKVALGHML LKYDWKLAPA TDKTPDCRGM LAKASPTTDV MIRRRHDEAD TGAAARE | 537 |

SEQ ID NO: 15

| | |
|---|---|
| atggtcaaca aagaagaaat caccattcca accgctgatt tgtctccatt cttgaaagaa | 60 |
| ttggaccagg gttcttattc ctacgatgat gatgatgacg accaaaagaa aaaaaaggct | 120 |
| gccgccattg aaattattgg taaggcttgt tctgagttcg gtttcttcca agttgttaat | 180 |
| catggtgttc cattgcactt gatgcaaaag gctttgttgt tgtctaatca gttcttcggt | 240 |
| tacccattgg acagaaaatt gcaagcttct ccattgccag gtgctccaat gccagctggt | 300 |
| tatggtagac aaccagatca ttctccagat aagaacgagt tctttatgat gttcccacca | 360 |
| cattctacct tcaacgtttt tccatctcat ccacaaggtt tcagagaagt tgttgaagag | 420 |
| ttgttctctt gcttcgttaa gaccgcttct gttatcgaaa acatcatcaa cgaatgtttg | 480 |
| ggtttgcctc caaatttctt gtctgagtac aacaacgata gaaagtggga tttgatgtcc | 540 |
| actttcagat acccaaacgc ctctgaaatt gaaaacgttg gtttgagaga acacaaggac | 600 |
| gttaacttca ttaccttgtt gttccaagat gaagtcggtg gtttggaagt taagactgaa | 660 |
| gatcatcaat ggatcccaat tatcccaaac cagaacacct tggttattaa cgttggtgat | 720 |
| gttatccagg tcttgtccaa tgatagatac aagtctgctt cccacagagt tgttagacaa | 780 |
| gaaggtagag aaagacactc ttacgctttc ttctacaata tcggtggtga taagttggtt | 840 |
| caaccattgc cacatttcac cacccatatt gatcaaccac caaactacaa gtccttcatc | 900 |
| tacaaagaat acttgcagtt gaggttgaga aacaagactc atccaccatc aaacccacaa | 960 |
| gatatcatca acatctctta ctactctacc acttaa | 996 |

SEQ ID NO: 16

| | |
|---|---|
| MVNKEEITIP TADLSPFLKE LDQGSYSYDD DDDDQKKKKA AAIEIIGKAC SEFGFFQVVN | 60 |
| HGVPLHLMQK ALLLSNQFFG YPLDRKLQAS PLPGAPMPAG YGRQPDHSPD KNEFFMMFPP | 120 |
| HSTFNVFPSH PQGFREVVEE LFSCFVKTAS VIENIINECL GLPPNFLSEY NNDRKWDLMS | 180 |
| TFRYPNASEI ENVGLREHKD VNFITLLFQD EVGGLEVKTE DHQWIPIIPN QNTLVINVGD | 240 |
| VIQVLSNDRY KSASHRVVRQ EGRERHSYAF FYNIGGDKLV QPLPHFTTHI DQPPNYKSFI | 300 |
| YKEYLQLRLR NKTHPPSNPQ DIINISYYST T | 331 |

SEQ ID NO: 17

| | |
|---|---|
| atgatcacct cctacgcagg ttcccaactt ttatcttttt atgtcacaat atttatcttt | 60 |
| acattagtac cttgggctat aagattgttc tggccaaaac ttagaaaggg cagtgtcgtt | 120 |
| ccattggcta atccacctga gagcttgttc ggtaccggta aaacaaggcg tagctttgta | 180 |
| aaattaagcc gtgaaatttt agctaaagca aggaacttat tcccagacga accttttaga | 240 |
| ctgattactg actggggcga ggtgcttatc cttcctccgg agttcgctga tgagatccgt | 300 |
| aatgatccgc gtctgtcatt ttcaaaggct gccatgcagg ataatcacgc aggtattcct | 360 |
| ggcttcgaaa ccgttgcgct tgtgggtaga gaagaccagc tgatacaaaa ggtggctagg | 420 |
| aaacaattga cgaaacatct tagtgccgtt attgaaccat tgagtagaga atcaactctg | 480 |
| gcagtcagtt taaacttcgg ggaatcaact gaatggcgta gtatcagatt aaaacccgca | 540 |
| attctggata ttatcgctag aatctccagc agaatttatt tgggcgatca attgtgtaga | 600 |
| aatgaagcat ggttaaaaat tactaaaacc tatactacaa acttttacac agccagcaca | 660 |
| aaccttagaa tgttcccgag accaattaga cctcttgccc attggttctt gcctgagtgt | 720 |
| agaaaactaa gacaagagag aaaggacgct gtcggtatca ttactccatt gatagagagg | 780 |
| cgtcgtgagt tacgtagagc tgcagtcgca gctggtcaac ctctacccgt ttttcacgat | 840 |
| gcaattgact ggagtgaaca ggaggccgag gcggcgggca gtgggtccgc atttgatcct | 900 |
| gttatttttc aattgacact ttctttgcta gccatccaca ccacctatga cctacttcaa | 960 |
| caaactatga tagacttggg aagacaccca gaatacattg atcccctacg tcaagaagtc | 1020 |
| gttcaattgt taagggaaga aggttggaaa aaaaccactc tgttcaaaat gaaattgctg | 1080 |
| gattctgcta tcaaagaaag tcagagaatg aaaccgggga gtattgttac tatgagaagg | 1140 |
| tatgtcactg aagatataac cttatcatcc ggattaacac ttcataaagg cactagatta | 1200 |
| aacgttgata acaggagact agatgaccca agaatctacg aaaatccgga agtctataat | 1260 |
| ccatatcgtt tttatgatat gaggtccgaa gcttctaagg accatggtgc acagttggta | 1320 |
| agtactggta gtaaccatat gggttttggg catggacaac attcttgtcc cggtagattt | 1380 |
| ttcgcagcta acgagattaa agtagcgttg tgccatatac ttgtaaaata tgattggaaa | 1440 |
| ttatgtccaa atactgagac gaaacctgat acaaggggta tgattgctaa atctagtcct | 1500 |
| gtcacggata ttctaattaa gagaagggaa agcgtggaat tggatttaga agcaatgtaa | 1560 |

SEQ ID NO: 18

| | |
|---|---|
| MVNKEEITIP TADLSPFLKE LDQGSYSYDD DDDDQKKKKA AAIEIIGKAC SEFGFFQVVN | 60 |
| HGVPLHLMQK ALLLSNQFFG YPLDRKLQAS PLPGAPMPAG YGRQPDHSPD KNEFFMMFPP | 120 |
| HSTFNVFPSH PQGFREVVEE LFSCFVKTAS VIENIINECL GLPPNFLSEY NNDRKWDLMS | 180 |
| TFRYPNASEI ENVGLREHKD VNFITLLFQD EVGGLEVKTE DHQWIPIIPN QNTLVINVGD | 240 |
| VIQVLSNDRY KSASHRVVRQ EGRERHSYAF FYNIGGDKLV QPLPHFTTHI DQPPNYKSFI | 300 |
| YKEYLQLRLR NKTHPPSNPQ DIINISYYST T | 331 |

SEQ ID NO: 19

| | |
|---|---|
| atgtctccaa ctcaatctac tactactcca gctacaaaac cagttatggc ttctattcca | 60 |
| tattactccg gtccttttaa tccaccagat accatttctg ctgtttccac taagagatac | 120 |
| tgtgattgga gatccgttaa catcaacgat gttagatctt ccactaagga tttcaccttg | 180 |
| gataagaatg gtttccagta catgaagcac tcttcagctt tatcttctcc accacatact | 240 |
| ttggcttcat ggaaagataa cgaaaccaga aagagagtta acgacgccga aattttggaa | 300 |
| ttgggtaaag ctgttactgg tgccaaaaag gttttggttg ttttggctat tggtagagat | 360 |
| gctgctttta ctgatccatt ggatcaaact tctagaccag atgtctacgg taatcaaact | 420 |
| gatactttgc cagctactag acagttgggt ttttatggtg gtgctaatat tggtccagct | 480 |
| agaaaacctc atgttgattg gggtccagat ggtgttagat ctattttgag aaactggtcc | 540 |
| catgaattgg ctgatgaagc caaggatatt attgatgctg aagatgaagc catctctttg | 600 |
| ccaggtggta ttgaagaaaa ttacaagggt agaagatggg gcttgtataa tacttggagg | 660 |
| ccattgaaac cagtcagaag agatccattg gcttgtgttg atttcgtgtc ctctaagaat | 720 |
| gataagtccg ccattttgtt gagaaagatc ccaggtattc atggtccatg tactgttgat | 780 |
| gctttgttta ctccagctaa tccaaaacat gaatggtact ggatgtctga tcaacaacca | 840 |
| gatgatatct tgttcatgaa gatcttcgat tccgctcacg aaagagatcc aaaaactatt | 900 |
| gctggtggtg ttcatcactg ttcttttcat catccaggta ctgaagatga ggaagtcaga | 960 |
| gaatctttgg agactaagtt tatggctttc tggtaa | 996 |

SEQ ID NO: 20

| | |
|---|---|
| MSPTQSTTTP ATKPVMASIP YYSGPFNPPD TISAVSTKRY CDWRSVNIND VRSSTKDFTL | 60 |
| DKNGFQYMKH SSALSSPPHT LASWKDNETR KRVNDAEILE LGKAVTGAKK VLVVLAIGRD | 120 |
| AAFTDPLDQT SRPDVYGNQT DTLPATRQLG FYGGANIGPA RKPHVDWGPD GVRSILRNWS | 180 |
| HELADEAKDI IDAEDEAISL PGGIEENYKG RRWGLYNTWR PLKPVRRDPL ACVDFVSSKN | 240 |
| DKSAILLRKI PGIHGPCTVD ALFTPANPKH EWYWMSDQQP DDILFMKIFD SAHERDPKTI | 300 |
| AGGVHHCSFH HPGTEDEEVR ESLETKFMAF W | 331 |

SEQ ID NO: 21

| | |
|---|---|
| atgccacata aggatactcc attggaatct ccagttggta agaatgttac tgctaccatt | 60 |
| gcttatcatt ctggtccagc tttgccaact tctccaattg ctggtgttac tactttacaa | 120 |
| gattgcaccc aacaagttgt tgccgttact gatattagac catccgtttc ttcattcacc | 180 |
| ttggatggta atggtttcca agttgtcaaa catgcttctg ctgttggttc tcctccttac | 240 |
| aatcattctt cttggactga tccagtcgtc agaaaagaag tttacgatcc agaaattatc | 300 |
| gaattggcca agtctttgac tggtgccaaa aaggttatga ttttgttggc ctcttctagg | 360 |
| aacgtccctt ttaaagaacc agaattggct ccaccatatc caatgccagg taaatctaat | 420 |
| tccggttcta aagaaggtgg cgctaatcca gctaatgaat tgccaactac tagagctaag | 480 |
| ggtttccaaa aaggtgaaga agaaggtcca gttagaaaac cacacaaaga ttggggtcca | 540 |
| tctggtgctt ggaatacttt gagaaattgg tcccaagaat tgatcgatga agccggtgat | 600 |
| attatcaaag ctggtgatga agctgctaaa ttgccaggtg gtagagctaa gaattatcaa | 660 |
| ggtagaagat gggccttgta tacaacttgg aggccattga aaccagttaa gagggatcca | 720 |
| atggcttatg ttgattattg gactgctgat ggtgaagatg gtgtttcatt ttggagaaat | 780 |
| ccaccaggtg ttcatggtac ttttgaatcc gatgttttgt tgactaaggc taacccaaaa | 840 |
| cataagtggt actggatttc tgatcaaacc ccagatgaag tcttgttgat gaagattatg | 900 |
| gacaccgaat ctgaaaagga tggttctggt attgctggtg gtgttcatca ctgttctttt | 960 |
| catttgccag gtactgaaaa agaagaggtc agagaatcca tcgaaactaa gtttattgcc | 1020 |
| ttctggtaa | 1029 |

SEQ ID NO: 22

| | |
|---|---|
| MPHKDTPLES PVGKNVTATI AYHSGPALPT SPIAGVTTLQ DCTQQVVAVT DIRPSVSSFT | 60 |
| LDGNGFQVVK HASAVGSPPY NHSSWTDPVV RKEVYDPEII ELAKSLTGAK KVMILLASSR | 120 |
| NVPFKEPELA PPYPMPGKSN SGSKEGGANP ANELPTTRAK GFQKGEEEGP VRKPHKDWGP | 180 |
| SGAWNTLRNW SQELIDEAGD IIKAGDEAAK LPGGRAKNYQ GRRWALYTTW RPLKPVKRDP | 240 |
| MAYVDYWTAD GEDGVSFWRN PPGVHGTFES DVLLTKANPK HKWYWISDQT PDEVLLMKIM | 300 |
| DTESEKDGSG IAGGVHHCSF HLPGTEKEEV RESIETKFIA FW | 342 |

SEQ ID NO: 23

| | |
|---|---|
| atgccacatc aacaaactcc attggaatct ccagttggta agaatgttac tgctaccatt | 60 |
| gcttaccata atggtccagc tttgccaact tctccaattg ctggtgttac tactttggaa | 120 |
| gattgcaccc aacatgttgt tgctgttact gatattagac catccgtttc ttcattcacc | 180 |
| ttggatggta atggtttcca agttgttaag cacgtttccg aagtttcttc tcctccatac | 240 |
| aatcattctt catggactga tccagtcgtc agaaaagaag tttacgatcc agaaattatc | 300 |
| gaattggcca agtctgttac tggtgccaaa aaggttatga ttttgttggc ttctgctagg | 360 |
| aacgtccctt ttaaagaacc agaattggct ccaccatatc caatgccatc taaaggtggt | 420 |
| aaagaaggtg gcgctggtca aactgttcaa ggtcaacatg aattgccaac tactagagct | 480 |
| aagggttttc aaaagggtga agaagaaggt ccagttagaa aaccacataa ggattggggt | 540 |
| ccatctggtg cttggaatac tttgttgaat tggtcccaag aattgatcga tgaagccgat | 600 |
| gatattatca aggctggtga tgaagctgct gaattgccag gtggtagagc taagaattat | 660 |
| caaggtagaa gatgggcctt gtatacaact tggaggccat tgaaaccagt taagagggat | 720 |
| ccaatggctt ttgttgatta ttggactgct gatgaagagg acggtgtttc attttggaga | 780 |
| aatccaccag gtgttcatgg tacttttgaa tccgatgttt tgttgactag agctaaccca | 840 |
| aaacataagt ggtactggat ttctgatcaa accccagatg aagtcttgtt gatgaagatt | 900 |
| atggacaccg aatctgaaaa ggacggttct gatattgctg gtggtgttca ttactgttct | 960 |
| ttccatttgc cagtctccga aaaagaagaa gtcagagaat ccatcgaaac gaagtttatt | 1020 |
| gctttctggt aa | 1032 |

SEQ ID NO: 24

| | |
|---|---|
| MPHQQTPLES PVGKNVTATI AYHNGPALPT SPIAGVTTLE DCTQHVVAVT DIRPSVSSFT | 60 |
| LDGNGFQVVK HVSEVSSPPY NHSSWTDPVV RKEVYDPEII ELAKSVTGAK KVMILLASAR | 120 |
| NVPFKEPELA PPYPMPSKGG KEGGAGQTVQ GQHELPTTRA KGFQKGEEEG PVRKPHKDWG | 180 |
| PSGAWNTLLN WSQELIDEAD DIIKAGDEAA ELPGGRAKNY QGRRWALYTT WRPLKPVKRD | 240 |
| PMAFVDYWTA DEEDGVSFWR NPPGVHGTFE SDVLLTRANP KHKWYWISDQ TPDEVLLMKI | 300 |
| MDTESEKDGS DIAGGVHYCS FHLPVSEKEE VRESIETKFI AFW | 343 |

SEQ ID NO: 25

| | |
|---|---|
| atgccacaca aggataactt gttggaatct ccagttggta aatctgttac tgctaccatt | 60 |
| gcttatcatt ctggtccagc tttgccaact tctccaattg ctggtgttac tactttacaa | 120 |
| gattgcaccc aacaagctgt tgctgttact gatattagac catccgtttc ttcattcacc | 180 |
| ttggatggta atggtttcca agttgttaag cacacttctg ctgttggttc acctccatat | 240 |
| gatcattctt catggactga tccagtcgtc agaaaagaag tttacgatcc agaaattatc | 300 |
| gaattggcca agtctttgac tggtgccaaa aaggttatga ttttgttggc ctcttctagg | 360 |
| aacgtccctt ttaaagaacc agaattggct ccaccatatc caatgccagg taaatcttct | 420 |
| tcaggttcca aagaaagaga agctattcca gctaatgaat tgccaactac tagagctaag | 480 |
| ggtttccaaa aaggtgaaga agaaggtcca gttagaaagc cacataagga ttggggtcca | 540 |
| tctggtgctt ggaatacttt gagaaattgg tcccaagaat tgatcgatga agccggtgat | 600 |
| attatcaaag ctggtgatga agctgctaaa ttgccaggtg gtagagctaa gaattatcaa | 660 |
| ggtagaagat gggccttgta tacaacttgg aggccattga aaactgttaa gagggatcca | 720 |
| atggcttacg ttgattattg gactgctgat gaagaggatg gtgtttcatt ttggagaaat | 780 |
| ccaccaggtg ttcatggtac ttttgaatcc gatgttttgt tgactaaggc taacccaaaa | 840 |
| cataagtggt actggatttc tgatcaaacc ccagatgaag tcttgttgat gaagattatg | 900 |
| gacaccgaat ctgaaaagga cggttctgaa attgctggtg gtgttcatca ctgttctttt | 960 |
| catttgccag gtactgaaaa agaagaggtc agagaatcca tcgaaactaa gtttattgcc | 1020 |
| ttctggtaa | 1029 |

SEQ ID NO: 26

| | |
|---|---|
| MPHKDNLLES PVGKSVTATI AYHSGPALPT SPIAGVTTLQ DCTQQAVAVT DIRPSVSSFT | 60 |
| LDGNGFQVVK HTSAVGSPPY DHSSWTDPVV RKEVYDPEII ELAKSLTGAK KVMILLASSR | 120 |
| NVPFKEPELA PPYPMPGKSS SGSKEREAIP ANELPTTRAK GFQKGEEEGP VRKPHKDWGP | 180 |
| SGAWNTLRNW SQELIDEAGD IIKAGDEAAK LPGGRAKNYQ GRRWALYTTW RPLKTVKRDP | 240 |
| MAYVDYWTAD EEDGVSFWRN PPGVHGTFES DVLLTKANPK HKWYWISDQT PDEVLLMKIM | 300 |
| DTESEKDGSE IAGGVHHCSF HLPGTEKEEV RESIETKFIA FW | 342 |

SEQ ID NO: 27

| | |
|---|---|
| atggccgatc aagaaattac tactgctcca ccatcttctc cattggttcc attggatttt | 60 |
| tcttcatctc acgaaaccgt tccagaatcc catatttggg ttgattccat tgaattgtct | 120 |
| ccagctatgg atttggacga gaaattgtct ttgccagtta tcgatttgtt ggatgatacc | 180 |
| actgcctctg aattgattgg taaagcttgt caacaatggg gtatgttcca attgattaac | 240 |
| catggtgttc caaagtccat tattgccgaa actgaagatg aagctagaag gttgtttgct | 300 |
| ttgccaacta ctcaaaagat gaagactttt ggtccaggta atactggtta tggtatggtt | 360 |
| ccattgtcca agtaccattc taaatccatg tggcatgaag gtttcaccat ttttggttct | 420 |
| ccattggatg atgctaaaaa gttgtggcca tctgactaca agagattctg tgatgttatg | 480 |
| gaagaatacc aaagaaagat gaagggtttg gccgatagat tgatgagatt gatcttgaag | 540 |
| ttcttggaca tctccgaaga agagatcatg aagttgatgt tcactccaga ggattcctct | 600 |
| aaaatctaca ctgctttgag gttgaacttg tatccaccat gtccagatcc agatagagtt | 660 |
| gttggtatgg cttctcatac tgatacttca ttcttcacca ttatccacca agctagaaat | 720 |
| gatggcttgc aaatctttaa ggatgaagct ggttgggttc cattatctcc aacatctggt | 780 |
| actttgatgg ttaacgttgg tgacttgttg cagattttgt ctaatggtag attcccatcc | 840 |
| atcttgcaca gagttatgat ccaagaaaag atggaagata ggttgtcctt ggcttacttt | 900 |
| tacactccac caccacatat ctatattgct ccatactgta agccattgtc cgaatctcca | 960 |
| caaatcccat tatacagatg tgtcaccgtc aaagaatact ccacttctaa gtctaacaac | 1020 |
| aacttcaagg gtttgtctac cgtcaagatc tcctctttga tttga | 1065 |

SEQ ID NO: 28

| | |
|---|---|
| MADQEITTAP PSSPLVPLDF SSSHETVPES HIWVDSIELS PAMDLDEKLS LPVIDLLDDT | 60 |
| TASELIGKAC QQWGMFQLIN HGVPKSIIAE TEDEARRLFA LPTTQKMKTF GPGNTGYGMV | 120 |
| PLSKYHSKSM WHEGFTIFGS PLDDAKKLWP SDYKRFCDVM EEYQRKMKGL ADRLMRLILK | 180 |
| FLDISEEEIM KLMFTPEDSS KIYTALRLNL YPPCPDPDRV VGMASHTDTS FFTIIHQARN | 240 |
| DGLQIFKDEA GWVPLSPTSG TLMVNVGDLL QILSNGRFPS ILHRVMIQEK MEDRLSLAYF | 300 |
| YTPPPHIYIA PYCKPLSESP QIPLYRCVTV KEYSTSKSNN NFKGLSTVKI SSLI | 354 |

SEQ ID NO: 29

| | |
|---|---|
| atggcttcta ccttgtctca agttttcaga gataatccat tgccattgaa ccacatcatc | 60 |
| ccattggatt ttacctctgt tcattccttg ccagaatctc atgtttggcc agcttttgat | 120 |
| ggttttccat ttggtactac ttacccaggt gaaaagttct ccattccaat catcgatttg | 180 |
| atggatccaa atgctgctca attggttggt catgcttgtg aaaaatgggg tgcttttcaa | 240 |
| ttgacttctc atggtttgcc atccatcttg actgatgatg ttgaatctca aaccagaagg | 300 |
| ttgtttgctt tgccagctca cgaaaaaatg aaggctttga gattgccatc tggtggtact | 360 |
| ggttatggtc aagctagaat ttctccattc tacccaaagt tcatgtggca tgaaggtttc | 420 |
| actattatgg gttctgctgt tgatcatgct agaaaattgt ggccagatga ttacaagggt | 480 |
| ttctgtgatg ttatggaaga ttaccaaaag aagatgaagg aattggccga atccttgttg | 540 |
| catatcttct tggaatcctt ggacatctcc aaagaagagt acagatctac cactattcaa | 600 |
| agaggtcata aggcttgtaa taccgccttg caattgaatt cttatccacc atgtccagat | 660 |
| ccaaatagag ctatgggttt ggctccacat actgattctt tgttgttcac catcgttcat | 720 |
| caatctcaca cctccggttt acaaattttg agagatggtg ttggttggat cactgttttt | 780 |
| ccattggaag gtgctttggt tgttaacgtt ggtgatttgt tgcacatctt gtctaatggt | 840 |
| agatacccat ctgttttaca cagagccgtt gttaatcaag ccgaacacag aatttctttg | 900 |
| gcttactttt atggtccacc agccgattct ttgatttctc cattgtgtaa cttggtttct | 960 |
| tccggtcaac aagttgttgc tccaagatat agatccgtgt ctgtcaaaga atacgtcgat | 1020 |
| ttgaaagaga agcacaaaga aaaggccttg tccttgttga gattgtga | 1068 |

SEQ ID NO: 30

| | |
|---|---|
| MASTLSQVFR DNPLPLNHII PLDFTSVHSL PESHVWPAFD GFPFGTTYPG EKFSIPIIDL | 60 |
| MDPNAAQLVG HACEKWGAFQ LTSHGLPSIL TDDVESQTRR LFALPAHEKM KALRLPSGGT | 120 |
| GYGQARISPF YPKFMWHEGF TIMGSAVDHA RKLWPDDYKG FCDVMEDYQK KMKELAESLL | 180 |
| HIFLESLDIS KEEYRSTTIQ RGHKACNTAL QLNSYPPCPD PNRAMGLAPH TDSLLFTIVH | 240 |
| QSHTSGLQIL RDGVGWITVF PLEGALVVNV GDLLHILSNG RYPSVLHRAV VNQAEHRISL | 300 |
| AYFYGPPADS LISPLCNLVS SGQQVVAPRY RSVSVKEYVD LKEKHKEKAL SLLRL | 355 |

SEQ ID NO: 31

| | |
|---|---|
| atgtccatgg ttgtccaaca agaacaagaa gttgtttttg acgctgctgt tttgtctggt | 60 |
| caaactgaaa ttccatccca attcatttgg ccagctgaag aatctccagg ttctgttgct | 120 |
| gttgaagaat tggaagttgc cttgattgat gttggtgctg gtgctgaaag atcttctgtt | 180 |
| gttagacaag ttggtgaagc ttgtgaaaga cacggttttt tcttggttgt taaccatggt | 240 |
| attgaagccg ctttgttgga agaggctcat agatgtatgg atgctttttt cactttgcca | 300 |
| ttgggtgaaa aacaaagagc acagagaagg gctggtgaat cttgtggtta tgcttcatct | 360 |
| tttactggta gattcgcttc taagttgcca tggaaagaaa ctttgtcctt cagatattct | 420 |
| tccgctggtg atgaagaagg tgaagagggc gttggtgaat atttggttag aaaattgggt | 480 |
| gccgaacacg gtagaagatt gggtgaagtt tattctagat actgccacga aatgtccagg | 540 |
| ttgtctttgg aattgatgga agttttgggt gagtctttgg gtatagttgg tgatagaagg | 600 |
| cattacttca gaagattctt ccagagaaac gactccatca tgagattgaa ttattaccca | 660 |
| gcttgccaaa gaccattgga tactttgggt actggtccac attgtgatcc aacatctttg | 720 |
| actatcttgc accaagatca tgttggtggt ttggaagttt gggctgaggg aaggtggaga | 780 |
| gctattagac caagaccagg tgctttggtt gttaatgttg gtgatacttt catggctttg | 840 |
| tccaacgcta gatatagatc ttgcttgcat agagccgttg ttaattctac tgctccaaga | 900 |
| agatctttgg cattcttttt gtgtccagaa atggataccg ttgttagacc acctgaagaa | 960 |
| ttggttgatg atcaccatcc aagagtttac ccagatttta cttggagagc tttgttggat | 1020 |
| ttcacccaaa gacattacag agctgatatg aggttgttcc aagctttttc tgattggttg | 1080 |
| aaccatcata gacacttgca acctactatc tactcctga | 1119 |

SEQ ID NO: 32

| | |
|---|---|
| MSMVVQQEQE VVFDAAVLSG QTEIPSQFIW PAEESPGSVA VEELEVALID VGAGAERSSV | 60 |
| VRQVGEACER HGFFLVVNHG IEAALLEEAH RCMDAFFTLP LGEKQRAQRR AGESCGYASS | 120 |
| FTGRFASKLP WKETLSFRYS SAGDEEGEEG VGEYLVRKLG AEHGRRLGEV YSRYCHEMSR | 180 |
| LSLELMEVLG ESLGIVGDRR HYFRRFFQRN DSIMRLNYYP ACQRPLDTLG TGPHCDPTSL | 240 |
| TILHQDHVGG LEVWAEGRWR AIRPRPGALV VNVGDTFMAL SNARYRSCLH RAVVNSTAPR | 300 |
| RSLAFFLCPE MDTVVRPPEE LVDDHHPRVY PDFTWRALLD FTQRHYRADM RLFQAFSDWL | 360 |
| NHHRHLQPTI YS | 372 |

SEQ ID NO: 33

| | |
|---|---|
| atggattctt ccgcttctac cattttgatg ccaccaccat tggaattgaa agacgaaaga | 60 |
| aaaaagggct ccgttgtttt cgattcctct aagatgcaaa agcaagaaaa gttgccaacc | 120 |
| gaattcattt ggccagatgc tgatttggtt agagcacaac aagaattgaa cgaaccattg | 180 |
| atcgatttgg acggtttttt caaaggtgat gaagctgcta ctgctcatgc tgctgaattg | 240 |
| attagaatgg cttgtttgaa ccacggtttc ttccaagtta ctaatcacgg tgttgatttg | 300 |
| gatttgatta gagctgctca agaagatatg ggcgcttttt tcaaattgcc attgtccaga | 360 |
| aagttgtccg tcaaaaaaaa gccaggtgaa ttgtctggtt attctggtgc tcatgctgat | 420 |
| agatacactt ctaaattgcc atggaaagaa accttgtcct tcgtttactg ttacgactct | 480 |
| ggttctaaac ctatggttgc tgattacttc aaaaccgctt tgggtgaaga tttcgaacaa | 540 |
| attggttgga tctaccaaaa gtactgcgac gctttgaaag aattgtcctt gggtatcatg | 600 |
| cagttgttgg ctatttcttt ggatgtcgac tcttcctact acagaaagtt gtttgaagat | 660 |
| ggttactcca tcatgaggtg taattcttac ccaccatgta aagaagctgg tttggttatg | 720 |
| ggtactggtc cacattgtga tccagttgct ttgaccattt tacaccaaga tcaagtcaag | 780 |
| ggtttggaag ttttcgttga taacaaatgg caatccgtta agccaagacc aggtgctttg | 840 |
| gttgttaata ttggtgatac tttcatggcc ttgtctaacg gcaagtacaa gtcttgtatt | 900 |
| catagagccg ttgtcaacat ggacaaagaa agaagatctt tgaccttctt catgtcccca | 960 |
| aaggatgata aggttgtttc tccaccacaa gaattgatcg ttagagaagg tcctagaaag | 1020 |
| tacccagatt ttaagtggtc tgagttgttg gaattcaccc aaaaacatta cagaccaaac | 1080 |
| aacgacacct tgcaatcttt tgttgagtgg agattatctt cccagaccaa gtaa | 1134 |

SEQ ID NO: 34

| | |
|---|---|
| MDSSASTILM PPPLELKDER KKGSVVFDSS KMQKQEKLPT EFIWPDADLV RAQQELNEPL | 60 |
| IDLDGFFKGD EAATAHAAEL IRMACLNHGF FQVTNHGVDL DLIRAAQEDM GAFFKLPLSR | 120 |
| KLSVKKKPGE LSGYSGAHAD RYTSKLPWKE TLSFVYCYDS GSKPMVADYF KTALGEDFEQ | 180 |
| IGWIYQKYCD ALKELSLGIM QLLAISLDVD SSYYRKLFED GYSIMRCNSY PPCKEAGLVM | 240 |
| GTGPHCDPVA LTILHQDQVK GLEVFVDNKW QSVKPRPGAL VVNIGDTFMA LSNGKYKSCI | 300 |
| HRAVVNMDKE RRSLTFFMSP KDDKVVSPPQ ELIVREGPRK YPDFKWSELL EFTQKHYRPN | 360 |
| NDTLQSFVEW RLSSQTK | 377 |

SEQ ID NO: 35

| | |
|---|---|
| atggctacta ctattgccga cgtttttaag tctttcccag ttcatattcc agcccacaag | 60 |
| aatttggatt tcgattcctt gcatgaattg ccagattctt acgcttggat tcaaccagat | 120 |
| tcttttccat ctccaactca taagcaccac aactccattt tggattccga ttctgattcc | 180 |
| gttccattga tcgatttgtc tttgccaaat gctgctgctt tgattggtaa tgcttttaga | 240 |
| tcttggggtg ccttccaagt tattaaccat ggtgttccaa tttctttgtt gcaatccatt | 300 |
| gaatcctctg ccgatacttt gttttctttg ccaccatctc ataagttgaa ggctgctaga | 360 |
| actccagatg gtatttctgg ttatggtttg gtcagaatct cttcattctt cccaaaaagg | 420 |
| atgtggtctg aaggttttac tatagtcggt tctccattgg atcacttcag acaattgtgg | 480 |
| ccacatgatt accacaaaca ttgcgaaatc gttgaagaat acgacaggga aatgagatct | 540 |
| ttgtgtggta gattgatgtg gttgggtttg ggtgaattgg gtattactag agatgatatg | 600 |
| aagtgggctg gtccagatgg tgattttaag acttctccag ctgctactca attcaactct | 660 |
| tatccagttt gtccagatcc agatagagct atgggtttgg gtccacatac tgatacttca | 720 |
| ttattgacca tcgtctacca gtctaacacc agaggtttac aagttttgag agaaggtaag | 780 |
| agatgggtta ctgttgaacc agttgctggt ggtttggttg ttcaagttgg tgatttgttg | 840 |
| catattttga ccaatggctt gtacccatct gctttacatc aagctgttgt taacagaacc | 900 |
| agaaagagat tgtctgttgc ttacgttttt ggtccaccag aatctgctga aatttctcca | 960 |
| ttgaaaaagt tgttgggtcc aactcaacca ccattataca gaccagttac ttggactgaa | 1020 |
| tacttgggta aaaaggccga acatttcaac aacgctttgt ctactgttag attgtgtgct | 1080 |
| ccaattaccg gtttgttgga tgttaacgat cactccagag ttaaggttgg ttga | 1134 |

SEQ ID NO: 36

| | |
|---|---|
| MATTIADVFK SFPVHIPAHK NLDFDSLHEL PDSYAWIQPD SFPSPTHKHH NSILDSDSDS | 60 |
| VPLIDLSLPN AAALIGNAFR SWGAFQVINH GVPISLLQSI ESSADTLFSL PPSHKLKAAR | 120 |
| TPDGISGYGL VRISSFFPKR MWSEGFTIVG SPLDHFRQLW PHDYHKHCEI VEEYDREMRS | 180 |
| LCGRLMWLGL GELGITRDDM KWAGPDGDFK TSPAATQFNS YPVCPDPDRA MGLGPHTDTS | 240 |
| LLTIVYQSNT RGLQVLREGK RWVTVEPVAG GLVVQVGDLL HILTNGLYPS ALHQAVVNRT | 300 |
| RKRLSVAYVF GPPESAEISP LKKLLGPTQP PLYRPVTWTE YLGKKAEHFN NALSTVRLCA | 360 |
| PITGLLDVND HSRVKVG | 377 |

SEQ ID NO: 39

| | |
|---|---|
| atgcacgttg ttacttctac acctgaagct agacatgatg gtgcaccttt ggtttttgat | 60 |
| gcttctgttt tgagacacca acacaacatt ccaaagcaat tcatttggcc agatgaagaa | 120 |
| aaaccagctg ctacttgtcc agaattggaa gttccattga ttgacttgtc tggtttcttg | 180 |
| tctggtgaaa aagatgctgc tgctgaagct gttagattgg ttggtgaagc ttgtgaaaaa | 240 |
| cacggttttt tcttggttgt taaccacggt gttgacagaa agttgattgg tgaagctcat | 300 |
| aagtacatgg acgaattctt tgagttgcca ttgtcccaaa aacaatccgc tcaaagaaaa | 360 |
| gctggtgaac attgtggtta cgcttcatct tttactggta ggttctcttc taaattgcca | 420 |
| tggaaagaaa ccttgtcctt tagatttgct gccgacgaat ctttgaacaa cttggtcttg | 480 |
| cattacttga acgataagtt gggtgatcaa ttcgctaagt tcggtagagt ttaccaagat | 540 |
| tactgtgaag ctatgtccgg tttgtctttg ggtatcatgg aattgctagg taagtctttg | 600 |
| ggtgttgaag aacaatgctt caagaacttc ttcaaggaca acgactccat catgagattg | 660 |
| aatttttacc caccatgcca aaagccacat ttgactttgg gtactggtcc acattgtgat | 720 |
| ccaacatctt tgactatctt gcaccaagat caagtcggtg gtttacaagt ttttgttgat | 780 |
| aaccagtgga gattgatcac cccaaatttt gatgctttcg ttgttaacat cggtgatacc | 840 |
| tttatggctt tgtctaacgg tagatacaag tcctgcttgc atagagctgt tgttaactct | 900 |
| gaaagaacga gaaagtcttt ggcattcttc ttgtgtccaa gaaacgataa ggttgttaga | 960 |
| ccaccaagag aattggttga tactcaaaac ccaagaagat acccagattt cacttggtct | 1020 |
| atgttgttga gattcaccca aactcattac agagctgata tgaagacttt ggaagctttt | 1080 |
| tctgcttggt tgcaacaaga acaacaagag cagcaagaac aacagttcaa catctga | 1137 |

SEQ ID NO: 40

| | |
|---|---|
| MHVVTSTPEA RHDGAPLVFD ASVLRHQHNI PKQFIWPDEE KPAATCPELE VPLIDLSGFL | 60 |
| SGEKDAAAEA VRLVGEACEK HGFFLVVNHG VDRKLIGEAH KYMDEFFELP LSQKQSAQRK | 120 |
| AGEHCGYASS FTGRFSSKLP WKETLSFRFA ADESLNNLVL HYLNDKLGDQ FAKFGRVYQD | 180 |
| YCEAMSGLSL GIMELLGKSL GVEEQCFKNF FKDNDSIMRL NFYPPCQKPH LTLGTGPHCD | 240 |
| PTSLTILHQD QVGGLQVFVD NQWRLITPNF DAFVVNIGDT FMALSNGRYK SCLHRAVVNS | 300 |
| ERTRKSLAFF LCPRNDKVVR PPRELVDTQN PRRYPDFTWS MLLRFTQTHY RADMKTLEAF | 360 |
| SAWLQQEQQE QQEQQFNI | 378 |

SEQ ID NO: 41

| | |
|---|---|
| atggctaccg aatgtattgc tactgttcca caaatcttct ccgagaacaa gaccaaagaa | 60 |
| gattcctcta ttttcgacgc caagttgttg aatcaacatt cccatcatat cccacaacaa | 120 |
| ttcgtttggc cagatcacga aaaaccatct actgatgttc aaccattgca agttccattg | 180 |
| attgatttgg ctggtttctt gtctggtgat tcttgtttgg cttctgaagc tactagattg | 240 |
| gtttctaaag ctgctaccaa acacggcttt ttcttgatta ctaatcacgg tgttgacgaa | 300 |
| tccttgttgt ctagagctta cttgcatatg gactcatttt tcaaagctcc agcttgcgaa | 360 |
| aaacaaaagg ctcaaagaaa atggggtgaa tcttctggtt acgcttcttc atttgttggc | 420 |
| agattctctt ctaaattgcc atggaaagaa accttgtcct tcaagttttc tccagaagaa | 480 |
| aagatccatt cccaaaccgt taaggacttc gtgtctaaaa agatgggtga tggttacgaa | 540 |
| gatttcggta aggtttatca agaatacgct gaagctatga acaccttgtc cttgaagatc | 600 |
| atggaattgc taggtatgtc tttgggtgtc gaaagaaggt acttcaaaga attcttcgag | 660 |
| gactccgatt ccatcttcag attgaattat tacccacaat gcaagcaacc agaattggct | 720 |
| ttgggtactg gtccacattg tgatccaaca tctttgacta tcttgcacca agatcaagtc | 780 |
| ggtggtttac aagttttcgt tgataacaag tggcaatcca ttccaccaaa tccacatgct | 840 |
| ttcgttgtta acattggtga tactttcatg gctttgacca acggtagata caaatcttgc | 900 |
| ttgcatagag ccgttgtcaa ctctgaaaga gaaagaaaga ctttcgcatt cttcttgtgt | 960 |
| ccaaagggtg aaaaagttgt taagccacct gaagaattgg ttaacggtgt taagtctggt | 1020 |
| gaaagaaagt acccagattt cacttggtct atgttcttgg aattcaccca aaaacattac | 1080 |
| agagccgaca tgaacacttt ggacgaattt tctatttggt tgaagaacag aagatccttt | 1140 |
| taa | 1143 |

SEQ ID NO: 42

| | |
|---|---|
| MATECIATVP QIFSENKTKE DSSIFDAKLL NQHSHHIPQQ FVWPDHEKPS TDVQPLQVPL | 60 |
| IDLAGFLSGD SCLASEATRL VSKAATKHGF FLITNHGVDE SLLSRAYLHM DSFFKAPACE | 120 |
| KQKAQRKWGE SSGYASSFVG RFSSKLPWKE TLSFKFSPEE KIHSQTVKDF VSKKMGDGYE | 180 |
| DFGKVYQEYA EAMNTLSLKI MELLGMSLGV ERRYFKEFFE DSDSIFRLNY YPQCKQPELA | 240 |
| LGTGPHCDPT SLTILHQDQV GGLQVFVDNK WQSIPPNPHA FVVNIGDTFM ALTNGRYKSC | 300 |
| LHRAVVNSER ERKTFAFFLC PKGEKVVKPP EELVNGVKSG ERKYPDFTWS MFLEFTQKHY | 360 |
| RADMNTLDEF SIWLKNRRSF | 380 |

SEQ ID NO: 43

| | |
|---|---|
| atgccatcta gaccatcaag agtcgtcaaa gaacaacatc caactaagaa gtccttcttg | 60 |
| gacttggaat ctttgaacga attgccagat tcttttgctt ggggttcttt tgaagatcca | 120 |
| tgctctattg ataacccatc tggttatggt ccagattctg ttccagttat caacttgcaa | 180 |
| gatccacaag ctcaacaatt ggttggtttg gcttgtagat cttggggtgt tttccaagtt | 240 |
| accaaccatg gtattcaaaa gtccttgttg gatgatattg aagctgctgg taagtctttg | 300 |
| tttgccttgc cagttaatca aaagttgaag gctgctagat cttcttgtgg tgttactggt | 360 |
| tacggtccag ctggtatttc ttcatttttc ccaaaaagga tgtggtccga aggtttcact | 420 |
| attttgggtt ctccattgga tcatgctaga caattgtggc caaacaacta caacaagttc | 480 |
| tgcgatatca tcgaaaagta ccaaaaagaa atgaaccagt tggccaaaaa gttgatgcaa | 540 |
| ttggttgttg gttccttggg tatttccaac caggatatta tgaattgggc cgatttgttg | 600 |
| gaaggtgcta atggtgctat gcaattgaac tcttatccaa tcagaccaga tccaaataga | 660 |
| gctatgggtt tggctgctca tactgattct actttgttga ccatcttgca ccaatctaac | 720 |
| actaccggtt tacaggtttt cagagaaaga tctggttggg ttactgttcc accaatttct | 780 |
| ggtggtttgg ttattaacat cggtgacttg ttgcacatct tgtctaatgg tagataccca | 840 |
| tccgtttacc atagagccat ggttaataga gttcagcaca gattgtctgt tgcttacttg | 900 |
| tatggtccag cttcaggtgt tagagttcaa ccattgccaa aattgattga tgctactcac | 960 |
| ccaccattat acagaccagt tacttggtct gaatacttgg gtatcaagtc tgaacatttg | 1020 |
| accaaggcct tgtccttgat tagaatcaac cataacacta acccatcctt gactggtttg | 1080 |
| attggtaatg atgaacctaa gtccatcaac gttgactccg ataagactat tttggctgtt | 1140 |
| ttcggttaa | 1149 |

SEQ ID NO: 44

| | |
|---|---|
| MPSRPSRVVK EQHPTKKSFL DLESLNELPD SFAWGSFEDP CSIDNPSGYG PDSVPVINLQ | 60 |
| DPQAQQLVGL ACRSWGVFQV TNHGIQKSLL DDIEAAGKSL FALPVNQKLK AARSSCGVTG | 120 |
| YGPAGISSFF PKRMWSEGFT ILGSPLDHAR QLWPNNYNKF CDIIEKYQKE MNQLAKKLMQ | 180 |
| LVVGSLGISN QDIMNWADLL EGANGAMQLN SYPIRPDPNR AMGLAAHTDS TLLTILHQSN | 240 |
| TTGLQVFRER SGWVTVPPIS GGLVINIGDL LHILSNGRYP SVYHRAMVNR VQHRLSVAYL | 300 |
| YGPASGVRVQ PLPKLIDATH PPLYRPVTWS EYLGIKSEHL TKALSLIRIN HNTNPSLTGL | 360 |
| IGNDEPKSIN VDSDKTILAV FG | 382 |

SEQ ID NO: 45

| | |
|---|---|
| atgaagtaca ccacctgtca gatgaacatt tttccatctt tgtggtccat gaagaccagt | 60 |
| tttagatggc caagaacttc taagtggtcc tctgtttcat tatacgacat gatgttgaga | 120 |
| accgttgctt tgttgtctgg tagagctttt gttggtttgc cattgtgtag agatgaaggt | 180 |
| tggttgcaag cttctattgg ttacactgtt caatgcgtgt ctatcagaga tcagttgttt | 240 |
| acttggtccc cagttttgag gccaattatt ggtccatttt tgccatccgt tagatctgtt | 300 |
| agaaggcatt tgagattcgc tgctgaaatt atggctccat tgatttctca agccttgcaa | 360 |
| gacgaaaaac aacatagagc tgataccttg ttggctgatc aaactgaagg tagaggtact | 420 |
| ttcatttcct ggttgttgag acatttgcca gaagaattga gaaccccaga acaagttggt | 480 |
| ttggatcaaa tgttggtttc ctttgctgct attcatacca ctactatggc tttgacaaag | 540 |
| gttgtttggg aattggtaaa aaggccagag tacattgaac cattgagaac cgaaatgcaa | 600 |
| gatgtttttg gtccagatgc tgtttctcca gatatctgca ttaacaaaga agccttgtcc | 660 |
| agattgcaca agttggattc tttcatcaga gaagttcaaa gatggtgtcc atctactttc | 720 |
| gttactccat ctagaagagt catgaagtct atgactttgt ccaacggtat caagttgcaa | 780 |
| agaggtactt ctattgcttt tccagctcat gccattcaca tgtctgaaga aactccaaca | 840 |
| ttttccccag atttctcttc cgattttgaa aacccatccc caagaatttt cgacggtttt | 900 |
| agatacttga acttgaggtc cattaagggt caaggttcac aacatcaagc tgctactact | 960 |
| ggtccagatt acttgatttt caatcatggt aaacatgcct gcccaggtag attttttgct | 1020 |
| atctctgaaa tcaagatgat tttgatcgag ttgttggcca agtacgactt cagattggaa | 1080 |
| gatggtaaac caggtccaga attgatgaga gttggtactg aaactagatt ggataccaaa | 1140 |
| gctggtttgg aaatgagaag aaggtga | 1167 |

SEQ ID NO: 46

| | |
|---|---|
| MKYTTCQMNI FPSLWSMKTS FRWPRTSKWS SVSLYDMMLR TVALLSGRAF VGLPLCRDEG | 60 |
| WLQASIGYTV QCVSIRDQLF TWSPVLRPII GPFLPSVRSV RRHLRFAAEI MAPLISQALQ | 120 |
| DEKQHRADTL LADQTEGRGT FISWLLRHLP EELRTPEQVG LDQMLVSFAA IHTTTMALTK | 180 |
| VVWELVKRPE YIEPLRTEMQ DVFGPDAVSP DICINKEALS RLHKLDSFIR EVQRWCPSTF | 240 |
| VTPSRRVMKS MTLSNGIKLQ RGTSIAFPAH AIHMSEETPT FSPDFSSDFE NPSPRIFDGF | 300 |
| RYLNLRSIKG QGSQHQAATT GPDYLIFNHG KHACPGRFFA ISEIKMILIE LLAKYDFRLE | 360 |
| DGKPGPELMR VGTETRLDTK AGLEMRRR | 388 |

SEQ ID NO: 47

| | |
|---|---|
| atggccgaat tggatacctt ggatatcgtt gttttgggtg ttatcttctt gggtactgtt | 60 |
| gcttacttca ccaaaggtaa attgtggggt gttactaagg atccatacgc taatggtttt | 120 |
| gctgctggtg gtgcttctaa accaggtaga actagaaata tcgttgaagc catggaagaa | 180 |
| tctggtaaga actgtgttgt tttctacggt tctcaaactg gtactgctga agattatgct | 240 |
| tccagattgg ctaaagaagg taagagtaga ttcggtttga acaccatgat tgccgatttg | 300 |
| gaagattacg atttcgataa cttggatacc gtcccatctg ataacatcgt tatgtttgtt | 360 |
| ttggctacct acggtgaagg tgaacctact gataatgctg ttgacttcta cgaattcatt | 420 |
| accggtgaag atgcttcttt caacgaaggt aatgatccac cattgggtaa cttgaattac | 480 |
| gttgcttttg gtttgggtaa caacacctac gaacattaca actccatggt tagaaacgtc | 540 |
| aacaaggctt tggaaaaatt gggtgctcat agaattggtg aagctggtga aggtgatgat | 600 |
| ggtgctggta ctatggaaga agattttttg gcttggaaag acccaatgtg ggaagccttg | 660 |
| gctaaaaaga tgggtttgga agaaagagaa gctgtctacg aacctatttt cgccattaac | 720 |
| gaaagagatg atttgacccc tgaagccaat gaagtttatt tgggtgaacc taacaagttg | 780 |
| cacttggaag gtactgctaa aggtccattc aattctcaca acccatatat tgctccaatc | 840 |
| gccgaatctt acgaattatt ctctgctaag gatagaaact gcttgcacat ggaaattgac | 900 |
| atctctggtt ctaatttgaa gtacgaaacc ggtgatcata ttgccatttg gccaactaat | 960 |
| ccaggtgaag aagttaacaa gttcttggac atcttggact tgtccggtaa acaacattct | 1020 |
| gttgttactg ttaaggcctt ggaacctaca gctaaagttc cttttccaaa tccaactacc | 1080 |
| tacgatgcca ttttgagata ccatttggaa atttgcgctc cagtctctag acaattcgtt | 1140 |
| tctactttgg ctgcttttgc tccaaacgat gatattaagg ctgaaatgaa cagattgggt | 1200 |
| tccgataagg attacttcca cgaaaaaact ggtccacact actacaacat tgctagattt | 1260 |
| ttggcctctg tctctaaagg tgaaaagtgg actaagattc cattctccgc tttcattgaa | 1320 |
| ggtttgacta agttgcaacc tagatattac tccatctcct cctcatcttt ggttcaacct | 1380 |
| aagaagatct ctattaccgc cgttgttgaa tcccaacaaa ttccaggtag agatgatcct | 1440 |
| tttagaggtg ttgctaccaa ttacttgttc gccttgaaac aaaagcaaaa cggtgatcca | 1500 |
| aatcctgctc catttggtca atcttatgaa ttgactggtc caagaaacaa gtacgatggt | 1560 |
| attcatgttc cagttcacgt tagacactct aactttaagt tgccatctga tccaggtaag | 1620 |
| ccaattatca tgattggtcc aggtactggt gttgctccat tcagaggttt tgttcaagaa | 1680 |
| agagctaagc aagctagaga tggtgttgaa gttggtaaaa ccttgttgtt cttcggttgt | 1740 |
| agaaagtcca ctgaagattt catgtaccaa aaagaatggc aagaatacaa agaagcctta | 1800 |
| ggtgacaagt tcgaaatgat tactgccttc tcaagagaag gttctaagaa ggtttacgtc | 1860 |
| caacacagat tgaaagaaag atccaaagaa gtctccgatt tgttgtctca aaaggcctac | 1920 |
| ttttacgttt gtggtgatgc tgctcatatg gccagagaag ttaatactgt tttggcccaa | 1980 |
| attatcgctg aaggtagagg tgtatctgaa gctaagggtg aagaaatcgt taagaacatg | 2040 |
| agatccgcca atcaatacca agtttgctct gattttgtta ccttgcactg taaagaaacc | 2100 |
| acctacgcta attccgaatt gcaagaagat gtttggtcct aa | 2142 |

SEQ ID NO: 48

| | |
|---|---|
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQVCS DFVTLHCKET TYANSELQED VWS | 713 |

SEQ ID NO: 49

| | |
|---|---|
| atggccgaac aacaaatctc caacttgttg tctatgttca acgccttcca taccaatcag | 60 |
| aagttggaaa tctctgttca agttaccgat tccttccagt atagagatac tcctccagat | 120 |
| tcttcatctt ctgaaggtgg ttctttgtcc agatacgaag aaagaagagt ttctttgcca | 180 |
| ttggctagaa attctccatc tccagatatc gttttccagt tgtgtttttc taccgccacc | 240 |
| atttctgaat tgaaccatag atggaagtcc cagagattga aagttgctga ttctccatac | 300 |
| aactacatct tgactttgcc atccaaaggt attagaggtg ccttcattga ttctttgaac | 360 |
| gtttggttgg atgttccaga agataaggcc caagttatca aggatgttat cgatatgttg | 420 |
| cacaactcct cattgatcat cgatgacttt caagatggtt ccccattgag aagaggtaaa | 480 |
| ccatctactc atactgtttt tggtccagct caagctatta acactgctac ctacattatc | 540 |
| gttaaggcca tcgaaagaat ccaagagatc gtttctcatg atgctttggc tgatattacc | 600 |
| ggtactatta ccactatctt tcaaggtcaa gctatggatt tgtggtggac tgctaacacc | 660 |
| atattgcaat ccattcaaga atacttgttg atggtcaacg ataagactgg tgctttgttc | 720 |
| agattgtctt tggaattatt ggccttgaac tccgaagctc caatttctga ttctaccttg | 780 |
| gaatccttgt cctccgttgt ttctttgttg ggtcaatact tccaaatcag ggatgactac | 840 |
| atgaacttga tcgataacaa gtacaccgac caaaagggtt tctgtgaaga tttggatgag | 900 |
| ggcaaatact ccttgacttt gattcatgct ttacaaaccg actcctccga tttgttgatt | 960 |
| aacgttttgt ccatgagaag agtccaaggt aaattgacta cccaacaaaa gatgttggtc | 1020 |
| ttggaagtta tgaagaccaa cggttctttg gattggactt ctaagttgtt gggtatgttg | 1080 |
| catacaagag ttgttgccga aatcgactcc ttggaaattt ctatgaagag agataaccca | 1140 |
| gctttgagag ctttggttga aagattgaag ccagaaacct ga | 1182 |

SEQ ID NO: 50

| | |
|---|---|
| MAEQQISNLL SMFNAFHTNQ KLEISVQVTD SFQYRDTPPD SSSSEGGSLS RYEERRVSLP | 60 |
| LARNSPSPDI VFQLCFSTAT ISELNHRWKS QRLKVADSPY NYILTLPSKG IRGAFIDSLN | 120 |
| VWLDVPEDKA QVIKDVIDML HNSSLIIDDF QDGSPLRRGK PSTHTVFGPA QAINTATYII | 180 |
| VKAIERIQEI VSHDALADIT GTITTIFQGQ AMDLWWTANT ILQSIQEYLL MVNDKTGALF | 240 |
| RLSLELLALN SEAPISDSTL ESLSSVVSLL GQYFQIRDDY MNLIDNKYTD QKGFCEDLDE | 300 |
| GKYSLTLIHA LQTDSSDLLI NVLSMRRVQG KLTTQQKMLV LEVMKTNGSL DWTSKLLGML | 360 |
| HTRVVAEIDS LEISMKRDNP ALRALVERLK PET | 393 |

SEQ ID NO: 51

| | |
|---|---|
| atgaagggtt tggttgttgt tggtgcttct tatgctggtg ttcaagctgc tttgactgct | 60 |
| agagatgctg gttttgctaa acctattgct atcgttggtg atgaaccatg tttgccatat | 120 |
| caaagaccac cattgtctaa ggattacttg ttggataacg cctccgaaca atctttgttc | 180 |
| ttgagagata atgctttctt cggtgccaag ggtattgaat tgattttggg ttccagagtt | 240 |
| atcgacatcg atttgagaga tagaagggcc attttggaaa gaggttctgt tttgggtttc | 300 |
| gagcaattgg ttattgctgc tggttctaga gctagaagat tggaagttcc aggtggtcat | 360 |
| ttggaaggtg tttgttattt gagatccttg tctgatgctg cccatttgaa aatgagattg | 420 |
| aagcaagctg aagatgtcgt tattattggt ggtggtttca tcggtttgga agttgctgct | 480 |
| tctgctacaa aattgggtaa gaaggttgtt ttgattgaag ccggtcacag attattggaa | 540 |
| agagctactt ctccagttgt ctcctctttt ttgttggatg ctcatttgag agccggtgtt | 600 |
| gaaattagat tgctagaaac tgttgctgct ttcgaaggtg ctagaggtaa attgtctact | 660 |
| gtcttgttat cctccggttc caaagttaga gctgatatgg ttgttgtagg tattggtggt | 720 |
| attgccaatg atgaattggc tagaaaagct ggtttgaact gtactaatgg tgttaccgtt | 780 |
| tctgctcatg gtatgactga tgttgatggt gtttttgctt gtggtgattg tgcttaccat | 840 |
| ttcaacagat tctctaagac ttggaccaga ttggaatctg ttcaaaacgc tcaagatcaa | 900 |
| gctaaagctg ctggtttggc tattgctggt aaacattctc cagatatctc tgttccaaga | 960 |
| ttctggtctg atcaattcga cttgaagttg caaactactg gtattgctgg ttcttttgat | 1020 |
| gctgctgttg ttagaggtac tgttgatact ggtagattct ctaccttcta cttcaaggat | 1080 |
| ggttgtttgt tggctgttga ctctattaac agaccaggtg atcaattggt tgccagaaga | 1140 |
| ttgattgcag ctggtgtttc tccatctcaa ggtgaagctg ctgatatttc tttcgacttg | 1200 |
| aaatctttgg tcactccata a | 1221 |

SEQ ID NO: 52

| | |
|---|---|
| MKGLVVVGAS YAGVQAALTA RDAGFAKPIA IVGDEPCLPY QRPPLSKDYL LDNASEQSLF | 60 |
| LRDNAFFGAK GIELILGSRV IDIDLRDRRA ILERGSVLGF EQLVIAAGSR ARRLEVPGGH | 120 |
| LEGVCYLRSL SDAAHLKMRL KQAEDVVIIG GGFIGLEVAA SATKLGKKVV LIEAGHRLLE | 180 |
| RATSPVVSSF LLDAHLRAGV EIRLLETVAA FEGARGKLST VLLSSGSKVR ADMVVVGIGG | 240 |
| IANDELARKA GLNCTNGVTV SAHGMTDVDG VFACGDCAYH FNRFSKTWTR LESVQNAQDQ | 300 |
| AKAAGLAIAG KHSPDISVPR FWSDQFDLKL QTTGIAGSFD AAVVRGTVDT GRFSTFYFKD | 360 |
| GCLLAVDSIN RPGDQLVARR LIAAGVSPSQ GEAADISFDL KSLVTP | 406 |

SEQ ID NO: 53

| | |
|---|---|
| atgagagtcg aaaaccacaa cagagatgtt atcggtgttt ctgttgctcc aactcacttg | 60 |
| gataatttgt catctgctat cttgcaacaa ggtggtatgg ctagagtttc tttgccaggt | 120 |
| gatgttgtta cttgggctgc tggtggtcat caaactttga gaagaatttt gtccgaccag | 180 |
| agattcaaca gagattggag acagtggaga gctttacaag atggtgaaat tccagaagat | 240 |
| catccattga ttggtatgtg caaggttgat aacatggtta ctgctcatgg tgctgatcat | 300 |
| agaagattga gaggtttgtt gtctagatct ttcccaccat ctagaattgc tttgttggct | 360 |
| ccaagaattg aacaatgggt tgatagatta ttggccgaaa tggctcaaag aggtggttct | 420 |
| gctgatttga tgtgtgaatt tgctgttcca ttgcctacca atgttattgc tgaattattc | 480 |
| ggtttgccag acgaacagag agaagaaata gttgctttga cttactcttt ggctaacact | 540 |
| tctgctactg ctgctgaagt tagacaaacc agacaaagaa ttccagagtt cttcagaaga | 600 |
| ttgatcgctt tgaaaagggg tcaattgggt gatgatttgg cttctgcttt gatagttgct | 660 |
| agagataacg gtgaattggt ttccgatacc gaattgatcg atatgttgtt catggttttg | 720 |
| tccgctggtt tcgttactac tactggtgtt attggtaatg gtgttttggc tttgttgacc | 780 |
| catccacaac aattgcattt ggttagagct ggtcaagttc catggtcaca agctattgaa | 840 |
| gaaattttga gatggggttc ctctgttgct aatttgcctt ttagatacgc taccgaagat | 900 |
| gttgaaattg atggttgcat ggttagaaga ggtgatgctg ttttgatggc ttttcatgct | 960 |
| gctaatagag atgagaaagc ttttggtcca ggtgctgata gatttgatgt tactagaagg | 1020 |
| cataacccac acttgtcttt tggtgaaggt ccacattttt gtttgggtgc tgctttggct | 1080 |
| agattggaat tgagatgtgc ttttccagct ttgtttgcca gattggaaga tttggctttg | 1140 |
| actattgctg ctgaagatgt tgtttacatg ccatcctacg ttattagatg cccacaaaga | 1200 |
| ttgccagtta ctttcagacc atctattgcc tga | 1233 |

SEQ ID NO: 54

| | |
|---|---|
| MRVENHNRDV IGVSVAPTHL DNLSSAILQQ GGMARVSLPG DVVTWAAGGH QTLRRILSDQ | 60 |
| RFNRDWRQWR ALQDGEIPED HPLIGMCKVD NMVTAHGADH RRLRGLLSRS FPPSRIALLA | 120 |
| PRIEQWVDRL LAEMAQRGGS ADLMCEFAVP LPTNVIAELF GLPDEQREEI VALTYSLANT | 180 |
| SATAAEVRQT RQRIPEFFRR LIALKRGQLG DDLASALIVA RDNGELVSDT ELIDMLFMVL | 240 |
| SAGFVTTTGV IGNGVLALLT HPQQLHLVRA GQVPWSQAIE EILRWGSSVA NLPFRYATED | 300 |
| VEIDGCMVRR GDAVLMAFHA ANRDEKAFGP GADRFDVTRR HNPHLSFGEG PHFCLGAALA | 360 |
| RLELRCAFPA LFARLEDLAL TIAAEDVVYM PSYVIRCPQR LPVTFRPSIA | 410 |

SEQ ID NO: 55

| | |
|---|---|
| atgggtttgg cttcttcttg ggtcttgtac actgctattt ttgctggtgc tttggctttg | 60 |
| agatgggttt tgttgagagt taacaagtgg gtttacgagg gtagattgaa gggtaaatct | 120 |
| tatcatttgc caccaggtga tttgggttgg ccattgattg gtaatatgtg gacttttttg | 180 |
| agagccttca agaccaagaa tccagactct ttcatttcca acatcgtcga aagatatggt | 240 |
| aagggtggta tctacaagac tttcatgttt ggtaacccat ccatcttggt tacttctcca | 300 |
| gaaggttgta gaaaggtttt gaccgatgat gataatttca aaccaggttg gccaacttct | 360 |
| accgaagaat tgataggtaa gaagtccttc gtcagcatct cttacgaaga acataagaga | 420 |
| ttgagaagat tgacctctgc tccagttaat ggtcatgaag ctttgtcctt gtacatccct | 480 |
| tacatcgaaa agaacgttat ctccgatttg gagaagtggt ctaagatggg taacattgaa | 540 |
| ttcttgaccg gtgttagaaa gttgaccttc aagatcatca tgtacatttt cttgtccgcc | 600 |
| gaatctggtg atgttatgga agctttggaa aaagagtaca ccatcttgaa ctatggtgtt | 660 |
| agagctttgg ccattaacat tccaggtttt gcttttcata aggccttcaa ggctagaaag | 720 |
| aatttggttg ctactttaca agctaccgtt gacgaaagaa ggcaaagaga aagagaaaac | 780 |
| tcttccgcta gagaaaagga tatgttggat gctttgttgc acgttgaaga tgagaatggt | 840 |
| agaaaattga ccgacgaaga aatcatcgac ttgttgatca tgtacttgaa cgctggtcat | 900 |
| gaatcttcag gtcatgttac tatgtgggct actttgttgt tgcaaggtca tccagaaatt | 960 |
| ttccaaagag ctaaggctga acaagaagag atcgttaaga atagaccacc aactcaaaag | 1020 |
| ggtttgacct tgagagaagt taggaagatg gaatacttgt cccaagttat tgacgaaacc | 1080 |
| ttgagatggt tgaccttctc attgatggtt ttcagagaag ctaaggccga tgttaatatt | 1140 |
| ggtggttact tgtttccaaa gggttggaaa gttttggttt ggttcagagc tgttcattac | 1200 |
| gatccagaaa tctacccaaa tccagaagtt ttcaatccat ccagatggga taatttcact | 1260 |
| ccaaaggctg gtactttttt gccatttggt gctggttcta gattgtgtcc aggtaatgat | 1320 |
| ttggccaagt tggaaatctc tatcttcttg cactacttct tgttgaacta cagattggaa | 1380 |
| agggttaacc caggttgtga attgatgtat ttgccacatc caagaccagt tgataactgt | 1440 |
| ttggctagag ttagaaaggt tgcctga | 1467 |

SEQ ID NO: 56

| | |
|---|---|
| MGLASSWVLY TAIFAGALAL RWVLLRVNKW VYEGRLKGKS YHLPPGDLGW PLIGNMWTFL | 60 |
| RAFKTKNPDS FISNIVERYG KGGIYKTFMF GNPSILVTSP EGCRKVLTDD DNFKPGWPTS | 120 |
| TEELIGKKSF VSISYEEHKR LRRLTSAPVN GHEALSLYIP YIEKNVISDL EKWSKMGNIE | 180 |
| FLTGVRKLTF KIIMYIFLSA ESGDVMEALE KEYTILNYGV RALAINIPGF AFHKAFKARK | 240 |
| NLVATLQATV DERRQREREN SSAREKDMLD ALLHVEDENG RKLTDEEIID LLIMYLNAGH | 300 |
| ESSGHVTMWA TLLLQGHPEI FQRAKAEQEE IVKNRPPTQK GLTLREVRKM EYLSQVIDET | 360 |
| LRWLTFSLMV FREAKADVNI GGYLFPKGWK VLVWFRAVHY DPEIYPNPEV FNPSRWDNFT | 420 |
| PKAGTFLPFG AGSRLCPGND LAKLEISIFL HYFLLNYRLE RVNPGCELMY LPHPRPVDNC | 480 |
| LARVRKVA | 488 |

SEQ ID NO: 57

| | |
|---|---|
| atgatcttgg agatgggttc tatgtgggtt gttttgatgg ctattggtgg tgctttgttg | 60 |
| gttttgagat ccatcttgaa gaatgtcaac tggtggttgt acgaatctaa gttgggtgtt | 120 |
| aagcaatact ctttgccacc aggtgatatg ggttggcctt ttattggtaa tatgtggtct | 180 |
| ttcttgaggg ccttcaaatc taaagatcca gactccttca tctcctccat cgtttctaga | 240 |
| tatggttctt ctggtatcta caaggctttg atgtttggta acccatctgt tatcgttact | 300 |
| actccagaag gttgtaagag ggttttgact gatgacgaaa agtttactac tggttggcca | 360 |
| caatctacca ttgaattgat tggtaagaac tccttcattg ccatgactta cgaagaacac | 420 |
| aagagattga gaaggttgac ctcctcttct attaacggta tggaagcttt gtccttgtac | 480 |
| ttgaagtaca tcgaagagaa cgtgatcatc tctttggaaa agtggtctaa catgggtcag | 540 |
| attgaattct tgaccgagat taggaagttg accttcaaga tcatcatgca cattttcttg | 600 |
| tcctccgaat ctgaaccagt tatggaagcc ttggaaaaag agtacaccat tttgaaccat | 660 |
| ggtgttagag ctatgcaaat caatgttcca ggtttcgctt actacaaagc tttgaaggct | 720 |
| agaaagaact tggtcggtat tttccaatcc atcgttgatg acagaagaaa catcagaaag | 780 |
| gtctactccc aaaaaaaggc caaggatatg atggattcct tgatcgatgt tgaagatgac | 840 |
| aacggtagaa agttgaacga cgaagatatc atcgacatca tgttgatgta cttgaacgct | 900 |
| ggtcatgaat cctctggtca tattactatg tgggctactt acttcttgca aaagcaccca | 960 |
| gaatacttga agaaggccaa agaagaacaa gaagaaatca tcaagagaag gccatctact | 1020 |
| caaaagggtt tgaccttgaa agaaatcaga ggtatggact tcttgtacaa ggttattgac | 1080 |
| gaaaccatga gggtgattac cttctcattg gttgttttca gagaagccaa gtctgatgtt | 1140 |
| accattaacg gttacactat tccaaagggt tggaaggttt tgacctggtt tagatctgtt | 1200 |
| catttggacc cagaaatcta cccaaaccca aaagaattca acccaaacag gtggaacaaa | 1260 |
| gaacataagg ctggtgaatt tttgccattt ggtgctggta ctagattgtg tccaggtaat | 1320 |
| gatttggcca agatggaaat tgctgttttc ttgcatcatt tcaccttgaa ctacaggttg | 1380 |
| gaacaattga atccaaagtg cccaattaga tacttgccac atacaagacc aatggataac | 1440 |
| tgtttgggta gagttaagaa gtgttaa | 1467 |

SEQ ID NO: 58

| | |
|---|---|
| MILEMGSMWV VLMAIGGALL VLRSILKNVN WWLYESKLGV KQYSLPPGDM GWPFIGNMWS | 60 |
| FLRAFKSKDP DSFISSIVSR YGSSGIYKAL MFGNPSVIVT TPEGCKRVLT DDEKFTTGWP | 120 |
| QSTIELIGKN SFIAMTYEEH KRLRRLTSSS INGMEALSLY LKYIEENVII SLEKWSNMGQ | 180 |
| IEFLTEIRKL TFKIIMHIFL SSESEPVMEA LEKEYTILNH GVRAMQINVP GFAYYKALKA | 240 |
| RKNLVGIFQS IVDDRRNIRK VYSQKKAKDM MDSLIDVEDD NGRKLNDEDI IDIMLMYLNA | 300 |
| GHESSGHITM WATYFLQKHP EYLKKAKEEQ EEIIKRRPST QKGLTLKEIR GMDFLYKVID | 360 |
| ETMRVITFSL VVFREAKSDV TINGYTIPKG WKVLTWFRSV HLDPEIYPNP KEFNPNRWNK | 420 |
| EHKAGEFLPF GAGTRLCPGN DLAKMEIAVF LHHFTLNYRL EQLNPKCPIR YLPHTRPMDN | 480 |
| CLGRVKKC | 488 |

SEQ ID NO: 59

| | |
|---|---|
| atgactgaaa ccggtttgat cttgatgtgg ttcccattga ttatcttggg tttgttcgtt | 60 |
| ttgaagtggg ttttgaagag agttaacgtc tggatctacg tttctaagtt gggtgaaaaa | 120 |
| aagcactatt tgccaccagg tgatttgggt tggccagtta ttggtaatat gtggtctttt | 180 |
| ttgagagcct tcaagacctc tgatccagaa tctttcattc agtcctacat taccagatac | 240 |
| ggtagaactg gtatctacaa ggctcatatg tttggttacc catgtgtttt ggttactact | 300 |
| ccagaaacct gtagaagagt tttgactgat gatgatgcct tccatattgg ttggccaaaa | 360 |
| tctaccatga agttgatcgg tagaaagtcc ttcgttggta tctctttcga agaacacaag | 420 |
| agattgagaa gattgacttc tgctccagtt aatggtccag aagctttgtc tgtttacatc | 480 |
| cagttcattg aagaaaccgt taacaccgat ttggagaagt ggtctaaaat gggtgaaatc | 540 |
| gaattcttgt cccacttgag aaagttgacc ttcaaggtta ttatgtacat cttcttgtcc | 600 |
| tccgaatccg aacatgttat ggattctttg gaaagagagt acaccaactt gaactatggt | 660 |
| gttagagcta tgggtattaa cttgccaggt tttgcttatc atagagcttt gaaggctaga | 720 |
| aagaaattgg ttgctgcttt ccaatctatc gtcaccaaca gaagaaatca gagaaagcag | 780 |
| aacatctcct ccaacagaaa agatatgttg gacaacttga tcgacgtcaa ggacgaaaat | 840 |
| ggtagagttt tggatgacga agaaatcatc gacttgttgt tgatgtactt gaacgctggt | 900 |
| catgaatctt caggtcattt gactatgtgg gctaccattt tgatgcaaga acatccaatg | 960 |
| atcttgcaga aggccaaaga agaacaagaa agaatcgtta agaaaagagc cccaggtcaa | 1020 |
| aagttgactt tgaaagaaac tagggaaatg gtctacttgt cccaagttat tgacgaaacc | 1080 |
| ttgagagtga ttaccttctc attgactgct ttcagagaag ccaaatccga tgttcaaatg | 1140 |
| gatggttaca ttatcccaaa gggttggaaa gttttgacgt ggtttagaaa cgttcacttg | 1200 |
| gatccagaaa tctacccaga tccaaaaaag ttcgatccat caagatggga aggttacact | 1260 |
| ccaaaagctg gtactttttt gccatttggt ttgggttctc atttgtgtcc aggtaatgat | 1320 |
| ttggccaagt tggaaatctc catcttcttg catcatttct tgttgaagta cagggtcgaa | 1380 |
| agatctaatc caggttgtcc agttatgttc ttgccacata atagaccaaa ggataactgc | 1440 |
| ttggctagaa ttactagaac catgccatga | 1470 |

SEQ ID NO: 60

| | |
|---|---|
| MTETGLILMW FPLIILGLFV LKWVLKRVNV WIYVSKLGEK KHYLPPGDLG WPVIGNMWSF | 60 |
| LRAFKTSDPE SFIQSYITRY GRTGIYKAHM FGYPCVLVTT PETCRRVLTD DDAFHIGWPK | 120 |
| STMKLIGRKS FVGISFEEHK RLRRLTSAPV NGPEALSVYI QFIEETVNTD LEKWSKMGEI | 180 |
| EFLSHLRKLT FKVIMYIFLS SESEHVMDSL EREYTNLNYG VRAMGINLPG FAYHRALKAR | 240 |
| KKLVAAFQSI VTNRRNQRKQ NISSNRKDML DNLIDVKDEN GRVLDDEEII DLLLMYLNAG | 300 |
| HESSGHLTMW ATILMQEHPM ILQKAKEEQE RIVKKRAPGQ KLTLKETREM VYLSQVIDET | 360 |
| LRVITFSLTA FREAKSDVQM DGYIIPKGWK VLTWFRNVHL DPEIYPDPKK FDPSRWEGYT | 420 |
| PKAGTFLPFG LGSHLCPGND LAKLEISIFL HHFLLKYRVE RSNPGCPVMF LPHNRPKDNC | 480 |
| LARITRTMP | 489 |

SEQ ID NO: 61

| | |
|---|---|
| atggctgaaa ctacttcttg gattccagtt tggtttccat tgatggtttt gggttgtttt | 60 |
| ggtttgaact ggttggttag aaaggttaac gtttggttgt acgaatcttc cttgggtgaa | 120 |
| aacagacatt atttgccacc aggtgatttg ggttggcctt ttattggtaa tatgttgtcc | 180 |
| ttcttgagag ccttcaaaac ctctgatcca gattctttca ctaggacctt gattaagaga | 240 |
| tacggtccaa aaggtatcta caaggctcat atgtttggta acccatctat tatcgttacc | 300 |
| acctctgata cctgtagaag agttttgact gatgatgatg cttttaaacc aggttggcca | 360 |
| acttctacca tggaattgat tggtagaaag tccttcgttg gtatctcttt cgaagaacac | 420 |
| aagagattga gaagattgac tgctgctcca gttaatggtc atgaagcttt gtctacctac | 480 |
| atcccttaca tcgaagaaaa cgttattacc gttttggaca agtggactaa gatgggtgaa | 540 |
| tttgaattct tgacccactt gagaaagttg accttcagaa tcatcatgta cattttcttg | 600 |
| tcctccgaat ccgaaaacgt tatggatgct ttggaaagag agtacactgc tttgaattat | 660 |
| ggtgttagag ctatggccgt taacattcca ggttttgctt atcatagagc tttgaaggct | 720 |
| agaaagactt tggttgctgc tttccaatct atcgttaccg aaagaagaaa tcagaggaag | 780 |
| cagaacatct tgtccaacaa aaaggatatg ttggacaact tgttgaacgt taaggacgaa | 840 |
| gatggtaaga ccttggatga tgaagaaatc atcgatgtct tgttgatgta cttgaacgct | 900 |
| ggtcatgaat cttccggtca tacaattatg tgggctactg ttttcttaca agaacaccca | 960 |
| gaagttctac aaagagctaa agctgaacaa gaaatgatct tgaagtctag accagaaggt | 1020 |
| caaaagggct tgtctttgaa agaaaccaga aagatggaat tcttgtccca agttgttgac | 1080 |
| gaaaccttga gagttattac cttctcattg accgctttca gagaagctaa aaccgatgtt | 1140 |
| gaaatgaacg gttacttgat tccaaagggt tggaaagttt tgacgtggtt cagagatgtt | 1200 |
| catatcgatc cagaagtttt cccagatcca agaaaatttg atccagctag atgggataat | 1260 |
| ggtttcgttc caaaagctgg tgcttttttg ccatttggtg ctggttctca tttgtgtcca | 1320 |
| ggtaatgatt tggccaagtt ggaaatctcc atcttcttgc atcacttttt gttgaagtac | 1380 |
| caggtcaaga gatctaaccc agaatgtcca gttatgtact tgccacatac aagaccaact | 1440 |
| gataactgct tggctagaat ctcttaccag tga | 1473 |

SEQ ID NO: 62

| | |
|---|---|
| MAETTSWIPV WFPLMVLGCF GLNWLVRKVN VWLYESSLGE NRHYLPPGDL GWPFIGNMLS | 60 |
| FLRAFKTSDP DSFTRTLIKR YGPKGIYKAH MFGNPSIIVT TSDTCRRVLT DDDAFKPGWP | 120 |
| TSTMELIGRK SFVGISFEEH KRLRRLTAAP VNGHEALSTY IPYIEENVIT VLDKWTKMGE | 180 |
| FEFLTHLRKL TFRIIMYIFL SSESENVMDA LEREYTALNY GVRAMAVNIP GFAYHRALKA | 240 |
| RKTLVAAFQS IVTERRNQRK QNILSNKKDM LDNLLNVKDE DGKTLDDEEI IDVLLMYLNA | 300 |
| GHESSGHTIM WATVFLQEHP EVLQRAKAEQ EMILKSRPEG QKGLSLKETR KMEFLSQVVD | 360 |
| ETLRVITFSL TAFREAKTDV EMNGYLIPKG WKVLTWFRDV HIDPEVFPDP RKFDPARWDN | 420 |
| GFVPKAGAFL PFGAGSHLCP GNDLAKLEIS IFLHHFLLKY QVKRSNPECP VMYLPHTRPT | 480 |
| DNCLARISYQ | 490 |

SEQ ID NO: 63

| | |
|---|---|
| atggcttcct tgtggtttat tttcggtgct attgctggtg ctttgttggt tttgagatct | 60 |
| ttgttgaaga acgtcaactg gttcttgtac gaagctaaat tgggtgacaa gcaatattct | 120 |
| ttgccaccag gtgatatggg ttggccaatt attggtaata tgtggtcttt cttgagggcc | 180 |
| ttcaaatctt ctaagccaga ttctttcatg gactccatcg ttaagagatt tggtaacact | 240 |
| ggtatctaca aggtgttcat gtttggtttc ccatctgtta tcgttacttc tccagaagct | 300 |
| tgcaaaaagg ttttgactga tgacgaaaat ttcgaaccag gttggccaca atctaccgtt | 360 |
| gaattgattg gtgaaaagtc cttcatcaag atgccattcg aagaacatag aaggttgaga | 420 |
| agattgacct ccgcttctat taacggttat gaagctttgt ccgtctactt gaagtacatc | 480 |
| gaagaaatcg tcatctcctc attggaaaag tggactcaaa tgggtgaaat cgaattcttg | 540 |
| acccagatga gaaagttgac cttcaagatc atcatccaca ttttcttggg ttccgaatct | 600 |
| gaaccagtta tggaagcttt ggaaagagag tacactgttt tgaacttggg tgttagagct | 660 |
| atgagaatca acattccagg tttcgctttc cacaaatctt tgaaggctag aaagaacttg | 720 |
| gttgccatct tccaatctat cgttgacaag agaagaaacg agagaagagg taaagaacca | 780 |
| gctccaggta aaaaagctaa ggatatgatg gattccttga tcgatgctgt tgacgaaaat | 840 |
| ggtagaaaat tgggtgatga cgaaatcatc gacatcatgt tgatgtactt gaacgctggt | 900 |
| catgaatcct ctggtcatat tactatgtgg gctacttact tcttgcaaag acatccagaa | 960 |
| ttcttcagaa aggccaaaga agaacaagtc gagatgttga aaagaaggcc accatctcaa | 1020 |
| aaaggtttga agttggaaga tgtgagaaag atggaatact tgtccaaggt tattgacgaa | 1080 |
| accatgagag ttgttacctt cagcttgatg gttttcagac aagctagaaa cgatgttaag | 1140 |
| gtcaacggtt acttgattcc aaaaggttgg agagttttga cgtggttcag atctgttcat | 1200 |
| ttcgattccg aattataccc agacccaaga gaattcaatc cagaaaactt ctccgttgtt | 1260 |
| agaaaggctg gtgaattttt gccatttggt gctggtacta gattgtgtcc aggtaatgat | 1320 |
| ttggccaagt tggaaatctc tgttttcttg catcacttct tgttgaagta cgaattggaa | 1380 |
| cagttgaacc caaagtcccc aattagattt ttgccacata caagaccatt ggataactgc | 1440 |
| ttggctagaa tcaaaaaaca agaagctgcc taa | 1473 |

SEQ ID NO: 64

| | |
|---|---|
| MASLWFIFGA IAGALLVLRS LLKNVNWFLY EAKLGDKQYS LPPGDMGWPI IGNMWSFLRA | 60 |
| FKSSKPDSFM DSIVKRFGNT GIYKVFMFGF PSVIVTSPEA CKKVLTDDEN FEPGWPQSTV | 120 |
| ELIGEKSFIK MPFEEHRRLR RLTSASINGY EALSVYLKYI EEIVISSLEK WTQMGEIEFL | 180 |
| TQMRKLTFKI IIHIFLGSES EPVMEALERE YTVLNLGVRA MRINIPGFAF HKSLKARKNL | 240 |
| VAIFQSIVDK RRNERRGKEP APGKKAKDMM DSLIDAVDEN GRKLGDDEII DIMLMYLNAG | 300 |
| HESSGHITMW ATYFLQRHPE FFRKAKEEQV EMLKRRPPSQ KGLKLEDVRK MEYLSKVIDE | 360 |
| TMRVVIFSLM VFRQARNDVK VNGYLIPKGW RVLTWFRSVH FDSELYPDPR EFNPENFSVV | 420 |
| RKAGEFLPFG AGTRLCPGND LAKLEISVFL HHFLLKYELE QLNPKSPIRF LPHTRPLDNC | 480 |
| LARIKKQEAA | 490 |

SEQ ID NO: 65

| | |
|---|---|
| atggaatcta cttgggctgt tgctgctgtt gttacagctg ttgttgcagt tgctactgtt | 60 |
| ttctctgttt tgaaatgggc tgctaagtct ttgaacgaat ggatctatga agctaagttg | 120 |
| ggtgatagaa gattggcttt gccaccaggt gatttgggtt ggccattgat tggtaatatg | 180 |
| ttgggttttt tgagggcctt caagtctaag aatccagaaa ctttcatcga cggttacgtt | 240 |
| tctagatacg gtaaaactgg tgtttacaag gttcacttgt ttggtaaccc atctgttgtt | 300 |
| gttactactc cagaaacctg tagaaaggtt ttgactgatg atgaagcttt tcaaccaggt | 360 |
| tggccaagag ctgctgttga attgattggt gaaaagtcct tcatccagat gccacaagaa | 420 |
| gaacataaga gattgagaag attgacctct gctccagtta atggttttga agctttgtcc | 480 |
| aactacatcc cttacatcga aaagaacgtc ttggaatctt tggagaagtg gtctaaaatg | 540 |
| ggtccaattg aattcttgac ccagttgaga aagttgacct tcaccgttat tatgtacatc | 600 |
| ttcttgtcct ccgaatccga accagttatg gaaatgttgg aaaaagagta caccaggttg | 660 |
| aactacggtg ttagagatat gagaatcaac ttgccaggtt tcgcttatca taaggctttg | 720 |
| aaggctagaa agaatttggt tgctgctttg aagggtatcg ttactgaaag aagaaggcaa | 780 |
| aagttggata agtgggctcc aaaaagaaag gatatgatgg accaattgat cgacatcgtt | 840 |
| gacgaaaatg gtagaaagtt ggatgacgaa gaaatcatcg acatcttgat catgtacttg | 900 |
| aacgctggtc atgaatcttc aggtcataca atgatgtggg ctaccatctt gttgaatcaa | 960 |
| catccagaag ttttgaagaa ggccagggaa gaacaagaag ctatcgttag aaatagacca | 1020 |
| gcaggtcaaa ctggcttgac tttgaaagaa tgtagagaca tggaatactt gtccaaggtt | 1080 |
| gttgacgaaa ccttgagata cgtttccttc tcattggtcg ttttcagaga agctcaaatg | 1140 |
| gatgttaact tgaacggtta cttgattcca aagggttgga aagttttggc ctggttcaga | 1200 |
| tctattcact acgattctga agtttaccca gacccaaaaa agttcgaacc atcaagatgg | 1260 |
| gatggttttg ttccaaaagc tggtgaattt ttgccatttg gtgctggttc tagattgtgt | 1320 |
| ccaggtaatg atttggctaa gttggaaatc tgcatcttcg tccactactt tttgttgaac | 1380 |
| tacaacttgg aatggttgac cccagattgt gaaatcttgt atttgccaca ttccagacca | 1440 |
| aaggataact gcatggctaa gattaccaag aaatcttctg ttgctgccta a | 1491 |

SEQ ID NO: 66

| | |
|---|---|
| MESTWAVAAV VTAVVAVATV FSVLKWAAKS LNEWIYEAKL GDRRLALPPG DLGWPLIGNM | 60 |
| LGFLRAFKSK NPETFIDGYV SRYGKTGVYK VHLFGNPSVV VTTPETCRKV LTDDEAFQPG | 120 |
| WPRAAVELIG EKSFIQMPQE EHKRLRRLTS APVNGFEALS NYIPYIEKNV LESLEKWSKM | 180 |
| GPIEFLTQLR KLTFTVIMYI FLSSESEPVM EMLEKEYTRL NYGVRDMRIN LPGFAYHKAL | 240 |
| KARKNLVAAL KGIVTERRRQ KLDKWAPKRK DMMDQLIDIV DENGRKLDDE EIIDILIMYL | 300 |
| NAGHESSGHT MMWATILLNQ HPEVLKKARE EQEAIVRNRP AGQTGLTLKE CRDMEYLSKV | 360 |
| VDETLRYVSF SLVVFREAQM DVNLNGYLIP KGWKVLAWFR SIHYDSEVYP DPKKFEPSRW | 420 |
| DGFVPKAGEF LPFGAGSRLC PGNDLAKLEI CIFVHYFLLN YNLEWLTPDC EILYLPHSRP | 480 |
| KDNCMAKITK KSSVAA | 496 |

SEQ ID NO: 67

| | |
|---|---|
| atgggtgaag gtgcttggtg ggctgttgct gctgttgttg ctgctttggc tgttgttgca | 60 |
| ttggatgctg ctgttagaac tgctcatgct tggtattgga ctgcttcttt gggtgctggt | 120 |
| agaagaggta gattgccacc aggtgatatg ggttggccat tggttggtgg tatgtgggct | 180 |
| tttttgagag cttttaaatc tggtagacca gactccttca ttgattcttt tgctagaaga | 240 |
| tttggtagag ccggcttgta tagagctttt atgttttctt ctccaaccat tatggctact | 300 |
| actccagaag cttgtaagca agttttgatg gatgatgatg ctttcgttac tggttggcca | 360 |
| aaagctactg ttgctttgat tggtccaaag tcctttgtta acatgggtta cgatgaacac | 420 |
| agaaggttga gaaaattgac tgctgctcca atcaatggtt tcgatgcttt gacttcttac | 480 |
| ttgggtttca tcgatgatac tgttgttact actttgaggg gttggtctga aaggggtggt | 540 |
| gatggtcatt ttgaattctt gactgaattg agaaggatga ccttcagaat catcgtccaa | 600 |
| attttcatgg gtggtgctga cgaaagaact gctgctgaat tggaaagaac ttacaccgaa | 660 |
| ttgaactacg gtatgagagc tatggctatt gatttgccag gttttgctta ccataaggct | 720 |
| attagagcta gaagaagatt ggttgctgct ttacaaagag ttttggacga gagaagggct | 780 |
| agaggtggta aaactgctgc tggtgctgct gctccagttg atatgatgga tagattgatt | 840 |
| gccgttgaag atgaaggtgg tagaagattg caagatgacg aaatcatcga tgtcttggtc | 900 |
| atgtatttga acgctggtca tgaatcctct ggtcatatta ctatgtgggc tactgttttc | 960 |
| ttgcaagaga acccagaaat tttggctaaa gctaaagctg aacaagaggc cattatgaga | 1020 |
| tctattccac caggtcaaaa aggcttgact ttgagagatt ttagaaagat ggcctacttg | 1080 |
| tcccaagttg ttgacgaaac tttgagattc gtcaacatct ccttcgtgtc ttttagacaa | 1140 |
| gctaccagag atgttttcgt caacggttac ttgattccaa aaggttggaa agtccaattg | 1200 |
| tggtacagat ccgttcatat ggatccacaa gtttatccag atccaaagaa gttcgatcca | 1260 |
| tcaagatggg aaggtccacc accaagagct ggtacttttt tgccatttgg tttgggtact | 1320 |
| agattgtgtc caggtaatga tttggccaag ttggaaatct cagttttctt gcatcatttc | 1380 |
| ttgttgggct acaagttgac tagaaagaac ccaaactgta gagtcagata tttgccacat | 1440 |
| ccaagaccag ttgataactg cttggctaag attaccagat tgtcatcttc tcacggttaa | 150 |

SEQ ID NO: 68

| | |
|---|---|
| MGEGAWWAVA AVVAALAVVA LDAAVRTAHA WYWTASLGAG RRGRLPPGDM GWPLVGGMWA | 60 |
| FLRAFKSGRP DSFIDSFARR FGRAGLYRAF MFSSPTIMAT TPEACKQVLM DDDAFVTGWP | 120 |
| KATVALIGPK SFVNMGYDEH RRLRKLTAAP INGFDALTSY LGFIDDTVVT TLRGWSERGG | 180 |
| DGHFEFLTEL RRMTFRIIVQ IFMGGADERT AAELERTYTE LNYGMRAMAI DLPGFAYHKA | 240 |
| IRARRRLVAA LQRVLDERRA RGGKTAAGAA APVDMMDRLI AVEDEGGRRL QDDEIIDVLV | 300 |
| MYLNAGHESS GHITMWATVF LQENPEILAK AKAEQEAIMR SIPPGQKGLT LRDFRKMAYL | 360 |
| SQVVDETLRF VNISFVSFRQ ATRDVFVNGY LIPKGWKVQL WYRSVHMDPQ VYPDPKKFDP | 420 |
| SRWEGPPPRA GTFLPFGLGT RLCPGNDLAK LEISVFLHHF LLGYKLTRKN PNCRVRYLPH | 480 |
| PRPVDNCLAK ITRLSSSHG | 499 |

SEQ ID NO: 69

| | |
|---|---|
| atgccacaag ctattccagc tcataagatg atgccaattc caggtgttgg tgtttacgtt | 60 |
| tttactgttt tgtgggctgc tactatctac attgcttcat ctttgttgag atggtccttg | 120 |
| gattccttga aacatttgcc aatcgtcaac aacaaagaat ggtactcttt gtctggtaga | 180 |
| aaggccaagt tgagattttt ggctgaatcc aagtctttgt tggaagaagc tagaaagaga | 240 |
| tacccacaac aaccattcag aatcttgtct aattggggtg ttttgttggt tttgccatct | 300 |
| tgttttgccg acgaaatcag aaacgatcag agattgtctt tttcaaaggc tgccttgcaa | 360 |
| gattcccatg gtcatattcc aggtttggaa actgttaagt tggttgccag agatgaccaa | 420 |
| ttgattcaaa ccgttgctag aaagcacttg accaaacatt tggccaaagt tatccaacca | 480 |
| ttgtccgaag aaactgaatt cgctttggat caaaacttcg gtcataaccc agccatcttg | 540 |
| gatattattg ccagaatctc ttccaggatc tacttgggtg atgaattgtg tagaaatact | 600 |
| gcttggttgg ctactactaa ggtttacact tctgcttttt ttgctgcccc agttaagttg | 660 |
| ggtttgattc cagctccatt gagaagattg gctcattggt tgattccaga atgcaagatc | 720 |
| ttgagagaac aagttcaaga agccagaaga atcatcgaac cattggttag aagaaggcaa | 780 |
| gctttgagag ctaaagcttt ggctgaaggt tgtccaactc cacaattcaa tgatgctttg | 840 |
| ggttgggctg ctgaagaatc tgctaaaaat ggtaaagatt acgatccagc cattacccaa | 900 |
| ttggctttgt ctatgttggc tattcatacc acctacgact tgttccaaca atgcatttta | 960 |
| gatttggccc aaaacccaca tttcatcgaa cctttgagac aagaagccat cgaagtcatt | 1020 |
| caacaatatg gttggacaaa gcaaggcttg taccatatga agttgttgga ttccgctttg | 1080 |
| aaagaaaccc aaagattgaa accaggttcc atggttacta tgagaagata tgtcttggag | 1140 |
| gacttgcaat tgtccaacgg tttgattttg aaaaagggca ccagaatcaa catcgacact | 1200 |
| caaagaatga gagatccaga cttgcatgaa gatccattga agtacgatgc tttcaggttc | 1260 |
| tacaagatga gacaacaacc aggtggtgaa catactgctc aattggtttc tacttctcca | 1320 |
| gatcatttgg gttttggtca tggtgaacat tcttgtccag gtagattttt tgctgctaac | 1380 |
| gaaatcaaag ttgccatggc tcatatgttg atcaagtacg aatggaaacc agctggtcat | 1440 |
| tcttctgctg gtccagatgt taagggtttg ttgatgaagt ctggtgctgg tgctcaaatt | 1500 |
| gatatcagaa gaagagaaac cgttgagatc gcttga | 1536 |

SEQ ID NO: 70

| | |
|---|---|
| MPQAIPAHKM MPIPGVGVYV FTVLWAATIY IASSLLRWSL DSLKHLPIVN NKEWYSLSGR | 60 |
| KAKLRFLAES KSLLEEARKR YPQQPFRILS NWGVLLVLPS CFADEIRNDQ RLSFSKAALQ | 120 |
| DSHGHIPGLE TVKLVARDDQ LIQTVARKHL TKHLAKVIQP LSEETEFALD QNFGHNPAIL | 180 |
| DIIARISSRI YLGDELCRNT AWLATTKVYT SAFFAAPVKL GLIPAPLRRL AHWLIPECKI | 240 |
| LREQVQEARR IIEPLVRRRQ ALRAKALAEG CPTPQFNDAL GWAAEESAKN GKDYDPAITQ | 300 |
| LALSMLAIHT TYDLFQQCIL DLAQNPHFIE PLRQEAIEVI QQYGWTKQGL YHMKLLDSAL | 360 |
| KETQRLKPGS MVTMRRYVLE DLQLSNGLIL KKGTRINIDT QRMRDPDLHE DPLKYDAFRF | 420 |
| YKMRQQPGGE HTAQLVSTSP DHLGFGHGEH SCPGRFFAAN EIKVAMAHML IKYEWKPAGH | 480 |
| SSAAGPDVKL LMKSGAGAQI DIRRRETVEI A | 511 |

SEQ ID NO: 71

| | |
|---|---|
| atggaagtcg gtatggttat gaaggcttct ttgtctttgt gttgtgttgg tgcttgttgt | 60 |
| ttggccttgt acttgtatta tatcgtttgg gttgttccac aaaggttgtt ggctggtttt | 120 |
| agaaggcaag gtattggtgg tccaagacca tcttttccat atggtaattt ggccgatatg | 180 |
| aaggaagctg ttgctgctgc taaagttgct tctagaggtg ttggtggtat cgttcatgat | 240 |
| tatagaccag ctgttttgcc attctacgag aagtggagaa aagaacatgg tccagttttc | 300 |
| acttactcca tgggtaatgt tgttttcttg cacgtttcta gaccagatgt tgttagagat | 360 |
| atcaacttgt gcgtttcctt ggacttgggt aaatcttctt acttgaaggc tactcacgaa | 420 |
| cctttgtttg gtagaggtat tttgaagtct aatggtcaag cttgggctca ccaaagaaag | 480 |
| attattgctc cagcattctt cttggataag gttaagggta tggttgattt gatggttgat | 540 |
| tctgctcaaa ccttgttgaa gtcttgggaa gaaagggttg atggtaatgg tggtactgtt | 600 |
| aacatcaaga tcgatgatga tatcagagct tactccgccg atgttatttc tagaacttgt | 660 |
| ttcggttcct cctacatcaa gggtaagaag atctttttga agttgagaga attgcagaag | 720 |
| gccgtttcta agccaaatgt tttggctgaa atgactggtt tgaggttgtt tccaactaag | 780 |
| aagaatagac aagcctggga attgcataga caagttcata agttgatctt ggaaatcgtc | 840 |
| aaagaatccg gtgaggataa gaacttgttg tctactattt tacactccgc ctcttcatct | 900 |
| aaagttggtt tgggtgaagc tgaaaacttc atcgttgata actgcaagtc tatctacttc | 960 |
| gctggttatg aatctactgc tgttactgct gcttggtgtt tgatgttgtt gggtttacat | 1020 |
| ccagaatggc aagataaggt tagagaagag gttcaagagg tttgtggtgg tagaccaatt | 1080 |
| gattctcaat ccttgcaaaa gatgaagaac ctaaccatgg tcatccaaga aactttgaga | 1140 |
| ttatatccag ctggtgcctt cgtttctaga atggctttac aagaattgaa gttgggtggt | 1200 |
| gttaacatcc caaagggtgt taatatctac atcccagttt ctaccatgca cttggatcca | 1260 |
| aaattgtggg gtgctgatgt caaagaattc aacccagaaa gattctctga tgccagacca | 1320 |
| caattgcatt cttatttgcc atttggtgct ggtgctagaa catgtttggg tcaaggtttt | 1380 |
| gctactgccg aattgaagat tttgatctcc ttgatcattt ccaagttcgc cttgaagttg | 1440 |
| tccccattat atgaacattc tccaaccttg aagttggtcg ttgaaccaga atttggtgtt | 1500 |
| gatttgactt tgaccaaagt tcaaggtgct tgtagatgct ga | 1542 |

SEQ ID NO: 72

| | |
|---|---|
| MEVGMVMKAS LSLCCVGACC LALYLYYIVW VVPQRLLAGF RRQGIGGPRP SFPYGNLADM | 60 |
| KEAVAAAKVA SRGVGGIVHD YRPAVLPFYE KWRKEHGPVF TYSMGNVVFL HVSRPDVVRD | 120 |
| INLCVSLDLG KSSYLKATHE PLFGRGILKS NGQAWAHQRK IIAPAFFLDK VKGMVDLMVD | 180 |
| SAQTLLKSWE ERVDGNGGTV NIKIDDDIRA YSADVISRTC FGSSYIKGKK IFLKLRELQK | 240 |
| AVSKPNVLAE MTGLRLFPTK KNRQAWELHR QVHKLILEIV KESGEDKNLL STILHSASSS | 300 |
| KVGLGEAENF IVDNCKSIYF AGYESTAVTA AWCLMLLGLH PEWQDKVREE VQEVCGGRPI | 360 |
| DSQSLQKMKN LTMVIQETLR LYPAGAFVSR MALQELKLGG VNIPKGVNIY IPVSTMHLDP | 420 |
| KLWGADVKEF NPERFSDARP QLHSYLPFGA GARTCLGQGF ATAELKILIS LIISKFALKL | 480 |
| SPLYEHSPTL KLVVEPEFGV DLTLTKVQGA CRC | 513 |

SEQ ID NO: 73

| | |
|---|---|
| atggctttca ctgctcaatc ctacttcgat attggtgaac acttgagagt ttccgtcatt | 60 |
| ttgttgttga ctaccgttgt tttgttgttg gtgttctctt tgaaggccag aaagaaatct | 120 |
| ttgttgccat tggttaatgg taacagatgg actgatccat tgggtattga agccaagaaa | 180 |
| aagttcatga cctccgccag atctattatt gctgaacaat tggaaaaagc cccaggtaaa | 240 |
| cctttcagag ttgtttctga tgttggtgaa ttggttgttt tgccaccaga atttgctcca | 300 |
| gaaatcagaa accacaagga cttttctttt accatggctg cttacaagtg gttctatgct | 360 |
| catttgccag gtatggaagg ttttagagaa ggtactaccg aatcccaaat catgaagttg | 420 |
| gttgctagac atcaattgac tcaccaattg actgttgtta ctgctccagt tgctgaagaa | 480 |
| tctgctagag ctttgagaga tgttttcggt tgtgatgaag gttggagaga attgggtact | 540 |
| agacaagctt gcttgcaagt tattgctaga gtctcctcta gaatcttctt gggtcaagaa | 600 |
| ttgtgtagaa acccagattg gttgagagtt acttctacct attctgtttt ggctttcaga | 660 |
| gccgttgttg ttttgagatt ttggccagct ccattgagaa atttggttca ttggtttttg | 720 |
| ccagcttgta aggctgctag agatttggtt caagaagcta gagacttggt taaccctttg | 780 |
| ttgcaagaaa gaaacgaaga aagaagggct caagctaaag gtgaatctgt cttgtataga | 840 |
| aacgatgcca ttgactggtt ggaagaatta gctactgata agaacttgaa ctacgatcca | 900 |
| gctgcttctc aattgtcttt gtctactgct gctttacact cttctactga ttttttcgct | 960 |
| cagttgttgt tggatttggc tgaaagacca ggtttggctg aagaattgag acaagaagct | 1020 |
| gctaaggttg ttaatactga aggttggtct aagggttcct tgttcgattt gaaattgatg | 1080 |
| gactccgtca tgaaggaatc ccaaagattg aaacctattt ccttggcctc tatgagaaga | 1140 |
| tacactactg ctgatgttaa gatgtcctcc ggtgatgtta ttccaaaagg ttctttgaca | 1200 |
| gttgtctccg cttatagaca ttgggacgaa aaaacttacg aaaggccaga tgaattcgat | 1260 |
| ggtcatagat tcttgaggat gagatcccaa gaaggtaaag aacatcaagc ccatttggtt | 1320 |
| tctgctaccc aagatcattt tggtttcggt tatggtttac atgcttgtcc aggtagattt | 1380 |
| ttcgctgctg aagaagttaa gatcgttttg gctcaaatgt tgttgcagta cgaaattaga | 1440 |
| ttggttgccg gttctgattc tagaccagtt catgctggtt tgaatatgta tgctaatcca | 1500 |
| gcctccaaga tctccgttag atatagaggt tcttcctttt aa | 1542 |

SEQ ID NO: 74

| | |
|---|---|
| MAFTAQSYFD IGEHLRVSVI LLLTTVVLLL VFSLKARKKS LLPLVNGNRW TDPLGIEAKK | 60 |
| KFMTSARSII AEQLEKAPGK PFRVVSDVGE LVVLPPEFAP EIRNHKDFSF TMAAYKWFYA | 120 |
| HLPGMEGFRE GTTESQIMKL VARHQLTHQL TVVTAPVAEE SARALRDVFG CDEGWRELGT | 180 |
| RQACLQVIAR VSSRIFLGQE LCRNPDWLRV TSTYSVLAFR AVVVLRFWPA PLRNLVHWFL | 240 |
| PACKAARDLV QEARDLVNPL LQERNEERRA QAKGESVLYR NDAIDWLEEL ATDKNLNYDP | 300 |
| AASQLSLSTA ALHSSTDFFA QLLLDLAERP GLAEELRQEA AKVVNTEGWS KGSLFDLKLM | 360 |
| DSVMKESQRL KPISLASMRR YTTADVKMSS GDVIPKGSLT VVSAYRHWDE KTYERPDEFD | 420 |
| GHRFLRMRSQ EGKEHQAHLV SATQDHFGFG YGLHACPGRF FAAEEVKIVL AQMLLQYEIR | 480 |
| LVAGSDSRPV HAGLNMYANP ASKISVRYRG SSF | 513 |

SEQ ID NO: 75

| | |
|---|---|
| atgagagtta tggttgatca agacttgtgt ggtacttctg gtcaatgtgt tttgactttg | 60 |
| ccaggtactt ttagacaaag ggaaccagat ggtgttgctg aagtttgtgt tgctactgtt | 120 |
| ccacatgctt tacatgctgc tgttagattg gctgcttctc aatgtccagt tgctcattct | 180 |
| ggtcatagaa aaagaaggtg gaggtggaga gctagacaag ctccaacttt gagattattg | 240 |
| cagagaaggc catgtggtat gccaagaaaa acttctacca tctaa | 285 |

SEQ ID NO: 76

| | |
|---|---|
| MRVMVDQDLC GTSGQCVLTL PGTFRQREPD GVAEVCVATV PHALHAAVRL AASQCPVAHS | 60 |
| GHRKRRWRWR ARQAPTLRLL QRRPCGMPRK TSTI | 94 |

SEQ ID NO: 77

| | |
|---|---|
| atgatggaca tggaaatgga agttggtatg gttatgaagg tcttgttggg tttgtgttgt | 60 |
| gttggtgctt gttctttggc actatacttg tattacaccg tttgggttgt cccacaaaga | 120 |
| ttattggctg gttttagaag gcaaggtatt ggtggtccaa gaccatcttt tccatatggt | 180 |
| aatatggccg atatgagaga agctgttgct gctgctaaat ctgctagaag atctggtggt | 240 |
| agaatgagaa tcgttcatga ttatagacca gccgttttgc cattttacga gaagtggaga | 300 |
| aaagaacatg gtccagtttt cacttactcc atgggtaatg ttgttttctt gcacgtttct | 360 |
| agaccagatg ttgttagaga tatcaacttg tgcgtttcct tggacttggg taaatcttct | 420 |
| tacttgaagg ctactcacga acctttgttt ggtagaggta ttttgaagtc taatggtgaa | 480 |
| gcttgggctc accaaagaaa gattattgct ccagaattct tcttggacaa ggttaagggt | 540 |
| atggttgatt tgatggttga ttctgctcaa accttgttgg aatcttggga agctagagtt | 600 |
| gataagtctg gtggtactgt tgatatcaag atcgatgatg atatcagagc ttactccgcc | 660 |
| gatgttattt ctagaacttg tttcggttcc tcctacgtta agggtaagaa gatctttttg | 720 |
| aagttgagag aattgcagaa ggccgtttct aagccaaatg ttttggctga aatgaccggt | 780 |
| ttgagattct ttccaactaa gaagaataga caagcctggg gtttacacaa gcaagttcat | 840 |
| agattgatct tggaaatcgt caaagaatcc ggtgaggata agaatttgtt gagagctatt | 900 |
| ttacactccg cctcttcatc taaagttggt ttgggtgaag ctgaaaactt catcgttgat | 960 |
| aactgcaagt ctatctactt cgctggttat gaatctactg ctgttactgc tgcttggtgt | 1020 |
| ttgatgttgt tgggtttaca tccagaatgg caagatagag ttagacaaga ggttttggaa | 1080 |
| gtttgtggtg gtagaccatt ggattctcaa tccttgcaaa agatgaagaa cctaaccatg | 1140 |
| gtcatccaag aaactttgag attatatcca gctggtgcct tcgtttctag aatggcttta | 1200 |
| caagaattga agttgggtgg tgttcatatc ccaaagggtg ttaatatcta catcccagtt | 1260 |
| tctaccatgc acttggatcc aaaattgtgg ggtccagatg ctaaagaatt caatccagct | 1320 |
| agattctctg atgccagacc acaattgcat tcttatttgc catttggtgc tggtgctaga | 1380 |
| acatgtttgg gtcaaggttt tgctactgcc gaattgaaga ttttgatctc cttgatcatt | 1440 |
| tccaagttcg ccttgagatt gtccccatta tatcaacatt ctccagcctt gaagttgatc | 1500 |
| gttgaaccag aatttggtgt tgatatcacc ttgactaagg ttcaaactgc ttctactact | 1560 |
| acctactaa | 1569 |

SEQ ID NO: 78

| | |
|---|---|
| MMDMEMEVGM VMKVLLGLCC VGACSLALYL YYTVWVVPQR LLAGFRRQGI GGPRPSFPYG | 60 |
| NMADMREAVA AAKSARRSGG RMRIVHDYRP AVLPFYEKWR KEHGPVFTYS MGNVVFLHVS | 120 |
| RPDVVRDINL CVSLDLGKSS YLKATHEPLF GRGILKSNGE AWAHQRKIIA PEFFLDKVKG | 180 |
| MVDLMVDSAQ TLLESWEARV DKSGGTVDIK IDDDIRAYSA DVISRTCFGS SYVKGKKIFL | 240 |
| KLRELQKAVS KPNVLAEMTG LRFFPTKKNR QAWGLHKQVH RLILEIVKES GEDKNLLRAI | 300 |
| LHSASSSKVG LGEAENFIVD NCKSIYFAGY ESTAVTAAWC LMLLGLHPEW QDRVRQEVLE | 360 |
| VCGGRPLDSQ SLQKMKNLTM VIQETLRLYP AGAFVSRMAL QELKLGGVHI PKGVNIYIPV | 420 |
| STMHLDPKLW GPDAKEFNPA RFSDARPQLH SYLPFGAGAR TCLGQGFATA ELKILISLII | 480 |
| SKFALRLSPL YQHSPALKLI VEPEFGVDIT LTKVQTASTT TY | 522 |

SEQ ID NO: 79

| | |
|---|---|
| atgtccatct tcaacatgat tacctcttat gctggttctc agttgttgcc attctacatt | 60 |
| gctatcttcg ttttcacttt ggttccatgg gctattagat tctcttggtt ggaattgaga | 120 |
| aagggttctt ttgttccatt ggctaatcca ccagattctt tgtttggtac tggtaagact | 180 |
| agaaggtcct tcgttaagtt gtccagagaa attttggcta aggccagatc tttgtttcca | 240 |
| aacgaaccat tcagattgat taccgattgg ggtgaagttt tgattttgcc accagatttt | 300 |
| gccgacgaaa ttagaaatga tccaagattg tctttctcaa aggctgccat gcaagataat | 360 |
| catgctggta ttccaggttt cgaaactgtt gctttggttg gtagagaaga tcagttgatt | 420 |
| caaaaggttg ccagaaagca attgaccaaa catttgtccg ctgttatcga accattgtct | 480 |
| agagaatcta ctttggccgt ttctttgaac ttcggtgaaa ctactgagtg gagagctatt | 540 |
| agattgaagc cagccatttt ggatattatc gccagaatct cttccaggat ctatttgggt | 600 |
| gatcaattgt gtagaaacga agcctggttg aagattacta agacttacac taccaacttc | 660 |
| tacaccgctt ctaccaattt gagaatgttc ccaagatcca ttagaccatt ggctcattgg | 720 |
| tttttgccag aatgtagaaa gttgagacaa gaaagaaagg atgccattgg tattatcacc | 780 |
| ccattgatcg aaagaagaag agaattgaga agggctgcta ttgctgctgg tcaaccattg | 840 |
| ccagtttttc atgatgctat tgactggtct gaacaagaag ctgaagctgc tggtactggt | 900 |
| gcttctttgt atccagttat tttccagttg accttgtcct tgttggctat tcatacaacc | 960 |
| tacgatttgt tgcaacagac catgattgat ttgggtagac atccagagta cattgaacca | 1020 |
| ttaagacaag aagttgtcca gttgttgaga gaagaaggtt ggaaaaagac taccttgttc | 1080 |
| aagatgaagt tgttggactc cgctatcaaa gaatcccaaa gaatgaagcc aggttctatc | 1140 |
| gttactatga gaagatacgt taccgaagat atcaccttgt catctggttt gactttgaaa | 1200 |
| aagggtacta gattgaacgt cgataacaga agattggacg atccaaagat ctacgataac | 1260 |
| ccagaagttt acaacccata cagattctac gacatgagat ctgaagctgg taaagatcat | 1320 |
| ggtgctcaat tggtttctac tggttctaat catatgggtt tcggtcatgg tcaacattct | 1380 |
| tgtccaggta gattttttgc tgccaacgaa atcaaagttg ccttgtgtca tatcttggtt | 1440 |
| aagtacgatt ggaagttgtg tccagatact gaaactaagc cagataccag aggtatgatt | 1500 |
| gctaaatctt ctccagttac cgacattttg atcaagagaa gagaatccgt tgaattggat | 1560 |
| ttggaagcca tctga | 1575 |

SEQ ID NO: 80

| | |
|---|---|
| MSIFNMITSY AGSQLLPFYI AIFVFTLVPW AIRFSWLELR KGSVVPLANP PDSLFGTGKT | 60 |
| RRSFVKLSRE ILAKARSLFP NEPFRLITDW GEVLILPPDF ADEIRNDPRL SFSKAAMQDN | 120 |
| HAGIPGFETV ALVGREDQLI QKVARKQLTK HLSAVIEPLS RESTLAVSLN FGETTEWRAI | 180 |
| RLKPAILDII ARISSRIYLG DQLCRNEAWL KITKTYTTNF YTASTNLRMF PRSIRPLAHW | 240 |
| FLPECRKLRQ ERKDAIGIIT PLIERRRELR RAAIAAGQPL PVFHDAIDWS EQEAEAAGTG | 300 |
| ASFDPVIFQL TLSLLAIHTT YDLLQQTMID LGRHPEYIEP LRQEVVQLLR EEGWKKTTLF | 360 |
| KMKLLDSAIK ESQRMKPGSI VTMRRYVTED ITLSSGLTLK KGTRLNVDNR RLDDPKIYDN | 420 |
| PEVYNPYRFY DMRSEAGKDH GAQLVSTGSN HMGFGHGQHS CPGRFFAANE IKVALCHILV | 480 |
| KYDWKLCPDT ETKPDTRGMI AKSSPVTDIL IKRRESVELD LEAI | 524 |

SEQ ID NO: 81

| | |
|---|---|
| atgaacaagt ccaactctat gaacaacacc tctttggaaa ggttgttcca acaattggtt | 60 |
| ttgggtttgg atggtatccc attgatggat gttcattggt tgatctacgt tgcttttggt | 120 |
| gcttggttgt gctcttacgt tattcacgtt ttgtcctctt catccactgt taaggttcca | 180 |
| gttgttggtt acagatctgt ttttgaacct acctggttgt tgagattgag atttgtttgg | 240 |
| gaaggtggtt ccattattgg tcaaggttac aacaagttca aggactccat tttccaagtc | 300 |
| agaaagttgg gtactgatat cgttattatc ccaccaaact tcatcgacga agtgagaaaa | 360 |
| ttgtctcaag acaagaccag atctgtcgaa ccattcatta acgattttgc tggtcagtac | 420 |
| actaggggta tggttttttt acaatccgac ttgcaaaaca gagtcatcca acaaagattg | 480 |
| accccaaagt tggtttcttt gaccaaggtt atgaaggaag aattggatta cgccttgacc | 540 |
| aaagaaatcc cagatatgaa ggatgatgaa tgggttgaag ttgacatctc ctccattatg | 600 |
| gttagattga tctccagaat ttccgccaga gtttttttgg gtccagaaca ttgcagaaat | 660 |
| caagaatggt tgactaacac cgctgaatac tctgaatctt tgttcattac cggtttcatc | 720 |
| ttgagagttg tcccacatat cttgaggcct tttattgctc cattattgcc atcttacaga | 780 |
| accttgttga ggaacgtttc ttctggtaga agagttatcg gtgacatcat cagatctcaa | 840 |
| caaggtgatg gtaacgagga tattttgtct tggatgagag atgctgctac tggtgaagaa | 900 |
| aagcaaattg ataacattgc ccagagaatg ttgatcttgt ccttggcttc tattcatacc | 960 |
| actgctatga ctatgactca tgccatgtat gatttgtgtg ctagaccaga gtatatcgaa | 1020 |
| ccattgagag atgaagttaa gggtgttgtt gatgcttctg gttgggataa gactgctttg | 1080 |
| aatagattgc acagattgga ctcattcttg aaagaatccc aaagattcaa cccagtgttc | 1140 |
| ttgttgactt tcaacagaat ctaccaccag tctatgactt tgtctgatgg tactaatttg | 1200 |
| ccatccggta ctagaattgc tgttccatct catgctatgt tgcaagattc tgctcatgtt | 1260 |
| ccaggtccaa ctccaccaac tgaatttgat ggtttcaggt actccaagat caggtctgat | 1320 |
| tctaattacg cccaaaagta cttgttctcc atgaccgatt cttctaatat ggctttcggt | 1380 |
| tacggtaaat atgcttgtcc aggtagattt tacgcctcca acgaaatgaa gttgaccttg | 1440 |
| gctattttgt tgttgcagtt cgaattcaag ttgccagatg gtaaaggtag accaagaaac | 1500 |
| attaccatcg attccgatat gattccagat ccaagagcta gattgtgcgt cagaaaaaga | 1560 |
| tctttgaggg acgaatga | 1578 |

SEQ ID NO: 82

| | |
|---|---|
| MNKSNSMNNT SLERLFQQLV LGLDGIPLMD VHWLIYVAFG AWLCSYVIHV LSSSSTVKVP | 60 |
| VVGYRSVFEP TWLLRLRFVW EGGSIIGQGY NKFKDSIFQV RKLGTDIVII PPNFIDEVRK | 120 |
| LSQDKTRSVE PFINDFAGQY TRGMVFLQSD LQNRVIQQRL TPKLVSLTKV MKEELDYALT | 180 |
| KEIPDMKDDE WVEVDISSIM VRLISRISAR VFLGPEHCRN QEWLTNTAEY SESLFITGFI | 240 |
| LRVVPHILRP FIAPLLPSYR TLLRNVSSGR RVIGDIIRSQ QGDGNEDILS WMRDAATGEE | 300 |
| KQIDNIAQRM LILSLASIHT TAMTMTHAMY DLCARPEYIE PLRDEVKGVV DASGWDKTAL | 360 |
| NRLHRLDSFL KESQRFNPVF LLTFNRIYHQ SMTLSDGTNL PSGTRIAVPS HAMLQDSAHV | 420 |
| PGPTPPTEFD GFRYSKIRSD SNYAQKYLFS MTDSSNMAFG YGKYACPGRF YASNEMKLTL | 480 |
| AILLLQFEFK LPDGKGRPRN ITIDSDMIPD PRARLCVRKR SLRDE | 525 |

SEQ ID NO: 83

| | |
|---|---|
| atggcagaat tagatacact tgatatagta gtattaggtg ttatcttttt gggtactgtg | 60 |
| gcatacttta ctaagggtaa attgtggggt gttaccaagg atccatacgc taacggattc | 120 |
| gctgcaggtg gtgcttccaa gcctggcaga actagaaaca tcgtcgaagc tatggaggaa | 180 |
| tcaggtaaaa actgtgttgt tttctacggc agtcaaacag gtacagcgga ggattacgca | 240 |
| tcaagacttg caaaggaagg aaagtccaga ttcggtttga acactatgat cgccgatcta | 300 |
| gaagattatg acttcgataa cttagacact gttccatctg ataacatcgt tatgtttgta | 360 |
| ttggctactt acggtgaagg cgaaccaaca gataacgccg tggatttcta tgagttcatt | 420 |
| actggcgaag atgcctcttt caatgagggc aacgatcctc cactaggtaa cttgaattac | 480 |
| gttgcgttcg gtctgggcaa caatacctac gaacactaca actcaatggt caggaacgtt | 540 |
| aacaaggctc tagaaaagtt aggagctcat agaattggag aagcaggtga gggtgacgac | 600 |
| ggagctggaa ctatggaaga ggacttttta gcttggaaag atccaatgtg ggaagccttg | 660 |
| gctaaaaaga tgggcttgga ggaaagagaa gctgtatatg aacctatttt cgctatcaat | 720 |
| gagagagatg atttgacccc tgaagcgaat gaggtatact tgggagaacc taataagcta | 780 |
| cacttggaag gtacagcgaa aggtccattc aactcccaca acccatatat cgcaccaatt | 840 |
| gcagaatcat acgaactttt ctcagctaag gatagaaatt gtctgcatat ggaaattgat | 900 |
| atttctggta gtaatctaaa gtatgaaaca ggcgaccata tcgcgatctg gcctaccaac | 960 |
| ccaggtgaag aggtcaacaa atttcttgac attctagatc tgtctggtaa gcaacattcc | 1020 |
| gtcgtaacag tgaaagcctt agaacctaca gccaaagttc cttttccaaa tccaactacc | 1080 |
| tacgatgcta tattgagata ccatctggaa atatgcgctc cagtttctag acagtttgtc | 1140 |
| tcaactttag cagcattcgc ccctaatgat gatatcaaag ctgagatgaa ccgtttggga | 1200 |
| tcagacaaag attacttcca cgaaaagaca ggaccacatt actacaatat cgctagattt | 1260 |
| ttggcctcag tctctaaagg tgaaaaatgg acaaagatac cattttctgc tttcatagaa | 1320 |
| ggccttacaa aactacaacc aagatactat tctatctctt cctctagttt agttcagcct | 1380 |
| aaaaagatta gtattactgc tgttgtcgaa tctcagcaaa ttccaggtag agatgaccca | 1440 |
| ttcagaggtg tagcgactaa ctacttgttc gctttgaagc agaaacaaaa cggtgatcca | 1500 |
| aatccagctc cttttggcca atcatacgag ttgacaggac caaggaataa gtatgatggt | 1560 |
| atacatgttc cagtccatgt aagacattct aactttaagc taccatctga tccaggcaaa | 1620 |
| cctattatca tgatcggtcc aggtaccggt gttgcccctt ttagaggctt cgtccaagag | 1680 |
| agggcaaaac aagccagaga tggtgtagaa gttggtaaaa cactgctgtt ctttggatgt | 1740 |
| agaaagagta cagaagattt catgtatcaa aaagagtggc aagagtacaa ggaagctctt | 1800 |
| ggcgacaaat tcgaaatgat tacagctttt tcaagagaag gatctaaaaa ggtttatgtt | 1860 |
| caacacagac tgaaggaaag atcaaaggaa gtttctgatc ttctatccca aaaagcatac | 1920 |
| ttctacgttt gcggagacgc cgcacatatg gcacgtgaag tgaacactgt gttagcacag | 1980 |
| atcatagcag aaggccgtgg tgtatcagaa gccaagggtg aggaaattgt caaaaacatg | 2040 |
| agatcagcaa atcaatacca agtgtgttct gatttcgtaa ctttacactg taaagagaca | 2100 |
| acatacgcga attcagaatt gcaagaggat gtctggagtt aa | 2142 |

SEQ ID NO: 84

| | |
|---|---|
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQVCS DFVTLHCKET TYANSELQED VWS | 713 |

SEQ ID NO: 85

| | |
|---|---|
| atgatggatg ataccacttc tccatactct acctaccatt ccgttaggtc cattagaaat | 60 |
| caatctgctt gggctttggc tccaattgct gttttcattt gttacgttgt cttgagacac | 120 |
| aacagaaagt ctgttccagc tgcttctgct ggttctcatt ctattttgga accattgtgg | 180 |
| ttggccagat tgagattcat tagagactcc agattcatca tcggtcaagg ttactctaag | 240 |
| ttcaaggata ccattttcaa ggttaccaag gttggtgccg atattatagt tgttgctcct | 300 |
| aagtacgtcg aagagatcag aagattgtct agagatactg gtagatccgt tgaaccattc | 360 |
| attcatgatt tcgccggtga attattgggt ggtttgaatt ttttggagtc cgacttgcaa | 420 |
| accagagttg ttcaacaaaa gttgacccca aacttgaaaa ccatcgttcc agttatggaa | 480 |
| gatgagatgc attacgcttt ggtttccgaa ttggattctt gtttggatgg ttctgaacat | 540 |
| tggaccagag ttgatatgat ccacatgttg tctagaatcg tgtccagaat ttccgccaga | 600 |
| attttcttgg gtcctaagta ctgtagaaac gacttgtggt tgaaaactac tgctgagtac | 660 |
| actgagaact tgttcttgac tggtactttg ttgagattcg tcccaagaat gttgcaaaaa | 720 |
| tggattgctc cattgctacc atccttcaga caattgcaag aaaacagaca agctgccaga | 780 |
| aagatcatct ctgaaatttt gactgatcac cagccagaaa aacatgacga aacatctgat | 840 |
| aatggtgatc catacccaga tatcttgacc ttgatgtttc aagctgctag gggtaaagaa | 900 |
| aaggacattg aagatattgc ccaacacacc ttgttgttgt ccttatcttc tattcatacc | 960 |
| accgctttga ctatgactca agccttgtat gatttgtgtg cttacccaca atatttggat | 1020 |
| ccagttaagc acgaaattgc cgataccttg caatctgaag gttcttggtc taaagctatg | 1080 |
| ttggataagt tgcacatgat ggacagtttg ttgagagaat cccaaagatt gtctccagtt | 1140 |
| ttcttgttga ccttcaacag aatcttgcat actccattga ctttgtccaa cggtattcat | 1200 |
| ttgccaaagg gtactagaat tgctgctcca tctgatgcta ttttgaacga tccatctttg | 1260 |
| gttccaggtc cacaaccagc tgatactttt gatcctttca ggtacattaa ccactctact | 1320 |
| ggtgatgcta aaaagaccaa gactaacttc caaactacct ccttgcaaaa catggctttt | 1380 |
| ggttatggta aatacgcttg tccaggtaga ttttacgttg ccaacgaaat caaattggtc | 1440 |
| ttgggtcatt tgttgatgca ctacgaattc aaatttccac caggtatggg tagaccagtt | 1500 |
| aactctactg ttgatactga tatgtaccca gatttgggtg ccagattatt ggtcagaaaa | 1560 |
| agaaagatgg aagaatga | 1578 |

SEQ ID NO: 86

| | |
|---|---|
| MMDDTTSPYS TYHSVRSIRN QSAWALAPIA VFICYVVLRH NRKSVPAASA GSHSILEPLW | 60 |
| LARLRFIRDS RFIIGQGYSK FKDTIFKVTK VGADIIVVAP KYVEEIRRLS RDTGRSVEPF | 120 |
| IHDFAGELLG GLNFLESDLQ TRVVQQKLTP NLKTIVPVME DEMHYALVSE LDSCLDGSEH | 180 |
| WTRVDMIHML SRIVSRISAR IFLGPKYCRN DLWLKTTAEY TENLFLTGTL LRFVPRMLQK | 240 |
| WIAPLLPSFR QLQENRQAAR KIISEILTDH QPEKHDETSD NGDPYPDILT LMFQAARGKE | 300 |
| KDIEDIAQHT LLLSLSSIHT TALTMTQALY DLCAYPQYLD PVKHEIADTL QSEGSWSKAM | 360 |
| LDKLHMMDSL LRESQRLSPV FLLTFNRILH TPLTLSNGIH LPKGTRIAAP SDAILNDPSL | 420 |
| VPGPQPADTF DPFRYINHST GDAKKTKTNF QTTSLQNMAF GYGKYACPGR FYVANEIKLV | 480 |
| LGHLLMHYEF KFPPGMGRPV NSTVDTDMYP DLGARLLVRK RKMEE | 525 |

SEQ ID NO: 87

| | |
|---|---|
| atgaccaacc actcttcatc ctactactac gaattctaca aggatcactc ccacaccttt | 60 |
| agaagatcta tgtctgagaa taccttgatc tcttcttgtt tggctttggc tacttgcgct | 120 |
| attttgttgt ctattcaatg gttgaagcca caaccattga tcatggttaa tggtagaaag | 180 |
| ttcggtgagt tgtccaatgt tagagctaag agggatttta cttttggtgc tagacagttg | 240 |
| ttggagaagg gttttaagat gtctccagat aagccattca gaatcatggg tgatgttggt | 300 |
| gaattgcata ttttgccacc aaagtacgct tacgaagtca gaaacaacga aaagttgtct | 360 |
| ttcactatgg ctgctttcaa gtggttttat gctcatttgc caggtttcga aggtttcaga | 420 |
| gaaggtacta atgaatccca catcatgaag ttggttgcca gacatcaatt gactcatcaa | 480 |
| ttgacattgg ttaccggtgc tgtttctgaa gaatgtgctt tggttttgaa ggatgtttac | 540 |
| accgattctc cagaatggca tgatattact gctaaggatg ctaacatgaa gttcatggct | 600 |
| agaatcacct tcagagtgtt cttgggtaaa gaaatgtgta gaaacccaca gtggttgaga | 660 |
| attacttcta cctatgctgt tattgccttc agagctgttg aagaattgag attgtggcca | 720 |
| tcttggttaa gaccagttgt tcaatggttt atgccacatt gcactcaatc tagagctttg | 780 |
| gttcaagaag ctagagattt gatcaaccct ttgttggaaa gaagaagaga agaaaaggct | 840 |
| gaagctgaaa gaactggtga aaaggttact tacaacgatg ctgttgaatg gttggatgat | 900 |
| ttggctagag aaaaaggtgt tggttatgat ccagcttgtg ctcaattgtc tttgtctgtt | 960 |
| gctgctttac attctaccac tgatttcttc acccaagtca tgttcgatat tgctcaaaac | 1020 |
| ccagaattga tcgaaccatt gagagaagaa atcatctccg ttttgggtaa acaaggttgg | 1080 |
| tctaagaact ccttgtacaa cttgaagttg atggactccg tcttgaaaga atcccaaaga | 1140 |
| ttgaagccaa ttgccattgc ttctatgaga agattcacta cccataacgt tgaattgtcc | 1200 |
| gatggtgtta ttttgccaaa gaacaagttg accttggttt ccgctcatca acattgggat | 1260 |
| ccagaatatt acaaggaccc attgaagttc gatggttaca gattcttcaa catgagaagg | 1320 |
| gaaccaggta aagaatctaa ggctcaattg gtttctgcta ccccagatca tatgggtttt | 1380 |
| ggttatggtt tacatgcttg tccaggtaga tttttcgctt ccgaagaaat caagattgcc | 1440 |
| ttgtcccata tcttgttgaa gtacgatttt aagccagtcg agggttcttc tatggaacct | 1500 |
| agaaagtatg gtttgaacat gaacgctaat ccaaccgcta aattgtccgt cagaagaaga | 1560 |
| aaagaagaga tcgccatttg a | 1581 |

SEQ ID NO: 88

| | |
|---|---|
| MTNHSSSYYY EFYKDHSHTF RRSMSENTLI SSCLALATCA ILLSIQWLKP QPLIMVNGRK | 60 |
| FGELSNVRAK RDFTFGARQL LEKGFKMSPD KPFRIMGDVG ELHILPPKYA YEVRNNEKLS | 120 |
| FTMAAFKWFY AHLPGFEGFR EGTNESHIMK LVARHQLTHQ LTLVTGAVSE ECALVLKDVY | 180 |
| TDSPEWHDIT AKDANMKFMA RITFRVFLGK EMCRNPQWLR ITSTYAVIAF RAVEELRLWP | 240 |
| SWLRPVVQWF MPHCTQSRAL VQEARDLINP LLERRREEKA EAERTGEKVT YNDAVEWLDD | 300 |
| LAREKGVGYD PACAQLSLSV AALHSTTDFF TQVMFDIAQN PELIEPLREE IISVLGKQGW | 360 |
| SKNSLYNLKL MDSVLKESQR LKPIAIASMR RFTTHNVELS DGVILPKNKL TLVSAHQHWD | 420 |
| PEYYKDPLKF DGYRFFNMRR EPGKESKAQL VSATPDHMGF GYGLHACPGR FFASEEIKIA | 480 |
| LSHILLKYDF KPVEGSSMEP RKYGLNMNAN PTAKLSVRRR KEEIAI | 526 |

SEQ ID NO: 89

| | |
|---|---|
| atggccaacc attcttcatc ctactaccat gaattctaca aggatcattc ccataccgtt | 60 |
| ttgaccttga tgtctgaaaa gccagttatc ttgccatcct tgattttggg tacttgtgct | 120 |
| gttttgttgt gcatccaatg gttgaaacca caaccattga ttatggtcaa cggtagaaag | 180 |
| ttcggtgaat tgtctaatgt tagagccaag agggatttta cttttggtgc cagacaattg | 240 |
| ctagagaagg gtttgaaaat gtctccagat aagccattca gaatcatggg tgatgttggt | 300 |
| gaattgcata ttttgccacc aaagtacgct tacgaagtca gaaacaacga aaagttgtct | 360 |
| ttcactatgg ctgctttcaa gtggttttat gctcatttgc caggtttcga aggtttcaga | 420 |
| gaaggtacta atgaatccca catcatgaag ttggttgcca gacatcaatt gactcatcaa | 480 |
| ttgacattgg ttaccggtgc tgtttctgaa gaatgtgctt tggttttgaa ggatgtttac | 540 |
| accgattctc cagaatggca tgatattact gctaaggatg ctaacatgaa gttgatggct | 600 |
| agaatcacct ctagagtgtt cttgggtaaa gaaatgtgta gaaacccaca gtggttgaga | 660 |
| attacttcta cctatgctgt tattgccttc agagctgttg aagaattgag attgtggcca | 720 |
| tcttggttaa gaccagttgt tcaatggttt atgccacatt gcactcaatc tagagctttg | 780 |
| gttcaagaag ctagagattt gatcaaccct ttgttggaaa gaagaagaga agaaaaggct | 840 |
| gaagctgaaa gaactggtga aaaggttact tacaacgatg ctgttgaatg gttggatgat | 900 |
| ttggctagag aaaaaggtgt tggttatgat ccagcttgtg ctcaattgtc tttgtctgtt | 960 |
| gctgctttac attctaccac tgatttcttc acccaagtca tgttcgatat tgctcaaaac | 1020 |
| ccagaattga tcgaaccatt gagggaagaa attattgccg ttttgggtaa acaaggctgg | 1080 |
| tctaagaatt ccttgtacaa cttgaagttg atcgactccg tcttgaaaga atcccaaaga | 1140 |
| ttgaagccaa ttgccattgc ttctatgaga agattcacta cccataacgt taagttgtcc | 1200 |
| gatggtgtta ttttgccaaa gaacaagttg accttggttt ccgctcatca acattgggat | 1260 |
| ccagaatatt acaaggaccc attgaagttc gatggttaca gattcttcaa catgagaagg | 1320 |
| gaaccaggta aagaatctaa ggctcaattg gtttctgcta ccccagatca tatgggtttt | 1380 |
| ggttatggtt tacatgcttg tccaggtaga tttttcgctt ccgaagaaat caagattgcc | 1440 |
| ttgtcccata tcttgttgaa gtacgatttt aagccagtcg agggttcttc tatggaacct | 1500 |
| agaaagtatg gtttgaacat gaacgctaat ccaaccgcta aattgtccgt cagaagaaga | 1560 |
| aaagaagaga tcgccatttg a | 1581 |

SEQ ID NO: 90

| | |
|---|---|
| MANHSSSYYH EFYKDHSHTV LTLMSEKPVI LPSLILGTCA VLLCIQWLKP QPLIMVNGRK | 60 |
| FGELSNVRAK RDFTFGARQL LEKGLKMSPD KPFRIMGDVG ELHILPPKYA YEVRNNEKLS | 120 |
| FTMAAFKWFY AHLPGFEGFR EGTNESHIMK LVARHQLTHQ LTLVTGAVSE ECALVLKDVY | 180 |
| TDSPEWHDIT AKDANMKLMA RITSRVFLGK EMCRNPQWLR ITSTYAVIAF RAVEELRLWP | 240 |
| SWLRPVVQWF MPHCTQSRAL VQEARDLINP LLERRREEKA EAERTGEKVT YNDAVEWLDD | 300 |
| LAREKGVGYD PACAQLSLSV AALHSTTDFF TQVMFDIAQN PELIEPLREE IIAVLGKQGW | 360 |
| SKNSLYNLKL IDSVLKESQR LKPIAIASMR RFTTHNVKLS DGVILPKNKL TLVSAHQHWD | 420 |
| PEYYKDPLKF DGYRFFNMRR EPGKESKAQL VSATPDHMGF GYGLHACPGR FFASEEIKIA | 480 |
| LSHILLKYDF KPVEGSSMEP RKYGLNMNAN PTAKLSVRRR KEEIAI | 526 |

SEQ ID NO: 91

| | |
|---|---|
| atgtccgcct tccaaaaaga aaccgttttg tctgttagac actggaccga atctttgttt | 60 |
| tcattcactg ctactagaga tccaggtttc agatttcaaa atggtcaatt cgccatgatc | 120 |
| ggtttggaag ttgaaggtaa accattgatg agagcttact ctatggcttc tgctaatcat | 180 |
| gaagaagcct tggaattctt ctcaatcaag gttcaagatg gtccattgac ttccagattg | 240 |
| caaaagatta gagaaggcga tatcatcttg gttggtagaa aagctactgg tactttgatt | 300 |
| accggtaact tgattccagg taagaggttg ttgttgttgt ctactggtac tggtttggct | 360 |
| ccatttgctt cattgattaa ggatccagat gtctacgaaa actacgaaac tatcgttttg | 420 |
| gctcatggtt gcagacaagt ttctgaattg gcttatggtg aacacttggt tgaaggtttg | 480 |
| agaaaccatg aatttttcgg tccattgatc agagacaagt tggtttatta cccaaccgtt | 540 |
| actagagagc cattcagaaa tagaggtaga atcaccgatt tgattgcctc taatcagttg | 600 |
| ttcgatgata ttggtcaagg tggtttggat atcgaaaccg atagaattat gttgtgtggt | 660 |
| tctccaggta tgttggaaga attgcatgct atgtttgctg ctagaggttt tgttgaaggt | 720 |
| aatcattctc aaccaggtca cttcgttatt gaaaaggctt tcgttcagag gtaa | 774 |

SEQ ID NO: 92

| | |
|---|---|
| MSAFQKETVL SVRHWTESLF SFTATRDPGF RFQNGQFAMI GLEVEGKPLM RAYSMASANH | 60 |
| EEALEFFSIK VQDGPLTSRL QKIREGDIIL VGRKATGILI TGNLIPGKRL LLLSTGTGLA | 120 |
| PFASLIKDPD VYENYETIVL AHGCRQVSEL AYGEHLVEGL RNHEFFGPLI RDKLVYYPTV | 180 |
| TREPFRNRGR ITDLIASNQL FDDIGQGGLD IETDRIMLCG SPGMLEELHA MFAARGFVEG | 240 |
| NHSQPGHFVI EKAFVQR | 257 |

SEQ ID NO: 93

| | |
|---|---|
| atgaagcaca tcgatgtcat gaacttcatc tccaagattt gctcttggtc taaagattct | 60 |
| ccaggtttcg ttttgttgat ctccatcttg gttatcttgg gttccgttac tttcattcca | 120 |
| aagtgtggta gaagatctgc ttttgatgct ttgccaatcg ttaacaagcc aaagtttggt | 180 |
| ccaatcttct ccattattgc taggtggaga ttcatccacc aatccaagaa aattttggaa | 240 |
| gagggtcaaa agtgctactc caatagacct tttagaattt ggactgattg gggtgaagtt | 300 |
| ttgatgttga ctccagatta tgcccacgaa attagaaacg atccacactt gtctttttca | 360 |
| ggtgccgtta agattgatgg tcatgctgat attccaggtt tcgaaactgt taagttgatc | 420 |
| tcccatccag acaacttgat tcaattggtt gctagaaagc aattgaccag acatttggct | 480 |
| gctgttattc aaccattgtc ctctgttact gaagaagcct tgattaagaa cttgggcaaa | 540 |
| tctcaagaat ggtccgaaat ctacttgaag tacgccgttt tggatattat cgccagattg | 600 |
| tcatctagga tctactttgg tgagttgttg taccaaaacg aagagtggtt gtctatcgtt | 660 |
| aagaattacg ctactcattt cttcaccgcc tcttccgatt tgagaaaagt tccatgggct | 720 |
| ttcagatctt tggttcattg gtttgttcca tcttgcagag ctttgagatt ggaaagatac | 780 |
| aacgctagaa gagttttgga accagttatc tctcaaagaa ggcaattgaa agaagctgct | 840 |
| aaaactgctg gtggtactcc attgcatttt gaagatgcta ttgaatgggc tgaagttgaa | 900 |
| gctagagtta agggtactaa gtacgatcca gttatcttcc aattgacctt gtccttgttg | 960 |
| gctattcata ccacttacga cttgttggaa atgtgcatga ttgatttggc taaaagacca | 1020 |
| gactgcatcg aggacttgag aaaagaagtt attaccgtct tgagaaagga tggttggaca | 1080 |
| aaaaatgcct tgtacaacat gaagttgttg gactccgcta tcaaagaatc ccaaagattg | 1140 |
| aaaccaggtt ccatcacttc tatgagaaga tacgctactt ccgatgtcca attgagagat | 1200 |
| ggtgttgttt tgaaaaaggg caacagattg aacgttttga ccttgcatag atccccagat | 1260 |
| ttgtttccat ctccagatac ttatgaccca tacaggttct acaacattag aggtcaacca | 1320 |
| ggtaaagaaa actgggctca attggtttct acctccgttg aacatatggg ttttggtcat | 1380 |
| ggtgaacatt cttgtccagg tagatttttt gctgccaacg aaatcaaagt tgccttggct | 1440 |
| catattttgg ttaagtacga ttggaagttg tccgatgaag ctggtggttg tactgaagtt | 1500 |
| aagggtatgg ttgaaaaagc tggttccaag gttaagatct tggtcagaca aagacaagat | 1560 |
| gtcgaatccg ttttggatga agcttga | 1587 |

SEQ ID NO: 94

| | |
|---|---|
| MKHIDVMNFI SKICSWSKDS PGFVLLISIL VILGSVTFIP KCGRRSAFDA LPIVNKPKFG | 60 |
| PIFSIIARWR FIHQSKKILE EGQKCYSNRP FRIWTDWGEV LMLTPDYAHE IRNDPHLSFS | 120 |
| GAVKIDGHAD IPGFETVKLI SHPDNLIQLV ARKQLTRHLA AVIQPLSSVT EEALIKNLGK | 180 |
| SQEWSEIYLK YAVLDIIARL SSRIYFGELL YQNEEWLSIV KNYATHFFTA SSDLRKVPWA | 240 |
| FRSLVHWFVP SCRALRLERY NARRVLEPVI SQRRQLKEAA KTAGGTPLHF EDAIEWAEVE | 300 |
| ARVKGTKYDP VIFQLTLSLL AIHTTYDLLE MCMIDLAKRP DCIEDLRKEV ITVLRKDGWT | 360 |
| KNALYNMKLL DSAIKESQRL KPGSITSMRR YATSDVQLRD GVVLKKGNRL NVLTLHRSPD | 420 |
| LFPSPDTYDP YRFYNIRGQP GKENWAQLVS TSVEHMGFGH GEHSCPGRFF AANEIKVALA | 480 |
| HILVKYDWKL SDEAGGCTEV KGMVEKAGSK VKILVRQRQD VESVLDEA | 528 |

SEQ ID NO: 95

| | |
|---|---|
| atggatgttc aagatacaac cgctgcttgt catgatgctt ttgctgaatt ggcttctcca | 60 |
| gcttgtattc aagatccata tcctttcatg agatggttga gagaacatga tccagttcat | 120 |
| agagctgctt caggtttgtt tttgttgtct agacatgctg atatctactg ggcttttaaa | 180 |
| gctactggtg atgcttttag aggtccagct ccatctgaat tggctagata ttttccaaga | 240 |
| gctgcctctt ctttgtcctt gaatttgttg gcttctacct tggctatgaa ggaaccacca | 300 |
| actcatacaa gattgagaag attgatctcc agagatttca ccgttggtca aattgataat | 360 |
| ttgaggccat ccattgctag aatcgttgct gctagattgg atggtatggc tccagctttg | 420 |
| gaaagaggtg aagctgttga cttgcataga gaatttgctt tggctttgcc aatgttggtt | 480 |
| tttgctgaac tatttggtat gccacaagac gacgtttttg aattgtctgc tatcgtttcc | 540 |
| gctatcttgg aaggtttgtc tccacatgct tcagatccac aattggctgc tgctgatgtt | 600 |
| gcttctgcta gagttaaggc ttatttcggt gatttgatct tgagaaagag agccgatcca | 660 |
| agaagagata tcgtttctac tttggttggt gctcatactg atgatgctga tactttgtct | 720 |
| gatgccgaat tgatttctat gttgtggggt atgttgttgg gtggttttgc tactactgct | 780 |
| gctactattg atcatgctgt tttggctatg ttggcttacc cagaagaaag acattggttg | 840 |
| caaggtgatg ctgctggtgt tgaagctttt gttgaagagg ttttgagatg tgaagctcca | 900 |
| gctatgtttt cctcaattcc aagaattgcc caaagggata ttgaattgca tggtgttgtt | 960 |
| attccaaagg atgccgatgt tagagttttg attgctgctg gtaatagaga tccagatgca | 1020 |
| tttgctgatc cagatagatt tgatccagtt aggttttacg gtactagacc aggtatgtca | 1080 |
| tctgatggta agatcatgtt gtctttcggt catggtattc atttctgttt gggtgctcaa | 1140 |
| ttggctagag ttcaattggc tgaatctttg ccacaaattc aagctagatt tccaactttg | 1200 |
| gctttggctg aacaacctac tagagaacca tctgcttttt tgagaacttt cagagctttg | 1260 |
| ccagttagat tgcatgctca agctgctgct gaagttagag ttgttgttga tcaagatttg | 1320 |
| tgtggtacta ccggtcaatg tgttttgact ttgccaggta cttttagaca aagggaacca | 1380 |
| gatggtgttg ctgaagtatg tatggctact gttccacaag ctttacatgc tgctgttaga | 1440 |
| ttggctgctt ctcaatgtcc agttgctgct attagagtta ttgaatctga agctggtgat | 1500 |
| gatcattgca ctaatccagg tccaacacca tctccagctg atgctgaaag acatgctgct | 1560 |
| aaagatttga gaaatccagg tgaacatgac ggcactattt ga | 1602 |

SEQ ID NO: 96

| | |
|---|---|
| MDVQDTTAAC HDAFAELASP ACIQDPYPFM RWLREHDPVH RAASGLFLLS RHADIYWAFK | 60 |
| ATGDAFRGPA PSELARYFPR AASSLSLNLL ASTLAMKEPP THTRLRRLIS RDFTVGQIDN | 120 |
| LRPSIARIVA ARLDGMAPAL ERGEAVDLHR EFALALPMLV FAELFGMPQD DVFELSAIVS | 180 |
| AILEGLSPHA SDPQLAAADV ASARVKAYFG DLILRKRADP RRDIVSTLVG AHTDDADTLS | 240 |
| DAELISMLWG MLLGGFATTA ATIDHAVLAM LAYPEERHWL QGDAAGVEAF VEEVLRCEAP | 300 |
| AMFSSIPRIA QRDIELHGVV IPKDADVRVL IAAGNRDPDA FADPDRFDPV RFYGTRPGMS | 360 |
| SDGKIMLSFG HGIHFCLGAQ LARVQLAESL PQIQARFPTL ALAEQPTREP SAFLRTFRAL | 420 |
| PVRLHAQAAA EVRVVVDQDL CGTTGQCVLT LPGTFRQREP DGVAEVCMAT VPQALHAAVR | 480 |
| LAASQCPVAA IRVIESEAGD DHCTNPGPTP SPADAERHAA KDLRNPGEHD GTI | 533 |

SEQ ID NO: 97

| | |
|---|---|
| atggttgttg ttgttgctgc agctatggct gctgcttctt tgtgttgtgg tgttgctgct | 60 |
| tacttgtatt acgttttgtg gttggctcca gaaagattga gagcacattt gagaaggcaa | 120 |
| ggtattggtg gtccaactcc atcttttcca tatggtaatt tggccgatat gagatcacat | 180 |
| gctgctgctg cagctggtgg taaagctact ggtgaaggta gacaagaggg tgatatagtt | 240 |
| catgattaca gacaagctgt gttcccattc tacgaaaatt ggagaaaaca atacggtcca | 300 |
| gtgttcactt actctgttgg taatatggtt ttcttgcacg tttccagacc agatatcgtt | 360 |
| agagaattgt ctttgtgcgt ttccttggac ttgggtaaat cttcttatat gaaggctacc | 420 |
| caccaacctt tgtttggtga aggtattttg aagtctaatg gtaacgcttg ggctcaccaa | 480 |
| agaaaattga ttgctccaga attcttccca gataaggtta agggtatggt tgatttgatg | 540 |
| gttgattccg ctcaagtctt ggtttcttca tgggaagata gaatcgatag atctggtggt | 600 |
| aatgccttgg atttgatgat cgatgatgat atcagagctt actccgccga tgttatttct | 660 |
| agaacttgtt tcggttcctc ctacgttaag ggtaagcaaa ttttcgacat gatcagagag | 720 |
| ttgcaaaaga ccgtttctac caagaagcaa aacttgttgg ctgaaatgac tggcttgtct | 780 |
| tttttgtttc caaaggcttc tggtagagct gcttggagat tgaatggtag agttagagct | 840 |
| ttgattttgg acttggttgg tgaaaatggt gaagaggatg gtggtaattt gttgtctgct | 900 |
| atgttgagat ctgctagagg tggtggtggt ggcggtggtg aagttgcagc tgctgctgaa | 960 |
| gattttgttg ttgataactg caagaacatc tacttcgctg gttatgaatc tactgctgtt | 1020 |
| actgctgctt ggtgtttgat gttgttggct ttacatccag aatggcaaga tagagttaga | 1080 |
| gatgaagttc aagctgcttg ttgcggtggt ggtggaagat ctccagattt tccagcttta | 1140 |
| caaaagatga agaacttgac catggtgatc caagaaactt tgagattata tccagctggt | 1200 |
| gccgttgttt ctagacaagc tttgagagaa ttatccttgg gtggtgttag agttccaaga | 1260 |
| ggtgttaata tctacgttcc agtttctacc ttgcatttgg atgctgaatt gtggggtggt | 1320 |
| ggtgctggtg ctgctgaatt tgatccagct agatttgctg atgctagacc accattgcat | 1380 |
| gcttatttgc catttggtgc cggtgctaga acatgtttgg gtcaaacttt tgctatggcc | 1440 |
| gaattgaagg ttttgttgtc tttggttttg tgcagattcg aagttgcttt gtctccagaa | 1500 |
| tatgttcatt ctccagctca caagttgatc gttgaagctg aacatggtgt tagattggtc | 1560 |
| ttgaagaaag tcagatctaa gtgtgattgg gctggtttcg attga | 1605 |

SEQ ID NO: 98

| | |
|---|---|
| MVVVVAAAMA AASLCCGVAA YLYYVLWLAP ERLRAHLRRQ GIGGPTPSFP YGNLADMRSH | 60 |
| AAAAAGGKAT GEGRQEGDIV HDYRQAVFPF YENWRKQYGP VFTYSVGNMV FLHVSRPDIV | 120 |
| RELSLCVSLD LGKSSYMKAT HQPLFGEGIL KSNGNAWAHQ RKLIAPEFFP DKVKGMVDLM | 180 |
| VDSAQVLVSS WEDRIDRSGG NALDLMIDDD IRAYSADVIS RTCFGSSYVK GKQIFDMIRE | 240 |
| LQKTVSTKKQ NLLAEMTGLS FLFPKASGRA AWRLNGRVRA LILDLVGENG EEDGGNLLSA | 300 |
| MLRSARGGGG GGGEVAAAAE DFVVDNCKNI YFAGYESTAV TAAWCLMLLA LHPEWQDRVR | 360 |
| DEVQAACCGG GGRSPDFPAL QKMKNLTMVI QETLRLYPAG AVVSRQALRE LSLGGVRVPR | 420 |
| GVNIYVPVST LHLDAELWGG GAGAAEFDPA RFADARPPLH AYLPFGAGAR TCLGQTFAMA | 480 |
| ELKVLLSLVL CRFEVALSPE YVHSPAHKLI VEAEHGVRLV LKKVRSKCDW AGFD | 534 |

SEQ ID NO: 99

| | |
|---|---|
| atggctcaat tggatacctt ggatatcgtt gttttggctg ctttgccatt gggtactgtt | 60 |
| gcttatttta ctaagggtac ttactgggct gtttctgctg atccatatgc taatccattg | 120 |
| actaatgcta atggtgctgc tagagctggt aagtccagaa acattattga aaagttggaa | 180 |
| gaatccgaca agaactgcgt tgttttttac ggttctcaaa ctggtactgc tgaagattat | 240 |
| gcttccaggt tgtctaaaga aggtcattct agattcggtt tgaacaccat ggttgctgat | 300 |
| ttggaagaat acgatttcga caacttggac tcattcccag aagataagtt ggctgttttt | 360 |
| gttttggcta cttatggtga aggtgaacct actgataatg ccgttgaatt ctacgaattc | 420 |
| atcggttccg aagatatcac tttttctgat ggtggttcca tcgatgataa gccattgtct | 480 |
| aagttgaact acgttgcttt tggtttgggt aacaacacct acgaacatta caactccatg | 540 |
| gttagaaacg tcgataagta cttgacaaag ttgggtgcta ctagattggg ttctgccggt | 600 |
| gaaggtgatg atggtgctgg tactatggaa gaagattttt tggcttggaa agaacctatg | 660 |
| tgggctgctg ttgctgaaaa gatgaatttg gaagaaagag aagctgaata cgaagccgtt | 720 |
| ttcgaagtta ctgaaaagcc agatttgaac gctcaagatg atactgttta tttgggtgag | 780 |
| ccaaacaaga accacttgga aggtaatcaa aagggtccat tcaatgctaa caacccattc | 840 |
| attgctccaa tcgttgaatc tcatgaacta ttcaccacca aagaaagaaa ctgcttgcac | 900 |
| atggaaatta gcattggtgg ttctaacttg tcttacacta ccggtgatca tattgctatt | 960 |
| tggccaaaca atgccggtaa agaagttgac agattcttca aggttttggg caaagaagat | 1020 |
| aagagacata ccgttattgc tgtcagaggt ttggatccaa ctgctaaagt tccatttcca | 1080 |
| tctccaacta cttatgatgc tgctgttaga ttccatttgg aaattggtgc tgctgtctct | 1140 |
| agacaattgg tttctactat tgctcaattc gccccaaacg aagatattaa ggctgaaatg | 1200 |
| gctaaattgg gttccgataa ggattacttc aagttgcaag ttaccgacag aaacttgaat | 1260 |
| ttggctcagt tgttggaaat ttgcggtaaa ggtcaaccat ggactaagat tccattctcc | 1320 |
| tttatgttcg aatccttgtt gaagattcag ccaaggtact actccatctc ttcttcatct | 1380 |
| ttggttcaga aggacaaggt ttctattacc gctgttgttg aatctttgga aagaccaggt | 1440 |
| gctccacatg ttttgaaagg tgttactacc aattacttgt tggccttgaa gcaaaagcaa | 1500 |
| catggtgatc caaatccaga tccacatggt ttgaattacg ctattactgg tccaagaaac | 1560 |
| aagtacgatg gtatccatgt tccagttcat gttagacact ctaacttcaa attgccatcc | 1620 |
| gatccatcta agccaatagt tatggttggt ccaggtactg gtgttgctcc ttttagaggt | 1680 |
| tttgttcaag aaagagctgc tcaagctaaa gctggtcata atgttggtaa gaccattttg | 1740 |
| ttcttcggtt gcagaaaagc ctctgaggat ttcttgtatc aaaatgaatg ggcccagtac | 1800 |
| aaagaagctt tgggagataa tttcgaaatc tacaccgctt tctctagaga tggtccaaaa | 1860 |
| aaggtttacg tccagaacca tttggaagaa catggtgaag aagttaacag gttgttggaa | 1920 |
| aaaaaggcct acttctacgt ttgtggtgat gctgctcata tggctagaga tgttaatacc | 1980 |
| ttgttgggca agttgatctc caagtacaga aatgtctctg aaactaaggg tgaagaaatc | 2040 |
| gttaaggcta tgagagcctc taatcagtac caagaagatg tttggtctta a | 2091 |

SEQ ID NO: 100

| | |
|---|---|
| MAQLDTLDIV VLAALPLGTV AYFTKGTYWA VSADPYANPL TNANGAARAG KSRNIIEKLE | 60 |
| ESDKNCVVFY GSQTGTAEDY ASRLSKEGHS RFGLNTMVAD LEEYDFDNLD SFPEDKLAVF | 120 |
| VLATYGEGEP TDNAVEFYEF IGSEDITFSD GGSIDDKPLS KLNYVAFGLG NNTYEHYNSM | 180 |
| VRNVDKYLTK LGATRLGSAG EGDDGAGTME EDFLAWKEPM WAAVAEKMNL EEREAEYEAV | 240 |
| FEVTEKPDLN AQDDTVYLGE PNKNHLEGNQ KGPFNANNPF IAPIVESHEL FTTKERNCLH | 300 |
| MEISIGGSNL SYTTGDHIAI WPNNAGKEVD RFFKVLGKED KRHTVIAVRG LDPTAKVPFP | 360 |
| SPTTYDAAVR FHLEIGAAVS RQLVSTIAQF APNEDIKAEM AKLGSDKDYF KLQVTDRNLN | 420 |
| LAQLLEICGK GQPWTKIPFS FMFESLLKIQ PRYYSISSSS LVQKDKVSIT AVVESLERPG | 480 |
| APHVLKGVTT NYLLALKQKQ HGDPNPDPHG LNYAITGPRN KYDGIHVPVH VRHSNFKLPS | 540 |
| DPSKPIVMVG PGTGVAPFRG FVQERAAQAK AGHNVGKTIL FFGCRKASED FLYQNEWAQY | 600 |
| KEALGDNFEI YTAFSRDGPK KVYVQNHLEE HGEEVNRLLE KKAYFYVCGD AAHMARDVNT | 660 |
| LLGKLISKYR NVSETKGEEI VKAMRASNQY QEDVWS | 696 |

SEQ ID NO: 101

| | |
|---|---|
| atgccaggta agattgaaaa cggtactcca aaggatttga aaaccggtaa cgattttgtt | 60 |
| tccgctgcta agtctttgtt ggatagagct tttaagtccc accattctta ctacggtttg | 120 |
| tgttctactt cttgccaagt ttatgatact gcttgggttg ctatgattcc aaagactaga | 180 |
| gataacgtca agcaatggtt gttcccagaa tgtttccact acttgttgaa aactcaagct | 240 |
| gctgatggtt cttggggttc tttgccaact actcaaactg ctggtatttt ggatactgct | 300 |
| tctgctgttt tggctttgtt gtgtcatgct caagaaccat tgcaaatctt ggatgtttct | 360 |
| ccagacgaaa tgggtttgag aattgaacat ggtgttacca gcttgaagag acaattggct | 420 |
| gtttggaatg atgtcgaaga taccaaccat atcggtgtcg aattcattat tccagccttg | 480 |
| ttgtccatgt tggaaaaaga attggatgtc ccatctttcg aattcccatg cagatctatt | 540 |
| ttggaaagaa tgcacggtga aaagttgggt catttcgatt tggaacaagt ttacggtaag | 600 |
| ccatcctctt tgttgcattc tttggaagct ttcttgggca agttggattt cgatagattg | 660 |
| tctcatcact tgtaccacgg ttctatgatg gcttctccat cttctactgc tgcttatttg | 720 |
| attggtgcta ctaagtggga tgatgaagct gaagattact tgagacacgt tatgagaaat | 780 |
| ggtgctggtc atggtaatgg tggtatttct ggtacttttc caactaccca tttcgaatgc | 840 |
| tcttggatta ttgctacctt gttgaaggtt ggtttcacct tgaaacaaat cgatggtgat | 900 |
| ggtttgagag gtttgtctac cattttgttg gaagctttga gagatgagaa cggtgttatt | 960 |
| ggttttgctc caagaactgc tgatgttgat gatactgcta aagctttgtt ggccttgtcc | 1020 |
| ttggttaatc aaccagtttc tccagatatc atgatcaagg ttttcgaagg taaggatcat | 1080 |
| ttcactacct tcggttctga aagagatcca tctttgactt ccaacttgca cgttttgttg | 1140 |
| tccttgttga agcagtctaa cttgtctcaa taccacccac aaattctaaa gactaccttg | 1200 |
| ttcacttgta gatggtggtg gggttctgat cattgtgtta aggataagtg gaacttgtct | 1260 |
| cacttgtacc caactatgtt gttggttgaa gctttcactg aagtcttgca tttgattgac | 1320 |
| ggtggtgaat tgtcctcttt gttcgatgaa tctttcaagt gcaagatcgg cttgtctatt | 1380 |
| ttccaagctg ttttgagaat catcttgacc caagataatg acggttcttg gagaggttat | 1440 |
| agagaacaaa cttgctacgc tatcttggct ttggttcaag ctagacatgt ttgtttcttc | 1500 |
| acccacatgg ttgatagatt gcaatcctgt gttgatagag gtttctcttg gttgaagtct | 1560 |
| tgctctttcc attcccaaga tttgacttgg acttctaaga ctgcttacga agttggtttt | 1620 |
| gttgctgaag cttacaaatt ggctgcttta caatctgcct ctttggaagt tccagctgct | 1680 |
| actattggtc attctgttac ttcagctgtt ccatcttctg atttggagaa gtacatgaga | 1740 |
| ttggttagaa agaccgcttt gttctctcca ttggatgaat ggggtttgat ggcctctatt | 1800 |
| atcgaatctt ctttcttcgt gccattgcta caagctcaaa gagttgaaat ctacccaaga | 1860 |
| gataacatca aggtcgacga agataagtac ttgtccatta ttccattcac ctgggttggt | 1920 |
| tgtaacaaca gatctagaac tttcgcttct aacagatggt tgtacgacat gatgtacttg | 1980 |
| tctttgttgg gttaccaaac cgatgagtat atggaagctg ttgctggtcc agtttttggt | 2040 |
| gatgtttctt tgttgcacca aaccatcgat aaggttattg ataacaccat gggtaacttg | 2100 |
| gctagagcta atggtactgt tcattctggt aatggtcatc aacatgagtc tccaaacatt | 2160 |
| ggtcaagttg aagatacttt gaccaggttc actaactctt ttttgaacca caaggatgtc | 2220 |
| ttgaactcct catcttctga tcaagatacc ttgagaagag aattcagaac cttcatgcat | 2280 |
| gcccatatta cccaaatcga agataactcc agattctcca aacaagcttc ttctgatgct | 2340 |
| ttctcatctc cagaacaatc ttacttccaa tgggttaatt ctaccggtgg ttctcatgtt | 2400 |
| gcttgtgctt attcttttgc tttctccaac tgtttgatgt ccgctaattt gttgcaaggt | 2460 |
| aaggatgctt ttccatccgg tactcaaaag tacttgatct cctctgttat gagacatgct | 2520 |
| accaacatgt gtagaatgta caacgatttc ggttccattg ctagagataa tgccgaaaga | 2580 |
| aacgttaact ccattcactt cccagaattc actttgtgta acggtacttc tcaaaacttg | 2640 |
| gacgaaagaa aagagaggtt gttgaagatt gctacctacg aacaaggtta cttggataga | 2700 |
| gcattggaag ccttggaaag acaatctaga gatgatgctg gtgatagagc tggttctaaa | 2760 |
| gatatgagaa agttgaagat cgtcaagttg ttctgtgatg ttaccgactt gtatgatcag | 2820 |
| ttgtacgtta tcaaggactt gtcctcttca atgaagtaa | 2859 |

SEQ ID NO: 102

| | |
|---|---|
| MPGKIENGTP KDLKTGNDFV SAAKSLLDRA FKSHHSYYGL CSTSCQVYDT AWVAMIPKTR | 60 |
| DNVKQWLFPE CFHYLLKTQA ADGSWGSLPT TQTAGILDTA SAVLALLCHA QEPLQILDVS | 120 |
| PDEMGLRIEH GVTSLKRQLA VWNDVEDTNH IGVEFIIPAL LSMLEKELDV PSFEFPCRSI | 180 |
| LERMHGEKLG HFDLEQVYGK PSSLLHSLEA FLGKLDFDRL SHHLYHGSMM ASPSSTAAYL | 240 |
| IGATKWDDEA EDYLRHVMRN GAGHGNGGIS GTFPTTHFEC SWIIATLLKV GFTLKQIDGD | 300 |
| GLRGLSTILL EALRDEGNIV GFAPRTADVD DTAKALLALS LVNQPVSPDI MIKVFEGKDH | 360 |
| FTTFGSERDP SLTSNLHVLL SLLKQSNLSQ YHPQILKTTL FTCRWWWGSD HCVKDKWNLS | 420 |
| HLYPTMLLVE AFTEVLHLID GGELSSLFDE SFKCKIGLSI FQAVLRIILT QDNDGSWRGY | 480 |
| REQTCYAILA LVQARHVCFF THMVDRLQSC VDRGFSWLKS CSFHSQDLTW TSKTAYEVGF | 540 |
| VAEAYKLAAL QSASLEVPAA TIGHSVTSAV PSSDLEKYMR LVRKTALFSP LDEWGLMASI | 600 |
| IESSFFVPLL QAQRVEIYPR DNIKVDEDKY LSIIPFTWVG CNNRSRTFAS NRWLYDMMYL | 660 |
| SLLGYQTDEY MEAVAGPVFG DVSLLHQTID KVIDNTMGNL ARANGTVHSG NGHQHESPNI | 720 |
| GQVEDTLTRF TNSVLNHKDV LNSSSSDQDT LRREFRTFMH AHITQIEDNS RFSKQASSDA | 780 |
| FSSPEQSYFQ WVNSTGGSHV ACAYSFAFSN CLMSANLLQG KDAFPSGTQK YLISSVMRHA | 840 |
| TNMCRMYNDF GSIARDNAER NVNSIHFPEF TLCNGTSQNL DERKERLLKI ATYEQGYLDR | 900 |
| ALEALERQSR DDAGDRAGSK DMRKLKIVKL FCDVTDLYDQ LYVIKDLSSS MK | 952 |

SEQ ID NO: 103

| | |
|---|---|
| atgcacattt tgacttaccc atccggtaag attgaaaacg gtactccaaa ggatttgaaa | 60 |
| accggtaacg attttgtttc cgctgctaag tctttgttgg atagagcttt taagtcccac | 120 |
| cattcttact acggtttgtg ttctacttct tgccaagttt atgatactgc ttgggttgct | 180 |
| atgattccaa agactagaga taacgtcaag caatggttgt tcccagaatg tttccactac | 240 |
| ttgttgaaaa ctcaagctgc tgatggttct tggggttctt tgccaactac tcaaactgct | 300 |
| ggtattttgg atactgcttc tgctgttttg gctttgttgt gtcatgctca agaaccattg | 360 |
| caaatcttgg atgtttctcc agacgaaatg ggtttgagaa ttgaacatgg tgttaccagc | 420 |
| ttgaagagac aattggctgt ttggaatgat gtcgaagata ccaaccatat cggtgtcgaa | 480 |
| ttcattattc cagccttgtt gtccatgttg gaaaaagaat tggatgtccc atctttcgaa | 540 |
| ttcccatgca gatctatttt ggaaagaatg cacggtgaaa agttgggtca tttcgatttg | 600 |
| gaacaagttt acggtaagcc atcctctttg ttgcattctt tggaagcttt cttgggcaag | 660 |
| ttggatttcg atagattgtc tcatcacttg taccacggtt ctatgatggc ttctccatct | 720 |
| tctactgctg cttatttgat tggtgctact aagtgggatg atgaagctga agattacttg | 780 |
| agacacgtta tgagaaatgg tgctggtcat ggtaatggtg gtatttctgg tacttttcca | 840 |
| actacccatt tcgaatgctc ttggattatt gctactttgt tgaagggtgg tttcaccttg | 900 |
| aaacaaattg atggtgatgg tttgagaggc ttgtctacca ttttgttgga agctttgaga | 960 |
| gatgagaacg gtgttattgg ttttgctcca agaactgctg atgttgatga tactgctaaa | 1020 |
| gctttgttgg ccttgtcctt ggttaatcaa ccagtttctc cagatatcat gatcaagggt | 1080 |
| tttgaaggta aggatcattt cactaccttc ggttctgaaa gagatccatc tttgacttcc | 1140 |
| aacttgcacg ttttgttgtc tttgccaggt aagcaatcta acttgtctca ataccatcca | 1200 |
| cagatcttga aaactacctt gttcacttgt agatggtggt ggggttctga tcattgtgtt | 1260 |
| aaggataagt ggaacttgtc tcacttgtac ccaactatgt tgttggttga agctttcact | 1320 |
| gaagtcttgc atttgattga cggtggtgaa ttgtcctctt tgttcgatga atctttcaag | 1380 |
| tgcaagatcg gcttgtctat tttccaagct gttttgagaa tcatcttgac ccaagataat | 1440 |
| gacggttctt ggagaggtta tagagaacaa acttgctacg ctatcttggc tttggttcaa | 1500 |
| gctagacatg tttgtttctt cacccacatg gttgatagat tgcaatcctg tgttgataga | 1560 |
| ggtttctctt ggttgaagtc ttgctctttc cattcccaag atttgacttg gacttctaag | 1620 |
| actgcttacg aagttggttt tgttgctgaa gcttacaaat tggctgcttt acaatctgcc | 1680 |
| tctttggaag ttccagctgc tactattggt cattctgtta cttcagctgt tccatcttct | 1740 |
| gatttggaga agtacatgag attggttaga aagaccgctt tgttctctcc attggatgaa | 1800 |
| tggggtttga tggcctctat tatcgaatct tctttcttcg tgccattgct acaagctcaa | 1860 |
| agagttgaaa tctacccaag agataacatc aaggtcgacg aagataagta cttgtccatt | 1920 |
| attccattca cctgggttgg ttgtaacaac agatctagaa ctttcgcttc taacagatgg | 1980 |
| ttgtacgaca tgatgtactt gtctttgttg ggttaccaaa ccgatgagta tatggaagct | 2040 |
| gttgctggtc cagtttttgg tgatgtttct ttgttgcacc aaaccatcga taaggttatt | 2100 |
| gataacacca tgggtaactt ggctagagct aatggtactg ttcattctgg taatggtcat | 2160 |
| caacatgagt ctccaaacat tggtcaagtt gaagatactt tgaccaggtt cactaactct | 2220 |
| gttttgaacc acaaggatgt cttgaactcc tcatcttctg atcaagatac cttgagaaga | 2280 |
| gaattcagaa ccttcatgca tgcccatatt acccaaatcg aagataactc cagattctcc | 2340 |
| aaacaagctt cttctgatgc tttctcatct ccagaacaat cttacttcca atgggttaat | 2400 |
| tctaccggtg gttctcatgt tgcttgtgct tattcttttg ctttctccaa ctgtttgatg | 2460 |
| tccgctaatt tgttgcaagg taaggatgct tttccatccg gtactcaaaa gtacttgatc | 2520 |
| tcctctgtta tgagacatgc taccaacatg tgtagaatgt acaacgattt cggttccatt | 2580 |
| gctagagata atgccgaaag aaacgttaac tccattcact tcccagaatt cactttgtgt | 2640 |
| aacggtactt ctcaaaactt ggacgaaaga aaagagaggt tgttgaagat tgctacctac | 2700 |
| gaacaaggtt acttggatag agcattggaa gccttggaaa gacaatctag agatgatgct | 2760 |
| ggtgatagag ctggttctaa agatatgaga aagttgaaga tcgtcaagtt gttctgtgat | 2820 |
| gttaccgact t tatgatca gttgtacgtt atcaaggact tgtcctcttc aatgaagtaa | 2880 |

SEQ ID NO: 104

| | |
|---|---|
| MHILTYPSGK IENGTPKDLK TGNDFVSAAK SLLDRAFKSH HSYYGLCSTS CQVYDTAWVA | 60 |
| MIPKTRDNVK QWLFPECFHY LLKTQAADGS WGSLPTTQTA GILDTASAVL ALLCHAQEPL | 120 |
| QILDVSPDEM GLRIEHGVTS LKRQLAVWND VEDTNHIGVE FIIPALLSML EKELDVPSFE | 180 |
| FPCRSILERM HGEKLGHFDL EQVYGKPSSL LHSLEAFLGK LDFDRLSHHL YHGSMMASPS | 240 |
| STAAYLIGAT KWDDEAEDYL RHVMRNGAGH GNGGISGTFP TTHFECSWII ATLLKGGFTL | 300 |
| KQIDGDGLRG LSTILLEALR DENGVIGFAP RTADVDDTAK ALLALSLVNQ PVSPDIMIKG | 360 |
| FEGKDHFTTF GSERDPSLTS NLHVLLSLPG KQSNLSQYHP QILKTTLFTC RWWWGSDHCV | 420 |
| KDKWNLSHLY PTMLLVEAFT EVLHLIDGGE LSSLFDESFK CKIGLSIFQA VLRIILTQDN | 480 |
| DGSWRGYREQ TCYAILALVQ ARHVCFFTHM VDRLQSCVDR GFSWLKSCSF HSQDLTWTSK | 540 |
| TAYEVGFVAE AYKLAALQSA SLEVPAATIG HSVTSAVPSS DLEKYMRLVR KTALFSPLDE | 600 |
| WGLMASIIES SFFVPLLQAQ RVELYPRDNI KVDEDKYLSI IPFTWVGCNN RSRTFASNRW | 660 |
| LYDMMYLSLL GYQTDEYMEA VAGPVFGDVS LLHQTIDKVI DNTMGNLARA NGTVHSGNGH | 720 |
| QHESPNIGQV EDTLTRFTNS VLNHKDVLNS SSSDQDTLRR EFRTFMHAHI TQIEDNSRFS | 780 |
| KQASSDAFSS PEQSYFQWVN STGGSHVACA YSFAFSNCLM SANLLQGKDA FPSGTQKYLI | 840 |
| SSVMRHATNM CRMYNDFGSI ARDNAERNVN SIHFPEFTLC NGTSQNLDER KERLLKIATY | 900 |
| EQGYLDRALE ALERQSRDDA GDRAGSKDMR KLKIVKLFCD VTDLYDQLYV IKDLSSSMK | 959 |

SEQ ID NO: 105

| | |
|---|---|
| atgttggaag gtattggtat cggttcttct ccacaatctt tgttggattc tgccaaggat | 60 |
| ttgattgctg aagcttgttc tagaaccgat ccattttatg gtttgtctac cgcttcttgt | 120 |
| caaacttatg atactgcttg ggttgccatg gttgttaaga gattggaatc tggtgaagat | 180 |
| gcttgggctt tcccacaatc ttttaggtat attttggaag cccaaacttc tggtggtggt | 240 |
| tggggtgatc caaaagcttc taaaactgtt ggtattttgg ataccgctgc tgctttgttg | 300 |
| gcattataca gacatttgga tagaccattg caaatcaccg aagttaccag aatggatgtc | 360 |
| gaatccagaa ttgaaaaggc ttctacctct ttggtgtccc aattgcaaca atgggatgat | 420 |
| ttggttgaat ccaaccatat cggtgtcgaa ttgattttgc cttccttgtt ggaacaattg | 480 |
| agacaaatca atccagtctt gcactctact agattcaagg ctgaacaaga tttgaccaga | 540 |
| atgcacgaag aaaagttgag acacttcgat gtctctagct tgtattcttc tagaccatct | 600 |
| tctgccttgc attctttgga agcttttttg ggtaaattgg acttcgatag agttggtcat | 660 |
| cacttgtatc atggttctat gatggcttct ccatcttcta ctgctgctta tttgattggt | 720 |
| gcttctactt acgattctac cgctgaagct tacttgtccc atattttgaa atgtaccgct | 780 |
| aaaggttctc caggtggtat tccaggtact ttcccaatta ctaatttcga atactcctgg | 840 |
| attaccgcca ctttgttgag agattgcttt gcttatgaag atttggctgg tccatctttg | 900 |
| gattgcattg gtcaaacttt ggaagaagct ttacaagctg gtaagggtgt tattggtttt | 960 |
| gctccaagaa ctgctgatgt tgatgatact gctaaaggtt tgttggcttt gacctctatg | 1020 |
| agaagatatg gtcatgctga tccaaagcca atgatcaagg ttttcgaaag agaagatcat | 1080 |
| ttcaccacct tcggttctga aagagatcca tcttttacct ctaactgcca cgttttgttg | 1140 |
| tctttgttgg ctcaagaatc tgacttgcca ttatacagag cccaaatcta caaggctact | 1200 |
| aagttcttgt gtgacttctt gttctatagg gatggtccat tgaaagataa gtggcatatg | 1260 |
| acttcatcct acccatctat gttgttggtt gaagcttttt ccgagttgtt gagattgcaa | 1320 |
| gacgaacaaa agttcgaaca gttgttgacc tctgatgaac aacacagagt tttcatcgtt | 1380 |
| ttgttccaaa cctgtttgag aaccttgttg gtccaatctg aagatggttc ttggtctggt | 1440 |
| tgtactgaac aaacttctca tgctgtatgt actttggcta gagcttggag attgaacttg | 1500 |
| ttcattgact taagaccaga cttgcaagtt gctattcaag ccggtattca atacttggat | 1560 |
| agacctgaag ctcaaatggg tcaaaactgg acttctaaga ctgtttactc cgttgatttg | 1620 |
| gttggtaagg cttacatttt ggctgctaga aaaatggccc aagatttgtc tgatagaact | 1680 |
| ccatttggtc ctaagaggga agatttcatg tctttgaaga agttcaccac ctacttggaa | 1740 |
| acctctaaaa gattgccatt attgcaagct actccacctt ggcaaattat tgcttctttg | 1800 |
| actgaatccg ctttgttctt gcctttgttg aggaaagaaa aagaagccat ctttccaagg | 1860 |
| gatggtacta tgttgactcc agatgattac ttggatatca ttccatacac ctgggttatt | 1920 |
| tgcggtaaca gaattgatgt tcatacctct ccatctttgg ccttggatat gatgttgttg | 1980 |
| tctatgtacg gttaccagaa cgacgaattc tttgaaactc atgctatggc tggtcattac | 2040 |
| caatctggtt ctgatttgaa gagattggtt gatgatgtct tgcaacagaa cattccaaaa | 2100 |
| tgtgctgaac catctaacgg ttcttctaaa catgatactg gtaaccagtc tagaactgct | 2160 |
| caagaagctg ctatttcttt gccagaaatg tctgctggtt tgaacagatt catctcctac | 2220 |
| atcttgaaac atccattggt tgctcaagct catccaaact ctaaatccga attgcacaga | 2280 |
| gaattgcaag ctttcttgca tgctcattct gatcaatctg acgaaaacag aagattcgct | 2340 |
| gcccaagaag aaaaagacga attgcaatct ccatcccaaa ccttgtttca atacgttaga | 2400 |
| tctactggtg gtgatcatgt tgcttgtgct tactctttgt ctttcatgtt gtgcatcatc | 2460 |
| tcttcatcct tgtgtgatgg tggtgaagtt tttcaaactg ccgaagaaaa gtatttggct | 2520 |
| gctgctgctg caagacattt ggctactatg tgtagaatct acaacgacta tggttccttg | 2580 |
| gctagagata ctgctgaaag aaatgttaac tccatgcact acccagaatt cagacaaact | 2640 |
| actgctcaag ctgaagatcc aactatggct aaaaagaagg ctttgttgtc attgggtgaa | 2700 |
| tacgaacacg atttcttgag agataccttg gacagattgg aaaaagctgt tgctactcca | 2760 |
| ccaccaggtg gtatggttga atctaaaaga ttaagagtcg tcaggttgtt cagatacttc | 2820 |
| tgtgatgtta ctgacttgta cgatcagttg tacgtcttga aggatttgtc ctcttcattg | 2880 |
| agaacctaa | 2889 |

SEQ ID NO: 106

| | |
|---|---|
| MLEGIGIGSS PQSLLDSAKD LIAEACSRTD PFYGLSTASC QTYDTAWVAM VVKRLESGED | 60 |
| AWAFPQSFRY ILEAQTSGGG WGDPKASKTV GILDTAAALL ALYRHLDRPL QITEVTRMDV | 120 |
| ESRIEKASTS LVSQLQQWDD LVESNHIGVE LILPSLLEQL RQINPVLHST RFKAEQDLTR | 180 |
| MHEEKLRHFD VSSLYSSRPS SALHSLEAFL GKLDFDRVGH HLYHGSMMAS PSSTAAYLIG | 240 |
| ASTYDSTAEA YLSHILKCTA KGSPGGIPGT FPITNFEYSW ITATLLRDCF AYEDLAGPSL | 300 |
| DCIGQTLEEA LQAGKGVIGF APRTADVDDT AKGLLALTSM RRYGHADPKP MIKVFEREDH | 360 |
| FTTFGSERDP SFTSNCHVLL SLLAQESDLP LYRAQIYKAT KFLCDFLFYR DGPLKDKWHM | 420 |
| TSSYPSMLLV EAFSELLRLQ DEQKFEQLLT SDEQHRVFIV LFQTCLRTLL VQSEDGSWSG | 480 |
| CTEQTSHAVC TLARAWRLNL FIDLRPDLQV AIQAGIQYLD RPEAQMGQNW TSKTVYSVDL | 540 |
| VGKAYILAAR KMAQDLSDRT PFGPKREDFM SLKKFTTYLE TSKRLPLLQA TPPWQIIASL | 600 |
| TESALFLPLL RKEKEAIFPR DGTMLTPDDY LDIIPYTWVI CGNRIDVHTS PSLALDMMLL | 660 |
| SMYGYQNDEF FETHAMAGHY QSGSDLKRLV DDVLQQNIPK CAEPSNGSSK HDTGNQSRTA | 720 |
| QEAAISLPEM SAGLNRFISY ILKHPLVAQA HPNSKSELHR ELQAFLHAHS DQSDENRRFA | 780 |
| AQEEKDELQS PSQTLFQYVR STGGDHVACA YSLSFMLCII SSSLCDGGEV FQTAEEKYLA | 840 |
| AAAARHLATM CRIYNDYGSL ARDTAERNVN SMHYPEFRQT TAQAEDPTMA KKKALLSLGE | 900 |
| YEHDFLRDTL DRLEKAVATP PPGGMVESKR LRVVRLFRYF CDVTDLYDQL YVLKDLSSSL | 960 |
| RT | 962 |

SEQ ID NO: 107

| | |
|---|---|
| atgtacgaga ggtacttgtt gttgttgcat atcttgactc acaagtccgg taagattgaa | 60 |
| aatggtactc caaagtactt gaaaaccggt gatgatttgg tttctgctgc taagtctttg | 120 |
| ttggatagag ctttcaagtc ccatcattct tactacggtt tgtgttctac ctcttgccaa | 180 |
| gtttatgata ctgcttgggt tgccatgatt agaaagacta ctgaaaatgt caagcactgg | 240 |
| ttgttcccag aatgtttcca ttacttgttg aaaacccaag ctgctgatgg ttcttggggt | 300 |
| gctttgccaa ctactcaaac tgctggtatt ttggatactg cttctgctgt tttggctttg | 360 |
| ttgtctcatg ttagaaagcc attgcaaatc ttggatgttt ccccagacga aattggtcca | 420 |
| agaattgaac atggtgttgc ctcattgaaa agacaattgg ctgtttggaa ggacgtcgaa | 480 |
| gaaactaatc atatcggtgt tgaattgatc gttccagcct tgttgtctac cttggaaaaa | 540 |
| gaattgggtg agtcctcttt tgaattccca tgtaagggta tcttggagaa gatgtacgaa | 600 |
| gaaaagttgg gtaacttcga cttgaagaag gtttacggta aaccatcctc tttgttgcat | 660 |
| tctttggaag ctttcttggg tcaaatcgat ttcgatagat tgtcccatca cttgtacaga | 720 |
| ggttctatga tggcttctcc atcttctact gctgcttatt tgattggtgc tactaagtgg | 780 |
| gatgatgaag ctgaagatta cttgagacac atcgttagaa atggtgctgg tcatggtgat | 840 |
| ggtggtattt ctggtacttt tccaactacc catttcgaat gctcttggat tttggctact | 900 |
| ttgttgcaag gtggtttcac catgaagcaa attgattcta atggtttgag aggtttggct | 960 |
| accattttgg ctgatgcttt gagagatgag aatggtgtta ttggttttgc tccaagaact | 1020 |
| gccgatgttg atgatactgc taaagctttg ttggccttgt ccttgatcaa tcaaccagtt | 1080 |
| tctccagaca tcatgatcaa ggtttttgaa ggtaaggatc acttcactac cttcggttct | 1140 |
| gaaagagatc catctttgac ttccaacttg catgttttgt tgtgcttgtt gaagcagcca | 1200 |
| aacgtttctc aataccatcc acaaattcta aagaccacct tgttcacttg tagatggtgg | 1260 |
| tggggttctg atcattgtgt taaggataag tggaacttgt ctcacttgta cccaactatg | 1320 |
| ttgttggttg aagctttcac tgaagtcttg catttgattg atgctggtga gttgtcatcc | 1380 |
| ttgttcgata agtctttgaa gtgcaagatc ggcttgtcta ttttccaagc tgttttgaga | 1440 |
| atcatcttga cccaagataa tgacggttct tggagagctt atagagaaca aacttgctac | 1500 |
| gctatcttgg ctttggttca agctagacat gtttgtttct tcacccacat ggttgataga | 1560 |
| ttgcagtctt gtattgatag aggtgtctct tggttgaagt cctgtagatt tcattcccaa | 1620 |
| gatttgactt ggacttctaa gactgcttac gaagttggtt ttgttgctga agcttacaaa | 1680 |
| ttggctgctt tacaatctgc ctctttggaa gttccagctg ctactattgg tcattctgtt | 1740 |
| acttcagctg ttccatcttc tgatttggag aagtacatga gattggttag aaagaccgct | 1800 |
| ttgttctctc cattggatga atggggtttg agagcttctg ttatcgaatc ttctttcttc | 1860 |
| gtgccattat tgcaagccca aagagttgaa atctacccaa gagataacat caagatcgat | 1920 |
| gaggacaagt atttgagcat tattccattc acttgggtcg gttgtaacaa cagatctaga | 1980 |
| acttttgctt ccaacagatg gttgtacgac atgatgtatt tgtccttgtt gggttaccaa | 2040 |
| accgatgagt atatggaagc tgttgctggt ccagtttttt ccgatgtttc tttgttgaga | 2100 |
| ttggccatcg ataaggttat tgataacacc agagttaact tggctggtac aaatggtact | 2160 |
| gttcataatg gtaacggtca ccaacatgaa tccccaaaca ttagacaagt tgaagatacc | 2220 |
| ttgaccagat tcgctaactc tgttttgaac cacaaggatg tcttgaactc ctcatcttct | 2280 |
| gatcaagaca ctttgagaag agaattcaga gcttttatgc atgctcatac cacccaaatc | 2340 |
| gaagataact ctagattctc taagcaagcc tctggtgatg ttttttcatc tccagaacaa | 2400 |
| tcctacttcc aatgggttaa ttctactggt ggttctcatg ttgcttgtgc ttactctttt | 2460 |
| gctttctcta actgtttgat gtccgctaat ttgccacaag gtaaagaagc ttttccatct | 2520 |
| gctacacaga agtacttgat ctcttctgtt atgagacatg ctaccaacat gtgcagaatg | 2580 |
| tacaatgatt tcggttccat tgccagagat aacgttgaaa gaaacgttaa ctctatgcac | 2640 |
| ttcccagaat tcgctttgtg taagggtatt tcccaaacca tcgatgacag aaagaagaga | 2700 |
| ttgtcccaaa ttgccatgta cgaacaaggt tgtttggata gagcattgga agctttggaa | 2760 |
| agacaatcta gagatgatgc cggtgattct gctggttcta aagatgttag aaagatcaag | 2820 |
| atcgtcaagt tgttctgtga agttaccgac ttgtatgatc agttgtacgt tatcaaggac | 2880 |
| ttgtcctctt caatgaagta a | 2901 |

SEQ ID NO: 108

| | |
|---|---|
| MYERYLLLLH ILTHKSGKIE NGTPKYLKTG DDLVSAAKSL LDRAFKSHHS YYGLCSTSCQ | 60 |
| VYDTAWVAMI RKTTENVKHW LFPECFHYLL KTQAADGSWG ALPTTQTAGI LDTASAVLAL | 120 |
| LSHVRKPLQI LDVSPDEIGP RIEHGVASLK RQLAVWKDVE ETNHIGVELI VPALLSTLEK | 180 |
| ELGESSFEFP CKGILEKMYE EKLGNFDLKK VYGKPSSLLH SLEAFLGQID FDRLSHHLYR | 240 |
| GSMMASPSST AAYLIGATKW DDEAEDYLRH IVRNGAGHGD GGISGTFPTT HFECSWILAT | 300 |
| LLQGGFTMKQ IDSNGLRGLA TILADALRDE NGVIGFAPRT ADVDDTAKAL LALSLINQPV | 360 |
| SPDIMIKVFE GKDHFTTFGS ERDPSLTSNL HVLLCLLKQP NVSQYHPQIL KTTLFTCRWW | 420 |
| WGSDHCVKDK WNLSHLYPTM LLVEAFTEVL HLIDAGELSS LFDKSLKCKI GLSIFQAVLR | 480 |
| IILTQDNDGS WRAYREQTCY AILALVQARH VCFFTHMVDR LQSCIDRGVS WLKSCRFHSQ | 540 |
| DLTWTSKTAY EVGFVAEAYK LAALQSASLE VPAATIGHSV TSAVPSSDLE KYMRLVRKTA | 600 |
| LFSPLDEWGL RASVIESSFF VPLLQAQRVE IYPRDNIKID EDKYLSIIPF TWVGCNNRSR | 660 |
| TFASNRWLYD MMYLSLLGYQ TDEYMEAVAG PVFSDVSLLR LAIDKVIDNT RVNLAGTNGT | 720 |
| VHNGNGHQHE SPNIRQVEDT LTRFANSVLN HKDVLNSSSS DQDTLRREFR AFMHAHTTQI | 780 |
| EDNSRFSKQA SGDVFSSPEQ SYFQWVNSTG GSHVACAYSF AFSNCLMSAN LPQGKEAFPS | 840 |
| ATQKYLISSV MRHATNMCRM YNDFGSIARD NVERNVNSMH FPEFALCKGI SQTIDDRKKR | 900 |
| LSQIAMYEQG CLDRALEALE RQSRDDAGDS AGSKDVRKIK IVKLFCEVTD LYDQLYVIKD | 960 |
| LSSSMK | 966 |

SEQ ID NO: 109

| | |
|---|---|
| atgaagactg tattgcaacc agataagcac tcccacaagt tgattttgtc atctcaacaa | 60 |
| ccagttccaa ctccatctca tccacaagat gttttggtta aggttcatgc tacttgtcca | 120 |
| tgtaagggtg aattggattg ggctttgtgg gctccagaat tcattggtga taagattcca | 180 |
| attccaggtc aagatttggc tggtactgtt gtttctgctc cagaaaattc tggtttcaag | 240 |
| ccagatgatg aagtttacgc tagaattgaa gctaatagac caggtgctgc tgctgaatat | 300 |
| gttttggcta gagtttctga attggccatc agaccaaaga atttgacttg ggctgaaact | 360 |
| gctgcttctc caatttctgc tttgactgct tatcaaggtt tgttcactag aggtggttta | 420 |
| gatccaaaag ctttggctgg tgatgaagct gctagagaaa aaaatggtaa ggtcagagtt | 480 |
| ttgatcaacg gttctgctgg tggtgttggt tcttgggctg ttcaattggc tagattggct | 540 |
| ggtgttaaga ctattgccgg tgttgttggt actcaaaaca tcgattttgt cagacaattg | 600 |
| ggtgctaccg aaaccattga ttacaaaaag caatccattg gtgaatgggc tactcaagat | 660 |
| ccatcttcta gacaattcga tttggttttc gattgcatcg gtttgccatc tttgtctcaa | 720 |
| acttggtatg ctgttagaga aggtggtact ttggtttctg tttgtgctcc accagaacaa | 780 |
| aacagaccag aagatgttaa gaaagaagtc aactccatct tcttcgttat cgatccagtt | 840 |
| ggtaaggatt tggaagttat caccaagttg ttggaagctg gtcaaatcaa gccacatatc | 900 |
| gattctgttg ttggtttgga tgatttcgaa gaagcttggg aaaaagtcga atctggtaga | 960 |
| actaagggta aggttgttgt tatggttatg aaggacgagt aa | 1002 |

SEQ ID NO: 110

| | |
|---|---|
| MKTVLQPDKH SHKLILSSQQ PVPTPSHPQD VLVKVHATCP CKGELDWALW APEFIGDKIP | 60 |
| IPGQDLAGTV VSAPENSGFK PDDEVYARIE ANRPGAAAEY VLARVSELAI RPKNLTWAET | 120 |
| AASPISALTA YQGLFTRGGL DPKALAGDEA AREKNGKVRV LINGSAGGVG SWAVQLARLA | 180 |
| GVKTIAGVVG TQNIDFVRQL GATETIDYKK QSIGEWATQD PSSRQFDLVF DCIGLPSLSQ | 240 |
| TWYAVREGGT LVSVCAPPEQ NRPEDVKKEV NSIFFVIDPV GKDLEVITKL LEAGQIKPHI | 300 |
| DSVVGLDDFE EAWEKVESGR TKGKVVVMVM KDE | 333 |

SEQ ID NO: 111

| | |
|---|---|
| atgggtagat tcgaaggtaa ggttgctgtt gttactggtg ctggtgctgg tattggtaaa | 60 |
| gcttgtgctt tggctattgc tagagaaggt ggtagagttg ttgttgctga tattgatggt | 120 |
| tctgctgcta ttgcttgtac tgctcaaatt gctgctgaag ctggtcatgc tttggcttta | 180 |
| gctattgata ttgctgatgc tcaagctgtt gctgctttgt ttgaaactgc tgaaagacat | 240 |
| tttggtggtg ttgatttgtt ggttaacaac gcttctgcta tgcatttgac tccaagagat | 300 |
| agagccattt tggaattgga attggctgtt tgggatcaaa ctatggctag aaatttgagg | 360 |
| ggtactttgt tgtgttgcag acaagctatt ccaagaatga ttgctagagg tggtggtgct | 420 |
| atagttaaca tgtcatcttg tcaaggtttg tctggtgata ctgctttgac ttcttatgct | 480 |
| gcttctaagg ctgctatgaa catgttgtca tcttcattgg ctactcaata cggtcatgct | 540 |
| caaattagat gtaatgctgt tgctccaggt ttgatcatga ctgaaagatt gagaatgcaa | 600 |
| acccatttga gaaggcacca attattgcca agagttggta gaccaagaac ttggccaaga | 660 |
| tggtggagat cttgttctcc aactatgttg agatcttcta ctggtcaagt tgtctgtatt | 720 |
| gatggtggta tgttggctca tgttccaact tatgctgatg gtggtaattc tagagctgct | 780 |
| agaccagctg gtgaaacagc tgaagctgat gctgctccaa gatgttaa | 828 |

SEQ ID NO: 112

| | |
|---|---|
| MGRFEGKVAV VTGAGAGIGK ACALAIAREG GRVVVADIDG SAAIACTAQI AAEAGHALAL | 60 |
| AIDIADAQAV AALFETAERH FGGVDLLVNN ASAMHLTPRD RAILELELAV WDQTMARNLR | 120 |
| GTLLCCRQAI PRMIARGGGA IVNMSSCQGL SGDTALTSYA ASKAAMNMLS SSLATQYGHA | 180 |
| QIRCNAVAPG LIMTERLRMQ THLRRHQLLP RVGRPRTWPR WWRSCSPTML RSSTGQVVCI | 240 |
| DGGMLAHVPT YADGGNSRAA RPAGETAEAD AAPRC | 275 |

SEQ ID NO: 113

| | |
|---|---|
| atgtccttcc cagatgaaca aaaggttgat ttccaaacct tccagaacgt tatcaacaat | 60 |
| caattgtctc caacctccga atccagacat ggtatttgtc catctactga agaatccttg | 120 |
| tgggaatctc cagtttctac tcaagatgat gttgatagag ctgtttctgc tgctaaagct | 180 |
| gcttatccag cttggagaaa attgtcttgg gacgaaagag cttcttactt ggttaagttt | 240 |
| gctgatgcta ttgaagccca caagcaagaa ttcattgatt tgttgggtag agaagctggt | 300 |
| aaaccaccac aagctggtgg ttttgaattg atgttggtta tggaacacgt tagggaaact | 360 |
| ccaaagttga gaattggtga agttaagcca gaagataacg aagatagaac cgctgttgtt | 420 |
| agatacgttc caattggtgt tggtgttggt atagttccat ggaattttcc aatgttgttg | 480 |
| ggtattggta aagcttaccc agctatgttg gctggtaata cttttatttg gaagccatct | 540 |
| ccatacaccc catactctgc tttgaaattg gctgaaattg gtgctaaagt tttgccacca | 600 |
| ggtgttttac aagctttgtc tggtggtgat gatttgggtc caatgttgac tgctcatcca | 660 |
| gatgttgcta aggtttcttt tactggttct actgaaaccg gtaaaaagat tatggctgct | 720 |
| tgtgctgcta ctttgaagag agttactttg gaattgggtg gtaatgatgc tgctatcgtt | 780 |
| tgtgaagatg ttgatattcc aggtgttgct ggtaaggttg cttttttggc ttatgttcat | 840 |
| tctggtcaga tctgcatgaa catcaagaga atctacgttc acgaatccat ctacgacaag | 900 |
| ttcgtttccg aagttatcaa gttcttgcat gctttgaaaa ccggtgattt ctctgatcca | 960 |
| gaagcttttt ttggtccaat ccaaaacaag atgcagtacg aaaaattgca gaggttgtac | 1020 |
| gaacaaatcg ataagcaagg ttggaagtgt gcttttggtt ctgcttctcc agctacttct | 1080 |
| gaaaaaggtt attttgttcc accagtcttg gttgataatc caccagaaga ttctgaaatc | 1140 |
| gtccaaatgg aaccatttgg tccaatagtt ccagttatga agtggcaatc tgaagatgat | 1200 |
| gttattgcta gagctaacgc ttctgattat ggtttgggtg cttctgtttg gtctaaagat | 1260 |
| gttgctagag caagaagaat ggctgaatta ttggaagctg gttctgtttg ggttaacacc | 1320 |
| cattttgaag ttgctccaaa tgttcctttt ggtggtcata agcaatctgg tattggtatg | 1380 |
| gattggggtg aagttggttt gaaaggttgg tgtaatccac aagcttattg ggtcaaacat | 1440 |
| tccggttaa | 1449 |

SEQ ID NO: 114

| | |
|---|---|
| MSFPDEQKVD FQTFQNVINN QLSPTSESRH GICPSTEESL WESPVSTQDD VDRAVSAAKA | 60 |
| AYPAWRKLSW DERASYLVKF ADAIEAHKQE FIDLLGREAG KPPQAGGFEL MLVMEHVRET | 120 |
| PKLRIGEVKP EDNEDRTAVV RYVPIGVGVG IVPWNFPMLL GIGKAYPAML AGNTFIWKPS | 180 |
| PYTPYSALKL AEIGAKVLPP GVLQALSGGD DLGPMLTAHP DVAKVSFTGS TETGKKIMAA | 240 |
| CAATLKRVIL ELGGNDAAIV CEDVDIPGVA GKVAFLAYVH SGQICMNIKR IYVHESIYDK | 300 |
| FVSEVIKFLH ALKTGDFSDP EAFFGPIQNK MQYEKLQRLY EQIDKQGWKC AFGSASPATS | 360 |
| EKGYFVPPVL VDNPPEDSEI VQMEPFGPIV PVMKWQSEDD VIARANASDY GLGASVWSKD | 420 |
| VARARRMAEL LEAGSVWVNT HFEVAPNVPF GGHKQSGIGM DWGEVGLKGW CNPQAYWVKH | 480 |
| SG | 482 |

SEQ ID NO: 115

| | |
|---|---|
| atgggtagat tcgaaggtaa agttgctgtc gtcactggtg ctggtgccgg tattggtaag | 60 |
| gcttgtgcct tggctattgc tagagaaggt ggtcgtgttg tcgtcgccga catcgatggt | 120 |
| tccgctgcta tcgcttgtac tgctcaaatc gctgctgaag ctggtcatgc tttggctttg | 180 |
| gctatcgata tcgctgatgc tcaagccgtc gccgccttat tcgaaaccgc cgaaagacat | 240 |
| ttcggtggtg ttgacttgtt ggttaataac gcttccgcta tgcacttgac tcctagagac | 300 |
| agagctattt tagaattgga attggctgtt tgggatcaaa ccatggctac caacttgaga | 360 |
| ggtactttgt tgtgctgtcg tcaagccatc cctcgtatga ttgctagagg tggtggtgct | 420 |
| atcgttaaca tgtcttcttg tcaaggttta tctggtgaca ccgctttgac ttcctacgct | 480 |
| gcttctaagg ccgccatgaa catgttgtcc tcttctttgg ccacccaata tggtcacgcc | 540 |
| caaatcagat gtaacgccgt tgctccaggt ttaatcatga ctgaaagatt gttggctaaa | 600 |
| ttggatgctt gtatgcaaac tcatttgaga agacaccaat tgttgccaag agtcggtaga | 660 |
| cctgaagacg ttgctgcctt ggttgctttt ttgttatctg acgacgctgc tttcatcact | 720 |
| ggtcaagttg tctgtatcga tggtggtatg ttggctcacg ttccaaccta cgctgacggt | 780 |
| ggtaactctc gtgctgccag accagctggt gaaactgctg aagccgatgc tgctccaaga | 840 |
| tgctaa | 846 |

SEQ ID NO: 116

| | |
|---|---|
| MGRFEGKVAV VTGAGAGIGK ACALAIAREG GRVVVADIDG SAAIACTAQI AAEAGHALAL | 60 |
| AIDIADAQAV AALFETAERH FGGVDLLVNN ASAMHLTPRD RAILELALAV WDQTMATNLR | 120 |
| GTLLCCRQAI PRMIARGGGA IVNMSSCQGL SGDTALTSYA ASKAAMNMLS SSLATQYGHA | 180 |
| QIRCNAVAPG LIMTERLLAK LDACMQTHLR RHQLLPRVGR PEDVAALVAF LLSDDAAFIT | 240 |
| GQVVCIDGGM LAHVPTYADG GNSRAARPAG ETAEADAAPR C | 281 |

SEQ ID NO: 117

| | |
|---|---|
| atgagagttg ttatcgatca agatttgtgt ggtactactg gtcaatgtgt cttgactttg | 60 |
| ccaggtactt ttagacaaag agaaccagac ggtgtcgccg aagtctgtgt tgctactgtc | 120 |
| ccacaagctt tacacgctgc tgctagattg gctgcttccc aatgtcctgt tgctgccatt | 180 |
| cgtgtcatcg agtctgacgc tggtgaaaga gcctctgctg atccagctcc atccccagct | 240 |
| caagccgaaa gacatgctgc taaggatcaa agaaatccag gtggtagatt cgaaggtaag | 300 |
| gttgctgttg tcaccggtgc tggtgctggt attggtaaag cttgtgcttt agccattgct | 360 |
| agagaaggtg gtagagttgt tgtcgctgat atcgacggtt ctgctgccgt cgcctgtact | 420 |
| gcccaaatcg ccgccgaggc tggtcatgct ttggctttgg ccatggatat tgctgatgcc | 480 |
| caagccgttg ctgctttgtt cgaaactgct gaaagacact ttggtggtgt tgatttgttg | 540 |
| gtcaacaacg cttctgctat gcacttgacc ccaagagata gaactatttt ggacttggac | 600 |
| ttggctgtct gggaccaaac catggctact aatttgcgtg gtaccttgtt gtgttgtaga | 660 |
| caagctatcc cacgtatgat cgcccgtggt ggtggtgcta tcgtcaacat gtcttcttgt | 720 |
| caaggtttat ctggtgacac cgctcaaact tcttacgctg cctctaaggc tgctatgaac | 780 |
| atgttgtccg cttctttggc tacccaatac ggtcacgctc aaattcgttg taacgctgtc | 840 |
| gctccaggtt tgattatgac tgaaagatta ttagctaagt tagatgaatg tatgcaaaga | 900 |
| cacttatcca gacaccaatt gttgcaacgt gtcggtagac cagaagatgt tgctgccttg | 960 |
| gtcgcttttt tattatctga cgacgctgct ttcattactg gtcaagtctt gtgtattgat | 1020 |
| ggtggtatgt tggctcacgt tccaacctac gctgacggtg gtaactctag agctgctaga | 1080 |
| ccagccggtg atactgccaa ggccgctgct ggtccaagat gttaa | 1125 |

SEQ ID NO: 118

| | |
|---|---|
| MRVVIDQDLC GTTGQCVLTL PGTFRQREPD GVAEVCVATV PQALHAAARL AASQCPVAAI | 60 |
| RVIESDAGER ASADPAPSPA QAERHAAKDQ RNPGGRFEGK VAVVTGAGAG IGKACALAIA | 120 |
| REGGRVVVAD IDGSAAVACT AQIAAEAGHA LALAMDIADA QAVAALFETA ERHFGGVDLL | 180 |
| VNNASAMHLT PRDRTILDLD LAVWDQTMAT NLRGTLLCCR QAIPRMIARG GGAIVNMSSC | 240 |
| QGLSGDTAQT SYAASKAAMN MLSASLATQY GHAQIRCNAV APGLIMTERL LAKLDECMQR | 300 |
| HLSRHQLLQR VGRPEDVAAL VAFLLSDDAA FITGQVLCID GGMLAHVPTY ADGGNSRAAR | 360 |
| PAGDTAKAAA GPRC | 374 |

SEQ ID NO: 119

| | |
|---|---|
| atggacgctg tcactggttt gttgactgtc ccagctactg ctatcaccat cggtggtacc | 60 |
| gctgttgctt tggctgtcgc cttgatcttt tggtacttaa aatccgatat gttgttgaat | 120 |
| ccattgaaca gaagacatag attgagacat gacatcccag ttgttccagg tgccttccca | 180 |
| ttggttggtc acttgcctgc tgttgtttgc gatttgccta gattattgag aagagctgaa | 240 |
| cgtaccttgg gttctcactt ctggttagat ttcggtccag ctggtcattt gatgacttct | 300 |
| ttggacccag atgctttggc tttgttgaga cacaaggacg tctcttccgg tttaattgaa | 360 |
| gatattgctc cagaattatt cggtggtact ttggtcgctc aagacggtat tgctcacaga | 420 |
| caagccagag acgctattca agctgccttg ttgcctaagg gtttaacttt ggctggtatc | 480 |
| ggtgaattgt tcgccccagt tattagagcc agagtccaaa gatggagaga aagaggtgat | 540 |
| gtcactatct tgagagaaac cggtgatttg atgttaaagt tgattttctc cttgatgggt | 600 |
| atccctgctc aagatttgcc tggttggcac agaaagtacc gtcaattatt gcaattgatc | 660 |
| gtcgctccac ctgtcgactt gccaggtttg ccattgagaa gaggtagagc cgctagagac | 720 |
| tggatcgacg ccagattgag agaatttgtc agagctgctc gtgagcacgc ctctcgtacc | 780 |
| ggtttaatca atgatatggt ttctgctttc gacagatccg acgacgcctt gtctgacgat | 840 |
| gttttggtcg ctaacatcag attgttgttg ttaggtggtc acgacaccac cgcttccact | 900 |
| atggcttgga tggttattga attggctcgt caaccaggtt tgtgggatgc tttagttgaa | 960 |
| gaagctcaaa gagttggtgc tgttccaact cgtcatgctg acttggctca atgtccagtt | 1020 |
| gccgaagcct tattcagaga aactttaaga gttcacccag ccactccatt attggtcaga | 1080 |
| agagctttga gagaattgag aatcggtcaa caacgtatcc caaccggtac tgacttgtgt | 1140 |
| attccattgt tgcacttctc cacctccgct ttgttgcatg aagctccaga tcaatttaga | 1200 |
| ttggctagat ggttacaaag aaccgaacca atcagaccag ttgatatgtt acaattcggt | 1260 |
| actggtccac acttttgtat gggttaccac ttagtttggt tggaattggt tcaattctgt | 1320 |
| attgctttgg ctttgaccat gcacgaagct ggtgttagac ctagattgtt atccggtgtt | 1380 |
| gaaaagggta gaagatatta cccaaccgcc catccatcca tgaccattag aattggtttt | 1440 |
| tcttaa | 1446 |

SEQ ID NO: 120

| | |
|---|---|
| MDAVTGLLTV PATAITIGGT AVALAVALIF WYLKSDMLLN PLNRRHRLRH DIPVVPGAFP | 60 |
| LVGHLPAVVC DLPRLLRRAE RTLGSHFWLD FGPAGHLMTS LDPDALALLR HKDVSSGLIE | 120 |
| DIAPELFGGT LVAQDGIAHR QARDAIQAAL LPKGLTLAGI GELFAPVIRA RVQRWRERGD | 180 |
| VTILRETGDL MLKLIFSLMG IPAQDLPGWH RKYRQLLQLI VAPPVDLPGL PLRRGRAARD | 240 |
| WIDARLREFV RAAREHASRT GLINDMVSAF DRSDDALSDD VLVANIRLLL LGGHDTTAST | 300 |
| MAWMVIELAR QPGLWDALVE EAQRVGAVPT RHADLAQCPV AEALFRETLR VHPATPLLVR | 360 |
| RALRELRIGQ QRIPTGIDLC IPLLHFSTSA LLHEAPDQFR LARWLQRTEP IRPVDMLQFG | 420 |
| TGPHFCMGYH LVWLELVQFC IALALTMHEA GVRPRLLSGV EKGRRYYPTA HPSMTIRIGF | 480 |
| S | 481 |

SEQ ID NO: 121

| | |
|---|---|
| atgttgttga atccattgaa cagaagacat agattgagac atgacatccc agttgttcca | 60 |
| ggtgccttcc cattggttgg tcacttgcct gctgttgttt gcgatttgcc tagattattg | 120 |
| agaagagctg aacgtacctt gggttctcac ttctggttag atttcggtcc agctggtcat | 180 |
| ttgatgactt ctttggaccc agatgctttg gctttgttga gacacaagga cgtctcttcc | 240 |
| ggtttaattg aagatattgc tccagaatta ttcggtggta ctttggtcgc tcaagacggt | 300 |
| attgctcaca gacaagccag agacgctatt caagctgcct tgttgcctaa gggtttaact | 360 |
| ttggctggta tcggtgaatt gttcgcccca gttattagag ccagagtcca aagatggaga | 420 |
| gaaagaggtg atgtcactat cttgagagaa accggtgatt tgatgttaaa gttgattttc | 480 |
| tccttgatgg gtatccctgc tcaagatttg cctggttggc acagaaagta ccgtcaatta | 540 |
| ttgcaattga tcgtcgctcc acctgtcgac ttgccaggtt tgccattgag aagaggtaga | 600 |
| gccgctagag actggatcga cgccagattg agagaatttg tcagagctgc tcgtgagcac | 660 |
| gcctctcgta ccggtttaat caatgatatg gtttctgctt tcgacagatc cgacgacgcc | 720 |
| ttgtctgacg atgttttggt cgctaacatc agattgttgt tgttaggtgg tcacgacacc | 780 |
| accgcttcca ctatggcttg gatggttatt gaattggctc gtcaaccagg tttgtgggat | 840 |
| gctttagttg aagaagctca aagagttggt gctgttccaa ctcgtcatgc tgacttggct | 900 |
| caatgtccag ttgccgaagc cttattcaga gaaactttaa gagttcaccc agccactcca | 960 |
| ttattggtca gaagagcttt gagagaattg agaatcggtc aacaacgtat cccaaccggt | 1020 |
| actgacttgt gtattccatt gttgcacttc tccacctccg ctttgttgca tgaagctcca | 1080 |
| gatcaattta gattggctag atggttacaa agaaccgaac caatcagacc agttgatatg | 1140 |
| ttacaattcg gtactggtcc acacttttgt atgggttacc acttagtttg gttggaattg | 1200 |
| gttcaattct gtattgcttt ggctttgacc atgcacgaag ctggtgttag acctagattg | 1260 |
| ttatccggtg ttgaaaaggg tagaagatat tacccaaccg cccatccatc catgaccatt | 1320 |
| agaattggtt tttcttaa | 1338 |

SEQ ID NO: 122

| | |
|---|---|
| MLLNPLNRRH RLRHDIPVVP GAFPLVGHLP AVVCDLPRLL RRAERTLGSH FWLDFGPAGH | 60 |
| LMTSLDPDAL ALLRHKDVSS GLIEDIAPEL FGGTLVAQDG IAHRQARDAI QAALLPKGLT | 120 |
| LAGIGELFAP VIRARVQRWR ERGDVTILRE TGDLMLKLIF SLMGIPAQDL PGWHRKYRQL | 180 |
| LQLIVAPPVD LPGLPLRRGR AARDWIDARL REFVRAAREH ASRTGLINDM VSAFDRSDDA | 240 |
| LSDDVLVANI RLLLLGGHDT TASTMAWMVI ELARQPGLWD ALVEEAQRVG AVPTRHADLA | 300 |
| QCPVAEALFR ETLRVHPATP LLVRRALREL RIGQQRIPTG TDLCIPLLHF STSALLHEAP | 360 |
| DQFRLARWLQ RTEPIRPVDM LQFGTGPHFC MGYHLVWLEL VQFCIALALT MHEAGVRPRL | 420 |
| LSGVEKGRRY YPTAHPSMTI RIGFS | 445 |

SEQ ID NO: 123

| | |
|---|---|
| atggatgctg tcaccggttt gttaaccgtt ccagctaccg ctattaccat cggtggtacc | 60 |
| gctgtcgcct tagctgttgc tttgattttc tggtacttaa agtcttctga acaacaacct | 120 |
| ttgccaacct tgccaatgtg gagagttgac cacattgaac cttctccaga aatgttggct | 180 |
| ttgagagcta atggtcctat ccatcgtgtt cgtttcccat ctggtcacga aggttggtgg | 240 |
| gtcaccggtt atgacgaagc taaggctgtt ttgtccgatg ccgccttccg tcctgctggt | 300 |
| atgcctccag ctgctttcac tccagactct gtcattttgg gttctccagg ttggttagtc | 360 |
| tctcacgaag gtagagaaca tgctagattg cgtgctattg ttgctccagc tttctctgat | 420 |
| agaagagtta aattgttggt ccaacaagtc gaagccattg ctgcccactt gttcgagact | 480 |
| ttagctgccc aacctcaacc tgccgatttg agaagacact tgtctttccc tttaccagcc | 540 |
| atggttattt ctgccttaat gggtgtctta tacgaggacc acgctttctt tgctggtttg | 600 |
| tctgacgaag ttatgactca ccaacatgaa tccggtccac gttctgcttc tagattggcc | 660 |
| tgggaggaat tgagagccta cattagaggt aagatgagag acaagagaca agacccagac | 720 |
| gataacttgt taactgattt gttggctgct gtcgatcaag gtaaggcttc cgaagaagaa | 780 |
| gctgttggtt tggccgctgg tatgttggtt gctggtcatg aatctactgt tgcccaaatc | 840 |
| gaatttggtt tgttggccat gttcagacac ccacaacaaa gagaaagatt agttggtgat | 900 |
| ccatctttgg ttgacaaggc tgttgaggaa attttgagaa tgtatccacc aggtgctggt | 960 |
| tgggatggta tcatgcgtta cccaagaact gatgttacta tcgctggtga acacattcca | 1020 |
| gccgaatcca aggttttggt cggtttgcca gctacctcct tcgatccaca ccactttgac | 1080 |
| gatccagaaa tcttcgacat cgaaagacaa gaaaaaccac acttagcctt ttcctacggt | 1140 |
| cctcacgctt gtatcggtgt tgctttggct agattggagt tgaaggttgt cttcggttct | 1200 |
| attttccaaa gattgcctgc tttacgttta gccgttgctc cagaacaatt gaagttgaga | 1260 |
| aaggaaatca tcaccggtgg ttttgaacaa ttcccagttt tgtggtaa | 1308 |

SEQ ID NO: 124

| | |
|---|---|
| MDAVTGLLTV PATAITIGGT AVALAVALIF WYLKSSEQQP LPTLPMWRVD HIEPSPEMLA | 60 |
| LRANGPIHRV RFPSGHEGWW VTGYDEAKAV LSDAAFRPAG MPPAAFTPDS VILGSPGWLV | 120 |
| SHEGREHARL RAIVAPAFSD RRVKLLVQQV EAIAAHLFET LAAQPQPADL RRHLSFPLPA | 180 |
| MVISALMGVL YEDHAFFAGL SDEVMTHQHE SGPRSASRLA WEELRAYIRG KMRDKRQDPD | 240 |
| DNLLTDLLAA VDQGKASEEE AVGLAAGMLV AGHESTVAQI EFGLLAMFRH PQQRERLVGD | 300 |
| PSLVDKAVEE ILRMYPPGAG WDGIMRYPRT DVTIAGEHIP AESKVLVGLP ATSFDPHHFD | 360 |
| DPEIFDIERQ EKPHLAFSYG PHACIGVALA RLELKVVFGS IFQRLPALRL AVAPEQLKLR | 420 |
| KEIITGGFEQ FPVLW | 435 |

SEQ ID NO: 125

| | |
|---|---|
| atggacgctg ttaccggttt gttgactgtt ccagctactg ctatcaccat tggtggtact | 60 |
| gctgttgctt tggctgtcgc tttaatcttc tggtatttaa agtccgacgt tcaagaaacc | 120 |
| actgctgctt gcagagacgc tttcgctgaa ttagcttccc cagcttgtat tcacgatcct | 180 |
| tacccattca tgagatggtt gcgtgaacac gacccagttc acagagctgc ctctggtttg | 240 |
| ttcttgttgt ccagacatgc tgatatcttt tgggctttca aggccaccgg tgatgctttc | 300 |
| agaggtccag ctccaggtga gttggctaga tacttttcta gagctgccac ctctccatcc | 360 |
| ttgaacttgt tggcctctac tttggctatg aaggatccac ctacccacac cagattgaga | 420 |
| agattgattt ctagagactt cactatgggt caaatcgaca acttgagacc atccattgcc | 480 |
| agaatcgttg ccgctagatt agatggtatt actccagcct tggaaagagg tgaagctgtc | 540 |
| gacttgcaca gagaatttgc tttggcctta cctatgttgg ttttcgctga attgtttggt | 600 |
| atgcctcaag atgatatgtt tgagttagct gccggtatcg gtactatttt ggaaggtttg | 660 |
| ggtccacatg cttctgatcc acaattggct gctgccgacg ctgcttctgc tagagtccaa | 720 |
| gcttacttcg gtgatttgat ccaaagaaaa cgtaccgatc ctagaagaga catcgtctcc | 780 |
| atgttggttg gtgctcacga tgacgatgcc gatactttgt ctgacgctga attaatttct | 840 |
| atgttgtggg gtatgttgtt aggtggtttc gttaccactg ctgcctccat cgatcatgct | 900 |
| gttttggcta tgttggctta tccagaacaa agacattggt tacaagctga cgctgctaga | 960 |
| gttagagctt ttgttgaaga agttttaaga tgtgacgctc cagctatgtt ttcctccatt | 1020 |
| ccaagaattg ctcaaagaga tatcgaattg ggtggtgtcg tcattcctaa gaacgctgac | 1080 |
| gttagagtct taatcgcctc cggtaacaga gatccagacg cttttgctga tccagataga | 1140 |
| ttcgatccag ctagattcta tggtacctcc ccaggtatgt ctactgacgg taaaattatg | 1200 |
| ttatctttcg gtcatggtat ccacttctgc ttaggtgccc aattggccag agtccaattg | 1260 |
| gctgaatctt tgcctagaat tcaagctaga tttccaactt tggcttttgc tggtcaacca | 1320 |
| accagagaac catccgcttt cttaagaact ttccgtactt tgccagtcag attgcatgcc | 1380 |
| caaggttcct aa | 1392 |

SEQ ID NO: 126

| | |
|---|---|
| MDAVTGLLTV PATAITIGGT AVALAVALIF WYLKSDVQET TAACRDAFAE LASPACIHDP | 60 |
| YPFMRWLREH DPVHRAASGL FLLSRHADIF WAFKATGDAF RGPAPGELAR YESRAATSPS | 120 |
| LNLLASTLAM KDPPTHTRLR RLISRDFTMG QIDNLRPSIA RIVAARLDGI TPALERGEAV | 180 |
| DLHREFALAL PMLVFAELFG MPQDDMFELA AGIGTILEGL GPHASDPQLA AADAASARVQ | 240 |
| AYFGDLIQRK RTDPRRDIVS MLVGAHDDDA DTLSDAELIS MLWGMLLGGF VTTAASIDHA | 300 |
| VLAMLAYPEQ RHWLQADAAR VRAFVEEVLR CDAPAMFSSI PRIAQRDIEL GGVVIPKNAD | 360 |
| VRVLIASGNR DPDAFADPDR FDPARFYGTS PGMSTDGKIM LSFGHGIHFC LGAQLARVQL | 420 |
| AESLPRIQAR FPTLAFAGQP TREPSAFLRT FRTLPVRLHA QGS | 463 |

SEQ ID NO: 127

| | |
|---|---|
| atgtctgaac aacaaccttt gccaaccttg ccaatgtgga gagttgacca cattgaacct | 60 |
| tctccagaaa tgttggcttt gagagctaat ggtcctatcc atcgtgttcg tttcccatct | 120 |
| ggtcacgaag gttggtgggt caccggttat gacgaagcta aggctgtttt gtccgatgcc | 180 |
| gccttccgtc ctgctggtat gcctccagct gctttcactc cagactctgt cattttgggt | 240 |
| tctccaggtt ggttagtctc tcacgaaggt agagaacatg ctagattgcg tgctattgtt | 300 |
| gctccagctt tctctgatag aagagttaaa ttgttggtcc aacaagtcga agccattgct | 360 |
| gcccacttgt tcgagacttt agctgcccaa cctcaacctg ccgatttgag aagacacttg | 420 |
| tctttccctt taccagccat ggttatttct gccttaatgg gtgtcttata cgaggaccac | 480 |
| gctttctttg ctggtttgtc tgacgaagtt atgactcacc aacatgaatc cggtccacgt | 540 |
| tctgcttcta gattggcctg ggaggaattg agagcctaca ttagaggtaa gatgagagac | 600 |
| aagagacaag acccagacga taacttgtta actgatttgt tggctgctgt cgatcaaggt | 660 |
| aaggcttccg aagaagaagc tgttggtttg gccgctggta tgttggttgc tggtcatgaa | 720 |
| tctactgttg cccaaatcga atttggtttg ttggccatgt tcagacaccc acaacaaaga | 780 |
| gaaagattag ttggtgatcc atctttggtt gacaaggctg ttgaggaaat tttgagaatg | 840 |
| tatccaccag gtgctggttg ggatggtatc atgcgttacc caagaactga tgttactatc | 900 |
| gctggtgaac acattccagc cgaatccaag gttttggtcg gtttgccagc tacctccttc | 960 |
| gatccacacc actttgacga tccagaaatc ttcgacatcg aaagacaaga aaaaccacac | 1020 |
| ttagcctttt cctacggtcc tcacgcttgt atcggtgttg ctttggctag attggagttg | 1080 |
| aaggttgtct tcggttctat tttccaaaga ttgcctgctt tacgtttagc cgttgctcca | 1140 |
| gaacaattga agttgagaaa ggaaatcatc accggtggtt ttgaacaatt cccagttttg | 1200 |
| tggtaa | 1206 |

SEQ ID NO: 128

| | |
|---|---|
| MSEQQPLPTL PMWRVDHIEP SPEMLALRAN GPIHRVRFPS GHEGWWVTGY DEAKAVLSDA | 60 |
| AFRPAGMPPA AFTPDSVILG SPGWLVSHEG REHARLRAIV APAFSDRRVK LLVQQVEAIA | 120 |
| AHLFETLAAQ PQPADLRRHL SFPLPAMVIS ALMGVLYEDH AFFAGLSDEV MTHQHESGPR | 180 |
| SASRLAWEEL RAYIRGKMRD KRQDPDDNLL TDLLAAVDQG KASEEEAVGL AAGMLVAGHE | 240 |
| STVAQIEFGL LAMFRHPQQR ERLVGDPSLV DKAVEEILRM YPPGAGWDGI MRYPRIDVTI | 300 |
| AGEHIPAESK VLVGLPATSF DPHHFDDPEI FDIERQEKPH LAFSYGPHAC IGVALARLEL | 360 |
| KVVFGSIFQR LPALRLAVAP EQLKLRKEII TGGFEQFPVL W | 401 |

SEQ ID NO: 129

| | |
|---|---|
| atgttgtcca gacatgctga tatcttttgg gctttcaagg ccaccggtga tgctttcaga | 60 |
| ggtccagctc caggtgagtt ggctagatac ttttctagag ctgccacctc tccatccttg | 120 |
| aacttgttgg cctctacttt ggctatgaag gatccaccta cccacaccag attgagaaga | 180 |
| ttgatttcta gagacttcac tatgggtcaa atcgacaact tgagaccatc cattgccaga | 240 |
| atcgttgccg ctagattaga tggtattact ccagccttgg aaagaggtga agctgtcgac | 300 |
| ttgcacagag aatttgcttt ggccttacct atgttggttt tcgctgaatt gtttggtatg | 360 |
| cctcaagatg atatgtttga gttagctgcc ggtatcggta ctattttgga aggtttgggt | 420 |
| ccacatgctt ctgatccaca attggctgct gccgacgctg cttctgctag agtccaagct | 480 |
| tacttcggtg atttgatcca aagaaaacgt accgatccta gaagagacat cgtctccatg | 540 |
| ttggttggtg ctcacgatga cgatgccgat actttgtctg acgctgaatt aatttctatg | 600 |
| ttgtggggta tgttgttagg tggtttcgtt accactgctg cctccatcga tcatgctgtt | 660 |
| ttggctattt tggcttatcc agaacaaaga cattggttac aagctgacgc tgctagagtt | 720 |
| agagcttttg ttgaagaagt tttaagatgt gacgctccag ctatgttttc ctccattcca | 780 |
| agaattgctc aaagagatat cgaattgggt ggtgtcgtca ttcctaagaa cgctgacgtt | 840 |
| agagtcttaa tcgcctccgg taacagagat ccagacgctt ttgctgatcc agatagattc | 900 |
| gatccagcta gattctatgg tacctcccca ggtatgtcta ctgacggtaa aattatgtta | 960 |
| tctttcggtc atggtatcca cttctgctta ggtgcccaat tggccagagt ccaattggct | 1020 |
| gaatctttgc ctagaattca agctagattt ccaactttgg cttttgctgg tcaaccaacc | 1080 |
| agagaaccat ccgctttctt aagaactttc cgtactttgc cagtcagatt gcatgcccaa | 1140 |
| ggttcctaa | 1149 |

SEQ ID NO: 130

| | |
|---|---|
| MLSRHADIFW AFKATGDAFR GPAPGELARY FSRAATSPSL NLLASTLAMK DPPTHTRLRR | 60 |
| LISRDFTMGQ IDNLRPSIAR IVAARLDGIT PALERGEAVD LHREFALALP MLVFAELFGM | 120 |
| PQDDMFELAA GIGTILEGLG PHASDPQLAA ADAASARVQA YFGDLIQRKR TDPRRDIVSM | 180 |
| LVGAHDDDAD TLSDAELISM LWGMLLGGFV TTAASIDHAV LAMLAYPEQR HWLQADAARV | 240 |
| RAFVEEVLRC DAPAMFSSIP RIAQRDIELG GVVIPKNADV RVLIASGNRD PDAFADPDRF | 300 |
| DPARFYGTSP GMSTDGKIML SFGHGIHFCL GAQLARVQLA ESLPRIQARF PTLAFAGQPT | 360 |
| REPSAFLRTF RTLPVRLHAQ GS | 382 |

SEQ ID NO: 131

| | |
|---|---|
| atgatccaaa ccgaaagagc cgttcaacaa gttttggaat ggggtagatc tttgactggt | 60 |
| tttgctgatg aacatgctgt tgaagctgtt agaggtggtc agtacatctt gcaaagaatt | 120 |
| catccatctt tgagaggtac atctgctaga actggtagag atccacaaga cgaaactttg | 180 |
| atcgttacct tctatagaga attggccttg ttgttttggt tggatgattg caatgatttg | 240 |
| ggcttgattt ccccagaaca attggctgct gttgaacaag ctttgggtca aggtgttcca | 300 |
| tgtgctttgc caggttttga aggttgtgct gttttgagag cttctttggc tactttggct | 360 |
| tacgatagaa gagattatgc tcagttgttg gatgatacca gatgttattc tgctgcttta | 420 |
| agagctggtc atgctcaagc tgttgctgct gaaagatggt cttatgctga atacttgcat | 480 |
| aacggtattg actccattgc ttacgctaac gttttctgtt gtttgtcttt gttgtggggt | 540 |
| ttggatatgg ctactttgag agctagacca gcttttagac aagtcttgag attgatttcc | 600 |
| gccatcggta gattgcaaaa tgacttgcat ggttgcgata aggatagatc tgctggtgaa | 660 |
| gctgataacg ctgttatttt gttgttgcaa agatacccag ctatgccagt tgttgaattc | 720 |
| ttgaatgatg aattggctgg tcacaccaga atgttgcata gagttatggc tgaagaaaga | 780 |
| tttccagctc catggggtcc attgattgaa gctatggctg ctattagagt tcagtactac | 840 |
| agaacttcta cctccagata tagatccgat gctgtaagag gtggacaaag agcaccagct | 900 |
| taa | 903 |

SEQ ID NO: 132

| | |
|---|---|
| MIQTERAVQQ VLEWGRSLTG FADEHAVEAV RGGQYILQRI HPSLRGTSAR TGRDPQDETL | 60 |
| IVTFYRELAL LFWLDDCNDL GLISPEQLAA VEQALGQGVP CALPGFEGCA VLRASLATLA | 120 |
| YDRRDYAQLL DDTRCYSAAL RAGHAQAVAA ERWSYAEYLH NGIDSIAYAN VFCCLSLLWG | 180 |
| LDMATLRARP AFRQVLRLIS AIGRLQNDLH GCDKDRSAGE ADNAVILLLQ RYPAMPVVEF | 240 |
| LNDELAGHTR MLHRVMAEER FPAPWGPLIE AMAAIRVQYY RTSTSRYRSD AVRGGQRAPA | 300 |

SEQ ID NO: 133

| | |
|---|---|
| atggctggtg attctcatga accatttgct actatagtcg agtctccttt gtcttacgtt | 60 |
| tcttccttgc catccaaaca tttcagagtt caattattgg aggccttgaa catctggtat | 120 |
| gaattgccac aaaacgaggt ttccaagatc ggtgatatct tgcagttgtt gcataactcc | 180 |
| tcattgatct tggatgactt ccaagataga tccccattga gaagaggtag accagctgct | 240 |
| catgctttgt ttggtgaagc tcaagctatt aactcttcct cttacggttt cattaaggct | 300 |
| gttgctttgg ctcaagaatc cttcgatttg gaatctacta aggctgttac taccgctatg | 360 |
| ttgagatctt ttgaaggtca agctgctgaa ttgcattgga ctcatacaaa aacttgccca | 420 |
| tccgttcaag aatacttgga aatggttaac ttctcctcct tgttgcattt ggctccacaa | 480 |
| ttgatgcaag ctaaaagagg ttctgctact ccagttgatc aaaggtctat ggtttccttg | 540 |
| atgagattgc taggtcaatt ctaccaaatc agggacgact atatgaactt gacttctgct | 600 |
| cattacgaaa aggataaggg tttctgcgaa gatttggacg aaggtaaata ttccttgcca | 660 |
| ttgattcatg ctttggccgt taagccaaga tctgttttgt tggcttctgc tttggctgct | 720 |
| tctggtgctc caggtggttt atctagacaa caaaaagtct gcatcttgga agaattggaa | 780 |
| aaggctagat ctttggcttg gacaaaagct actttgtgcg aattgcaagt tgccatgtct | 840 |
| gaagaaattg cccaattgga agatagattc ggtagaccaa acgagttgtt gcaaaccttg | 900 |
| atttctaagg ttgccattaa gtaa | 924 |

SEQ ID NO: 134

| | |
|---|---|
| MAGDSHEPFA TIVESPLSYV SSLPSKHFRV QLLEALNIWY ELPQNEVSKI GDILQLLHNS | 60 |
| SLILDDFQDR SPLRRGRPAA HALFGEAQAI NSSSYGFIKA VALAQESFDL ESTKAVTTAM | 120 |
| LRSFEGQAAE LHWTHTKTCP SVQEYLEMVN FSSLLHLAPQ LMQAKRGSAT PVDQRSMVSL | 180 |
| MRLLGQFYQI RDDYMNLTSA HYEKDKGFCE DLDEGKYSLP LIHALAVKPR SVLLASALAA | 240 |
| SGAPGGLSRQ QKVCILEELE KARSLAWTKA TLCELQVAMS EEIAQLEDRF GRPNELLQTL | 300 |
| ISKVAIK | 307 |

SEQ ID NO: 135

| | |
|---|---|
| atggctgcta gattgttaag agttgcctct gctgcactag gtgatactgc cggaagatgg | 60 |
| agactattag taagaccaag agctggcgcc ggtggattaa ggggctcaag aggtcctggt | 120 |
| ctaggaggcg gtgccgtcgc tacaagaacc ctttccgtga gtggaagggc acaaagctct | 180 |
| tcagaggaca aaattactgt tcactttatc aatagagatg gtgagacatt gaccactaag | 240 |
| ggcaaaatcg gtgactcctt attggatgta gtcgtgcaga acaacttaga cattgatgga | 300 |
| ttcggtgctt gtgaaggcac actagcctgc agtacctgtc accttatatt tgagcaacat | 360 |
| atcttcgaaa agttggaagc aattactgat gaggaaaacg acatgttaga tctagcttat | 420 |
| ggtttgacag acaggagcag attaggatgc cagatatgtc ttaccaaagc catggataat | 480 |
| atgactgtta gagtaccaga tgcagtctct gacgctaggg aatcaatcga tatgggtatg | 540 |
| aactccagta agattgagta a | 561 |

SEQ ID NO: 136

| | |
|---|---|
| MAARLLRVAS AALGDTAGRW RLLVRPRAGA GGLRGSRGPG LGGGAVATRT LSVSGRAQSS | 60 |
| SEDKITVHFI NRDGETLITK GKIGDSLLDV VVQNNLDIDG FGACEGTLAC STCHLIFEQH | 120 |
| IFEKLEAITD EENDMLDLAY GLTDRSRLGC QICLTKAMDN MTVRVPDAVS DARESIDMGM | 180 |
| NSSKIE | 186 |

SEQ ID NO: 137

| | |
|---|---|
| atgcttttga acacctttac ccaaactgcc agaagtgaca ggtgtgcttt ctatggaaat | 60 |
| gtcgaagtgg gcagagatgt tacagtacaa gaattaaggg tctacaggtt gaccgcagtt | 120 |
| gttctaagct atggtgccga agatcaccag gcacttgata ttccaggtga agagttgcca | 180 |
| ggagtttttt ctgcaagagc tttcgtaggc tggtacaacg gtttgccaga aaatagagaa | 240 |
| ttagcccctg acctatcatg cgatactgca gtcatattgg gacaaggtaa cgtggctttg | 300 |
| gacgttgcca ggatactttt gaccccacct gaccacttag agaaaactga tattaccgaa | 360 |
| gcagctctag gcgcccttag acagtccaga gtaaagacag tctggatagt tggtaggaga | 420 |
| ggaccattgc aagtggcctt tactatcaaa gagcttagag agatgattca acttcctggc | 480 |
| accaggccta tgttggaccc agctgatttc ttaggccttc aggatagaat tagggaagcc | 540 |
| gcaagaccta gaaagaggtt gatggagtta ctattgagaa cagctactga aaaaccaggt | 600 |
| gttgaagagg ccgcaagaag ggctagtgct agcagagctt ggggattaag gtttttcaga | 660 |
| agccctcaac aagtacttag gcttccagac ggtagggcaa gaagatcagc ttggcagtcc | 720 |
| cctgaattgg aaggcatagg agaggcccat ccaggtagcg cacactgggg ctgtggtgga | 780 |
| cctccatgcg gtttagtact ttcttcaatc ggctataagt ctaggcctat tgatccaagc | 840 |
| gtgccttttg acccaaaatt gggtgttgta ccaaatatgg aaggaagagt cgttgatgtg | 900 |
| cctggtttat actgttccgg ctgggttaag agaggaccaa caggtgtaat aaccactaca | 960 |
| atgactgata gttttctaac cggtcaaatt ttgctacagg accttaaagc tggccatttg | 1020 |
| ccttccggtc caaggcctgg ctcagccttc attaaggcac tattagattc taggggtgtc | 1080 |
| tggccagttt cctttagtga ctgggaaaaa ttggatgctg aggaagtgag cagaggccaa | 1140 |
| gcatctggaa agcctagaga aaaacttcta gatcctcaag agatgctaag attgttaggt | 1200 |
| cactga | 1206 |

SEQ ID NO: 138

| | |
|---|---|
| MLLNTFTQTA RSDRCAFYGN VEVGRDVTVQ ELRVYRLTAV VLSYGAEDHQ ALDIPGEELP | 60 |
| GVFSARAFVG WYNGLPENRE LAPDLSCDTA VILGQGNVAL DVARILLTPP DHLEKTDITE | 120 |
| AALGALRQSR VKTVWIVGRR GPLQVAFTIK ELREMIQLPG TRPMLDPADF LGLQDRIREA | 180 |
| ARPRKRLMEL LLRTATEKPG VEEAARRASA SRAWGLRFFR SPQQVLRLPD GRARRSAWQS | 240 |
| PELEGIGEAH PGSAHWGCGG PPCGLVLSSI GYKSRPIDPS VPFDPKLGVV PNMEGRVVDV | 300 |
| PGLYCSGWVK RGPTGVITTT MTDSFLTGQI LLQDLKAGHL PSGPRPGSAF IKALLDSRGV | 360 |
| WPVSFSDWEK LDAEEVSRGQ ASGKPREKLL DPQEMLRLLG H | 401 |

SEQ ID NO: 139

| | |
|---|---|
| atggtggaca caaacttatt ggcttctgtt gccgtcgctc tagtcgtcgt tttcgttgct | 60 |
| tacaagtact ttaatggtgg gctggaagtc caatcatcta atgctggatc tagtacacct | 120 |
| tttggtaatg caaaggctga cgaagacgga gattccagga acttcgtggc tttgatggaa | 180 |
| aaaaataata agaacgttat tgttttctat ggttcccaaa caggaacggc cgaggatttg | 240 |
| gctagcaaat tggccaagga gttaagctca aagtatggtc taaggacaat gaccgccgat | 300 |
| cccgaaaatt ttgatttcga caaatttgat acctttccag agagtcatct ggctgttttt | 360 |
| atcacagcca gttacggaga tggcgaacct acagacaatg cacaggattt atattccttc | 420 |
| ttaggtaatt caccaagttt ctcacaggat ggtgaaaccc ttgagaacct taattttgca | 480 |
| gtgttcggtt taggtaatgt actatatgaa ttctacaaca aggccggcag agatatgcac | 540 |
| aagtttctaa ctgatttagg cggtcactca ataggtccat acggggaagg tgatgactca | 600 |
| aaagggatgt tagaggaaga ttacatggca tggaaagatg aatttctagc tgccctagtt | 660 |
| acgaaatggg gtttgaagga aagagaagct gtctacgagc cagccattag tgtgaaggat | 720 |
| attgaagagg atgctcaatc acatgacgtt tacttgggtg aaccaaacct aaagcactta | 780 |
| caagctagca aggcccgtga agtccccaaa gggccgtata atgctagcaa tccaatgtta | 840 |
| gccaaggtta cagcagctca ggagttgttt actaacactg atcgtcattg tattcatatg | 900 |
| gagtttgata ctaccggcgc gaggtatacc acgggcgatc acctggcttt ctggtgtcaa | 960 |
| aataacgaag aggaagttca gagattcgct aaggcattag gtataaccaa cccgcagcaa | 1020 |
| ccaattgcaa tatcagtgct tgacaagact tcaacagtaa gaattcccag tccaactacc | 1080 |
| tatgagacca ttataagaca ttttttagag atcaacggcc cagtgagccg tcaagttctt | 1140 |
| agtagcattg caccgttcgc cccgagcgag gaagtcaaga aagctacgca acagctaggc | 1200 |
| tctaacaagg aactgtttgc tagtcatgtt gccgcaaaaa agtttaacat agcaagattg | 1260 |
| ttgttgcatt tatcaggcgg ccaaccttgg aaaaacgtcc ccttttcatt catcattgaa | 1320 |
| accattcccc atctacaacc caggtactac tctatttcct catcatcagt ccaaagccct | 1380 |
| aatactatct ctattactgc tgtcgtggaa agacaaaagt tagccggtgt agatcatgaa | 1440 |
| ttgagaggtg tagccacgaa tcaaattttg gccttgtccg aagcattgat aggtagacct | 1500 |
| tcaagcacat acagactaca gcagccccat gattttacag gttcattaaa ttcacaagat | 1560 |
| attagagtac cagtacatat tagacatagc ttatttaagc tacctgccaa acccacagtt | 1620 |
| ccaataataa tggtcggacc aggtaccggc gtcgcgccat tcagaggttt tgtgcatgaa | 1680 |
| agggcagctc aaaaggctgc cggtaaggaa gttggaaaag ctctattgtt caccggatca | 1740 |
| agacatgcaa atgaggattt tctatacaga gacgaatgga aacaatttag tgattttttg | 1800 |
| gatttggaaa cagctttttc tagagattcc aatactaagg tttatgtgca acacaagctg | 1860 |
| aaagaaagag ccaaggacgt gtttgctttg cttaatgaag gcgcggtttt ctatgtctgc | 1920 |
| ggtgacgcgg gtggaatgtc acatgatgtg catagcgcct tgttggaaat tgtagctcaa | 1980 |
| gagggtaact tgtctagcga agatgcagat aaatttgtca ggaaaatgag atcaagaaat | 2040 |
| aagtaccaag aggatgtatg gtaa | 2064 |

SEQ ID NO: 140

| | |
|---|---|
| MVDTNLLASV AVALVVVFVA YKYFNGGLEV QSSNAGSSTP FGNAKADEDG DSRNFVALME | 60 |
| KNNKNVIVFY GSQTGTAEDL ASKLAKELSS KYGLRTMTAD PENFDFDKFD TFPESHLAVF | 120 |
| ITASYGDGEP TDNAQDLYSF LGNSPSFSQD GETLENLNFA VFGLGNVLYE FYNKAGRDMH | 180 |
| KFLTDLGGHS IGPYGEGDDS KGMLEEDYMA WKDEFLAALV TKWGLKEREA VYEPAISVKD | 240 |
| IEEDAQSHDV YLGEPNLKHL QASKAREVPK GPYNASNPML AKVTAAQELF TNTDRHCIHM | 300 |
| EFDTTGARYT TGDHLAFWCQ NNEEEVQRFA KALGITNPQQ PIAISVLDKT STVRIPSPIT | 360 |
| YETIIRHFLE INGPVSRQVL SSIAPFAPSE EVKKATQQLG SNKELFASHV AAKKFNIARL | 420 |
| LLHLSGGQPW KNVPFSFIIE TIPHLQPRYY SISSSSVQSP NTISITAVVE RQKLAGVDHE | 480 |
| LRGVATNQIL ALSEALIGRP SSTYRLQQPH DFTGSLNSQD IRVPVHIRHS LFKLPAKPTV | 540 |
| PIIMVGPGTG VAPFRGFVHE RAAQKAAGKE VGKALLFTGS RHANEDFLYR DEWKQFSDFL | 600 |
| DLETAFSRDS NTKVYVQHKL KERAKDVFAL LNEGAVFYVC GDAGGMSHDV HSALLEIVAQ | 660 |
| EGNLSSEDAD KFVRKMRSRN KYQEDVW | 687 |

SEQ ID NO: 141

| | |
|---|---|
| atgtccatat tcaacatgat cacttcttac gctggcagtc aattactgcc attctatatt | 60 |
| gctatttttg tttttactct ggttccttgg gctatcaggt tttcttggct tgaattgagg | 120 |
| aaggggtctg tagtcccctt agcaaatcca cccgatagtc tttttggaac aggtaagaca | 180 |
| cgtagatcct ttgtaaaatt atctagggaa atattagcta aggctagatc attgtttccg | 240 |
| aacgaaccct ttagattaat cactgactgg ggcgaggtat taatattacc tcctgatttt | 300 |
| gctgacgaaa tacgtaatga tccgaggcta tcattcagta aagctgctat gcaagataat | 360 |
| cacgccggta ttccagggtt tgagacagtc gctttggtgg gacgtgaaga ccaattaata | 420 |
| caaaaagtcg ccagaaagca attgactaag catcttagcg cagtaattga acctctgagt | 480 |
| agggaaagta ccctagctgt atctctaaat tttggagaaa cgacagaatg gagagccatt | 540 |
| aggcttaagc cagcaattct tgatattatt gctaggatct cctcacgtat ctatctagga | 600 |
| gatcaactat gcaggaatga ggcatggcta aagattacta agacttacac aacaaacttt | 660 |
| tacactgcct ctaccaatct tagaatgttc cccagaagta taagaccttt agctcactgg | 720 |
| ttcttgccag aatgtagaaa gcttcgtcaa gagaggaagg atgcaattgg tattataacg | 780 |
| ccactaatcg aaaggagaag agaattgcgt agagcagcta ttgcagctgg acagccttta | 840 |
| ccagtttttc acgacgcaat cgattggtcc gaacaagagg ctgaagctgc cggtacaggt | 900 |
| gcatcatttg accctgtgat atttcaatta acattgtctt tgttggctat tcatacaacc | 960 |
| tatgacttat tgcaacagac catgatagac ttgggtaggc accctgaata tatagaacct | 1020 |
| ctgagacaag aagttgtgca actgttgaga gaagaaggtt ggaagaaaac tactttattt | 1080 |
| aagatgaagt tacttgattc cgcaataaag gaaagtcaaa gaatgaaacc aggatccatt | 1140 |
| gtcacgatgc gtcgttacgt gaccgaggac atcacactat cctctggttt aacgctaaaa | 1200 |
| aaaggcacca gattgaatgt tgacaatcgt aggttggatg atcccaagat ctatgacaat | 1260 |
| cctgaagtct ataatcctta tcgtttttat gatatgagat ccgaagcagg taaagatcat | 1320 |
| ggcgcccagc tggttagtac aggctctaat cacatgggtt ttggccatgg gcaacattca | 1380 |
| tgtccgggta gattttttgc cgcaaatgag atcaaagtag ccctatgtca tattttagtg | 1440 |
| aaatatgact ggaaattatg cccagataca gaaaccaaac ctgacactcg tgggatgata | 1500 |
| gctaagtcta gcccagttac tgacatcctt attaagagaa gagaatcagt agagttagat | 1560 |
| ttagaggcga tttaa | 1575 |

SEQ ID NO: 142

| | |
|---|---|
| MSIFNMITSY AGSQLLPFYI AIFVFTLVPW AIRFSWLELR KGSVVPLANP PDSLFGTGKT | 60 |
| RRSFVKLSRE ILAKARSLFP NEPFRLITDW GEVLILPPDF ADEIRNDPRL SFSKAAMQDN | 120 |
| HAGIPGFETV ALVGREDQLI QKVARKQLTK HLSAVIEPLS RESTLAVSLN FGETTEWRAI | 180 |
| RLKPAILDII ARISSRIYLG DQLCRNEAWL KITKTYTINF YTASTNLRMF PRSIRPLAHW | 240 |
| FLPECRKLRQ ERKDAIGIIT PLIERRRELR RAAIAAGQPL PVFHDAIDWS EQEAEAAGTG | 300 |
| ASFDPVIFQL TLSLLAIHTT YDLLQQTMID LGRHPEYIEP LRQEVVQLLR EEGWKKTTLF | 360 |
| KMKLLDSAIK ESQRMKPGSI VIMRRYVTED ITLSSGLTLK KGTRLNVDNR RLDDPKIYDN | 420 |
| PEVYNPYRFY DMRSEAGKDH GAQLVSTGSN HMGFGHGQHS CPGRFFAANE IKVALCHILV | 480 |
| KYDWKLCPDT ETKPDTRGMI AKSSPVTDIL IKRRESVELD LEAI | 524 |

SEQ ID NO: 143

| | |
|---|---|
| atgaaatata caacctgtca gatgaacatc ttcccttccc tatggtcaat gaaaacgtcc | 60 |
| ttcagatggc ctagaacatc caaatggtct tcagtttcac tatatgacat gatgttgagg | 120 |
| actgtagccc tgctgtcagg tagagctttc gttggcttac cactatgtag agatgaggga | 180 |
| tggttgcagg caagtatagg ttatacagtc caatgcgttt caataagaga tcagcttttt | 240 |
| acttggagcc ccgtattgag accaattatc gggccattct tgccctcagt tagaagtgtg | 300 |
| aggagacact tgagatttgc tgcagaaatt atggctcctc ttatcagtca ggctttacaa | 360 |
| gatgaaaagc aacacagggc tgatacactt ttagcagatc agaccgaagg tcgtggcacg | 420 |
| tttatttctt ggttactgag acacctgcca gaagaattac gtactcctga gcaagtagga | 480 |
| ctggaccaga tgcttgtatc ttttgccgca attcacacta caacaatggc tctaaccaaa | 540 |
| gtcgtgtggg aattagttaa gagaccagaa tacatcgaac ccttgagaac tgaaatgcaa | 600 |
| gatgtcttcg ggcccgatgc ggtttcacca gacatttgca ttaataaaga ggccctatcc | 660 |
| aggttgcata aattggattc ttttattagg gaggttcaaa gatggtgtcc ttccactttt | 720 |
| gttactccta gccgtagagt gatgaagtcc atgacgctga gcaacggaat taaactgcaa | 780 |
| cgtggtacga gtattgcttt tcctgctcat gctatacata tgtcagaaga aacacctact | 840 |
| ttttcacctg acttttcttc tgacttcgaa aatccttccc ctagaatttt tgatgggttc | 900 |
| cgttatttaa acttgaggtc aatcaaggga caaggaagcc agcatcaagc ggctactacc | 960 |
| ggtcctgatt acttaatttt taaccatggt aaacatgctt gccctggtag attttttgct | 1020 |
| atttcagaaa taaaaatgat cttgatagag ttactagcta agtacgattt caggttggaa | 1080 |
| gacggaaaac cagggcctga actaatgaga gttggtactg agacaagatt ggatacaaag | 1140 |
| gcaggtttgg agatgagacg tagataa | 1167 |

SEQ ID NO: 144

| | |
|---|---|
| MKYTTCQMNI FPSLWSMKTS FRWPRTSKWS SVSLYDMMLR TVALLSGRAF VGLPLCRDEG | 60 |
| WLQASIGYTV QCVSIRDQLF TWSPVLRPII GPFLPSVRSV RRHLRFAAEI MAPLISQALQ | 120 |
| DEKQHRADTL LADQTEGRGT FISWLLRHLP EELRTPEQVG LDQMLVSFAA IHTTIMALTK | 180 |
| VVWELVKRPE YIEPLRTEMQ DVFGPDAVSP DICINKEALS RLHKLDSFIR EVQRWCPSTF | 240 |
| VTPSRRVMKS MTLSNGIKLQ RGTSIAFPAH AIHMSEETPT FSPDFSSDFE NPSPRIFDGF | 300 |
| RYLNLRSIKG QGSQHQAATT GPDYLIFNHG KHACPGRFFA ISEIKMILIE LLAKYDFRLE | 360 |
| DGKPGPELMR VGTETRLDTK AGLEMRRR | 388 |

SEQ ID NO: 145

| | |
|---|---|
| atggctaacc attccagttc atactaccat gaattttaca aagatcattc tcacacagtc | 60 |
| ttgacgctaa tgtctgaaaa acctgtgatt ttgccatcct taatacttgg aacctgtgcc | 120 |
| gtgttgttat gtatacaatg gctgaaaccg cagcctttaa tcatggtcaa cggtagaaag | 180 |
| tttggagaat tgtctaatgt aagagccaag cgtgatttta ccttcggtgc gagacaattg | 240 |
| ttagaaaagg gtctgaaaat gtcacctgac aaacccttca gaataatggg tgatgttggt | 300 |
| gagttgcata tcttgccacc aaaatatgct tatgaagtac gtaacaatga aaaactatct | 360 |
| ttcaccatgg cagccttcaa atggttttac gcacacttgc ctggtttcga aggtttcaga | 420 |
| gaaggtacca atgaatcaca tattatgaag ttggtcgcaa ggcatcaact aacacatcaa | 480 |
| ctgacactag ttacaggtgc agtctccgaa gagtgtgctc ttgttttaaa ggatgtttac | 540 |
| accgatagtc ccgagtggca tgacatcacc gccaaggacg caaatatgaa actgatggct | 600 |
| aggataacta gtagagtttt ccttggtaaa gaaatgtgca gaaaccctca atggttacgt | 660 |
| atcacatcta catatgccgt gattgcattc agagcagtag aggaactaag attatggcca | 720 |
| tcatggttga gaccagttgt tcaatggttt atgccacact gtacgcagtc tagagccctt | 780 |
| gtgcaagaag caagggactt aattaatccg ttgttggaaa ggagaaggga agaaaaagcg | 840 |
| gaggctgaaa ggacgggtga gaaggtaact tacaatgacg ctgtggaatg gttggacgat | 900 |
| ttggccaggg aaaagggagt gggttatgat cctgcctgcg ctcaattaag cctaagtgtt | 960 |
| gccgccttac attcaactac tgacttcttc actcaagtta tgtttgatat tgctcaaaat | 1020 |
| cctgagttga tagaaccgtt aagagaagag atcatagcag tcttgggcaa acagggatgg | 1080 |
| tccaagaaca gtttgtataa tcttaaactg atggattctg tgttgaaaga gtcacaacgt | 1140 |
| ctaaagccaa tagccatcgc tagcatgagg agatttacta cacacaacgt taaattgtcc | 1200 |
| gatggcgtca tattacccaa gaacaagtta acgttagtta gcgcacatca gcactgggat | 1260 |
| ccagagtact acaaagaccc attaaaattt gatggctata gattctttaa catgagacgt | 1320 |
| gagcccggca aagaatcaaa agcacaacta gtctctgcga ccccagacca tatggggttc | 1380 |
| ggttatggcc tacatgcctg tcctggcagg ttttttgctt ctgaagaaat caaaatcgca | 1440 |
| ctgtcacaca tcttactgaa gtatgatttt aagcccgttg aaggtagttc catggagcca | 1500 |
| agaaagtatg gtttgaacat gaacgcaaac cctactgcga aactgagcgt tcgtagaaga | 1560 |
| aaggaagaga ttgctattta a | 1581 |

SEQ ID NO: 146

| | |
|---|---|
| MANHSSSYYH EFYKDHSHTV LTLMSEKPVI LPSLILGTCA VLLCIQWLKP QPLIMVNGRK | 60 |
| FGELSNVRAK RDFTFGARQL LEKGLKMSPD KPFRIMGDVG ELHILPPKYA YEVRNNEKLS | 120 |
| FTMAAFKWFY AHLPGFEGFR EGTNESHIMK LVARHQLTHQ LTLVTGAVSE ECALVLKDVY | 180 |
| TDSPEWHDIT AKDANMKLMA RITSRVFLGK EMCRNPQWLR ITSTYAVIAF RAVEELRLWP | 240 |
| SWLRPVVQWF MPHCTQSRAL VQEARDLINP LLERRREEKA EAERTGEKVT YNDAVEWLDD | 300 |
| LAREKGVGYD PACAQLSLSV AALHSTTDFF TQVMFDIAQN PELIEPLREE IIAVLGKQGW | 360 |
| SKNSLYNLKL MDSVLKESQR LKPIAIASMR RFTTHNVKLS DGVILPKNKL TLVSAHQHWD | 420 |
| PEYYKDPLKF DGYRFFNMRR EPGKESKAQL VSATPDHMGF GYGLHACPGR FFASEEIKIA | 480 |
| LSHILLKYDF KPVEGSSMEP RKYGLNMNAN PTAKLSVRRR KEEIAI | 526 |

SEQ ID NO: 147

| | |
|---|---|
| atgtctaagg ttgtgtacgt ttctcatgat ggtacccgta gggaactaga tgttgctgat | 60 |
| ggcgtttcat taatgcaagc tgcagtttca aatggtatat atgatattgt cggcgattgt | 120 |
| ggtggaagtg cttcctgtgc gacgtgtcat gtctatgtaa acgaagcttt tacggataag | 180 |
| gtcccagctg ctaatgaaag agagataggt atgttggaat gtgttacagc ggagttaaaa | 240 |
| ccaaattcca gactatgctg ccaaattatt atgacccctg aactggatgg aatagtagtt | 300 |
| gatgttcctg acagacaatg gtaa | 324 |

SEQ ID NO: 148

| | |
|---|---|
| MSKVVYVSHD GTRRELDVAD GVSLMQAAVS NGIYDIVGDC GGSASCATCH VYVNEAFTDK | 60 |
| VPAANEREIG MLECVTAELK PNSRLCCQII MTPELDGIVV DVPDRQW | 107 |

SEQ ID NO: 149

| | |
|---|---|
| atgaacgcga acgataatgt cgtaattgtg ggtactggat tagccggcgt agaggtggct | 60 |
| tttggattaa gagcgtctgg atgggaaggt aatatcaggc tggttgggga tgccactgtt | 120 |
| ataccacatc acttgcctcc tctgtctaaa gcttatttgg ccggcaaagc tactgctgag | 180 |
| tccttatact taaggactcc ggatgcctat gcagcccaaa atatccaatt gttgggtgga | 240 |
| acgcaggtga cggccatcaa cagagatcgt caacaagtta ttttgagtga tggaagagca | 300 |
| ttggactacg atagactggt tttggctaca ggtggtagac ctaggcctct accagttgca | 360 |
| agtggtgccg ttggaaaagc caacaatttc agatatttaa ggactctaga agacgctgaa | 420 |
| tgcattagga ggcagttgat agcagacaat agactagttg tgattggggg cgggtacatc | 480 |
| ggcttagaag tagcagcgac agcaataaaa gcgaacatgc acgttacact attagatacg | 540 |
| gccgcaagag tactagagag ggtaaccgca cctccagtgt ctgcatttta tgaacatcta | 600 |
| catagagagg cgggtgttga catcaggact ggaactcagg tgtgcggttt cgaaatgtcc | 660 |
| acagatcaac aaaaggtcac cgcggttttg tgtgaagatg ggacaagatt gccagcagat | 720 |
| ttggtcattg ccgggatcgg tctaatccct aattgtgaac tggcctctgc cgcaggcctg | 780 |
| caagttgata atggtatcgt tattaacgaa catatgcaaa ctagcgaccc gttaataatg | 840 |
| gcggtcggag attgtgctcg ttttcatagc cagctatacg accgttgggt tagaatagag | 900 |
| tcagttccta acgcattgga acaagccagg aaaatcgctg ccatactttg tggtaaagta | 960 |
| ccaagagatg aggcagctcc atggttctgg agcgatcaat acgaaatcgg tttgaaaatg | 1020 |
| gttggattga gcgagggcta cgacagaatt atcgttagag gtagcttggc ccaaccagat | 1080 |
| ttttcagttt tttacttaca aggtgataga gttctagcag tcgatactgt caacagaccg | 1140 |
| gtagagttca accaaagcaa gcaaatcatt actgatagac tacctgtcga accaaacctt | 1200 |
| cttggagatg aatccgtgcc attgaaagaa atcattgccg ccgcgaaggc cgaactttcc | 1260 |
| agtgcttga | 1269 |

SEQ ID NO: 150

| | |
|---|---|
| MNANDNVVIV GTGLAGVEVA FGLRASGWEG NIRLVGDATV IPHHLPPLSK AYLAGKATAE | 60 |
| SLYLRTPDAY AAQNIQLLGG TQVTAINRDR QQVILSDGRA LDYDRLVLAT GGRPRPLPVA | 120 |
| SGAVGKANNF RYLRTLEDAE CIRRQLIADN RLVVIGGGYI GLEVAATAIK ANMHVILLDT | 180 |
| AARVLERVTA PPVSAFYEHL HREAGVDIRT GTQVCGFEMS TDQQKVTAVL CEDGTRLPAD | 240 |
| LVIAGIGLIP NCELASAAGL QVDNGIVINE HMQTSDPLIM AVGDCARFHS QLYDRWVRIE | 300 |
| SVPNALEQAR KIAAILCGKV PRDEAAPWFW SDQYEIGLKM VGLSEGYDRI IVRGSLAQPD | 360 |
| FSVFYLQGDR VLAVDTVNRP VEFNQSKQII TDRLPVEPNL LGDESVPLKE IIAAAKAELS | 420 |
| SA | 422 |

SEQ ID NO: 151

| | |
|---|---|
| atggctaaca ctggtattcc aaccgttgat gtttctttgt tcttgtccga aggtgaaaac | 60 |
| gaagctaaga agcaagctat tcaaaccatt accgaagcct gttcttctta cggttttttc | 120 |
| caaatcgtta accacggtat cccaatcgaa tttttgaaag aagccttgca gttgtccaag | 180 |
| acattttttc attatccaga cgaaatcaag ttgcaatact ctccaaaacc aggtgctcca | 240 |
| ttattggctg gttttaacaa gcaaaagacc aactgcgttg acaagaacga atacgttttg | 300 |
| gtttttccac caggctctaa gtttaacatc tatccacaag aaccaccaca attcaaagaa | 360 |
| accttggaag agatgttctt gaagttgtct gatgtctcct tggtcatcga atccattttg | 420 |
| aatgtttgtt tgggtttgcc accaggtttc ttgaagcaat tcaacaatga tagatcctgg | 480 |
| gacttcatga ccaacttgta ttattaccca gctgctgatg ttggtgaaaa cggtttgatt | 540 |
| catcatgaag atgctaactg catcaccttg gttattcaag atgatgctgg tggtttacaa | 600 |
| gtccaaaaag attctgaatg gattccagtt actccagttg aaggtgctat cgttgttaac | 660 |
| gttggtgata tcatccaagt cttgtccaac aagaagttca agtctgctac tcacagagtt | 720 |
| gttagacaga agggtaaaga aagatactcc ttcgctttct tcagatcatt gcatggtgat | 780 |
| aagtgggttg aaccattgcc agaattcacc aaagaaattg gtgaaaagcc aaagtacaag | 840 |
| ggcttcgaat tcaatgaata cttggccttg agattgaaga acaagactca tccaccatct | 900 |
| agagttgaag atgagatttc catcaagcac tacgagatca actga | 945 |

SEQ ID NO: 152

| | |
|---|---|
| MANTGIPTVD VSLFLSEGEN EAKKQAIQTI TEACSSYGFF QIVNHGIPIE FLKEALQLSK | 60 |
| TFFHYPDEIK LQYSPKPGAP LLAGFNKQKT NCVDKNEYVL VFPPGSKFNI YPQEPPQFKE | 120 |
| TLEEMFLKLS DVSLVIESIL NVCLGLPPGF LKQFNNDRSW DFMTNLYYYP AADVGENGLI | 180 |
| HHEDANCITL VIQDDAGGLQ VQKDSEWIPV TPVEGAIVVN VGDIIQVLSN KKEKSATHRV | 240 |
| VRQKGKERYS FAFFRSLHGD KWVEPLPEFT KEIGEKPKYK GFEFNEYLAL RLKNKTHPPS | 300 |
| RVEDEISIKH YEIN | 314 |

SEQ ID NO: 153

| | |
|---|---|
| atgtcctcta gatctacccc aagaaaagaa cctatttgcg cttctggtat tttcccatcc | 60 |
| gttgataatc aagctttgga agttccacca ggtattcaaa agttgaccta ccaatctttg | 120 |
| acctcctcta cctctttcag attattgcaa gttttgtccg atggtggtag agatattttg | 180 |
| agatgcaaga tgttcgatgc tgatttggct gctagagaac caccaagata tattgctttg | 240 |
| tcttacacct ggcacgaaga atctttgcca aaaactttta gaccagtctt gatcaacgac | 300 |
| aagtacttga acgtttcttt gaacttgtgg aacttcttgc aaaactacag agaaacctcc | 360 |
| ggtgaaagaa ttatctggat tgatcaaatc tgcatcaatc aagaagataa ggacgaatgc | 420 |
| gttcaacaaa ttggtcaaat gtgcaagatc taccaatgcg cttctatgga tttgttctgg | 480 |
| attggtgaac caggtgaaaa tgctgaagct gttttggatt tgttgtcctc cttgaacaga | 540 |
| ttggaaacct acttgttgga atccggttct tctagaccag gtatttctgc tttgttgaac | 600 |
| ccaattttca tgagagctgt tggtttgcca gaacatgata atccaatttg gggttccttg | 660 |
| atgcaattca tttctagaac tgctttccaa agagcctgga tcattcaaga agttgctgtt | 720 |
| tctagaacca ccgctatttt ttgtggtttg ttgatgttgc cattcgatgt tgttggtaga | 780 |
| gctgctactt ttttggttga atcctcttgg attaaggttt tccacgaaat gtacaacgtt | 840 |
| tctggtgctg ctggttttat tactggtatg atgaactgca gagtcagaca tcaagaaggt | 900 |
| gaacatcaat ctttggactt gttgttggct tctaccagaa gattcaaagc tacaaagcca | 960 |
| gttgataaga tcttcgcctt gattaacttg gctgaatccg gtagaaaaga agctttgcca | 1020 |
| ccagctttaa gaccagatta cagaaaatct atcgtcggtg ttttcagaga tgtcaccttg | 1080 |
| tacttgatta gacaaggttc cttggatgtt ttgtccggtg ttgaagatgt taagttcaga | 1140 |
| caaatccacg aattgccatc ttggattcca gattactctg ttcatcaagt tgcctccatt | 1200 |
| ttgtgtatgc caccaagacc aggttggttg acattatatg ctgctgctgt tggtagagat | 1260 |
| gtttccgttc aaaattctcc agctgatcca aacattttga ccttgtctgc ttacaaggtt | 1320 |
| gacaccattt ctaagattgg ttccattgcc gaagaatcca tctacttgac tttggaaaaa | 1380 |
| tgggcctcta tggttgattt ttctgctgct tatccaactg ttaacggtaa cacttgtcca | 1440 |
| atgattgatg ctttttggag aaccttgatt ggtaacattg gtttgggtac ttctcaatac | 1500 |
| ccagtttctg aagattgggc tcattctttt gctgttttcg ctttacaagc cagagaagaa | 1560 |
| ttgcaacatc acttctcttc atcctctgat actgaaagag ctgctttgga atctccaata | 1620 |
| gttactccag gtatcgactc cattttgaga ttggttaagg atcattacca cggtaacaac | 1680 |
| gattctgatc aagatggtgg tttgtacgaa tctaccatgc atcatgtttc ttggtacaga | 1740 |
| agattattct tgaccaacgg tggttacttt ggtttggctc atccatcttc tcaaccaggt | 1800 |
| gatgaagttg ttttgttgtc tggtggtaga gttccattcg ttgttagaag agtttctgcc | 1860 |
| gaaagaagag aatgctattc tatcgttggt gaaacctacg ttcatggtat tatggacggt | 1920 |
| gaattattgg atgctactga cggtaaatgg gaagacttgc aattcaagtg a | 1971 |

SEQ ID NO: 154

| | |
|---|---|
| MSSRSTPRKE PICASGIFPS VDNQALEVPP GIQKLTYQSL TSSTSFRLLQ VLSDGGRDIL | 60 |
| RCKMFDADLA AREPPRYIAL SYTWHEESLP KTFRPVLIND KYLNVSLNLW NFLQNYRETS | 120 |
| GERIIWIDQI CINQEDKDEC VQQIGQMCKI YQCASMDLFW IGEPGENAEA VLDLLSSLNR | 180 |
| LETYLLESGS SRPGISALLN PIFMRAVGLP EHDNPIWGSL MQFISRTAFQ RAWIIQEVAV | 240 |
| SRTTAIFCGL LMLPFDVVGR AATFLVESSW IKVFHEMYNV SGAAGFITGM MNCRVRHQEG | 300 |
| EHQSLDLLLA STRRFKATKP VDKIFALINL AESGRKEALP PALRPDYRKS IVGVERDVIL | 360 |
| YLIRQGSLDV LSGVEDVKFR QIHELPSWIP DYSVHQVASI LCMPPRPGWL TLYAAAVGRD | 420 |
| VSVQNSPADP NILTLSAYKV DTISKIGSIA EESIYLTLEK WASMVDFSAA YPTVNGNTCP | 480 |
| MIDAFWRTLI GNIGLGTSQY PVSEDWAHSF AVFALQAREE LQHHFSSSSD TERAALESPI | 540 |
| VTPGIDSILR LVKDHYHGNN DSDQDGGLYE STMHHVSWYR RLFLTNGGYF GLAHPSSQPG | 600 |
| DEVVLLSGGR VPFVVRRVSA ERRECYSIVG ETYVHGIMDG ELLDATDGKW EDLQFK | 656 |

SEQ ID NO: 155

| | |
|---|---|
| atggcagata gtttggccgt tagacatgca gctgctttaa aattaatcga agatttaacg | 60 |
| tcttcattga atgatgtaga acctttagga gatattagca gagcgcaagc ggattatgat | 120 |
| gctgccgaag aaagacatag aagggaacaa gacccggcta ggaaaagggc attgtgcagg | 180 |
| gaactagtaa ggtacggcga tagactggag gaaattgaga agcaacataa ggaagctgaa | 240 |
| gccaagtgta aagaacaact agatctattt gacacaagac tagcgaaaga gggttaccgt | 300 |
| aaactagcca caagggcgtc ctctataact ggtactaacc aaactacaca ccaatcatcc | 360 |
| aatacttctg gaaacttaat tcagacacct gatccgaact acggacaact ttcaggcttt | 420 |
| acaaacgaca gagctactca tgaaaacacc gaatcaccag gaacgttgcc tcaatcttcc | 480 |
| actattagga acacaataga accgaggcta actcccagta gaacaaattc agctgctcca | 540 |
| tccagaggta tttccacgga tatcgatcaa caagtcagaa tagaacctac agtccaaaca | 600 |
| gacaggtcta atcaaaggcg tgacaacccc tctagttcta gaccagccaa gagacagaga | 660 |
| caaggcgcat ctagtgaaac agttacagaa aggacaataa ccttcgatga ggtatatcag | 720 |
| gggggaaagg ctaggtggaa atacaggatc accaaggtgc atggattata ttacgtattc | 780 |
| ggctgtgaga agcatgaaaa acattttggt aaagaaaatc cattacaatc agcaatgtcc | 840 |
| catttaaagg gtaaaggcca ttcttgtaag agacctaatg ctactcaagc tttacgtagt | 900 |
| ctgggaatac aagttttacc atgtacggat agagatcttg agctaaacaa caaagccgtt | 960 |
| gacaggtacc tggcagaaca agaagaaaag aataaaagaa gaaaggcgtc tgtaaaagat | 1020 |
| ttaagtcaag cacctcaaac tggtgaaatt tatatggcat ggttcggaga tgatgataaa | 1080 |
| ggctactggc tacacgcctt tctggtcata ccattctttc ctaggccagg cgacggcatg | 1140 |
| gacgttcaaa ctgtaacagg ctccaattta aacgatgata ttccagcctg ctataggttc | 1200 |
| gatgaaacta ctgatgggta taactggact gaggactaca aagaatatgg caaatacgca | 1260 |
| aataatagag tttacccaat tatgtgtctt gtgggtcaga tccctcataa agtcgattgg | 1320 |
| ttacctgtct gccatttcag aaaacttaat cttgaagatg aagacctaga ggacaaagat | 1380 |
| gtcattaaag cgtttatgcg taaaaatacc accgggaata caggttacgg caacgaggtt | 1440 |
| gatgatgaat cagaagatct atatggtgat tcctttgcag gtgatgatga tgtgcctaca | 1500 |
| agctctgaaa ggagacagtc tccgatagga aatagttctg aaaatatcaa tactgatcaa | 1560 |
| agcatacaag caggcgccac cgctgaaaat caagaaagcg gtaccttagg tccaaactta | 1620 |
| gcgactcaag aggttaaaga tgaattagcg acgatcggga gaggcgatgg tgctactagt | 1680 |
| gctgctgatc aaccggcaag agctaggcaa atgtctgtcc gtcgtcgttg gccttctgct | 1740 |
| agaaagggac caccggatat ggaaaccgtt agcgattcag agtaa | 1785 |

SEQ ID NO: 156

| | |
|---|---|
| MADSLAVRHA AALKLIEDLT SSLNDVEPLG DISRAQADYD AAEERHRREQ DPARKRALCR | 60 |
| ELVRYGDRLE EIEKQHKEAE AKCKEQLDLF DTRLAKEGYR KLATRASSIT GTNQTTHQSS | 120 |
| NTSGNLIQTP DPNYGQLSGF TNDRATHENT ESPGTLPQSS TIRNTIEPRL TPSRTNSAAP | 180 |
| SRGISTDIDQ QVRIEPTVQT DRSNQRRDNP SSSRPAKRQR QGASSETVTE RTITFDEVYQ | 240 |
| GGKARWKYRI TKVHGLYYVF GCEKHEKHFG KENPLQSAMS HLKGKGHSCK RPNATQALRS | 300 |
| LGIQVLPCTD RDLELNNKAV DRYLAEQEEK NKRRKASVKD LSQAPQTGEI YMAWFGDDDK | 360 |
| GYWLHAFLVI PFFPRPGDGM DVQTVTGSNL NDDIPACYRF DETTDGYNWT EDYKEYGKYA | 420 |
| NNRVYPIMCL VGQIPHKVDW LPVCHFRKLN LEDEDLEDKD VIKAFMRKNT TGNTGYGNEV | 480 |
| DDESEDLYGD SFAGDDDVPT SSERRQSPIG NSSENINTDQ SIQAGATAEN QESGTLGPNL | 540 |
| ATQEVKDELA TIGRGDGATS AADQPARARQ MSVRRRWPSA RKGPPDMETV SDSE | 594 |

SEQ ID NO: 157

| | |
|---|---|
| atggctcaat tggatacctt ggatttggtt gttttggccg ttttgttggt tggttctgtt | 60 |
| gcttatttta ccaagggtac ttattgggct gttgctaaag atccatatgc ttctactggt | 120 |
| ccagctatga atggtgctgc taaagctggt aaaaccagaa acattatcga aaagatggaa | 180 |
| gaaaccggta agaactgcgt tattttctac ggttctcaaa ctggtactgc tgaagattat | 240 |
| gcttccagat tggctaaaga aggttctcaa agattcggtt tgaaaaccat ggttgccgat | 300 |
| ttggaagaat acgactacga aaacttggac caattcccag aagataaggt tgcttttttc | 360 |
| gttttggcta cttacggtga aggtgaacct actgataatg ctgttgaatt ctaccaattc | 420 |
| ttcaccggtg atgatgttgc ttttgaatct gcttctgctg acgaaaaacc attgtctaag | 480 |
| ttgaagtacg ttgctttcgg tttgggtaac aacacttacg aacattacaa cgccatggtt | 540 |
| agacaagttg atgctgcttt tcaaaagttg ggtccacaaa gaattggttc tgctggtgaa | 600 |
| ggtgatgatg gtgctggtac tatggaagaa gattttttgg cttggaaaga acctatgtgg | 660 |
| gctgctttgt ctgaatctat ggacttggaa gaaagagaag ctgtttacga accagttttc | 720 |
| tgtgttaccg aaaacgaatc tttgtcccca gaagatgaaa ctgtttattt gggtgaacct | 780 |
| acccaatctc acttgcaagg tactccaaaa ggtccatatt ctgctcataa tccattcatt | 840 |
| gctccaatcg ctgaatccag agaattattc actgttaagg acagaaactg cttgcacatg | 900 |
| gaaatttcta ttgccggttc taacttgtct taccaaaccg gtgatcatat tgctgtttgg | 960 |
| ccaactaatg ctggtgctga agttgataga ttcttgcaag tttttggttt ggaaggtaag | 1020 |
| agagactccg ttattaacat caagggtatt gatgttaccg ccaaggttcc aattccaact | 1080 |
| ccaactactt atgatgctgc cgtcagatat tacatggaag tttgtgctcc agtctccaga | 1140 |
| caatttgttg ctactttggc tgcttttgct ccagatgaag aatctaaagc tgaaatcgtt | 1200 |
| agattgggtt cccacaagga ttactttcac gaaaaggtta ccaatcaatg cttcaatatg | 1260 |
| gctcaagcct tgcaatctat tacctctaaa ccattttctg ccgtcccatt ctctttgttg | 1320 |
| attgaaggta ttaccaagtt gcaacctaga tattactcca tctcctcctc ttcattggtt | 1380 |
| caaaaggata agatttccat caccgccgtt gttgaatctg ttagattgcc aggtgcttct | 1440 |
| catatggtta agggtgttac taccaattac ttgttggcct tgaagcaaaa gcaaaacggt | 1500 |
| gatccatctc cagatccaca tggtttgact tattctatta ctggtccaag aaacaagtac | 1560 |
| gatggtatcc atgttccagt tcatgttaga cactctaact tcaagttgcc atctgatcca | 1620 |
| tctagaccaa ttatcatggt tggtccaggt actggtgttg ctccttttag aggttttatt | 1680 |
| caagaaagag ctgctttggc tgctaagggt gaaaaagttg gtccaactgt tttgttcttc | 1740 |
| ggttgcagaa aatccgacga agatttcttg tacaaggacg aatggaaaac ctaccaagat | 1800 |
| caattgggtg acaacttgaa gattattacc gccttttcta gagaaggtcc acaaaaggtt | 1860 |
| tacgtccaac atagattgag agaacactcc gaattggttt ccgatttgtt gaaacaaaag | 1920 |
| gccacctttt acgtttgtgg tgatgctgct aatatggcca gagaagttaa tttggttttg | 1980 |
| ggtcaaatta tcgctgccca aagaggtttg ccagctgaaa aaggtgaaga aatggtcaaa | 2040 |
| cacatgagaa gaagaggtag ataccaagaa gatgtctggt cttaa | 2085 |

SEQ ID NO: 158

| | |
|---|---|
| MAQLDTLDLV VLAVLLVGSV AYFTKGTYWA VAKDPYASTG PAMNGAAKAG KTRNIIEKME | 60 |
| ETGKNCVIFY GSQTGTAEDY ASRLAKEGSQ RFGLKTMVAD LEEYDYENLD QFPEDKVAFF | 120 |
| VLATYGEGEP TDNAVEFYQF FTGDDVAFES ASADEKPLSK LKYVAFGLGN NTYEHYNAMV | 180 |
| RQVDAAFQKL GPQRIGSAGE GDDGAGTMEE DFLAWKEPMW AALSESMDLE EREAVYEPVF | 240 |
| CVTENESLSP EDETVYLGEP TQSHLQGTPK GPYSAHNPFI APIAESRELF TVKDRNCLHM | 300 |
| EISIAGSNLS YQTGDHIAVW PTNAGAEVDR FLQVFGLEGK RDSVINIKGI DVTAKVPIPT | 360 |
| PTTYDAAVRY YMEVCAPVSR QFVATLAAFA PDEESKAEIV RLGSHKDYFH EKVTNQCFNM | 420 |
| AQALQSITSK PFSAVPFSLL IEGITKLQPR YYSISSSSLV QKDKISITAV VESVRLPGAS | 480 |
| HMVKGVTTNY LLALKQKQNG DPSPDPHGLT YSITGPRNKY DGIHVPVHVR HSNFKLPSDP | 540 |
| SRPIIMVGPG TGVAPFRGFI QERAALAAKG EKVGPTVLFF GCRKSDEDFL YKDEWKTYQD | 600 |
| QLGDNLKIIT AFSREGPQKV YVQHRLREHS ELVSDLLKQK ATFYVCGDAA NMAREVNLVL | 660 |
| GQIIAAQRGL PAEKGEEMVK HMRRRGRYQE DVWS | 694 |

SEQ ID NO: 159

| | |
|---|---|
| atgtccgcca agaaagaatt caccatgcaa gatgttgctg aacacaatac ctcttccgat | 60 |
| atctacatgg ttgttcacga taaggtttac gattgcacca agttcttgga tgaacatcca | 120 |
| ggtggtgaag aagttatgtt ggacgttgct ggtcaagatg ctactgaagc ttttgaagat | 180 |
| gttggtcatt ctgatgaagc cagagaagtt ttggatggtt tgttggttgg tgaattgaaa | 240 |
| agattgccag gtgatgaagg tccaaagaga caaattgcta actccaatca aggttctggt | 300 |
| aaagctgatc cagctggttc ttctttgaat acttatgcta tcgttgttgc cgttggtttc | 360 |
| attgcttatg ttgcttacaa ctacttgcaa aagcaacaag aagctcaagg tcaagcttct | 420 |
| gcttaa | 426 |

SEQ ID NO: 160

| | |
|---|---|
| MSAKKEFTMQ DVAEHNTSSD IYMVVHDKVY DCTKFLDEHP GGEEVMLDVA GQDATEAFED | 60 |
| VGHSDEAREV LDGLLVGELK RLPGDEGPKR QIANSNQGSG KADPAGSSLN TYAIVVAVGF | 120 |
| IAYVAYNYLQ KQQEAQGQAS A | 141 |

SEQ ID NO: 161

| | |
|---|---|
| atggacttga agaatcaaac cttcaccttc catttcgata tggctaagga tactggtatt | 60 |
| ccaaccgttg atttgtctgt tttctctgct caaaacgaaa ccgaagctaa gaagaaggct | 120 |
| ttcgaaacta tctaccaagc ctgttcttct tacggtttct tccaaatcgt taaccatggt | 180 |
| gttccaatcg aattcttgga agaagctttg gaattgtcca gaacattctt ccattaccca | 240 |
| gatgacatca agttgaagta ctcttctaaa ccaggtgctc cattattggc tggttttaac | 300 |
| aagcaaaaga agaactgcgt tgacaagaac gaatacgttt tggtttttcc accaggctct | 360 |
| aactacaata tctatccaca agaaccacca caattcaaag aattattgga agaaatgttc | 420 |
| aagaagttgt ccaaggtctg cttgttgttg gaatctatcg ttaacgaatc tttgggtttg | 480 |
| ccaccagatt ttttgaagca gtacaacaac gatagatcct gggattttat gaccaccttg | 540 |
| tactactttt ctgctactga agaaggtgaa aacggtttga ctcatcatga agatggtaac | 600 |
| tgcattacct tggttttcca agatgatacc ggtggtttac aagttagaaa agatggtgaa | 660 |
| tggatcccag ttgttccagt tgaaggtgct atcgttgtta acattggtga tgttatccag | 720 |
| gtcttgtcca acaagaaatt caagtctgct acccacagag ttgttagaca aaagggtaaa | 780 |
| gaaagattct cctacgcctt cttccataac ttgcatggtg ataagtgggt tgaaccattg | 840 |
| ccacaattca ctgaagaaat tggtgaaaag ccaaagtaca agggtttcca attcaaggat | 900 |
| taccaagcct tgagattgaa gaacaaaact catccaccat ctagagttga ggacgaaatt | 960 |
| agaattaccc actacgagat cagctaa | 987 |

SEQ ID NO: 162

| | |
|---|---|
| MDLKNQTFTF HFDMAKDTGI PTVDLSVFSA QNETEAKKKA FETIYQACSS YGFFQIVNHG | 60 |
| VPIEFLEEAL ELSRTFFHYP DDIKLKYSSK PGAPLLAGFN KQKKNCVDKN EYVLVFPPGS | 120 |
| NYNIYPQEPP QFKELLEEMF KKLSKVCLLL ESIVNESLGL PPDFLKQYNN DRSWDFMTTL | 180 |
| YYFSATEEGE NGLTHHEDGN CITLVFQDDT GGLQVRKDGE WIPVVPVEGA IVVNIGDVIQ | 240 |
| VLSNKKFKSA THRVVRQKGK ERFSYAFFHN LHGDKWVEPL PQFTEEIGEK PKYKGFQFKD | 300 |
| YQALRLKNKT HPPSRVEDEI RITHYEIS | 328 |

SEQ ID NO: 163

| | |
|---|---|
| atggcctcca tcacccattt cttacaagat tttcaagcta ctccattcgc tactgctttt | 60 |
| gctgttggtg gtgtttcttt gttgatattc ttcttcttca tccgtggttt ccactctact | 120 |
| aagaaaaacg aatattacaa gttgccacca gttccagttg ttccaggttt gccagttgtt | 180 |
| ggtaatttgt tgcaattgaa agaaaagaag ccatacaaga ctttcttgag atgggctgaa | 240 |
| attcatggtc caatctactc tattagaact ggtgcttcta ccatggttgt tgttaactct | 300 |
| actcatgttg ccaaagaagc tatggttacc agattctctt caatctctac cagaaagttg | 360 |
| tccaaggctt tggaattatt gacctccaac aaatctatgg ttgccacctc tgattacaac | 420 |
| gaatttcaca agatggtcaa gaagtacatc ttggccgaat tattgggtgc taatgctcaa | 480 |
| aagagacaca gaattcatag agacaccttg atcgaaaacg tcttgaacaa attgcatgcc | 540 |
| cataccaaga attctccatt gcaagctgtt aacttcagaa agatcttcga atctgaatta | 600 |
| ttcggtttgg ctatgaagca agccttgggt tatgatgttg attccttgtt cgttgaagaa | 660 |
| ttgggtacta ccttgtccag agaagaaatc tacaacgttt tggtcagtga catgttgaag | 720 |
| ggtgctattg aagttgattg gagagacttt ttcccatact tgaaatggat cccaaacaag | 780 |
| tccttcgaaa tgaagattca aagattggcc tctagaagac aagccgttat gaactctatt | 840 |
| gtcaaagaac aaaagaagtc cattgcctct ggtaagggtg aaaactgtta cttgaattac | 900 |
| ttgttgtccg aagctaagac tttgaccgaa aagcaaattt ccattttggc ctgggaaacc | 960 |
| attattgaaa ctgctgatac aactgttgtt accactgaat gggctatgta cgaattggct | 1020 |
| aaaaacccaa agcaacaaga cagattatac aacgaaatcc aaaacgtctg cggtactgat | 1080 |
| aagattaccg aagaacattt gtccaagttg ccttacttgt ctgctgtttt tcacgaaacc | 1140 |
| ttgagaaagt attctccatc tccattggtt ccattgagat acgctcatga agatactcaa | 1200 |
| ttgggtggtt attatgttcc agccggtact gaaattgctg ttaatatcta cggttgcaac | 1260 |
| atggacaaga atcaatggga aactccagaa gaatggaagc cagaaagatt tttggacgaa | 1320 |
| aagtacgatc caatggacat gtacaagact atgtcttttg gttccggtaa aagagtttgc | 1380 |
| gctggttctt tacaagctag tttgattgct tgtacctcca tcggtagatt ggttcaagaa | 1440 |
| tttgaatgga gattgaaaga cggtgaagtt gaaaacgttg ataccttggg tttgactacc | 1500 |
| cataagttgt atccaatgca agctatcttg caacctagaa actga | 1545 |

SEQ ID NO: 164

| | |
|---|---|
| MASITHFLQD FQATPFATAF AVGGVSLLIF FFFIRGFHST KKNEYYKLPP VPVVPGLPVV | 60 |
| GNLLQLKEKK PYKTFLRWAE IHGPIYSIRT GASTMVVVNS THVAKEAMVT RFSSISTRKL | 120 |
| SKALELLTSN KSMVATSDYN EFHKMVKKYI LAELLGANAQ KRHRIHRDTL IENVLNKLHA | 180 |
| HTKNSPLQAV NFRKIFESEL FGLAMKQALG YDVDSLFVEE LGTTLSREEI YNVLVSDMLK | 240 |
| GAIEVDWRDF FPYLKWIPNK SFEMKIQRLA SRRQAVMNSI VKEQKKSIAS GKGENCYLNY | 300 |
| LLSEAKTLTE KQISILAWET IIETADTTVV TTEWAMYELA KNPKQQDRLY NEIQNVCGTD | 360 |
| KITEEHLSKL PYLSAVFHET LRKYSPSPLV PLRYAHEDTQ LGGYYVPAGT EIAVNIYGCN | 420 |
| MDKNQWETPE EWKPERFLDE KYDPMDMYKT MSEGSGKRVC AGSLQASLIA CTSIGRLVQE | 480 |
| FEWRLKDGEV ENVDTLGLTT HKLYPMQAIL QPRN | 514 |

SEQ ID NO: 165

| | |
|---|---|
| atgcaatcag attcagtcaa agtctctcca tttgatttgg tttccgctgc tatgaatggc | 60 |
| aaggcaatgg aaaagttgaa cgctagtgaa tctgaagatc caacaacatt gcctgcacta | 120 |
| aagatgctag ttgaaaatag agaattgttg acactgttca caacttcctt cgcagttctt | 180 |
| attgggtgtc ttgtatttct aatgtggaga cgttcatcct ctaaaaagct ggtacaagat | 240 |
| ccagttccac aagttatcgt tgtaaagaag aaagagaagg agtcagaggt tgatgacggg | 300 |
| aaaaagaaag tttctatttt ctacggcaca caaacaggaa ctgccgaagg ttttgctaaa | 360 |
| gcattagtcg aggaagcaaa agtgagatat gaaaagacct ctttcaaggt tatcgatcta | 420 |
| gatgactacg ctgcagatga tgatgaatat gaggaaaaac tgaaaaagga atccttagcc | 480 |
| ttcttcttct tggccacata cggtgatggt gaacctactg ataatgctgc taacttctac | 540 |
| aagtggttca cagaaggcga cgataaaggt gaatggctga aaaagttaca atacggagta | 600 |
| tttggtttag gtaacagaca atatgaacat ttcaacaaga tcgctattgt agttgatgat | 660 |
| aaacttactg aaatgggagc caaaagatta gtaccagtag gattagggga tgatgatcag | 720 |
| tgtatagaag atgacttcac cgcctggaag gaattggtat ggccagaatt ggatcaactt | 780 |
| ttaagggacg aagatgatac ttctgtgact accccataca ctgcagccgt attggagtac | 840 |
| agagtggttt accatgataa accagcagac tcatatgctg aagatcaaac ccatacaaac | 900 |
| ggtcatgttg ttcatgatgc acagcatcct tcaagatcta atgtggcttt caaaaaggaa | 960 |
| ctacacacct ctcaatcaga taggtcttgt actcacttag aattcgatat ttctcacaca | 1020 |
| ggactgtctt acgaaactgg cgatcacgtt ggcgtttatt ccgagaactt gtccgaagtt | 1080 |
| gtcgatgaag cactaaaact gttagggtta tcaccagaca catacttctc agtccatgct | 1140 |
| gataaggagg atgggacacc tatcggtggt gcttcactac caccaccttt tcctccttgc | 1200 |
| acattgagag acgctctaac cagatacgca gatgtcttat cctcacctaa aaaggtagct | 1260 |
| ttgctggcat tggctgctca tgctagtgat cctagtgaag ccgataggtt aaagttcctg | 1320 |
| gcttcaccag ccggaaaaga tgaatatgca caatggatcg tcgccaacca acgttctttg | 1380 |
| ctagaagtga tgcaaagttt tccatctgcc aagcctccat taggtgtgtt cttcgcagca | 1440 |
| gtagctccac gtttacaacc aagatactac tctatcagtt catctcctaa gatgtctcct | 1500 |
| aacagaatac atgttacatg tgctttggtg tacgagacta ctccagcagg cagaattcac | 1560 |
| agaggattgt gttcaacctg gatgaaaaat gctgtccctt taacagagtc acctgattgc | 1620 |
| tctcaagcat ccattttcgt tagaacatca aatttcagac ttccagtgga tccaaaagtt | 1680 |
| ccagtcatta tgataggacc aggcactggt cttgccccat tcaggggctt tcttcaagag | 1740 |
| agattggcct tgaaggaatc tggtacagaa ttgggttctt ctatcttttt ctttggttgc | 1800 |
| cgtaatagaa aagttgactt tatctacgag gacgagctta acaattttgt tgagacagga | 1860 |
| gcattgtcag aattgatcgt cgcattttca agagaaggga ctgccaaaga gtacgttcag | 1920 |
| cacaagatga gtcaaaaagc ctccgatata tggaaacttc taagtgaagg tgcctatctt | 1980 |
| tatgtctgtg gcgatgcaaa gggcatggcc aaggatgtcc atagaactct gcatacaatt | 2040 |
| gttcaggaac aagggagtct ggattcttcc aaggctgaat tgtacgtcaa aaacttacag | 2100 |
| atgtctggaa gatacttaag agatgtttgg taa | 2133 |

SEQ ID NO: 166

| | |
|---|---|
| MQSDSVKVSP FDLVSAAMNG KAMEKLNASE SEDPTTLPAL KMLVENRELL TLFTTSFAVL | 60 |
| IGCLVFLMWR RSSSKKLVQD PVPQVIVVKK KEKESEVDDG KKKVSIFYGT QTGTAEGFAK | 120 |
| ALVEEAKVRY EKTSFKVIDL DDYAADDDEY EEKLKKESLA FFFLATYGDG EPTDNAANFY | 180 |
| KWFTEGDDKG EWLKKLQYGV FGLGNRQYEH FNKIAIVVDD KLTEMGAKRL VPVGLGDDDQ | 240 |
| CIEDDFTAWK ELVWPELDQL LRDEDDTSVT TPYTAAVLEY RVVYHDKPAD SYAEDQTHTN | 300 |
| GHVVHDAQHP SRSNVAFKKE LHTSQSDRSC THLEFDISHT GLSYETGDHV GVYSENLSEV | 360 |
| VDEALKLLGL SPDTYFSVHA DKEDGTPIGG ASLPPPFPPC TLRDALTRYA DVLSSPKKVA | 420 |
| LLALAAHASD PSEADRLKFL ASPAGKDEYA QWIVANQRSL LEVMQSFPSA KPPLGVFFAA | 480 |
| VAPRLQPRYY SISSSPKMSP NRIHVTCALV YETTPAGRIH RGLCSTWMKN AVPLTESPDC | 540 |
| SQASIFVRTS NFRLPVDPKV PVIMIGPGTG LAPFRGFLQE RLALKESGTE LGSSIFFFGC | 600 |
| RNRKVDFIYE DELNNFVETG ALSELIVAFS REGTAKEYVQ HKMSQKASDI WKLLSEGAYL | 660 |
| YVCGDAKGMA KDVHRTLHTI VQEQGSLDSS KAELYVKNLQ MSGRYLRDVW | 710 |

SEQ ID NO: 167

| | |
|---|---|
| atgtcctcca actccgattt ggtcagaaga ttggaatctg ttttgggtgt ttctttcggt | 60 |
| ggttctgtta ctgattccgt tgttgttatt gctaccacct ctattgcttt ggttatcggt | 120 |
| gttttggttt tgttgtggag aagatcctct gacagatcta gagaagttaa gcaattggct | 180 |
| gttccaaagc cagttactat cgttgaagaa gaagatgaat tcgaagttgc ttctggtaag | 240 |
| accagagttt ctattttcta cggtactcaa actggtactg ctgaaggttt tgctaaggct | 300 |
| ttggctgaag aaatcaaagc cagatacgaa aaagctgccg ttaaggttat tgatttggat | 360 |
| gattacacag ccgaagatga caaatacggt gaaaagttga agaaagaaac tatggccttc | 420 |
| ttcatgttgg ctacttatgg tgatggtgaa cctactgata atgctgctag attttacaag | 480 |
| tggttcaccg aaggtactga tagaggtgtt tggttggaac atttgagata cggtgtattc | 540 |
| ggtttgggta acagacaata cgaacacttc aacaagattg ccaaggttgt tgatgatttg | 600 |
| ttggttgaac aaggtgccaa gagattggtt actgttggtt tgggtgatga tgatcaatgc | 660 |
| atcgaagatg atttctccgc ttggaaagaa gccttgtggc cagaattgga tcaattattg | 720 |
| caagatgata ccaacaccgt ttctactcca tacactgctg ttattccaga atacagagtt | 780 |
| gttatccacg atccatctgt tacctcttat gaagatccat actctaacat ggctaacggt | 840 |
| aatgcctctt acgatattca tcatccatgt agagctaacg ttgccgtcca aaaagaattg | 900 |
| cataagccag aatctgacag aagttgcatc catttggaat tcgatatttt cgctactggt | 960 |
| ttgacttacg aaaccggtga tcatgttggt gtttacgctg ataattgtga tgatactgta | 1020 |
| gaagaagccg ctaagttgtt gggtcaacca ttggatttgt tgttctccat tcataccgat | 1080 |
| aacaacgacg gtacttcttt gggttcttct ttgccaccac catttccagg tccatgtact | 1140 |
| ttgagaactg ctttggctag atatgccgat ttgttgaatc caccaaaaaa ggctgctttg | 1200 |
| attgctttag ctgctcatgc tgatgaacca tctgaagctg aaagattgaa gttcttgtca | 1260 |
| tctccacaag gtaaggacga atattctaaa tgggttgtcg gttcccaaag atccttggtt | 1320 |
| gaagttatgg ctgaatttcc atctgctaaa ccaccattgg gtgtattttt tgctgctgtt | 1380 |
| gttcctagat tgcaacctag atattactcc atctcttcca gtccaagatt tgctccacat | 1440 |
| agagttcatg ttacttgcgc tttggtttat ggtccaactc caactggtag aattcacaga | 1500 |
| ggtgtatgtt cattctggat gaagaatgtt gtcccattgg aaaagtctca aaactgttct | 1560 |
| tgggccccaa ttttcatcag acaatctaat ttcaagttgc cagccgatca ttctgttcca | 1620 |
| atagttatgg ttggtccagg tactggttta gctcctttta gaggtttctt acaagaaaga | 1680 |
| ttggccttga aagaagaagg tgctcaagtt ggtcctgctt tgttgttttt tggttgcaga | 1740 |
| aacagacaaa tggacttcat ctacgaagtc gaattgaaca actttgtcga acaaggtgct | 1800 |
| ttgtccgaat tgatcgttgc tttttcaaga gaaggtccat ccaaagaata cgtccaacat | 1860 |
| aagatggttg aaaaggcagc ttacatgtgg aacttgattt ctcaaggtgg ttacttctac | 1920 |
| gtttgtggtg atgctaaagg tatggctaga gatgttcata gaacattgca taccatcgtc | 1980 |
| caacaagaag aaaaggttga ttctaccaag gccgaatcca tcgttaagaa attgcaaatg | 2040 |
| gacggtagat acttgagaga tgtttggtga | 2070 |

SEQ ID NO: 168

| | |
|---|---|
| MSSNSDLVRR LESVLGVSFG GSVTDSVVVI ATTSIALVIG VLVLLWRRSS DRSREVKQLA | 60 |
| VPKPVTIVEE EDEFEVASGK TRVSIFYGTQ TGTAEGFAKA LAEEIKARYE KAAVKVIDLD | 120 |
| DYTAEDDKYG EKLKKETMAF FMLATYGDGE PTDNAARFYK WFTEGTDRGV WLEHLRYGVF | 180 |
| GLGNRQYEHF NKIAKVVDDL LVEQGAKRLV TVGLGDDDQC IEDDFSAWKE ALWPELDQLL | 240 |
| QDDTNTVSTP YTAVIPEYRV VIHDPSVTSY EDPYSNMANG NASYDIHHPC RANVAVQKEL | 300 |
| HKPESDRSCI HLEFDIFATG LTYETGDHVG VYADNCDDTV EEAAKLLGQP LDLLFSIHTD | 360 |
| NNDGTSLGSS LPPPFPGPCT LRTALARYAD LLNPPKKAAL IALAAHADEP SEAERLKFLS | 420 |
| SPQGKDEYSK WVVGSQRSLV EVMAEFPSAK PPLGVFFAAV VPRLQPRYYS ISSSPRFAPH | 480 |
| RVHVTCALVY GPTPTGRIHR GVCSFWMKNV VPLEKSQNCS WAPIFIRQSN FKLPADHSVP | 540 |
| IVMVGPGTGL APFRGFLQER LALKEEGAQV GPALLFFGCR NRQMDFIYEV ELNNFVEQGA | 600 |
| LSELIVAFSR EGPSKEYVQH KMVEKAAYMW NLISQGGYFY VCGDAKGMAR DVHRTLHTIV | 660 |
| QQEEKVDSTK AESIVKKLQM DGRYLRDVW | 689 |

SEQ ID NO: 169

| | |
|---|---|
| atggctacct tgttggaaca ttttcaagct atgccattcg ctattccaat tgctttggct | 60 |
| gctttgtctt ggttgttttt gttctacatc aaggtttctt tcttctccaa caaatccgct | 120 |
| caagctaaat tgccaccagt tccagttgtt ccaggtttgc cagttattgg taatttgttg | 180 |
| caattgaaag aaaagaagcc ataccaaacc ttcactagat gggctgaaga atatggtcca | 240 |
| atctactcta ttagaactgg tgcttctact atggttgtct tgaacactac tcaagttgcc | 300 |
| aaagaagcta tggttaccag atacttgtct atctctacca gaaagttgtc caacgccttg | 360 |
| aaaattttga ccgctgataa gtgcatggtt gccatttctg attacaacga tttccacaag | 420 |
| atgatcaaga gatatatctt gtctaacgtt ttgggtccat ctgcccaaaa aagacataga | 480 |
| tctaacagag ataccttgag agccaacgtt tgttctagat tgcattccca agttaagaac | 540 |
| tctccaagag aagctgtcaa ctttagaaga gttttcgaat gggaattatt cggtatcgct | 600 |
| ttgaaacaag ccttcggtaa ggatattgaa aagccaatct acgtcgaaga attgggtact | 660 |
| actttgtcca gagatgaaat cttcaaggtt ttggtcttgg acattatgga aggtgccatt | 720 |
| gaagttgatt ggagagattt tttcccatac ttgcgttgga ttccaaacac cagaatggaa | 780 |
| actaagatcc aaagattata ctttagaaga aaggccgtta tgaccgcctt gattaacgaa | 840 |
| caaaagaaaa gaattgcctc cggtgaagaa atcaactgct acatcgattt cttgttgaaa | 900 |
| gaaggtaaga ccttgaccat ggaccaaatc tctatgttgt tgtgggaaac cgttattgaa | 960 |
| actgctgata ccacaatggt tactactgaa tgggctatgt acgaagttgc taaggattct | 1020 |
| aaaagacaag acagattata ccaagaaatc caaaaggtct gcggttctga aatggttaca | 1080 |
| gaagaatact tgtcccaatt gccatacttg aatgctgttt tccacgaaac tttgagaaaa | 1140 |
| cattctccag ctgctttggt tccattgaga tatgctcatg aagatactca attgggtggt | 1200 |
| tattacattc cagccggtac tgaaattgcc attaacatct acggttgcaa catggacaaa | 1260 |
| caccaatggg aatctccaga agaatggaag ccagaaagat ttttggatcc taagtttgac | 1320 |
| ccaatggact tgtacaaaac tatggctttt ggtgctggta aaagagtttg cgctggttct | 1380 |
| ttacaagcta tgttgattgc ttgtccaacc atcggtagat tggttcaaga atttgaatgg | 1440 |
| aagttgagag atggtgaaga agaaaacgtt gatactgttg gtttgaccac ccataagaga | 1500 |
| tatccaatgc atgctatttt gaagccaaga tcttaa | 1536 |

SEQ ID NO: 170

| | |
|---|---|
| MATLLEHFQA MPFAIPIALA ALSWLFLFYI KVSFFSNKSA QAKLPPVPVV PGLPVIGNLL | 60 |
| QLKEKKPYQT FTRWAEEYGP IYSIRTGAST MVVLNITQVA KEAMVTRYLS ISTRKLSNAL | 120 |
| KILTADKCMV AISDYNDFHK MIKRYILSNV LGPSAQKRHR SNRDTLRANV CSRLHSQVKN | 180 |
| SPREAVNFRR VFEWELFGIA LKQAFGKDIE KPIYVEELGT TLSRDEIFKV LVLDIMEGAI | 240 |
| EVDWRDFFPY LRWIPNTRME TKIQRLYFRR KAVMTALINE QKKRIASGEE INCYIDFLLK | 300 |
| EGKILTMDQI SMLLWETVIE TADTTMVITE WAMYEVAKDS KRQDRLYQEI QKVCGSEMVT | 360 |
| EEYLSQLPYL NAVFHETLRK HSPAALVPLR YAHEDTQLGG YYIPAGTEIA INIYGCNMDK | 420 |
| HQWESPEEWK PERFLDPKFD PMDLYKTMAF GAGKRVCAGS LQAMLIACPT IGRLVQEFEW | 480 |
| KLRDGEEENV DTVGLITHKR YPMHAILKPR S | 511 |

SEQ ID NO: 171

| | |
|---|---|
| atggatgctg tgacgggttt gttaactgtc ccagcaaccg ctataactat tggtggaact | 60 |
| gctgtagcat tggcggtagc gctaatcttt tggtacctga aatcctacac atcagctaga | 120 |
| agatcccaat caaatcatct tccaagagtg cctgaagtcc caggtgttcc attgttagga | 180 |
| aatctgttac aattgaagga gaaaaagcca tacatgactt ttacgagatg ggcagcgaca | 240 |
| tatggaccta tctatagtat caaaactggg gctacaagta tggttgtggt atcatctaat | 300 |
| gagatagcca aggaggcatt ggtgaccaga ttccaatcca tatctacaag gaacttatct | 360 |
| aaagccctga aagtacttac agcagataag acaatggtcg caatgtcaga ttatgatgat | 420 |
| tatcataaaa cagttaagag acacatactg accgccgtct tgggtcctaa tgcacagaaa | 480 |
| aagcatagaa ttcacagaga tatcatgatg gataacatat ctactcaact tcatgaattc | 540 |
| gtgaaaaaca acccagaaca ggaagaggta gaccttagaa aaatctttca atctgagtta | 600 |
| ttcggcttag ctatgagaca agccttagga aaggatgttg aaagtttgta cgttgaagac | 660 |
| ctgaaaatca ctatgaatag agacgaaatc tttcaagtcc ttgttgttga tccaatgatg | 720 |
| ggagcaatcg atgttgattg gagagacttc tttccatacc taaagtgggt cccaaacaaa | 780 |
| aagttcgaaa atactattca acaaatgtac atcagaagag aagctgttat gaaatcttta | 840 |
| atcaaagagc acaaaaagag aatagcgtca ggcgaaaagc taaatagtta tatcgattac | 900 |
| cttttatctg aagctcaaac tttaaccgat cagcaactat tgatgtcctt gtgggaacca | 960 |
| atcattgaat cttcagatac aacaatggtc acaacagaat gggcaatgta cgaattagct | 1020 |
| aaaaacccta aattgcaaga taggttgtac agagacatta agtccgtctg tggatctgaa | 1080 |
| aagataaccg aagagcatct atcacagctg ccttacatta cagctatttt ccacgaaaca | 1140 |
| ctgagaagac actcaccagt tcctatcatt cctctaagac atgtacatga agataccgtt | 1200 |
| ctaggcggct accatgttcc tgctggcaca gaacttgccg ttaacatcta cggttgcaac | 1260 |
| atggacaaaa acgtttggga aaatccagag gaatggaacc cagaaagatt catgaaagag | 1320 |
| aatgagacaa ttgattttca aaagacgatg gccttcggtg gtggtaagag agtttgtgct | 1380 |
| ggttccttgc aagccctttt aactgcatct attgggattg ggagaatggt tcaagagttc | 1440 |
| gaatggaaac tgaaggatat gactcaagag gaagtgaaca cgataggcct aactacacaa | 1500 |
| atgttaagac cattgagagc tattatcaaa cctaggatct aa | 1542 |

SEQ ID NO: 172

| | |
|---|---|
| MDAVTGLLTV PATAITIGGT AVALAVALIF WYLKSYTSAR RSQSNHLPRV PEVPGVPLLG | 60 |
| NLLQLKEKKP YMTFTRWAAT YGPIYSIKTG ATSMVVVSSN EIAKEALVTR FQSISTRNLS | 120 |
| KALKVLTADK TMVAMSDYDD YHKTVKRHIL TAVLGPNAQK KHRIHRDIMM DNISTQLHEF | 180 |
| VKNNPEQEEV DLRKIFQSEL FGLAMRQALG KDVESLYVED LKITMNRDEI FQVLVVDPMM | 240 |
| GAIDVDWRDF FPYLKWVPNK KFENTIQQMY IRREAVMKSL IKEHKKRIAS GEKLNSYIDY | 300 |
| LLSEAQTLTD QQLLMSLWEP IIESSDTTMV TTEWAMYELA KNPKLQDRLY RDIKSVCGSE | 360 |
| KITEEHLSQL PYITAIFHET LRRHSPVPII PLRHVHEDTV LGGYHVPAGT ELAVNIYGCN | 420 |
| MDKNVWENPE EWNPERFMKE NETIDFQKTM AFGGGKRVCA GSLQALLTAS IGIGRMVQEF | 480 |
| EWKLKDMTQE EVNTIGLITQ MLRPLRAIIK PRI | 513 |

SEQ ID NO: 173

| | |
|---|---|
| atggccgaat tggatacctt ggatatcgtt gttttgggtg ttatcttctt gggtactgtt | 60 |
| gcttacttca ccaaaggtaa attgtggggt gttactaagg atccatacgc taatggtttt | 120 |
| gctgctggtg gtgcttctaa accaggtaga actagaaata tcgttgaagc catggaagaa | 180 |
| tctggtaaga actgtgttgt tttctacggt tctcaaactg gtactgctga agattatgct | 240 |
| tccagattgg ctaaagaagg taagagtaga ttcggtttga acaccatgat tgccgatttg | 300 |
| gaagattacg atttcgataa cttggatacc gtcccatctg ataacatcgt tatgtttgtt | 360 |
| ttggctacct acggtgaagg tgaacctact gataatgctg ttgacttcta cgaattcatt | 420 |
| accggtgaag atgcttcttt caacgaaggt aatgatccac cattgggtaa cttgaattac | 480 |
| gttgcttttg gtttgggtaa caacacctac gaacattaca actccatggt tagaaacgtc | 540 |
| aacaaggctt tggaaaaatt gggtgctcat agaattggtg aagctggtga aggtgatgat | 600 |
| ggtgctggta ctatggaaga agattttttg gcttggaaag acccaatgtg ggaagccttg | 660 |
| gctaaaaaga tgggtttgga agaaagagaa gctgtctacg aacctatttt cgccattaac | 720 |
| gaaagagatg atttgacccc tgaagccaat gaagtttatt tgggtgaacc taacaagttg | 780 |
| cacttggaag gtactgctaa aggtccattc aattctcaca acccatatat tgctccaatc | 840 |
| gccgaatctt acgaattatt ctctgctaag gatagaaact gcttgcacat ggaaattgac | 900 |
| atctctggtt ctaatttgaa gtacgaaacc ggtgatcata ttgccatttg gccaactaat | 960 |
| ccaggtgaag aagttaacaa gttcttggac atcttggact tgtccggtaa acaacattct | 1020 |
| gttgttactg ttaaggcctt ggaacctaca gctaaagttc cttttccaaa tccaactacc | 1080 |
| tacgatgcca ttttgagata ccatttggaa atttgcgctc cagtctctag acaattcgtt | 1140 |
| tctactttgg ctgcttttgc tccaaacgat gatattaagg ctgaaatgaa cagattgggt | 1200 |
| tccgataagg attacttcca cgaaaaaact ggtccacact actacaacat tgctagattt | 1260 |
| ttggcctctg tctctaaagg tgaaaagtgg actaagattc cattctccgc tttcattgaa | 1320 |
| ggtttgacta agttgcaacc tagatattac tccatctcct cctcatcttt ggttcaacct | 1380 |
| aagaagatct ctattaccgc cgttgttgaa tcccaacaaa ttccaggtag agatgatcct | 1440 |
| tttagaggtg ttgctaccaa ttacttgttc gccttgaaac aaaagcaaaa cggtgatcca | 1500 |
| aatcctgctc catttggtca atcttatgaa ttgactggtc caagaaacaa gtacgatggt | 1560 |
| attcatgttc cagttcacgt tagacactct aactttaagt tgccatctga tccaggtaag | 1620 |
| ccaattatca tgattggtcc aggtactggt gttgctccat tcagaggttt tgttcaagaa | 1680 |
| agagctaagc aagctagaga tggtgttgaa gttggtaaaa ccttgttgtt cttcggttgt | 1740 |
| agaaagtcca ctgaagattt catgtaccaa aaagaatggc aagaatacaa agaagcctta | 1800 |
| ggtgacaagt tcgaaatgat tactgccttc tcaagagaag gttctaagaa ggtttacgtc | 1860 |
| caacacagat tgaaagaaag atccaaagaa gtctccgatt tgttgtctca aaaggcctac | 1920 |
| ttttacgttt gtggtgatgc tgctcatatg gccagagaag ttaatactgt tttggcccaa | 1980 |
| attatcgctg aaggtagagg tgtatctgaa gctaagggtg aagaaatcgt taagaacatg | 2040 |
| agatccgcca atcaatacca agtttgctct gattttgtta ccttgcactg taaagaaacc | 2100 |
| acctacgcta attccgaatt gcaagaagat gtttggtcct aa | 2142 |

SEQ ID NO: 174

| | |
|---|---|
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQVCS DFVTLHCKET TYANSELQED VWS | 713 |

SEQ ID NO: 175

| | |
|---|---|
| atggcttcag aaaaagaaat taggagagag agattcttga acgttttccc taaattagta | 60 |
| gaggaattga acgcatcgct tttggcttac ggtatgccta aggaagcatg tgactggtat | 120 |
| gcccactcat tgaactacaa cactccaggc ggtaagctaa atagaggttt gtccgttgtg | 180 |
| gacacgtatg ctattctctc caacaagacc gttgaacaat tggggcaaga agaatacgaa | 240 |
| aaggttgcca ttctaggttg gtgcattgag ttgttgcagg cttacttctt ggtcgccgat | 300 |
| gatatgatgg acaagtccat taccagaaga ggccaaccat gttggtacaa ggttcctgaa | 360 |
| gttggggaaa ttgccatcaa tgacgcattc atgttagagg ctgctatcta caagcttttg | 420 |
| aaatctcact tcagaaacga aaaatactac atagatatca ccgaattgtt ccatgaggtc | 480 |
| accttccaaa ccgaattggg ccaattgatg gacttaatca ctgcacctga agacaaagtc | 540 |
| gacttgagta agttctccct aaagaagcac tccttcatag ttactttcaa gactgcttac | 600 |
| tattctttct acttgcctgt cgcattggcc atgtacgttg ccggtatcac ggatgaaaag | 660 |
| gatttgaaac aagccagaga tgtcttgatt ccattgggtg aatacttcca aattcaagat | 720 |
| gactacttag actgcttcgg taccccagaa cagatcggta agatcggtac agatatccaa | 780 |
| gataacaaat gttcttgggt aatcaacaag gcattggaac ttgcttccgc agaacaaaga | 840 |
| aagactttag acgaaaatta cggtaagaag gactcagtcg cagaagccaa atgcaaaaag | 900 |
| attttcaatg acttgaaaat tgaacagcta taccacgaat atgaagagtc tattgccaag | 960 |
| gatttgaagg ccaaaatttc tcaggtcgat gagtctcgtg gcttcaaagc tgatgtctta | 1020 |
| actgcgttct tgaacaaagt ttacaagaga agcaaatag | 1059 |

SEQ ID NO: 176

| | |
|---|---|
| MASEKEIRRE RFLNVFPKLV EELNASLLAY GMPKEACDWY AHSLNYNTPG GKLNRGLSVV | 60 |
| DTYAILSNKT VEQLGQEEYE KVAILGWCIE LLQAYFLVAD DMMDKSITRR GQPCWYKVPE | 120 |
| VGEIAINDAF MLEAAIYKLL KSHFRNEKYY IDITELFHEV TFQTELGQLM DLITAPEDKV | 180 |
| DLSKFSLKKH SFIVTFKTAY YSFYLPVALA MYVAGITDEK DLKQARDVLI PLGEYFQIQD | 240 |
| DYLDCFGTPE QIGKIGTDIQ DNKCSWVINK ALELASAEQR KTLDENYGKK DSVAEAKCKK | 300 |
| IFNDLKIEQL YHEYEESIAK DLKAKISQVD ESRGFKADVL TAFLNKVYKR SK | 352 |

SEQ ID NO: 177

| | |
|---|---|
| atggtcgcac aaactttcaa cctggatacc tacttatccc aaagacaaca acaagttgaa | 60 |
| gaggccctaa gtgctgctct tgtgccagct tatcctgaga gaatatacga agctatgaga | 120 |
| tactccctcc tggcaggtgg caaaagatta agacctatct tatgtttagc tgcttgcgaa | 180 |
| ttggcaggtg gttctgttga acaagccatg ccaactgcgt gtgcacttga aatgatccat | 240 |
| acaatgtcac taattcatga tgacctgcca gccatggata acgatgattt cagaagagga | 300 |
| aagccaacta atcacaaggt gttcggggaa gatatagcca tcttagcggg tgatgcgctt | 360 |
| ttagcttacg cttttgaaca tattgcttct caaacaagag gagtaccacc tcaattggtg | 420 |
| ctacaagtta ttgctagaat cggacacgcc gttgctgcaa caggcctcgt tggaggccaa | 480 |
| gtcgtagacc ttgaatctga aggtaaagct atttccttag aaacattgga gtatattcac | 540 |
| tcacataaga ctggagcctt gctggaagca tcagttgtct caggcggtat tctcgcaggg | 600 |
| gcagatgaag agcttttggc cagattgtct cattacgcta gagatatagg cttggctttt | 660 |
| caaatcgtcg atgatatcct ggatgttact gctacatctg aacagttggg gaaaaccgct | 720 |
| ggtaaagacc aggcagccgc aaaggcaact tatccaagtc tattgggttt agaagcctct | 780 |
| agacagaaag cggaagagtt gattcaatct gctaaggaag ccttaagacc ttacggttca | 840 |
| caagcagagc cactcctagc gctggcagac ttcatcacac gtcgtcagca ttaa | 894 |

SEQ ID NO: 178

| | |
|---|---|
| MVAQTFNLDT YLSQRQQQVE EALSAALVPA YPERIYEAMR YSLLAGGKRL RPILCLAACE | 60 |
| LAGGSVEQAM PTACALEMIH TMSLIHDDLP AMDNDDFRRG KPTNHKVFGE DIAILAGDAL | 120 |
| LAYAFEHIAS QTRGVPPQLV LQVIARIGHA VAATGLVGGQ VVDLESEGKA ISLETLEYIH | 180 |
| SHKTGALLEA SVVSGGILAG ADEELLARLS HYARDIGLAF QIVDDILDVT ATSEQLGKTA | 240 |
| GKDQAAAKAT YPSLLGLEAS RQKAEELIQS AKEALRPYGS QAEPLLALAD FITRRQH | 297 |

SEQ ID NO: 179

| | |
|---|---|
| atggcacagc acacatcaga atccgcagct gtcgcaaagg gcagcagttt gacccctata | 60 |
| gtgagaactg acgctgagtc aaggagaaca agatggccaa ccgatgacga tgacgccgaa | 120 |
| cctttagtgg atgagatcag ggcaatgctt acttccatgt ctgatggtga catttccgtg | 180 |
| agcgcatacg atacagcctg ggtcggattg gttccaagat tagacggcgg tgaaggtcct | 240 |
| caatttccag cagctgtgag atggataaga aataaccagt tgcctgacgg aagttggggc | 300 |
| gatgccgcat tattctctgc ctatgacagg cttatcaata cccttgcctg cgttgtaact | 360 |
| ttgacaaggt ggtccctaga accagagatg agaggtagag gactatcttt tttgggtagg | 420 |
| aacatgtgga aattagcaac tgaagatgaa gagtcaatgc ctattggctt cgaattagca | 480 |
| tttccatctt tgatagagct tgctaagagc ctaggtgtcc atgacttccc ttatgatcac | 540 |
| caggccctac aaggaatcta ctcttcaaga gagatcaaaa tgaagaggat tccaaaagaa | 600 |
| gtgatgcata ccgttccaac atcaatattg cacagtttgg agggtatgcc tggcctagat | 660 |
| tgggctaaac tacttaaact acagagcagc gacggaagtt ttttgttctc accagctgcc | 720 |
| actgcatatg ctttaatgaa taccggagat gacaggtgtt ttagctacat cgatagaaca | 780 |
| gtaaagaaat tcaacggcgg cgtccctaat gtttatccag tggatctatt tgaacatatt | 840 |
| tgggccgttg atagacttga aagattagga atctccaggt acttccaaaa ggagatcgaa | 900 |
| caatgcatgg attatgtaaa caggcattgg actgaggacg gtatttgttg ggcaaggaac | 960 |
| tctgatgtca aagaggtgga cgacacagct atggccttta gacttcttag gttgcacggc | 1020 |
| tacagcgtca gtcctgatgt gtttaaaaac ttcgaaaagg acggtgaatt tttcgcattt | 1080 |
| gtcggacagt ctaatcaagc tgttaccggt atgtacaact taaacagagc aagccagata | 1140 |
| tccttcccag gcgaggatgt gcttcataga gctggtgcct tctcatatga gttcttgagg | 1200 |
| agaaaagaag cagagggagc tttgagggac aagtggatca tttctaaaga tctacctggt | 1260 |
| gaagttgtgt atactttgga ttttccatgg tacggcaact tacctagagt cgaggccaga | 1320 |
| gactacctag agcaatacgg aggtggtgat gacgtttgga ttggcaagac attgtatagg | 1380 |
| atgccacttg taaacaatga tgtatatttg gaattggcaa gaatggattt caaccactgc | 1440 |
| caggctttgc atcagttaga gtggcaagga ctaaaaagat ggtatactga aaataggttg | 1500 |
| atggactttg gtgtcgccca agaagatgcc cttagagctt attttcttgc agccgcatct | 1560 |
| gtttacgagc cttgtagagc tgccgagagg cttgcatggg ctagagccgc aatactagct | 1620 |
| aacgccgtga gcacccactt aagaaatagc ccatcattca gagaaaggtt agagcattct | 1680 |
| cttaggtgta gacctagtga agagacagat ggctcctggt ttaactcctc aagtggctct | 1740 |
| gatgcagttt tagtaaaggc tgtcttaaga cttactgatt cattagccag ggaagcacag | 1800 |
| ccaatccatg gaggtgaccc agaagatatt atacacaagt tgttaagatc tgcttgggcc | 1860 |
| gagtgggtta gggaaaaggc agacgctgcc gatagcgtgt gcaatggtag ttctgcagta | 1920 |
| gaacaagagg gatcaagaat ggtccatgat aaacagacct gtctattatt ggctagaatg | 1980 |
| atcgaaattt ctgccggtag ggcagctggt gaagcagcca gtgaggacgg cgatagaaga | 2040 |
| ataattcaat taacaggctc catctgcgac agtcttaagc aaaaaatgct agtttcacag | 2100 |
| gaccctgaaa aaaatgaaga gatgatgtct cacgtggatg acgaattgaa gttgaggatt | 2160 |
| agagagttcg ttcaatattt gcttagacta ggtgaaaaaa agactggatc tagcgaaacc | 2220 |
| aggcaaacat ttttaagtat agtgaaatca tgttactatg ctgctcattg cccacctcat | 2280 |
| gtcgttgata gacacattag tagagtgatt ttcgagccag taagtgccgc aaagtaa | 2337 |

SEQ ID NO: 180

| | |
|---|---|
| MAQHTSESAA VAKGSSLTPI VRTDAESRRT RWPTDDDDAE PLVDEIRAML TSMSDGDISV | 60 |
| SAYDTAWVGL VPRLDGGEGP QFPAAVRWIR NNQLPDGSWG DAALFSAYDR LINTLACVVT | 120 |
| LTRWSLEPEM RGRGLSFLGR NMWKLATEDE ESMPIGFELA FPSLIELAKS LGVHDFPYDH | 180 |
| QALQGIYSSR EIKMKRIPKE VMHTVPTSIL HSLEGMPGLD WAKLLKLQSS DGSFLFSPAA | 240 |
| TAYALMNTGD DRCFSYIDRT VKKFNGGVPN VYPVDLFEHI WAVDRLERLG ISRYFQKEIE | 300 |
| QCMDYVNRHW TEDGICWARN SDVKEVDDTA MAFRLLRLHG YSVSPDVFKN FEKDGEFFAF | 360 |
| VGQSNQAVTG MYNLNRASQI SFPGEDVLHR AGAFSYEFLR RKEAEGALRD KWIISKDLPG | 420 |
| EVVYTLDFPW YGNLPRVEAR DYLEQYGGGD DVWIGKTLYR MPLVNNDVYL ELARMDFNHC | 480 |
| QALHQLEWQG LKRWYTENRL MDFGVAQEDA LRAYFLAAAS VYEPCRAAER LAWARAAILA | 540 |
| NAVSTHLRNS PSFRERLEHS LRCRPSEETD GSWFNSSSGS DAVLVKAVLR LTDSLAREAQ | 600 |
| PIHGGDPEDI IHKLLRSAWA EWVREKADAA DSVCNGSSAV EQEGSRMVHD KQTCLLLARM | 660 |
| IEISAGRAAG EAASEDGDRR IIQLTGSICD SLKQKMLVSQ DPEKNEEMMS HVDDELKLRI | 720 |
| REFVQYLLRL GEKKTGSSET RQTFLSIVKS CYYAAHCPPH VVDRHISRVI FEPVSAAK | 778 |

SEQ ID NO: 181

| | |
|---|---|
| atgtctatta atttgagatc ttccggttgt agctccccaa taagcgcaac tttggaaagg | 60 |
| ggtctagact ctgaagttca aacaagagca aacaatgtat cttttgagca gaccaaagag | 120 |
| aagatcagga aaatgcttga gaaggtcgag ttgagcgtga gtgcctatga cactagttgg | 180 |
| gtagctatgg tcccatcacc atccagtcaa aacgcacctc ttttcccaca gtgcgtcaaa | 240 |
| tggctacttg ataatcaaca tgaggacggc tcttggggat tggataacca cgaccatcag | 300 |
| agcttaaaga aagatgtgtt gtcatccaca ttagcctcta tcctagctct taagaaatgg | 360 |
| ggaataggcg aaagacagat caataagggt ctacagttca ttgaattaaa ctctgcacta | 420 |
| gttaccgatg aaactataca aaaacctaca ggtttcgaca tcatttttcc aggaatgatt | 480 |
| aagtacgcca gggaccttaa tttgaccata cctcttggct cagaagtagt cgacgatatg | 540 |
| atcaggaaaa gagatctaga cttaaagtgt gatagcgaga aattcagcaa aggtagagag | 600 |
| gcttatcttg cctatgttct tgaaggaact aggaacttga aggactggga cttaattgtg | 660 |
| aaatatcaga gaaagaacgg tagtctattt gatagtccag ctacaaccgc cgcagctttc | 720 |
| actcaatttg gcaatgacgg ttgcttgagg tacttatgtt cacttttaca gaaattcgag | 780 |
| gccgcagtgc ctagtgtata tccatttgat caatacgcta gattaagcat aatcgtcact | 840 |
| ttagaatcat tgggaattga cagagatttc aagactgaga taaaaagcat attggatgag | 900 |
| acctataggt actggcttag aggtgacgaa gaaatttgcc tagatttggc cacatgtgca | 960 |
| cttgctttta ggttgctttt agcccacggc tatgacgtgt catacgatcc tctaaagcca | 1020 |
| tttgcagagg aatctggttt cagcgatacc cttgagggat atgttaaaaa caccttttcc | 1080 |
| gtattagagc ttttcaaggc tgcccaaagt taccctcatg agagtgcttt gaaaaagcag | 1140 |
| tgttgctgga caaaacaata tctagaaatg gaactaagtt catgggttaa aacaagcgtt | 1200 |
| agggacaagt acttgaaaaa ggaagtggag gatgctttgg catttccatc atatgcctct | 1260 |
| ttagaaagaa gtgaccacag aaggaaaatt cttaatggct cagcagttga aaacacaaga | 1320 |
| gtaaccaaga cctcttacag gttgcataat atatgtacat cagatatctt aaaacttgct | 1380 |
| gtcgacgatt tcaacttttg ccaatctatt catagagagg aaatggaaag attggataga | 1440 |
| tggatagtgg agaatagact acaggaatta aagttcgcca gacaaaaatt ggcttactgt | 1500 |
| tactttagtg gcgctgccac actattctct ccagaattgt ctgacgcaag gatctcatgg | 1560 |
| gctaagggag gtgttctaac cacagtagtc gatgactttt ttgatgttgg cggtagtaaa | 1620 |
| gaagagcttg agaacttaat tcacttggtg gaaaagtggg atcttaatgg agttcctgaa | 1680 |
| tactcttcag agcatgtaga aataattttc tctgtcctaa gagacactat cttagaaacc | 1740 |
| ggtgataaag cctttacata tcagggcaga aacgttactc accatattgt gaaaatatgg | 1800 |
| ttggacttac ttaagagcat gctaagggag gctgaatggt ccagtgacaa atcaacccca | 1860 |
| tctttggaag attacatgga gaatgcctat atcagcttcg cattaggtcc tattgtattg | 1920 |
| ccagctacat accttatagg acctccacta cctgaaaaga ctgtcgactc ccaccaatat | 1980 |
| aatcaattat acaaattggt tagtaccatg ggtagactat taaacgatat ccagggcttt | 2040 |
| aagagggaat cagccgaggg aaaacttaat gcagtgtctc tacatatgaa gcatgaaaga | 2100 |
| gacaacagaa gcaaagaggt tattatagaa tccatgaaag gattggctga aaggaaaaga | 2160 |
| gaggaattac acaaacttgt actagaagag aaaggtagtg tcgttccaag agaatgcaag | 2220 |
| gaagccttct taaaaatgtc aaaagtgttg aacctttttt ataggaagga tgatggcttc | 2280 |
| acatctaacg acttgatgag ccttgtgaaa tccgtcatct acgagcctgt ttcacttcaa | 2340 |
| aaggagagtc taacttga | 2358 |

SEQ ID NO: 182

| | |
|---|---|
| MSINLRSSGC SSPISATLER GLDSEVQTRA NNVSFEQTKE KIRKMLEKVE LSVSAYDTSW | 60 |
| VAMVPSPSSQ NAPLFPQCVK WLLDNQHEDG SWGLDNHDHQ SLKKDVLSST LASILALKKW | 120 |
| GIGERQINKG LQFIELNSAL VIDETIQKPT GFDIIFPGMI KYARDLNLTI PLGSEVVDDM | 180 |
| IRKRDLDLKC DSEKFSKGRE AYLAYVLEGT RNLKDWDLIV KYQRKNGSLF DSPATTAAAF | 240 |
| TQFGNDGCLR YLCSLLQKFE AAVPSVYPFD QYARLSIIVT LESLGIDRDF KTEIKSILDE | 300 |
| TYRYWLRGDE EICLDLATCA LAFRLLLAHG YDVSYDPLKP FAEESGFSDT LEGYVKNTFS | 360 |
| VLELFKAAQS YPHESALKKQ CCWTKQYLEM ELSSWVKTSV RDKYLKKEVE DALAFPSYAS | 420 |
| LERSDHRRKI LNGSAVENTR VIKTSYRLHN ICTSDILKLA VDDFNFCQSI HREEMERLDR | 480 |
| WIVENRLQEL KFARQKLAYC YFSGAATLFS PELSDARISW AKGGVLTTVV DDFFDVGGSK | 540 |
| EELENLIHLV EKWDLNGVPE YSSEHVEIIF SVLRDTILET GDKAFTYQGR NVTHHIVKIW | 600 |
| LDLLKSMLRE AEWSSDKSTP SLEDYMENAY ISFALGPIVL PATYLIGPPL PEKTVDSHQY | 660 |
| NQLYKLVSTM GRLLNDIQGF KRESAEGKLN AVSLHMKHER DNRSKEVIIE SMKGLAERKR | 720 |
| EELHKLVLEE KGSVVPRECK EAFLKMSKVL NLFYRKDDGF TSNDLMSLVK SVIYEPVSLQ | 780 |
| KESLT | 785 |

SEQ ID NO: 183

| | |
|---|---|
| atgatgagta attttgttac tttgattgag ccattagaac ttaccggttc aagggttcta | 60 |
| agaatcgccg tggcgttcgc ggctttgtgt ggtgccaccg gtttgctggc cttttcctgg | 120 |
| tggatttata agcaaagctc tagtaagcca acgcttccgt accctgtagt tggcgataca | 180 |
| catgcacaaa gcttggaaaa aaatttaatc aaaggaatgc aacaatacag agacagtcca | 240 |
| tttttcctag ccggaagcag acctccgtta ctaattttgc ctatgtccgt ttttcatgag | 300 |
| atccataaca tgcctaacga atatatatct attatcgttg agcacgaaga caaattccaa | 360 |
| ggcaagtata cccatataac tacaataaga ccagaaattc ctgcaacaat aagacaagat | 420 |
| ttaacaagga acatgccaaa tatcatacta gaattgcaag atgaactaac atacgcctca | 480 |
| gaccaatggc ctagaacatc caaatggtct tcagtttcac tatatgacat gatgttgagg | 540 |
| actgtagccc tgctgtcagg tagagctttc gttggcttac cactatgtag agatgaggga | 600 |
| tggttgcagg caagtatagg ttatacagtc caatgcgttt caataagaga tcagcttttt | 660 |
| acttggagcc ccgtattgag accaattatc gggccattct tgccctcagt tagaagtgtg | 720 |
| aggagacact tgagatttgc tgcagaaatt atggctcctc ttatcagtca ggctttacaa | 780 |
| gatgaaaagc aacacagggc tgatacactt ttagcagatc agaccgaagg tcgtggcacg | 840 |
| tttatttctt ggttactgag acacctgcca gaagaattac gtactcctga gcaagtagga | 900 |
| ctggaccaga tgcttgtatc ttttgccgca attcacacta caacaatggc tctaaccaaa | 960 |
| gtcgtgtggg aattagttaa gagaccagaa tacatcgaac ccttgagaac tgaaatgcaa | 1020 |
| gatgtcttcg ggcccgatgc ggtttcacca gacatttgca ttaataaaga ggccctatcc | 1080 |
| aggttgcata aattggattc ttttattagg gaggttcaaa gatggtgtcc ttccactttt | 1140 |
| gttactccta gccgtagagt gatgaagtcc atgacgctga gcaacggaat taaactgcaa | 1200 |
| cgtggtacga gtattgcttt tcctgctcat gctatacata tgtcagaaga aacacctact | 1260 |
| ttttcacctg acttttcttc tgacttcgaa aatccttccc ctagaatttt tgatgggttc | 1320 |
| cgttatttaa acttgaggtc aatcaaggga caaggaagcc agcatcaagc ggctactacc | 1380 |
| ggtcctgatt acttaatttt taaccatggt aaacatgctt gccctggtag attttttgct | 1440 |
| atttcagaaa taaaaatgat cttgatagag ttactagcta agtacgattt caggttggaa | 1500 |
| gacggaaaac cagggcctga actaatgaga gttggtactg agacaagatt ggatacaaag | 1560 |
| gcaggtttgg agatgagacg tagataa | 1587 |

SEQ ID NO: 184

| | |
|---|---|
| MMSNFVTLIE PLELTGSRVL RIAVAFAALC GATGLLAFSW WIYKQSSSKP TLPYPVVGDT | 60 |
| HAQSLEKNLI KGMQQYRDSP FFLAGSRPPL LILPMSVFHE IHNMPNEYIS IIVEHEDKFQ | 120 |
| GKYTHITTIR PEIPATIRQD LTRNMPNIIL ELQDELTYAS DQWPRTSKWS SVSLYDMMLR | 180 |
| TVALLSGRAF VGLPLCRDEG WLQASIGYTV QCVSIRDQLF TWSPVLRPII GPFLPSVRSV | 240 |
| RRHLRFAAEI MAPLISQALQ DEKQHRADTL LADQTEGRGT FISWLLRHLP EELRTPEQVG | 300 |
| LDQMLVSFAA IHTTIMALTK VVWELVKRPE YIEPLRTEMQ DVFGPDAVSP DICINKEALS | 360 |
| RLHKLDSFIR EVQRWCPSTF VITSRRVMKS MTLSNGIKLQ RGTSIAFPAH AIHMSEETPT | 420 |
| FSPDFSSDFE NPSPRIFDGF RYLNLRSIKG QGSQHQAATT GPDYLIFNHG KHACPGRFFA | 480 |
| ISEIKMILIE LLAKYDFRLE DGKPGPELMR VGTETRLDTK AGLEMRRR | 528 |

SEQ ID NO: 185

| | |
|---|---|
| atgatgtcca acttcgttac cttgatcgaa ccattggaat tgactggttc tagagttttg | 60 |
| agaattgctg ttgcttttgc tgctttgtgt ggtgctactg gtttgttggc tttttcttgg | 120 |
| tggatctaca agcaatcttc ttcaaaacct actttgccat acccagttgt tggtgatact | 180 |
| catgctcaat ctttggaaaa gaacttgatt aagggtatgc aacaatacag agactcccca | 240 |
| ttctttttgg ctggttcaag accaccatta ttgatcttgc caatgtctgt tttccacgaa | 300 |
| atccataaca tgccaaacga atatatctcc atcatcgttg aacacgaaga taagttccaa | 360 |
| ggtaaataca cccatatcac taccatcaga ccagaaattc cagctaccat tagacaagat | 420 |
| ttgaccagaa acatgcctaa catcatcttg gaattgcaag acgaattgac ctacgcttct | 480 |
| gatcaatggc caagaacttc taagtggtcc tctgtttcat tatacgacat gatgttgaga | 540 |
| accgttgctt tgttgtctgg tagagctttt gttggtttgc cattgtgtag agatgaaggt | 600 |
| tggttgcaag cttctattgg ttacactgtt caatgcgtgt ctatcagaga tcagttgttt | 660 |
| acttggtccc cagttttgag gccaattatt ggtccatttt tgccatccgt tagatctgtt | 720 |
| agaaggcatt tgagattcgc tgctgaaatt atggctccat tgatttctca agccttgcaa | 780 |
| gacgaaaaac aacatagagc tgataccttg ttggctgatc aaactgaagg tagaggtact | 840 |
| ttcatttcct ggttgttgag acatttgcca gaagaattga gaaccccaga acaagttggt | 900 |
| ttggatcaaa tgttggtttc ctttgctgct attcatacca ctactatggc tttgacaaag | 960 |
| gttgtttggg aattggtaaa aaggccagag tacattgaac cattgagaac cgaaatgcaa | 1020 |
| gatgtttttg gtccagatgc tgtttctcca gatatctgca ttaacaaaga agccttgtcc | 1080 |
| agattgcaca agttggattc tttcatcaga gaagttcaaa gatggtgtcc atctactttc | 1140 |
| gttactccat ctagaagagt catgaagtct atgactttgt ccaacggtat caagttgcaa | 1200 |
| agaggtactt ctattgcttt tccagctcat gccattcaca tgtctgaaga aactccaaca | 1260 |
| ttttccccag atttctcttc cgattttgaa aacccatccc caagaatttt cgacggtttt | 1320 |
| agatacttga acttgaggtc cattaagggt caaggttcac aacatcaagc tgctactact | 1380 |
| ggtccagatt acttgatttt caatcatggt aaacatgcct gcccaggtag attttttgct | 1440 |
| atctctgaaa tcaagatgat tttgatcgag ttgttggcca agtacgactt cagattggaa | 1500 |
| gatggtaaac caggtccaga attgatgaga gttggtactg aaactagatt ggataccaaa | 1560 |
| gctggtttgg aaatgagaag aaggtga | 1587 |

SEQ ID NO: 186

| | |
|---|---|
| MMSNFVTLIE PLELTGSRVL RIAVAFAALC GATGLLAFSW WIYKQSSSKP TLPYPVVGDT | 60 |
| HAQSLEKNLI KGMQQYRDSP FFLAGSRPPL LILPMSVFHE IHNMPNEYIS IIVEHEDKFQ | 120 |
| GKYTHITTIR PEIPATIRQD LTRNMPNIIL ELQDELTYAS DQWPRTSKWS SVSLYDMMLR | 180 |
| TVALLSGRAF VGLPLCRDEG WLQASIGYTV QCVSIRDQLF TWSPVLRPII GPFLPSVRSV | 240 |
| RRHLRFAAEI MAPLISQALQ DEKQHRADTL LADQTEGRGT FISWLLRHLP EELRTPEQVG | 300 |
| LDQMLVSFAA IHTTIMALTK VVWELVKRPE YIEPLRTEMQ DVFGPDAVSP DICINKEALS | 360 |
| RLHKLDSFIR EVQRWCPSTF VTPSRRVMKS MTLSNGIKLQ RGTSIAFPAH AIHMSEETPT | 420 |
| FSPDFSSDFE NPSPRIFDGF RYLNLRSIKG QGSQHQAATT GPDYLIFNHG KHACPGRFFA | 480 |
| ISEIKMILIE LLAKYDFRLE DGKPGPELMR VGTETRLDTK AGLEMRRR | 528 |

SEQ ID NO: 187

| | |
|---|---|
| atggcggaat tagatacgtt agatatcgtt gttttaggcg ctttattgtt gggcacatta | 60 |
| gcgtatttta cgaagggcac attatggggt gtcactaagg atccttatgc aaacgctttc | 120 |
| gcaaatgcta acggagctaa agccggcaga tcaagaaata tcgttgaaaa aatggatgaa | 180 |
| tctggtaaaa actgcgtcat attctacggt tctcaaactg gaacggcaga ggattacgca | 240 |
| tcaagattag cgaaagaagg aaagtcaaga ttcgggttag ggactatggt tgcagattta | 300 |
| gaagaatatg attatgataa ccttgataca atgagcggcg ataaggttgc catgtttgtt | 360 |
| cttgctacct atggcgaggg cgaaccaact gacaacgcag tagagtttta tgaatttatt | 420 |
| actggtgaag gggttgcttt tagtgaagga aacgatcccc ccttaggcaa tctgaactac | 480 |
| gtggcctttg gactggggaa caatacttat gaacactaca attcaatggt cagaaatgtc | 540 |
| gataaagccc ttaggaatct gggtgctcat aggatcggag aggctggtga aggcgatgac | 600 |
| ggtgctggca caatggaaga agattttcta gcatggaagg aaccaatgtg ggccgcctta | 660 |
| gctgacaaaa tgggtttgga ggaaagggaa gcagtatatg accctgtgtt cagtatcgtt | 720 |
| gatcgtgata atttgactcc tgaaagccca gaagtctatt tgggtgaacc taataaaatg | 780 |
| catttagagg atgcggtcaa gggcccattt aattctcata atccatatat agcaccaata | 840 |
| gctgaatcta gagaattgtt tagtgttaaa gacaggaatt gcatccatat ggaaattgac | 900 |
| atagacggtt caaatttgag ctatcaaact ggggatcatg tggctatttg gcctaccaac | 960 |
| ccaggagatg aagtggatag atttttagac atcattgatt taaaggataa acgtgacaag | 1020 |
| gttataggag tgaaagcact tgaaccaact gcaaaggtcc cttttccaac accaacaaca | 1080 |
| tatgacgtta tcgccaggta tcatttagaa atctgtgcac cggtctctag acagtttgtg | 1140 |
| tccactctag cagcattctc cccaaatgat gaggtaaaag cagaaatgac tagattgggt | 1200 |
| aacgataagg attattttca tgataagacg ggcccacatt attataatat cgcccgtttt | 1260 |
| ctagctgcgg ttggtaaggg cgagaaatgg tcaaatatcc ctttttctgt ttttgtcgaa | 1320 |
| ggtttaacga aattacaacc aagatattat tcaatctcct cttcaagcct agtacaacca | 1380 |
| aaaaaaatat caataacggc agtaattgag tcacaggtta tacctgccag gcaagatcca | 1440 |
| tttagaggtg tagctacgaa ctacttattt gcattgaaac agaagcaaaa cggtgatcca | 1500 |
| aatccctccc catttggaca tacttatgca ttaaacggcc ctagaaataa atttgacggt | 1560 |
| atacacgtcc ccgtccacgt aaggcactcc aatttcaaac taccgagcga tccagcaaaa | 1620 |
| ccagttatta tggttggtcc aggaactgga gtggctccgt ttagaggttt catccaagag | 1680 |
| agagctaaac aggcccagga tggggccaca gtaggccgta ctatcttgtt cttcggttgc | 1740 |
| caacgtaggt ccgaagattt tttgtacgaa agtgaatgga aagaatacaa ggaagttcta | 1800 |
| ggagataccc ttgagatagt cactgccttc tccagggaaa catcaaagaa agtttatgtg | 1860 |
| cagcacaggt tgaaagagag atccaaagaa atcggagaac tattatcaca gaaagcatac | 1920 |
| ttttatgtgt gtggcgatgc tgctcatatg gctagagaag ttaatactgt attggctcaa | 1980 |
| attatcgctg aatctagggg tgtaagtgaa gccaagggtg aagagattgt taaaaatatg | 2040 |
| agggctgcta atcagtacca agttaggagg gggaacaatg tctttttttg ggctataagt | 2100 |
| ggttctattg atatgacggc caataccgcc aacttacaag aagatgtgtg gagctga | 2157 |

SEQ ID NO: 188

| | |
|---|---|
| MAELDTLDIV VLGALLLGTL AYFTKGTLWG VTKDPYANAF ANANGAKAGR SRNIVEKMDE | 60 |
| SGKNCVIFYG SQTGTAEDYA SRLAKEGKSR FGLGTMVADL EEYDYDNLDT MSGDKVAMFV | 120 |
| LATYGEGEPT DNAVEFFEFI TGEGVAFSEG NDPPLGNLNY VAFGLGNNTY EHYNSMVRNV | 180 |
| DKALRNLGAH RIGEAGEGDD GAGTMEEDFL AWKEPMWAAL ADKMGLEERE AVYDPVFSIV | 240 |
| DRDNLTPESP EVYLGEPNKM HLEDAVKGPF NSHNPYIAPI AESRELFSVK DRNCIHMEID | 300 |
| IDGSNLSYQT GDHVAIWPTN PGDEVDRFLD IIDLKDKRDK VIGVKALEPT AKVPFPTPTT | 360 |
| YDVIARYHLE ICAPVSRQFV STLAAFSPND EVKAEMTRLG NDKDYFHDKT GPHYYNIARF | 420 |
| LAAVGKGEKW SNIPFSVFVE GLTKLQPRYY SISSSSLVQP KKISITAVIE SQVIPARQDP | 480 |
| FRGVATNYLF ALKQKQNGDP NPSPFGHTYA LNGPRNKFDG IHVPVHVRHS NFKLPSDPAK | 540 |
| PVIMVGPGTG VAPFRGFIQE RAKQAQDGAT VGRTILFFGC QRRSEDFLYE SEWKEYKEVL | 600 |
| GDTLEIVTAF SRETSKKVYV QHRLKERSKE IGELLSQKAY FYVCGDAAHM AREVNTVLAQ | 660 |
| IIAESRGVSE AKGEEIVKNM RAANQYQVRR GNNVFFWAIS GSIDMTANTA NLQEDVWS | 718 |

SEQ ID NO: 189

| | |
|---|---|
| atggcggaat tagatacgtt agatatcgtt gttttaggcg ctttattgtt gggcacatta | 60 |
| gcgtatttta cgaagggcac attatggggt gtcactaagg atccttatgc aaacgctttc | 120 |
| gcaaatgcta acggagctaa agccggcaga tcaagaaata tcgttgaaaa aatggatgaa | 180 |
| tctggtaaaa actgcgtcat attctacggt tctcaaactg gaacggcaga ggattacgca | 240 |
| tcaagattag cgaaagaagg aaagtcaaga ttcgggttag ggactatggt tgcagattta | 300 |
| gaagaatatg attatgataa ccttgataca atgagcggcg ataaggttgc catgtttgtt | 360 |
| cttgctacct atggcgaggg cgaaccaact gacaacgcag tagagtttta tgaatttatt | 420 |
| actggtgaag gggttgcttt tagtgaagga aacgatcccc ccttaggcaa tctgaactac | 480 |
| gtggcctttg gactggggaa caatacttat gaacactaca attcaatggt cagaaatgtc | 540 |
| gataaagccc ttaggaatct gggtgctcat aggatcggag aggctggtga aggcgatgac | 600 |
| ggtgctggca caatggaaga agattttcta gcatggaagg aaccaatgtg ggccgcctta | 660 |
| gctgacaaaa tgggtttgga ggaaagggaa gcagtatatg accctgtgtt cagtatcgtt | 720 |
| gatcgtgata atttgactcc tgaaagccca gaagtctatt tgggtgaacc taataaaatg | 780 |
| catttagagg atgcggtcaa gggcccattt aattctcata atccatatat agcaccaata | 840 |
| gctgaatcta gagaattgtt tagtgttaaa gacaggaatt gcatccatat ggaaattgac | 900 |
| atagacggtt caaatttgag ctatcaaact ggggatcatg tggctatttg gcctaccaac | 960 |
| ccaggagatg aagtggatag atttttagac atcattgatt taaaggataa acgtgacaag | 1020 |
| gttataggag tgaaagcact tgaaccaact gcaaaggtcc cttttccaac accaacaaca | 1080 |
| tatgacgtta tcgccaggta tcatttagaa atctgtgcac cggtctctag acagtttgtg | 1140 |
| tccactctag cagcattctc cccaaatgat gaggtaaaag cagaaatgac tagattgggt | 1200 |
| aacgataagg attattttca tgataagacg ggcccacatt attataatat cgcccgtttt | 1260 |
| ctagctgcgg ttggtaaggg cgagaaatgg tcaaatatcc ctttttctgt ttttgtcgaa | 1320 |
| ggtttaacga aattacaacc aagatattat tcaatctcct cttcaagcct agtacaacca | 1380 |
| aaaaaaatat caataacggc agtaattgag tcacaggtta tacctgccag gcaagatcca | 1440 |
| tttagaggtg tagctacgaa ctacttattt gcattgaaac agaagcaaaa cggtgatcca | 1500 |
| aatccctccc catttggaca tacttatgca ttaaacggcc ctagaaataa atttgacggt | 1560 |
| atacacgtcc ccgtccacgt aaggcactcc aatttcaaac taccgagcga tccagcaaaa | 1620 |
| ccagttatta tggttggtcc aggaactgga gtggctccgt ttagaggttt catccaagag | 1680 |
| agagctaaac aggcccagga tggggccaca gtaggccgta ctatcttgtt cttcggttgc | 1740 |
| caacgtaggt ccgaagattt tttgtacgaa agtgaatgga aagaatacaa ggaagttcta | 1800 |
| ggagataccc ttgagatagt cactgccttc tccagggaaa catcaaagaa agtttatgtg | 1860 |
| cagcacaggt tgaaagagag atccaaagaa atcggagaac tattatcaca gaaagcatac | 1920 |
| ttttatgtgt gtggcgatgc tgctcatatg gctagagaag ttaatactgt attggctcaa | 1980 |
| attatcgctg aatctagggg tgtaagtgaa gccaagggtg aagagattgt taaaaatatg | 2040 |
| agggctgcta atcagtacca agaagatgtg tggagctga | 2079 |

SEQ ID NO: 190

| | |
|---|---|
| MAELDTLDIV VLGALLLGTL AYFTKGTLWG VTKDPYANAF ANANGAKAGR SRNIVEKMDE | 60 |
| SGKNCVIFYG SQTGTAEDYA SRLAKEGKSR FGLGTMVADL EEYDYDNLDT MSGDKVAMFV | 120 |
| LATYGEGEPT DNAVEFFEFI TGEGVAFSEG NDPPLGNLNY VAFGLGNNTY EHYNSMVRNV | 180 |
| DKALRNLGAH RIGEAGEGDD GAGTMEEDFL AWKEPMWAAL ADKMGLEERE AVYDPVFSIV | 240 |
| DRDNLTPESP EVYLGEPNKM HLEDAVKGPF NSHNPYIAPI AESRELFSVK DRNCIHMEID | 300 |
| IDGSNLSYQT GDHVAIWPTN PGDEVDRFLD IIDLKDKRDK VIGVKALEPT AKVPFPTPTT | 360 |
| YDVIARYHLE ICAPVSRQFV STLAAFSPND EVKAEMTRLG NDKDYFHDKT GPHYYNIARF | 420 |
| LAAVGKGEKW SNIPFSVFVE GLTKLQPRYY SISSSSLVQP KKISITAVIE SQVIPARQDP | 480 |
| FRGVATNYLF ALKQKQNGDP NPSPFGHTYA LNGPRNKFDG IHVPVHVRHS NFKLPSDPAK | 540 |
| PVIMVGPGTG VAPFRGFIQE RAKQAQDGAT VGRTILFFGC QRRSEDFLYE SEWKEYKEVL | 600 |
| GDTLEIVTAF SRETSKKVYV QHRLKERSKE IGELLSQKAY FYVCGDAAHM AREVNTVLAQ | 660 |
| IIAESRGVSE AKGEEIVKNM RAANQYQEDV WS | 692 |

SEQ ID NO: 191

| | |
|---|---|
| atggccgaat tggatacctt ggatatcgtt gttttgggtg ttatcttctt gggtactgtt | 60 |
| gcttacttca ccaaaggtaa attgtggggt gttactaagg atccatacgc taatggtttt | 120 |
| gctgctggtg gtgcttctaa accaggtaga actagaaata tcgttgaagc catggaagaa | 180 |
| tctggtaaga actgtgttgt tttctacggt tctcaaactg gtactgctga agattatgct | 240 |
| tccagattgg ctaaagaagg taagagtaga ttcggtttga acaccatgat tgccgatttg | 300 |
| gaagattacg atttcgataa cttggatacc gtcccatctg ataacatcgt tatgtttgtt | 360 |
| ttggctacct acggtgaagg tgaacctact gataatgctg ttgacttcta cgaattcatt | 420 |
| accggtgaag atgcttcttt caacgaaggt aatgatccac cattgggtaa cttgaattac | 480 |
| gttgcttttg gtttgggtaa caacacctac gaacattaca actccatggt tagaaacgtc | 540 |
| aacaaggctt tggaaaaatt gggtgctcat agaattggtg aagctggtga aggtgatgat | 600 |
| ggtgctggta ctatggaaga agattttttg gcttggaaag acccaatgtg ggaagccttg | 660 |
| gctaaaaaga tgggtttgga agaaagagaa gctgtctacg aacctatttt cgccattaac | 720 |
| gaaagagatg atttgacccc tgaagccaat gaagtttatt tgggtgaacc taacaagttg | 780 |
| cacttggaag gtactgctaa aggtccattc aattctcaca acccatatat tgctccaatc | 840 |
| gccgaatctt acgaattatt ctctgctaag gatagaaact gcttgcacat ggaaattgac | 900 |
| atctctggtt ctaatttgaa gtacgaaacc ggtgatcata ttgccatttg gccaactaat | 960 |
| ccaggtgaag aagttaacaa gttcttggac atcttggact tgtccggtaa acaacattct | 1020 |
| gttgttactg ttaaggcctt ggaacctaca gctaaagttc cttttccaaa tccaactacc | 1080 |
| tacgatgcca ttttgagata ccatttggaa atttgcgctc cagtctctag acaattcgtt | 1140 |
| tctactttgg ctgcttttgc tccaaacgat gatattaagg ctgaaatgaa cagattgggt | 1200 |
| tccgataagg attacttcca cgaaaaaact ggtccacact actacaacat tgctagattt | 1260 |
| ttggcctctg tctctaaagg tgaaaagtgg actaagattc cattctccgc tttcattgaa | 1320 |
| ggtttgacta agttgcaacc tagatattac tccatctcct cctcatcttt ggttcaacct | 1380 |
| aagaagatct ctattaccgc cgttgttgaa tcccaacaaa ttccaggtag agatgatcct | 1440 |
| tttagaggtg ttgctaccaa ttacttgttc gccttgaaac aaaagcaaaa cggtgatcca | 1500 |
| aatcctgctc catttggtca atcttatgaa ttgactggtc caagaaacaa gtacgatggt | 1560 |
| attcatgttc cagttcacgt tagacactct aactttaagt tgccatctga tccaggtaag | 1620 |
| ccaattatca tgattggtcc aggtactggt gttgctccat tcagaggttt tgttcaagaa | 1680 |
| agagctaagc aagctagaga tggtgttgaa gttggtaaaa ccttgttgtt cttcggttgt | 1740 |
| agaaagtcca ctgaagattt catgtaccaa aaagaatggc aagaatacaa agaagcctta | 1800 |
| ggtgacaagt tcgaaatgat tactgccttc tcaagagaag gttctaagaa ggtttacgtc | 1860 |
| caacacagat tgaaagaaag atccaaagaa gtctccgatt tgttgtctca aaaggcctac | 1920 |
| ttttacgttt gtggtgatgc tgctcatatg gccagagaag ttaatactgt tttggcccaa | 1980 |
| attatcgctg aaggtagagg tgtatctgaa gctaagggtg aagaaatcgt taagaacatg | 2040 |
| agatccgcca atcaatacca agaagatgtt tggtcctaa | 2079 |

SEQ ID NO: 192

| | |
|---|---|
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQEDV WS | 692 |

SEQ ID NO: 193

| | |
|---|---|
| atggcagaat tagatacact tgatatagta gtattaggtg ttatcttttt gggtactgtg | 60 |
| gcatacttta ctaagggtaa attgtggggt gttaccaagg atccatacgc taacggattc | 120 |
| gctgcaggtg gtgcttccaa gcctggcaga actagaaaca tcgtcgaagc tatggaggaa | 180 |
| tcaggtaaaa actgtgttgt tttctacggc agtcaaacag gtacagcgga ggattacgca | 240 |
| tcaagacttg caaaggaagg aaagtccaga ttcggtttga acactatgat cgccgatcta | 300 |
| gaagattatg acttcgataa cttagacact gttccatctg ataacatcgt tatgtttgta | 360 |
| ttggctactt acggtgaagg cgaaccaaca gataacgccg tggatttcta tgagttcatt | 420 |
| actggcgaag atgcctcttt caatgagggc aacgatcctc cactaggtaa cttgaattac | 480 |
| gttgcgttcg gtctgggcaa caatacctac gaacactaca actcaatggt caggaacgtt | 540 |
| aacaaggctc tagaaaagtt aggagctcat agaattggag aagcaggtga gggtgacgac | 600 |
| ggagctggaa ctatggaaga ggacttttta gcttggaaag atccaatgtg ggaagccttg | 660 |
| gctaaaaaga tgggcttgga ggaaagagaa gctgtatatg aacctatttt cgctatcaat | 720 |
| gagagagatg atttgacccc tgaagcgaat gaggtatact tgggagaacc taataagcta | 780 |
| cacttggaag gtacagcgaa aggtccattc aactcccaca acccatatat cgcaccaatt | 840 |
| gcagaatcat acgaactttt ctcagctaag gatagaaatt gtctgcatat ggaaattgat | 900 |
| atttctggta gtaatctaaa gtatgaaaca ggcgaccata tcgcgatctg gcctaccaac | 960 |
| ccaggtgaag aggtcaacaa atttcttgac attctagatc tgtctggtaa gcaacattcc | 1020 |
| gtcgtaacag tgaaagcctt agaacctaca gccaaagttc cttttccaaa tccaactacc | 1080 |
| tacgatgcta tattgagata ccatctggaa atatgcgctc cagtttctag acagtttgtc | 1140 |
| tcaactttag cagcattcgc ccctaatgat gatatcaaag ctgagatgaa ccgtttggga | 1200 |
| tcagacaaag attacttcca cgaaaagaca ggaccacatt actacaatat cgctagattt | 1260 |
| ttggcctcag tctctaaagg tgaaaaatgg acaaagatac cattttctgc tttcatagaa | 1320 |
| ggccttacaa aactacaacc aagatactat tctatctctt cctctagttt agttcagcct | 1380 |
| aaaaagatta gtattactgc tgttgtcgaa tctcagcaaa ttccaggtag agatgaccca | 1440 |
| ttcagaggtg tagcgactaa ctacttgttc gctttgaagc agaaacaaaa cggtgatcca | 1500 |
| aatccagctc cttttggcca atcatacgag ttgacaggac caaggaataa gtatgatggt | 1560 |
| atacatgttc cagtccatgt aagacattct aactttaagc taccatctga tccaggcaaa | 1620 |
| cctattatca tgatcggtcc aggtaccggt gttgcccctt ttagaggctt cgtccaagag | 1680 |
| agggcaaaac aagccagaga tggtgtagaa gttggtaaaa cactgctgtt ctttggatgt | 1740 |
| agaaagagta cagaagattt catgtatcaa aaagagtggc aagagtacaa ggaagctctt | 1800 |
| ggcgacaaat tcgaaatgat tacagctttt tcaagagaag gatctaaaaa ggtttatgtt | 1860 |
| caacacagac tgaaggaaag atcaaaggaa gtttctgatc ttctatccca aaaagcatac | 1920 |
| ttctacgttt gcggagacgc cgcacatatg gcacgtgaag tgaacactgt gttagcacag | 1980 |
| atcatagcag aaggccgtgg tgtatcagaa gccaagggtg aggaaattgt caaaaacatg | 2040 |
| agatcagcaa atcaatacca agaggatgtc tggagttaa | 2079 |

SEQ ID NO: 194

| | |
|---|---|
| MAELDTLDIV VLGVIFLGTV AYFTKGKLWG VTKDPYANGF AAGGASKPGR TRNIVEAMEE | 60 |
| SGKNCVVFYG SQTGTAEDYA SRLAKEGKSR FGLNTMIADL EDYDFDNLDT VPSDNIVMFV | 120 |
| LATYGEGEPT DNAVDFYEFI TGEDASFNEG NDPPLGNLNY VAFGLGNNTY EHYNSMVRNV | 180 |
| NKALEKLGAH RIGEAGEGDD GAGTMEEDFL AWKDPMWEAL AKKMGLEERE AVYEPIFAIN | 240 |
| ERDDLTPEAN EVYLGEPNKL HLEGTAKGPF NSHNPYIAPI AESYELFSAK DRNCLHMEID | 300 |
| ISGSNLKYET GDHIAIWPTN PGEEVNKFLD ILDLSGKQHS VVTVKALEPT AKVPFPNPTT | 360 |
| YDAILRYHLE ICAPVSRQFV STLAAFAPND DIKAEMNRLG SDKDYFHEKT GPHYYNIARF | 420 |
| LASVSKGEKW TKIPFSAFIE GLTKLQPRYY SISSSSLVQP KKISITAVVE SQQIPGRDDP | 480 |
| FRGVATNYLF ALKQKQNGDP NPAPFGQSYE LTGPRNKYDG IHVPVHVRHS NFKLPSDPGK | 540 |
| PIIMIGPGTG VAPFRGFVQE RAKQARDGVE VGKTLLFFGC RKSTEDFMYQ KEWQEYKEAL | 600 |
| GDKFEMITAF SREGSKKVYV QHRLKERSKE VSDLLSQKAY FYVCGDAAHM AREVNTVLAQ | 660 |
| IIAEGRGVSE AKGEEIVKNM RSANQYQEDV WS | 692 |

SEQ ID NO: 195

| | |
|---|---|
| atggcttcag aaaaagaaat taggagagag agattcttga acgttttccc taaattagta | 60 |
| gaggaattga acgcatcgct tttggcttac ggtatgccta aggaagcatg tgactggtat | 120 |
| gcccactcat tgaactacaa cactccaggc ggtaagctaa atagaggttt gtccgttgtg | 180 |
| gacacgtatg ctattctctc caacaagacc gttgaacaat tggggcaaga agaatacgaa | 240 |
| aaggttgcca ttctaggttg gtgcattgag ttgttgcagg cttacttctt ggtcgccgat | 300 |
| gatatgatgg acaagtccat taccagaaga ggccaaccat gttggtacaa ggttcctgaa | 360 |
| gttggggaaa ttgccatcaa tgacgcattc atgttagagg ctgctatcta caagcttttg | 420 |
| aaatctcact tcagaaacga aaaatactac atagatatca ccgaattgtt ccatgaggtc | 480 |
| accttccaaa ccgaattggg ccaattgatg gacttaatca ctgcacctga agacaaagtc | 540 |
| gacttgagta agttctccct aaagaagcac tccttcatag ttactttcaa gactgcttac | 600 |
| tattctttct acttgcctgt cgcattggcc atgtacgttg ccggtatcac ggatgaaaag | 660 |
| gatttgaaac aagccagaga tgtcttgatt ccattgggtg aatacttcca aattcaagat | 720 |
| gactacttag actgcttcgg taccccagaa cagatcggta agatcggtac agatatccaa | 780 |
| gataacaaat gttcttgggt aatcaacaag gcattggaac ttgcttccgc agaacaaaga | 840 |
| aagactttag acgaaaatta cggtaagaag gactcagtcg cagaagccaa atgcaaaaag | 900 |
| attttcaatg acttgaaaat tgaacagcta taccacgaat atgaagagtc tattgccaag | 960 |
| gatttgaagg ccaaaatttc tcaggtcgat gagtctcgtg gcttcaaagc tgatgtctta | 1020 |
| actgcgttct tgaacaaagt ttacaagaga agcaaaggtt ctagtactgg ttcatctaca | 1080 |
| tctactggag gtatggtcgc acaaactttc aacctggata cctacttatc ccaaagacaa | 1140 |
| caacaagttg aagaggccct aagtgctgct cttgtgccag cttatcctga gagaatatac | 1200 |
| gaagctatga gatactccct cctggcaggt ggcaaaagat taagacctat cttatgttta | 1260 |
| gctgcttgcg aattggcagg tggttctgtt gaacaagcca tgccaactgc gtgtgcactt | 1320 |
| gaaatgatcc atacaatgtc actaattcat gatgacctgc cagccatgga taacgatgat | 1380 |
| ttcagaagag gaaagccaac taatcacaag gtgttcgggg aagatatagc catcttagcg | 1440 |
| ggtgatgcgc ttttagctta cgcttttgaa catattgctt ctcaaacaag aggagtacca | 1500 |
| cctcaattgg tgctacaagt tattgctaga atcggacacg ccgttgctgc aacaggcctc | 1560 |
| gttggaggcc aagtcgtaga ccttgaatct gaaggtaaag ctatttcctt agaaacattg | 1620 |
| gagtatattc actcacataa gactggagcc ttgctggaag catcagttgt ctcaggcggt | 1680 |
| attctcgcag gggcagatga agagcttttg gccagattgt ctcattacgc tagagatata | 1740 |
| ggcttggctt ttcaaatcgt cgatgatatc ctggatgtta ctgctacatc tgaacagttg | 1800 |
| gggaaaaccg ctggtaaaga ccaggcagcc gcaaaggcaa cttatccaag tctattgggt | 1860 |
| ttagaagcct ctagacagaa agcggaagag ttgattcaat ctgctaagga agccttaaga | 1920 |
| ccttacggtt cacaagcaga gccactccta gcgctggcag acttcatcac acgtcgtcag | 1980 |
| cattaa | 1986 |

SEQ ID NO: 196

| | |
|---|---|
| MASEKEIRRE RFLNVFPKLV EELNASLLAY GMPKEACDWY AHSLNYNTPG GKLNRGLSVV | 60 |
| DTYAILSNKT VEQLGQEEYE KVAILGWCIE LLQAYFLVAD DMMDKSITRR GQPCWYKVPE | 120 |
| VGEIAINDAF MLEAAIYKLL KSHFRNEKYY IDITELFHEV TFQTELGQLM DLITAPEDKV | 180 |
| DLSKFSLKKH SFIVTFKTAY YSFYLPVALA MYVAGITDEK DLKQARDVLI PLGEYFQIQD | 240 |
| DYLDCFGTPE QIGKIGTDIQ DNKCSWVINK ALELASAEQR KTLDENYGKK DSVAEAKCKK | 300 |
| IFNDLKIEQL YHEYEESIAK DLKAKISQVD ESRGFKADVL TAFLNKVYKR SKGSSTGSST | 360 |
| STGGMVAQTF NLDTYLSQRQ QQVEEALSAA LVPAYPERIY EAMRYSLLAG GKRLRPILCL | 420 |
| AACELAGGSV EQAMPTACAL EMIHTMSLIH DDLPAMDNDD FRRGKPTNHK VFGEDIAILA | 480 |
| GDALLAYAFE HIASQTRGVP PQLVLQVIAR IGHAVAATGL VGGQVVDLES EGKAISLETL | 540 |
| EYIHSHKTGA LLEASVVSGG ILAGADEELL ARLSHYARDI GLAFQIVDDI LDVTATSEQL | 600 |
| GKTAGKDQAA AKATYPSLLG LEASRQKAEE LIQSAKEALR PYGSQAEPLL ALADFITRRQ | 660 |
| H | 661 |

SEQ ID NO: 197

| | |
|---|---|
| atgccaaaga ttGttatttt gcctcatcag gatctctgcc ctgatggcgc tgttctggaa | 60 |
| gctaatagcg gtgaaaccat tctcgacgca gctctgcgta acggtatcga gattgaacac | 120 |
| gcctgtgaaa aatcctgtgc ttgcaccacc tgccactgca tcgttcgtga aggttttgac | 180 |
| tcactgccgg aaagctcaga gcaggaagac gacatgctgg acaaagcctg gggactggag | 240 |
| ccggaaagcc gtttaagctg ccaggcgcgc gttaccgacg aagatttagt agtcgaaatc | 300 |
| ccgcgttaca ctatcaacca tgcgcgtgag cattaa | 336 |

SEQ ID NO: 198

| | |
|---|---|
| MPKIVILPHQ DLCPDGAVLE ANSGETILDA ALRNGIEIEH ACEKSCACTT CHCIVREGFD | 60 |
| SLPESSEQED DMLDKAWGLE PESRLSCQAR VTDEDLVVEI PRYTINHARE H | 111 |

SEQ ID NO: 199

| | |
|---|---|
| atggctgatt gggtaacagg caaagtcact aaagtgcaga actggaccga cgccctgttt | 60 |
| agtctcaccg ttcacgcccc cgtgcttccg tttaccgccg ggcaatttac caagcttggc | 120 |
| cttgaaatcg acggcgaacg cgtccagcgc gcctactcct atgtaaactc gcccgataat | 180 |
| cccgatctgg agttttacct ggtcaccgtc cccgatggca aattaagccc acgactggcg | 240 |
| gcactgaaac caggcgatga agtgcaggtg gttagcgaag cggcaggatt ctttgtgctc | 300 |
| gatgaagtgc cgcactgcga aacgctatgg atgctggcaa ccggtacagc gattggccct | 360 |
| tatttatcga ttctgcaact aggtaaagat ttagatcgct tcaaaaatct ggtcctggtg | 420 |
| cacgccgcac gttatgccgc cgacttaagc tatttgccac tgatgcagga actggaaaaa | 480 |
| cgctacgaag gaaaactgcg cattcagacg gtggtcagtc gggaaacggc agcggggtcg | 540 |
| ctcaccggac ggataccggc attaattgaa agtggggaac tggaaagcac gattggcctg | 600 |
| ccgatgaata aagaaaccag ccatgtgatg ctgtgcggca atccacagat ggtgcgcgat | 660 |
| acacaacagt tgctgaaaga gacccggcag atgacgaaac atttacgtcg ccgaccgggc | 720 |
| catatgacag cggagcatta ctggtaa | 747 |

SEQ ID NO: 200

| | |
|---|---|
| MADWVIGKVT KVQNWTDALF SLTVHAPVLP FTAGQFTKLG LEIDGERVQR AYSYVNSPDN | 60 |
| PDLEFYLVTV PDGKLSPRLA ALKPGDEVQV VSEAAGFFVL DEVPHCETLW MLATGTAIGP | 120 |
| YLSILQLGKD LDRFKNLVLV HAARYAADLS YLPLMQELEK RYEGKLRIQT VVSRETAAGS | 180 |
| LTGRIPALIE SGELESTIGL PMNKETSHVM LCGNPQMVRD TQQLLKETRQ MTKHLRRRPG | 240 |
| HMTAEHYW | 248 |

SEQ ID NO: 201

| | |
|---|---|
| atgtccttcc cagatgaaca aaaggttgat ttccaaacct tccagaacgt tatcaacaat | 60 |
| caattgtctc caacctccga atccagacat ggtatttgtc catctactga agaatccttg | 120 |
| tgggaatctc cagtttctac tcaagatgat gttgatagag ctgtttctgc tgctaaagct | 180 |
| gcttatccag cttggagaaa attgtcttgg gacgaaagag cttcttactt ggttaagttt | 240 |
| gctgatgcta ttgaagccca caagcaagaa ttcattgatt tgttgggtag agaagctggt | 300 |
| aaaccaccac aagctggtgg ttttgaattg atgttggtta tggaacacgt tagggaaact | 360 |
| ccaaagttga gaattggtga agttaagcca gaagataacg aagatagaac cgctgttgtt | 420 |
| agatacgttc caattggtgt tggtgttggt atagttccat ggaattttcc aatgttgttg | 480 |
| ggtattggta aagcttaccc agctatgttg gctggtaata cttttatttg gaagccatct | 540 |
| ccatacaccc catactctgc tttgaaattg gctgaaattg gtgctaaagt tttgccacca | 600 |
| ggtgttttac aagctttgtc tggtggtgat gatttgggtc caatgttgac tgctcatcca | 660 |
| gatgttgcca aagtttcttt tactggttct actgaaaccg gtaaaaagat tatggctgct | 720 |
| tgtgctgcta ctttgaagag agttactttg gaattgggtg gtaatgatgc tgctatcgtt | 780 |
| tgtgaagatg ttgatattcc aggtgttgct ggtaaggttg cttttttggc ttatgttcat | 840 |
| tctggtcaga tctgcatgaa catcaagaga atctacgttc acgaatccat ctacgacaag | 900 |
| ttcgtttccg aagttatcaa gttcttgcat gctttgaaaa ccggtgattt ctctgatcca | 960 |
| gaagcttttt ttggtccaat ccaaaacaag atgcagtacg aaaaattgca gaggttgtac | 1020 |
| gaacaaatcg ataagcaagg ttggaagtgt gcttttggtt ctgcttctcc agctacttct | 1080 |
| gaaaaaggtt attttgttcc accagtcttg gttgataatc caccagaaga ttctgaaatc | 1140 |
| gtccaaatgg aaccatttgg tccaatagtt ccagttatga agtggcaatc tgaagatgat | 1200 |
| gttattgcta gagctaacgc ttctgattat ggtttgggtg cttctgtttg gtctaaagat | 1260 |
| gttgctagag caagaagaat ggctgaatta ttggaagctg gttctgtttg ggttaacacc | 1320 |
| cattttgaag ttgctccaaa tgttcctttt ggtggtcata agcaatctgg tattggtatg | 1380 |
| gattggggtg aagttggttt gaaaggttgg tgtaatccac aagcttattg ggtcaaacat | 1440 |
| tccggttaa | 1449 |

SEQ ID NO: 202

| | |
|---|---|
| MSFPDEQKVD FQTFQNVINN QLSPTSESRH GICPSTEESL WESPVSTQDD VDRAVSAAKA | 60 |
| AYPAWRKLSW DERASYLVKF ADAIEAHKQE FIDLLGREAG KPPQAGGFEL MLVMEHVRET | 120 |
| PKLRIGEVKP EDNEDRTAVV RYVPIGVGVG IVPWNFPMLL GIGKAYPAML AGNTFIWKPS | 180 |
| PYTPYSALKL AEIGAKVLPP GVLQALSGGD DLGPMLTAHP DVAKVSFTGS TETGKKIMAA | 240 |
| CAATLKRVIL ELGGNDAAIV CEDVDIPGVA GKVAFLAYVH SGQICMNIKR IYVHESIYDK | 300 |
| FVSEVIKFLH ALKTGDFSDP EAFFGPIQNK MQYEKLQRLY EQIDKQGWKC AFGSASPATS | 360 |
| EKGYFVPPVL VDNPPEDSEI VQMEPFGPIV PVMKWQSEDD VIARANASDY GLGASVWSKD | 420 |
| VARARRMAEL LEAGSVWVNT HFEVAPNVPF GGHKQSGIGM DWGEVGLKGW CNPQAYWVKH | 480 |
| SG | 482 |

SEQ ID NO: 205

| | |
|---|---|
| atgtggtggc tgtttcgtgc cttgttttca tcaattttcc tgctttcaat cgttttaagt | 60 |
| attcctgttg cttttgatgt tggtgggaga gattcaggac ttgcctatag tttagctttg | 120 |
| ttcttattct acttcatcta ctctagttta gaacttctta cgcctgaaaa gtccagaagt | 180 |
| cgttatttct tatctggctt cttaagattg agccaatgga ttatcatacc tgcactatta | 240 |
| atttgggcgt taggtcagtt cgcggttgac gcagataaca ccaattgggt tgaacgtacc | 300 |
| gttggaggtc tgttcaattc caaatccacc tcttggagag aatggatgtt tggcaaggat | 360 |
| ggactggtgg aaactatcac tttaggcggc tgggataact tgttacgtta ttctggtcca | 420 |
| gtgttccaat tattagaggg attttgtaca cttcttgtaa tccaagctgc cggacaatta | 480 |
| accagatggc ttgtaaatag aggtcgttca gatacatggc taattgtatt gttagtgtta | 540 |
| agctcaagta tcatggcatc agctgtgtat tttctttggc gtgttgcaca gtttccccag | 600 |
| atcgggaatc tagacgcaac gttaataggt attgcgatga caaccgcagt atttttgtgt | 660 |
| gcgttcggca tcggttctgg caggggtaat cccattgaat catcattgtt gttcgcttac | 720 |
| attgtcttgt gtatttacca aatttttaca gactatctac catcagaaaa tgcagaccac | 780 |
| acgcaagatc atgatggctc agaaagcgat atccctcctc ttcctcctgt tatcatggct | 840 |
| agctacagca cgttccttca tatgttgggc tctttgccct ctgccgttca ttcatcattg | 900 |
| gcacttttgt atgctgcctt ccagactata actccatccg taattatttc actaacctat | 960 |
| aggagtcttg ttttttactg cgccactagg attataccta gcattagaga aagtggtgca | 1020 |
| caggctatga tgcaagaacc agactgggaa gatagcgaaa cagcttctaa atttttgggc | 1080 |
| tttttgagct ggttttcccc ctctatcttg atagctgtgt atacctcctt attacttcaa | 1140 |
| catttttcta cgagtgatgg tcctgatggt tggacgttga gaggcggaga tgttgagggt | 1200 |
| tctaattggc aatgggccaa cataggtctt accatggttt tgtacggagt cgaactgtac | 1260 |
| ctgggctctg atgagcatga tcattggaag gtggattaa | 1299 |

SEQ ID NO: 206

| | |
|---|---|
| MWWLFRALFS SIFLLSIVLS IPVAFDVGGR DSGLAYSLAL FLFYFIYSSL ELLTPEKSRS | 60 |
| RYFLSGFLRL SQWIIIPALL IWALGQFAVD ADNTNWVERT VGGLFNSKST SWREWMFGKD | 120 |
| GLVETITLGG WDNLLRYSGP VFQLLEGFCT LLVIQAAGQL TRWLVNRGRS DTWLIVLLVL | 180 |
| SSSIMASAVY FLWRVAQFPQ IGNLDATLIG IAMITAVELC AFGIGSGRGN PIESSLLFAY | 240 |
| IVLCIYQIFT DYLPSENADH TQDHDGSESD IPPLPPVIMA SYSTFLHMLG SLPSAVHSSL | 300 |
| ALLYAAFQTI TPSVIISLTY RSLVFYCATR IIPSIRESGA QAMMQEPDWE DSETASKFLG | 360 |
| FLSWFSPSIL IAVYTSLLLQ HFSTSDGPDG WTLRGGDVEG SNWQWANIGL TMVLYGVELY | 420 |
| LGSDEHDHWK VD | 432 |

SEQ ID NO: 207

| | |
|---|---|
| atggctgatt ctacattagc tgctaacggt aacagtttat tggaaactac aaaaacaaat | 60 |
| gcggcagctg cctaccaaag cgttgcgaac ggacccgttg cacagaatgt atacgatcac | 120 |
| acgcaaaagg catccaatga gttgtctaat ctagcagctg caaggagaac tccggctaat | 180 |
| ccagccgcta caggtcaacc attgacgcat tatcattctt ttttcagtga attactgagt | 240 |
| tggaataacc caagagcttc tgccatagct tacgttacaa ttattggtgc catttttacg | 300 |
| gctagatatc ttgatttgtt gagatgggga ttgaaagttt cttggatggt tttgggtgtt | 360 |
| actattcttg ccgaggtatt gggcaaggta attctaaaca atggactggc cacccaagtc | 420 |
| agacctagga ggtattatac agtacctaga gaaacactag atgctctaat cggcgatgtt | 480 |
| catgaactaa ttaatttttt cgtcatcgaa gcacaacgta tcatttttgc agaaaacgtc | 540 |
| tttgcaagtg cggctgcctt tattgctgct tttatatctt attttttggt gaaattagtt | 600 |
| ccctactggg gactagcagt tattggtacc actgttgcct tcgttgtccc attaatatac | 660 |
| acctcaaatc aagaattgat cgacgaacaa ctacaccatg ctagtgaact aataaatagc | 720 |
| caaacagcgc aaatacaatc cgttgcatct aaacaaatgg aacaagtttc caatatctcc | 780 |
| aaacaatatg caggagatta tagtggtaaa gtgcaagacc tgttaagagg aaagacgcct | 840 |
| agcaggcaga agatagacaa gcccgagcaa ccaattagcg ctaaacaacc ccaattcccc | 900 |
| agtccaccaa ccgaggatcc ggtgacagca acggaagctc ctcaaatacc tacccccgct | 960 |
| gcgcttaagg aagagcttaa tgctccaacc gcaatcgata ctgctgcacc tgaattaccc | 1020 |
| catgaggatg ttgtgccctc aaaagaacct atgttagcct cctaa | 1065 |

SEQ ID NO: 208

| | |
|---|---|
| MADSTLAANG NSLLETTKTN AAAAYQSVAN GPVAQNVYDH TQKASNELSN LAAARRTPAN | 60 |
| PAATGQPLTH YHSFFSELLS WNNPRASAIA YVTIIGAIFT ARYLDLLRWG LKVSWMVLGV | 120 |
| TILAEVLGKV ILNNGLATQV RPRRYYTVPR ETLDALIGDV HELINFFVIE AQRIIFAENV | 180 |
| FASAAAFIAA FISYFLVKLV PYWGLAVIGT TVAFVVPLIY TSNQELIDEQ LHHASELINS | 240 |
| QTAQIQSVAS KQMEQVSNIS KQYAGDYSGK VQDLLRGKTP SRQKIDKPEQ PISAKQPQFP | 300 |
| SPPTEDPVTA TEAPQIPTPA ALKEELNAPT AIDTAAPELP HEDVVPSKEP MLAS | 354 |

SEQ ID NO: 209

| | |
|---|---|
| MTERELHADV RRFYQHTSQT LTGLRPYPTE REVQDAAAAW QQKDNIENAI REAVRKGSPD | 60 |
| SGGITDIVIP LSAAEKRALI NEIDHSFSEN GMWMVIFTVS LSAFLQGFVQ SSQNGANLFA | 120 |
| DQWLKSQKHT VNSQFAYANA AVYFSAAVIG CPLAAPMSSL FGRRGVIIVA SFLIFAASVG | 180 |
| SACITLNDNA WLSLRSIRLI GGVGMGLKAT STPILAAETA VGSWRGSSVL LWQLWVSFGI | 240 |
| MMSFIVNICL NQIDDKNLKL RLILASPAVF ALMLMYTVAK CPESFRYYLM PGSRKYSPEK | 300 |
| AYASLLRLRN TKVGHNTSTH PFWLTPSFPF TICTVEQHLI QAVTATSSQR LVLDAAPKPR | 360 |
| TLVVGAVSHY VRQYWKILKV HRLRNAAITT GIVALSQQLS GINLMAFYGG TTLVGISPGN | 420 |
| QPTEDQISKA MLYNLIFGLS NFLFCLPAIH SIDVLGRRRV LLFTIPGMAL TLMAAAISFN | 480 |
| TANEDVRNGL VAFWIYFHTV FYSPGMGPVP FVLASESFPL AFRDTGASLA ISINLLFAGL | 540 |
| LAWLQPLLVT GIRFGGILGV FAGLNVVAFA LIFLLMEETS GVPLESLGSV FDQSKKDLIH | 600 |
| FQLFKFLPWF GRFILGRSSL AERPERTVDL SPSSVTAASV TDDDDEERIW NSDTVSSGVR | 660 |
| LADMLGGNGR G | 671 |

SEQ ID NO: 210

| | |
|---|---|
| MSSPLDAAGL AIATTELCRN LATGLYFIIR EIRDASKDAE IMQDTLAALH TRLDQVRALF | 60 |
| DANVPQSPLE KDYRNSIDRT LENIHRDLSL LTSKLHIDVI LEAKGSKRLE AWYVLQRKFQ | 120 |
| SDDIRNIKQR LAGSEELLQS HFEMLSIYIS YRIRDEVIDF KAFVRPILEK LLFHATLTEE | 180 |
| RQRYQAAESR SIKRLQHVTN ALGTGNTFPG EESDFEYHDA FKTWKDKSEA MIMSIADPPW | 240 |
| HQVSNSNYVP SIRNESRDGA SILPTVRDFR EKNGMYPSLR VTPNLSHVEE ILDESLHQDV | 300 |
| TEDLINWCKE QGFPVNVSNF RYDLIWEAAP VALKGTSPMH QAIKTNNMVV LEKMLSRDCN | 360 |
| IEVRLEDGSQ DPTPLLLAGS ELNAVAVKLL LTKGAKADAT DRIGKTGLHL CQSPKFEGRR | 420 |
| VAKLLLGDSR AEALDVNAQD QFGMTAAHIA ARVGDVKMLE YLLLDQYGKK VADANAQQQD | 480 |
| GSTPLMVALK SNIANKKQVI DVLSRCSDLS IKNKNGEDAK EVAAKHSPKD VRKYLLAHLD | 540 |
| QDSTRSRRIS ESTIVVSGIS VQMREESCSG CRRHCPQFTD CKLSIGDSAF SQDWKRSLRK | 600 |
| YSSDQSSIAM GSSSSIRQAR | 620 |

SEQ ID NO: 211

| | |
|---|---|
| MSGRESIAAA PLPEPEPYSV FDKRQTALIV TIVSIAATFS GFASNIYFPA LPTIAKDLNV | 60 |
| SIELINLTVT SYLIFQGLAP SLWGPISDVK GRRVAYLLTF IVFLGACIGL AEAKNYATMV | 120 |
| VLRCVQSTGS ASTIAIGSGV IGDITTRDNR GGLMGIFQAG LLVPVAVGPI IGGALAGSLG | 180 |
| WRSIFWFLTI YSGVFLIFLV LLLPETLRSI VGNGSREPKH VMAKYPLRVY QKTTKVKWIH | 240 |
| DATSPSPTEK KRIDITGPFR ILISKQAAPI IVFLAVYYAV WQMSITAMSS LFKDKYGLTE | 300 |
| TEIGLTFIAN GVGSMVGTLI TGKILNMDYR RFKARHDARI ASGSKENDVE TVNTRKNQEN | 360 |
| DFPLETARLR LVPVFSLLQC ASILLFGWTI QYPKQVHIAV PIISTFITGW SAVSMQSVVM | 420 |
| TYLVDVFHDR SAAASASLNL ARCLFAAGGT SFVMPLINSI GVGLAFTVCV VVQGVALVSL | 480 |
| AVQWKLGAKW RREAEDARSE P | 501 |

SEQ ID NO: 212

| | |
|---|---|
| atgttacgtt catctcctcc accaagcctg cccagagacg ccccaagcac tgtttttaaa | 60 |
| acttatacac cacacacgtt gttaccattt aacggagaag aggaccgtcc tgtttttctg | 120 |
| gccgttagag gcagagtctt tgatgtgtcc cctggcagaa atttttatgg tccaggaggt | 180 |
| ccctactcta attttgctgg tcgtgatgca tctagagggt tagcctgtgg tagcttcgat | 240 |
| gaagatatgt tgaccaagga tctagatggc ccactagata aactagaagg tttagacgcg | 300 |
| gaacaaatgg aagctttaca aggatgggag gaaagatttc tggaaaaata caatgtcgtg | 360 |
| ggtaaacttg tttctgttca ggattatgaa tctcagaagg cttaa | 405 |

SEQ ID NO: 213

| | |
|---|---|
| MLRSSPPPSL PRDAPSTVFK TYTPHTLLPF NGEEDRPVFL AVRGRVFDVS PGRNFYGPGG | 60 |
| PYSNFAGRDA SRGLACGSFD EDMLTKDLDG PLDKLEGLDA EQMEALQGWE ERFLEKYNVV | 120 |
| GKLVSVQDYE SQKA | 134 |

SEQ ID NO: 214

| | |
|---|---|
| atggagcatg ttgaacaaca catggctcaa caagcttccc aagaaacagc gtcattgttc | 60 |
| acaccattaa acttaatttt gctgtctgct gttttataca ccacttattc catgttacgt | 120 |
| tcatctcctc caccaagcct gcccagagac gccccaagca ctgtttttaa aacttataca | 180 |
| ccacacacgt tgttaccatt taacggagaa gaggaccgtc ctgtttttct ggccgttaga | 240 |
| ggcagagtct ttgatgtgtc ccctggcaga aatttttatg gtccaggagg tccctactct | 300 |
| aattttgctg gtcgtgatgc atctagaggg ttagcctgtg gtagcttcga tgaagatatg | 360 |
| ttgaccaagg atctagatgg cccactagat aaactagaag gtttagacgc ggaacaaatg | 420 |
| gaagctttac aaggatggga ggaaagattt ctggaaaaat acaatgtcgt gggtaaactt | 480 |
| gtttctgttc aggattatga atctcagaag gcttaa | 516 |

SEQ ID NO: 215

| | |
|---|---|
| MEHVEQHMAQ QASQETASLF TPLNLILLSA VLYTTYSMLR SSPPPSLPRD APSTVFKTYT | 60 |
| PHTLLPFNGE EDRPVFLAVR GRVFDVSPGR NFYGPGGPYS NFAGRDASRG LACGSFDEDM | 120 |
| LTKDLDGPLD KLEGLDAEQM EALQGWEERF LEKYNVVGKL VSVQDYESQK A | 171 |

SEQ ID NO: 216

| | |
|---|---|
| atggctggca agttcgaacc caaagtgccc gttaatttgg acccacctaa agatgacata | 60 |
| atctcaaggg aagagttagc aaaggcaaac ggtgctgatg ggaataagtg ttatgttgca | 120 |
| attaaaggca aggtgtatga cgtaaccggc aacaaagcct acttgccagg cgcaagctat | 180 |
| aatgtgtttg ctggcaaaga tgcctcaaga gctttgggta aaaccagcac caaacctgag | 240 |
| gatgctaggc ctgaatggca agacttagat gagaaagaaa agggtgtctt aaacgactgg | 300 |
| attactttct ttagcaaaag atacaatgtt gtgggggttg tggaaggcgc aacaaacatg | 360 |
| gattag | 366 |

SEQ ID NO: 217

| | |
|---|---|
| MAGKFEPKVP VNLDPPKDDI ISREELAKAN GADGNKCYVA IKGKVYDVTG NKAYLPGASY | 60 |
| NVFAGKDASR ALGKTSTKPE DARPEWQDLD EKEKGVLNDW ITFFSKRYNV VGVVEGATNM | 120 |
| D | 121 |

SEQ ID NO: 218

| | |
|---|---|
| atggctgacg aatcaacact tcgtcaaaga aaaccgcaac cgaagaacga aaccgaaagt | 60 |
| gaagtttctc gtcctagcac acctactaaa aaatcaaaaa agagatcatc cgcaaaagtt | 120 |
| gacgaggaag atccatggga tggttattcc ccatacttag atgtggtgag agtaattagc | 180 |
| tttattattg ttgcatctat gggattgagc tatgtcattt caggtggcga gtcattctgg | 240 |
| tggggtcata aaaacaagcc gaattggatg acacaacgtt tctacaaaga tttgatatta | 300 |
| ggacccccac ctccagtgta catgactttg gaggaacttt ctttacatga cggtactgat | 360 |
| cctgacagac cgcttttact tgcgatcaac ggtacaattt atgacgtgtc aaatggtagg | 420 |
| agaatgtacg gcccaggtgg ttcctattct tactttgcag ctacggatgc tgcaagggga | 480 |
| ttcgtcaccg gctgttttgc tgaagatcaa actgcagact tgagaggtta tgaagaaact | 540 |
| tttcttccac tggacgatcc agaagttgac agtcactgga ctcccgaagc tctggcagaa | 600 |
| ctgaagatca aagagcgtga agaagctaaa aaaagggctg atgctgcttt acaacactgg | 660 |
| gttgattttt ttgcaaattc caaaaaatac accaaagtcg gttatgttta tagagagccg | 720 |
| gggtggcttg aaaaagagaa accaaagaaa ttatgcgatc aggcccaaag atcaagaaag | 780 |
| accagaaaaa ttccaaaaaa ggattaa | 807 |

SEQ ID NO: 219

| | |
|---|---|
| MADESTLRQR KPQPKNETES EVSRPSTPTK KSKKRSSAKV DEEDPWDGYS PYLDVVRVIS | 60 |
| FIIVASMGLS YVISGGESFW WGHKNKPNWM TQRFYKDLIL GPPPPVYMTL EELSLHDGTD | 120 |
| PDRPLLLAIN GTIYDVSNGR RMYGPGGSYS YFAATDAARG FVTGCFAEDQ TADLRGYEET | 180 |
| FLPLDDPEVD SHWTPEALAE LKIKEREEAK KRADAALQHW VDFFANSKKY TKVGYVYREP | 240 |
| GWLEKEKPKK LCDQAQRSRK TRKIPKKD | 268 |

SEQ ID NO: 220

| | |
|---|---|
| atggcttcag aaaaagaaat taggagagag agattcttga acgttttccc taaattagta | 60 |
| gaggaattga acgcatcgct tttggcttac ggtatgccta aggaagcatg tgactggtat | 120 |
| gcccactcat tgaactacaa cactccaggc ggtaagctaa atagaggttt gtccgttgtg | 180 |
| gacacgtatg ctattctctc caacaagacc gttgaacaat tggggcaaga agaatacgaa | 240 |
| aaggttgcca ttctaggttg gtgcattgag ttgttgcagg cttacttctt ggtcgccgat | 300 |
| gatatgatgg acaagtccat taccagaaga ggccaaccat gttggtacaa ggttcctgaa | 360 |
| gttggggaaa ttgccatcaa tgacgcattc atgttagagg ctgctatcta caagcttttg | 420 |
| aaatctcact tcagaaacga aaaatactac atagatatca ccgaattgtt ccatgaggtc | 480 |
| accttccaaa ccgaattggg ccaattgatg gacttaatca ctgcacctga agacaaagtc | 540 |
| gacttgagta agttctccct aaagaagcac tccttcatag ttactttcaa gactgcttac | 600 |
| tattctttct acttgcctgt cgcattggcc atgtacgttg ccggtatcac ggatgaaaag | 660 |
| gatttgaaac aagccagaga tgtcttgatt ccattgggtg aatacttcca aattcaagat | 720 |
| gactacttag actgcttcgg taccccagaa cagatcggta agatcggtac agatatccaa | 780 |
| gataacaaat gttcttgggt aatcaacaag gcattggaac ttgcttccgc agaacaaaga | 840 |
| aagactttag acgaaaatta cggtaagaag gactcagtcg cagaagccaa atgcaaaaag | 900 |
| attttcaatg acttgaaaat tgaacagcta taccacgaat atgaagagtc tattgccaag | 960 |
| gatttgaagg ccaaaatttc tcaggtcgat gagtctcgtg gcttcaaagc tgatgtctta | 1020 |
| actgcgttct tgaacaaagt ttacaagaga agcaaaggtt ctagtactgg ttcatctaca | 1080 |
| tctactggaa tggtcgcaca aactttcaac ctggatacct acttatccca aagacaacaa | 1140 |
| caagttgaag aggccctaag tgctgctctt gtgccagctt atcctgagag aatatacgaa | 1200 |
| gctatgagat actccctcct ggcaggtggc aaaagattaa gacctatctt atgtttagct | 1260 |
| gcttgcgaat tggcaggtgg ttctgttgaa caagccatgc caactgcgtg tgcacttgaa | 1320 |
| atgatccata caatgtcact aattcatgat gacctgccag ccatggataa cgatgatttc | 1380 |
| agaagaggaa agccaactaa tcacaaggtg ttcggggaag atatagccat cttagcgggt | 1440 |
| gatgcgcttt tagcttacgc ttttgaacat attgcttctc aaacaagagg agtaccacct | 1500 |
| caattggtgc tacaagttat tgctagaatc ggacacgccg ttgctgcaac aggcctcgtt | 1560 |
| ggaggccaag tcgtagacct tgaatctgaa ggtaaagcta tttccttaga aacattggag | 1620 |
| tatattcact cacataagac tggagccttg ctggaagcat cagttgtctc aggcggtatt | 1680 |
| ctcgcagggg cagatgaaga gcttttggcc agattgtctc attacgctag agatataggc | 1740 |
| ttggcttttc aaatcgtcga tgatatcctg gatgttactg ctacatctga acagttgggg | 1800 |
| aaaaccgctg gtaaagacca ggcagccgca aaggcaactt atccaagtct attgggttta | 1860 |
| gaagcctcta gacagaaagc ggaagagttg attcaatctg ctaaggaagc cttaagacct | 1920 |
| tacggttcac aagcagagcc actcctagcg ctggcagact tcatcacacg tcgtcagcat | 1980 |
| taa | 1983 |

SEQ ID NO: 221

| | |
|---|---|
| MASEKEIRRE RFLNVFPKLV EELNASLLAY GMPKEACDWY AHSLNYNTPG GKLNRGLSVV | 60 |
| DTYAILSNKT VEQLGQEEYE KVAILGWCIE LLQAYFLVAD DMMDKSITRR GQPCWYKVPE | 120 |
| VGEIAINDAF MLEAAIYKLL KSHFRNEKYY IDITELFHEV TFQTELGQLM DLITAPEDKV | 180 |
| DLSKFSLKKH SFIVTFKTAY YSFYLPVALA MYVAGITDEK DLKQARDVLI PLGEYFQIQD | 240 |
| DYLDCFGTPE QIGKIGTDIQ DNKCSWVINK ALELASAEQR KTLDENYGKK DSVAEAKCKK | 300 |
| IFNDLKIEQL YHEYEESIAK DLKAKISQVD ESRGFKADVL TAFLNKVYKR SKGSSTGSST | 360 |
| STGMVAQTFN LDTYLSQRQQ QVEEALSAAL VPAYPERIYE AMRYSLLAGG KRLRPILCLA | 420 |
| ACELAGGSVE QAMPTACALE MIHTMSLIHD DLPAMDNDDF RRGKPTNHKV FGEDIAILAG | 480 |
| DALLAYAFEH IASQTRGVPP QLVLQVIARI GHAVAATGLV GGQVVDLESE GKAISLETLE | 540 |
| YIHSHKTGAL LEASVVSGGI LAGADEELLA RLSHYARDIG LAFQIVDDIL DVTATSEQLG | 600 |
| KTAGKDQAAA KATYPSLLGL EASRQKAEEL IQSAKEALRP YGSQAEPLLA LADFITRRQH | 660 |

SEQ ID NO: 222

| | |
|---|---|
| atgacagaga gggagctcca cgcggatgtg cgaaggttct atcaacacac ttctcaaact | 60 |
| ctaaccggcc tgcgacctta tcccaccgag cgagaagtcc aagatgcagc cgcggcgtgg | 120 |
| cagcaaaagg acaacatcga gaatgccatc cgcgaagcgg ttcgaaaggg cagcccagat | 180 |
| agcggcggca ctacggacac cgtcataccc ctcagtgccg ccgagaaacg cgctctgatc | 240 |
| aacgagattg accattcgtt ctctgagaac gggatgtgga tggtcatctt cactgtcagt | 300 |
| ctgagtgcct ttctccaggg ctttgtacag agtagtcaga acggtgcaaa tctctttgct | 360 |
| gatcagtggc ttaagtctca gaagcatact gtcaactccc agttcgctta tgccaacgca | 420 |
| gctgtttact tcagcgctgc tgttatagga tgtccactgg ctgcaccgat gagttcactg | 480 |
| tttggtcgcc gtggtgtcat tattgtcgcc tcatttctca tctttgcggc atccgttggc | 540 |
| tcggcttgca ttacactcaa tgacaacgca tggctgtctc ttaggagcat cagactaatc | 600 |
| ggcggtgtcg gcatgggctt aaaggctact agcaccccca tcctcgcagc ggaaacggca | 660 |
| gttggctcgt ggagaggctc ttcagttctg ttatggcagc tatgggtctc ttttggcatc | 720 |
| atgatgtctt ttattgtcaa tatttgcttg aaccagattg acgacaagaa tctaaagctc | 780 |
| cggttgattc tggcgtctcc agcagtgttt gcgcttatgc tgatgtatac tgtcgccaaa | 840 |
| tgccctgagt cattccgcta ctacttgatg ccaggttcga gaaagtatag ccctgagaag | 900 |
| gcatatgcct cgttgctacg attgcgcaac accaaggtcg gtcacaacac ttctacacat | 960 |
| cccttttggc ttaccccttc gttccccttc acaacctgca ccgtcgaaca acatctgata | 1020 |
| caagcggtca cagctacaag cagtcagaga cttgtacttg acgccgcccc gaaacctcga | 1080 |
| accctagtag ttggagctgt cagtcactac gtgcgacaat actggaagat cctgaaagtc | 1140 |
| catcgccttc ggaatgcagc tattacaaca gggattgtgg ctttgtcgca gcaactttct | 1200 |
| ggaattaacc tcatggcgtt ctacggtggg acaacacttg taggtattag tccaggcaat | 1260 |
| cagccaacag aagatcaaat ctccaaggcc atgctgtaca acttgatctt tggtctgtcg | 1320 |
| aacttcttat tctgcttacc cgccatccat tccatagacg ttctgggaag aaggagggtt | 1380 |
| ctactcttca caatcccagg tatggcctta accttgatgg cagcggctat aagcttcaat | 1440 |
| acggcaaatg aggatgtgag aaacggactt gtagccttct ggatctactt tcacacagta | 1500 |
| ttttatagcc cgggaatggg gccagtgccg ttcgtgttag cttcggaaag ctttcctttg | 1560 |
| gcctttcgtg acaccggcgc atcgcttgca atatccatca accttctatt cgctggcctc | 1620 |
| ctggcatggc tgcaacccct actggtcact ggtattagat tcgggggaac acttggggtg | 1680 |
| tttgctggct tgaacgtcgt tgcctttgct ctcatctttc tcctgatgga ggaaaccagc | 1740 |
| ggcgtacctc ttgagtctct aggatctgtc ttcgaccagt cgaagaagga tctgatccac | 1800 |
| ttccaactct tcaagttttt accatggttc ggtcggttca ttcttggtag gagtagtctt | 1860 |
| gccgaaagac cagaacgtac tgtcgacttg agtccgagct cggtgacagc tgcttcggtc | 1920 |
| actgatgatg acgatgagga acgcatttgg aatagcgata ctgtttcaag tggggtgagg | 1980 |
| ctcgccgata tgttgggggg aaacggaaga ggctga | 2016 |

SEQ ID NO: 223

| | |
|---|---|
| atgtccttca ttaaaaactt gttatttgga ggtgttaaaa caagtgagga tccaaccggg | 60 |
| ctcacaggta acggggcctc aaacacaaac gattctaata aaggtagtga accggtagta | 120 |
| gcgggtaatt tctttcctag gacgctttcc aaatttaacg gccacgacga tgaaaaaata | 180 |
| tttattgcta ttaggggcaa agtatacgac tgcacaagag ggaggcagtt ttacggtcca | 240 |
| agcgggccat acactaactt tgcaggccat gatgcgtcgc gtggtcttgc attgaactcc | 300 |
| ttcgatctgg acgttattaa agattgggat cagcctatcg atcccttaga tgatctgaca | 360 |
| aaagaacaga ttgacgcact ggatgagtgg caagagcatt ttgagaataa gtacccatgc | 420 |
| attggtactc tgattccgga gcctggcgtg aacgtatga | 459 |

SEQ ID NO: 224

| | |
|---|---|
| MSFIKNLLFG GVKTSEDPTG LTGNGASNTN DSNKGSEPVV AGNFFPRTLS KFNGHDDEKI | 60 |
| FIAIRGKVYD CTRGRQFYGP SGPYTNFAGH DASRGLALNS FDLDVIKDWD QPIDPLDDLT | 120 |
| KEQIDALDEW QEHFENKYPC IGTLIPEPGV NV | 152 |

SEQ ID NO: 225

| | |
|---|---|
| atgtcaagtc cattagacgc ggctggtcta gctattgcta caacagagtt gtgcagaaac | 60 |
| ttggcgactg ggctgtactt cattatcaga gagatcagag atgcttctaa agatgcagaa | 120 |
| attatgcaag atacattagc agcgttacat accagattgg accaagttag agctttattt | 180 |
| gacgccaacg ttccacagag tccattggaa aaagattata gaaactccat agacagaaca | 240 |
| ttagagaata ttcacagaga tctgtcttta ttaactagta aattgcatat tgacgtcatc | 300 |
| ttagaagcga aaggttcaaa aagattagag gcctggtacg tcttacagcg taaatttcaa | 360 |
| tcagatgata tcaggaatat caaacagaga cttgcagggt ccgaggaact gcttcagtct | 420 |
| catttcgaaa tgttgtcaat atatatctct tacaggacca gagacgaggt aacagacttt | 480 |
| aaagctttcg tcagaccgat tttagagaag ctgttgtttc atgcaacgtt gacagaggaa | 540 |
| agacaaagat accaagcagc tgagtcaaga tctattaaaa gattacaaca cgtcactaat | 600 |
| gccctaggca ctggtaatac attccctgga gaagaatcag atttcgaata tcatgacgca | 660 |
| tttaagactt ggaaagacaa gtctgaagct atgattatgt ctatcgcaga tccaccttgg | 720 |
| catcaagtgt caaattctaa ctacgtccca tcaattcgta atgaatctcg tgatggcgca | 780 |
| tcaatattgc caactgtacg tgattttaga gaaaaaaacg gcatgtatcc cagcttgcgt | 840 |
| gttacaccca acttaagcca cgttgaagag atcttagatg aatcattaca tcaggatgtt | 900 |
| acagaagatt tgattaattg gtgtaaagag cagggattcc cagtcaacgt ctcaaacttt | 960 |
| aggtacgatt taatttggga ggcggcacct gtggccctta aaggaacgtc acctatgcat | 1020 |
| caagccataa aaactaataa tatggttgtt ttggaaaaaa tgttgtccag agattgtaac | 1080 |
| atagaagtca ggctggagga cggttctcaa gatccaaccc cactactatt agctggctct | 1140 |
| gaactaaatg cagttgctgt taagctgtta ttaaccaaag gtgccaaagc agatgctaca | 1200 |
| gatagaactg gaaaaacagg tctacatttg tgccaatccc ctaaattcga gggtagaaga | 1260 |
| gtggctaaac tattgttggg tgactctaga gctgaggcgt tagatgttaa tgcacaagat | 1320 |
| cagtttggca tgactgcagc tcacatcgcc gctagagtag gcgatgttaa aatgttagag | 1380 |
| tacctactat tggaccagta cggaaagaag gtagctgatg ccaacgccca acagcaagat | 1440 |
| ggttcaaccc cgttgatggt cgcattgaaa agcaacatcg caaataaaaa acaagtgatt | 1500 |
| gatgtcttgt ctaggtgctc cgatttgtca attaaaaaca agaacggtga agacgcgaaa | 1560 |
| gaggtagccg caaagcatag tccgaaagat gtaagaaagt atcttttagc tcatctagac | 1620 |
| caagattcaa ctaggtctcg taggatctct gaatccacaa cagttgtgtc cggaatttct | 1680 |
| gtgcagatga gagaggaatc ttgttcaggg tgccgtaggc attgtccaca atttactgac | 1740 |
| tgtaaattgt caataggtga ctctgcattt tcccaggatt ggaagagatc cttgcgtaag | 1800 |
| tactcctctg atcaatcaag tatagcgatg gggtctagca gttccattcg tcaggccaga | 1860 |
| taa | 1863 |

SEQ ID NO: 226

| | |
|---|---|
| at tttgcca agttcgacat gctagaagaa gaggctagag cacttgttag aaaagtaggc | 60 |
| aatgctgttg atcccattta cggattcagt acaacctcct gccaaattta cgatacggca | 120 |
| tgggctgcta tgattagtaa ggaagaacat ggagataagg tttggttgtt tccagaatct | 180 |
| ttcaaatact tactagaaaa gcaaggtgag gatggtagtt gggaaaggca tccaaggagt | 240 |
| aaaacagtag gggtgctaaa tacagctgca gcgtgcttag ctttattgcg tcacgttaag | 300 |
| aatccacttc agcttcaaga tatagcagct caggatatag aacttagaat tcaaagaggt | 360 |
| ctaaggagtc tagaagaaca gcttattgcg tgggacgatg tccttgacac aaatcacatt | 420 |
| ggtgtcgaga tgattgtccc ggctctactt gattaccttc aagctgaaga tgaaaatgta | 480 |
| gattttgaat tcgagtcaca ctctttgctt atgcagatgt ataaggagaa gatggcccgt | 540 |
| ttctcaccag aatccttata tcgtgcaagg cccagttctg ctctgcataa tttagaagcg | 600 |
| ctaattggta agcttgattt tgataaggtg ggtcaccatc tgtacaatgg tagtatgatg | 660 |
| gcctcacctt catctactgc ggctttccta atgcatgcct caccttggtc acacgaggca | 720 |
| gaagcatatc taagacatgt ttttgaagct ggcactggga agggctccgg cggatttccc | 780 |
| ggtacatatc ctactacata ctttgaatta aattgggttc tatcaacctt gatgaaatca | 840 |
| gggtttactt tgtcagatct tgagtgcgat gaattatcaa gcatagcaaa cactatagca | 900 |
| gagggtttcg aatgcgacca tggagtgatc gggtttgccc caagagctgt tgatgttgat | 960 |
| gatactgcaa aaggactact tacccttacg ttattgggca tggacgaagg ggtgagccca | 1020 |
| gcacccatga ttgcgatgtt tgaagctaaa gatcatttcc taacgttcct gggtgaaaga | 1080 |
| gatccttcat tcaccagtaa ttgtcacgtt ctattatctt tactacaccg taccgactta | 1140 |
| ctgcaatatc tgccacagat tagaaaaact acaacttttc tatgcgaagc ctggtgggct | 1200 |
| tgtgatggtc aaataaaaga taaatggcat cttagtcatc tatatccgac tatgttgatg | 1260 |
| gtccaggcat ttgctgagat ccttctgaag tccgcagaag gtgaaccatt gcacgatgct | 1320 |
| ttcgacgcag ccactttgtc tagagtctca atttgtgttt ttcaagcttg tcttcgtact | 1380 |
| ttgttggcac aatcacaaga tggtagctgg cacggtcaac cggaggcgtc ttgctatgca | 1440 |
| gtattaacac tagctgagag cgggagactt gttcttttgc aagcgcttca accacagatt | 1500 |
| gcagccgcca tggagaaggc tgcggatgtt atgcaagcgg gaagatggtc ctgtagcgat | 1560 |
| cacgattgtg attggacttc caaaacagcc taccgtgtag atttggtggc tgcagcttac | 1620 |
| aggctggcag ccatgaaggc ttcctctaac ttgaccttta ctgttgatga caatgtgtca | 1680 |
| aagaggtcca acggttttca acagttggtg ggaagaacag atctattctc tggagtgcca | 1740 |
| gcatgggaac tgcaagcatc attcttagaa agtgcgcttt ttgttcccct attaaggaac | 1800 |
| catagactag atgtgtttga tagggacgac ataaaagttt caaaggatca ttatttagat | 1860 |
| atgattccat ttacgtgggt aggttgtaat aacaggtcta gaacatacgt gagtacgtcc | 1920 |
| ttcttattcg atatgatgat catctctatg ttaggttatc aaatagacga gttcttcgag | 1980 |
| gccgaagctg cacctgcttt cgcacaatgt ataggccaat tacaccaagt cgttgacaaa | 2040 |
| gtcgttgatg aagtcatcga cgaagttgtc gacaaggtgg tcggcaaggt tgtgggcaag | 2100 |
| gttgtaggta aggtggtgga cgagcgtgtc gactctccga cccatgaagc aatagcgata | 2160 |
| tgcaatattg aagcctcttt gaggagattt gtggatcatg ttctacatca ccaacatgta | 2220 |
| ttacacgcaa gccaacaaga gcaagacatt ttatggcgtg aattgagagc ttttttacac | 2280 |
| gctcacgttg tgcaaatggc tgacaattct actctggcgc ctcctggcag gacattcttc | 2340 |
| gactgggtta ggacaactgc tgctgatcat gtagcctgcg cttactcttt cgcattcgcc | 2400 |
| tgctgtatta cttccgcaac gatcggacag ggccaatcta tgttcgctac tgttaatgag | 2460 |
| ctgtatcttg ttcaagcagc agctagacat atgactacca tgtgcagaat gtgcaatgat | 2520 |
| attggtagtg ttgataggga tttcattgaa gccaatataa attctgttca tttccctgaa | 2580 |
| ttttctactc taagccttgt ggcagataag aaaaaagccc ttgcccgttt agcagcttat | 2640 |
| gaaaaatctt gtttgaccca taccttagat caatttgaaa atgaagttct acaatcccca | 2700 |
| agagtttcat ccgcagcctc cggcgatttt aggacaagga aagtggcagt ggtaaggttc | 2760 |
| ttcgcggatg tgaccgattt ttatgaccag ttatatattc tgagagatct ttcatcttct | 2820 |
| ttaaagcatg tcggcaccta a | 2841 |

SEQ ID NO: 227:

| | |
|---|---|
| MFAKFDMLEE EARALVRKVG NAVDPIYGFS TTSCQIYDTA WAAMISKEEH GDKVWLFPES | 60 |
| FKYLLEKQGE DGSWERHPRS KTVGVLNTAA ACLALLRHVK NPLQLQDIAA QDIELRIQRG | 120 |
| LRSLEEQLIA WDDVLDTNHI GVEMIVPALL DYLQAEDENV DFEFESHSLL MQMYKEKMAR | 180 |
| FSPESLYRAR PSSALHNLEA LIGKLDFDKV GHHLYNGSMM ASPSSTAAFL MHASPWSHEA | 240 |
| EAYLRHVFEA GTGKGSGGFP GTYPTTYFEL NWVLSTLMKS GFTLSDLECD ELSSIANTIA | 300 |
| EGFECDHGVI GFAPRAVDVD DTAKGLLTLT LLGMDEGVSP APMIAMFEAK DHFLTFLGER | 360 |
| DPSFTSNCHV LLSLLHRTDL LQYLPQIRKT TTFLCEAWWA CDGQIKDKWH LSHLYPTMLM | 420 |
| VQAFAEILLK SAEGEPLHDA FDAATLSRVS ICVFQACLRT LLAQSQDGSW HGQPEASCYA | 480 |
| VLTLAESGRL VLLQALQPQI AAAMEKAADV MQAGRWSCSD HDCDWTSKTA YRVDLVAAAY | 540 |
| RLAAMKASSN LTFTVDDNVS KRSNGFQQLV GRTDLFSGVP AWELQASFLE SALFVPLLRN | 600 |
| HRLDVFDRDD IKVSKDHYLD MIPFTWVGCN NRSRTYVSTS FLFDMMIISM LGYQIDEFFE | 660 |
| AEAAPAFAQC IGQLHQVVDK VVDEVIDEVV DKVVGKVVGK VVGKVVDERV DSPTHEAIAI | 720 |
| CNIEASLRRF VDHVLHHQHV LHASQQEQDI LWRELRAFLH AHVVQMADNS TLAPPGRTFF | 780 |
| DWVRTTAADH VACAYSFAFA CCITSATIGQ GQSMFATVNE LYLVQAAARH MTTMCRMCND | 840 |
| IGSVDRDFIE ANINSVHFPE FSTLSLVADK KKALARLAAY EKSCLTHTLD QFENEVLQSP | 900 |
| RVSSAASGDF RTRKVAVVRF FADVTDFYDQ LYILRDLSSS LKHVGT | 946 |

SEQ ID NO: 228:

| | |
|---|---|
| atgcctggta agatagaaaa tggcaccccg aaagatttaa aaactggtaa tgattttgtg | 60 |
| tctgccgcaa aatcattgct tgacagggct tttaaaagcc atcacagtta ttacggttta | 120 |
| tgctccacca gctgtcaggt ttacgatact gcgtgggtgg cgatgattcc aaaaacaaga | 180 |
| gacaatgtga agcaatggct atttccggag tgtttccatt acttgctgaa aacccaagct | 240 |
| gctgatggca gctggggttc tttgccaact acacaaactg caggtattct ggatactgca | 300 |
| tctgctgtac ttgccctgtt atgccacgct caggaaccat tacaaatctt agatgtttca | 360 |
| ccagacgaga tgggtttgcg tattgaacat ggggtgactt ctcttaagag acaattggct | 420 |
| gtttggaacg atgtcgagga cacaaatcac ataggtgtag aattcattat cccagcttta | 480 |
| cttagcatgt tggaaaagga attggatgtt ccctcattcg aatttccttg tcgttcaatt | 540 |
| ctggaaagaa tgcacgggga aaaacttggg cacttcgatc ttgaacaagt ctacggtaaa | 600 |
| ccgtcatcct tgttacactc tctagaggct tttttaggta aattggactt cgataggttg | 660 |
| tctcatcacc tataccacgg ttccatgatg gctagcccgt catctacggc tgcttacttg | 720 |
| attggtgcca caaaatggga tgatgaggca gaagattatc ttcgtcatgt tatgaggaac | 780 |
| ggcgccggtc acggcaacgg tggtatatct ggtacattcc cgactacaca cttcgagtgc | 840 |
| tcatggataa tagcaacttt actaaaagta ggttttacat taaaacagat tgatggtgat | 900 |
| ggcttgaggg ggctatctac tatcttactt gaagcattga gggatgagaa tggggtgata | 960 |
| ggattcgctc caagaacagc agatgtagat gatacagcta aagcgttgtt ggctttgagc | 1020 |
| ttggttaatc aaccagtttc ccctgacatc atgatcaagg ttttcgaggg gaaagatcac | 1080 |
| tttaccactt ttggcagcga aagggatcct tctttaacat ccaacttaca tgttctttta | 1140 |
| tctttgttga agcagtcaaa tttgagtcag taccatcccc agatcttaaa gaccacacta | 1200 |
| tttacatgta gatggtggtg gggttccgat cactgcgtaa aagataagtg gaacctttct | 1260 |
| catctatatc ctacaatgtt attagtcgag gcattcacgg aagttcttca cttaattgac | 1320 |
| ggcggtgaac tatccagcct atttgatgaa tcctttaagt gcaaaatagg tttatcaatc | 1380 |
| tttcaagcag tattgcgtat catactaaca caagataatg atggtagctg gcgtggatat | 1440 |
| agagaacaaa catgttacgc tatcttggct ttagttcagg ctagacacgt ctgtttcttc | 1500 |
| actcatatgg tagacagatt gcagagttgc gtggacagag gtttttcctg gcttaaatcc | 1560 |
| tgttcatttc attctcaaga tttaacgtgg acttctaaga cagcatatga agttgggttc | 1620 |
| gtagctgagg catataaatt agctgcattg cagtcagcgt ctcttgaagt gccggcagcc | 1680 |
| accatcggac atagtgttac gagtgcagta ccttcatctg atcttgaaaa atatatgagg | 1740 |
| ttagttagaa aaacggcctt gttttccccg ttggatgagt ggggtcttat ggcttccatt | 1800 |
| atagaatcta gtttttttgt gccactttta caagcccaga gagttgagat ttacccaaga | 1860 |
| gacaacatta aggttgatga ggacaagtac ttgagcatta tcccattcac ctgggtcgga | 1920 |
| tgtaacaacc gttctagaac tttcgcctct aacagatggt tatatgatat gatgtatttg | 1980 |
| tcattgttgg gttaccaaac tgatgagtac atggaagcag ttgccgggcc cgtgttcgga | 2040 |
| gacgtgtctt tattgcacca aactatagac aaggtgatag ataatactat gggtaatttg | 2100 |
| gctagagcaa acggtacggt tcatagtggt aatggtcacc agcacgaatc tccgaatata | 2160 |
| ggtcaggtcg aagacactct gacaagattt actaattccg ttctaaatca taaagacgta | 2220 |
| ttaaacagtt ccagttcaga tcaggatact ttaagaagag aattccgtac attcatgcat | 2280 |
| gcacatatta ctcaaattga ggacaattct aggttttcta agcaagcttc ctcagatgca | 2340 |
| ttctcatctc cagaacagtc ttatttccag tgggttaatt ccacaggagg ctctcatgtt | 2400 |
| gcctgcgcct atagcttcgc tttttcaaac tgtctgatga gtgcgaattt actacagggc | 2460 |
| aaggatgcat ttccttctgg tactcagaaa taccttatct catcagttat gagacatgcg | 2520 |
| actaatatgt gcagaatgta caatgatttt gggagtatag ccagagataa tgctgaaaga | 2580 |
| aatgttaata gtatccattt tcctgagttt acactgtgca atggaacaag ccagaaccta | 2640 |
| gacgaaagaa aagaaagatt attgaaaatc gcaacttacg agcagggcta cctagatagg | 2700 |
| gcattagaag cgttggaaag acagtcaaga gatgacgcag gtgacagggc aggatccaag | 2760 |
| gatatgagaa aactaaagat tgtaaaactt ttttgcgacg ttacagacct gtacgaccaa | 2820 |
| ttatacgtta ttaaagattt gtcttcttct atgaaatga | 2859 |

SEQ ID NO: 229:

| | |
|---|---|
| MPGKIENGTP KDLKTGNDFV SAAKSLLDRA FKSHHSYYGL CSTSCQVYDT AWVAMIPKTR | 60 |
| DNVKQWLFPE CFHYLLKTQA ADGSWGSLPT TQTAGILDTA SAVLALLCHA QEPLQILDVS | 120 |
| PDEMGLRIEH GVTSLKRQLA VWNDVEDTNH IGVEFIIPAL LSMLEKELDV PSFEFPCRSI | 180 |
| LERMHGEKLG HFDLEQVYGK PSSLLHSLEA FLGKLDFDRL SHHLYHGSMM ASPSSTAAYL | 240 |
| IGATKWDDEA EDYLRHVMRN GAGHGNGGIS GTFPTTHFEC SWIIATLLKV GFTLKQIDGD | 300 |
| GLRGLSTILL EALRDENGVI GFAPRTADVD DTAKALLALS LVNQPVSPDI MIKVFEGKDH | 360 |
| FTTFGSERDP SLTSNLHVLL SLLKQSNLSQ YHPQILKTTL FTCRWWWGSD HCVKDKWNLS | 420 |
| HLYPTMLLVE AFTEVLHLID GGELSSLFDE SFKCKIGLSI FQAVLRIILT QDNDGSWRGY | 480 |
| REQTCYAILA LVQARHVCFF THMVDRLQSC VDRGFSWLKS CSFHSQDLTW TSKTAYEVGF | 540 |
| VAEAYKLAAL QSASLEVPAA TIGHSVTSAV PSSDLEKYMR LVRKTALFSP LDEWGLMASI | 600 |
| IESSFFVPLL QAQRVEIYPR DNIKVDEDKY LSIIPFTWVG CNNRSRTFAS NRWLYDMMYL | 660 |
| SLLGYQTDEY MEAVAGPVFG DVSLLHQTID KVIDNTMGNL ARANGTVHSG NGHQHESPNI | 720 |
| GQVEDTLTRF TNSVLNHKDV LNSSSSDQDT LRREFRTFMH AHITQIEDNS RFSKQASSDA | 780 |
| FSSPEQSYFQ WVNSTGGSHV ACAYSFAFSN CLMSANLLQG KDAFPSGTQK YLISSVMRHA | 840 |
| TNMCRMYNDF GSIARDNAER NVNSIHFPEF TLCNGTSQNL DERKERLLKI ATYEQGYLDR | 900 |
| ALEALERQSR DDAGDRAGSK DMRKLKIVKL FCDVTDLYDQ LYVIKDLSSS MK | 952 |

SEQ ID NO: 230:

| | |
|---|---|
| atgagtaagt ctaatagtat gaattctaca tcacacgaaa ccctttttca acaattggtc | 60 |
| ttgggtttgg accgtatgcc attgatggat gttcactggt tgatctacgt tgctttcggc | 120 |
| gcatggttat gttcttatgt gatacatgtt ttatcatctt cctctacagt aaaagtgcca | 180 |
| gttgttggat acaggtctgt attcgaacct acatggttgc ttagacttag attcgtctgg | 240 |
| gaaggtggct ctatcatagg tcaagggtac aataagttta aagactctat tttccaagtt | 300 |
| aggaaattgg gaactgatat tgtcattata ccacctaact atattgatga agtgagaaaa | 360 |
| ttgtcacagg acaagactag atcagttgaa cctttcatta atgattttgc aggtcaatac | 420 |
| acaagaggca tggttttctt gcaatctgac ttacaaaacc gtgttataca acaaagacta | 480 |
| actccaaaat tggtttcctt gaccaaggtc atgaaggaag agttggatta tgctttaaca | 540 |
| aaagagatgc ctgatatgaa aaatgacgaa tgggtagaag tagatatcag tagtataatg | 600 |
| gtgagattga tttccaggat ctccgccaga gtctttctag ggcctgaaca ctgtcgtaac | 660 |
| caggaatggt tgactactac agcagaatat tcagaatcac ttttcattac agggtttatc | 720 |
| ttaagagttg tacctcatat cttaagacca ttcatcgccc ctctattacc ttcatacagg | 780 |
| actctactta gaaacgtttc aagtggtaga agagtcatcg gtgacatcat aagatctcag | 840 |
| caaggggatg gtaacgaaga tatactttcc tggatgagag atgctgccac aggagaggaa | 900 |
| aagcaaatcg ataacattgc tcagagaatg ttaattcttt ctttagcatc aatccacact | 960 |
| actgcgatga ccatgacaca tgccatgtac gatctatgtg cttgccctga gtacattgaa | 1020 |
| ccattaagag atgaagttaa atctgttgtt ggggcttctg gctgggacaa gacagcgtta | 1080 |
| aacagatttc ataagttgga ctccttccta aaagagtcac aaagattcaa cccagtattc | 1140 |
| ttattgacat tcaatagaat ctaccatcaa tctatgacct tatcagatgg cactaacatt | 1200 |
| ccatctggaa cacgtattgc tgttccatca cacgcaatgt tgcaagattc tgcacatgtc | 1260 |
| ccaggtccaa ccccacctac tgaatttgat ggattcagat atagtaagat acgttctgat | 1320 |
| agtaactacg cacaaaagta cctattctcc atgaccgatt cttcaaacat ggctttcgga | 1380 |
| tacggcaagt atgcttgtcc aggtagattt tacgcgtcta atgagatgaa actaacatta | 1440 |
| gccattttgt tgctacaatt tgagttcaaa ctaccagatg gtaaaggtcg tcctagaaat | 1500 |
| atcactatcg attctgatat gattccagac ccaagagcta gactttgcgt cagaaaaaga | 1560 |
| tcacttagag atgaatga | 1578 |

SEQ ID NO: 231

| | |
|---|---|
| MSKSNSMNST SHETLFQQLV LGLDRMPLMD VHWLIYVAFG AWLCSYVIHV LSSSSTVKVP | 60 |
| VVGYRSVFEP TWLLRLRFVW EGGSIIGQGY NKFKDSIFQV RKLGTDIVII PPNYIDEVRK | 120 |
| LSQDKTRSVE PFINDFAGQY TRGMVFLQSD LQNRVIQQRL TPKLVSLTKV MKEELDYALT | 180 |
| KEMPDMKNDE WVEVDISSIM VRLISRISAR VFLGPEHCRN QEWLTTTAEY SESLFITGFI | 240 |
| LRVVPHILRP FIAPLLPSYR TLLRNVSSGR RVIGDIIRSQ QGDGNEDILS WMRDAATGEE | 300 |
| KQIDNIAQRM LILSLASIHT TAMTMTHAMY DLCACPEYIE PLRDEVKSVV GASGWDKTAL | 360 |
| NRFHKLDSFL KESQRFNPVF LLTFNRIYHQ SMILSDGINI PSGTRIAVPS HAMLQDSAHV | 420 |
| PGPTPPTEFD GFRYSKIRSD SNYAQKYLFS MTDSSNMAFG YGKYACPGRF YASNEMKLTL | 480 |
| AILLLQFEFK LPDGKGRPRN ITIDSDMIPD PRARLCVRKR SLRDE | 525 |

SEQ ID NO: 232

| | |
|---|---|
| atgtctattt tcaacatgat tacttcatat gctgggagtc aactcttacc attttacata | 60 |
| gcaatattcg ttttcacatt ggttccatgg gctattagat tctcatggtt ggaacttaga | 120 |
| aaggggtcag tagtgccact ggccaaccca cctgactcat tattcggcac aggcaagaca | 180 |
| cgtagatctt tcgttaaact ttccagagaa atactagcca aggcaagatc tctatttcca | 240 |
| aacgaaccat ttagattgat cacagactgg ggagaggttc ttattcttcc tcctgatttt | 300 |
| gccgatgaaa ttagaaacga tcctagatta tctttctcta aagctgcaat gcaggataat | 360 |
| catgccggca tcccaggttt cgaaacagtc gcattagttg gtagagaaga tcaacttatt | 420 |
| caaaaagttg ctagaaaaca actcacaaag cacctgtctg cagtcataga gcctttatct | 480 |
| agagagtcaa ccctagccgt ttcattgaat tttggtgaaa ctactgaatg gagagctata | 540 |
| agactaaagc cagccatttt ggatatcatt gctagaatca gctccagaat ctacctaggg | 600 |
| gatcagttgt gcagaaatga ggcatggttg aagattacaa agacatatac aaccaacttc | 660 |
| tacactgctt ctacaaactt gcgtatgttc ccaagatcaa tcagaccatt agcgcactgg | 720 |
| ttcttgcctg aatgcagaaa gttgagacaa gagagaaaag atgctatagg tatcattaca | 780 |
| ccattgatcg aaagacgtag agagttacgt agagcagcaa tcgctgccgg tcaacctctc | 840 |
| ccagtgtttc atgatgcaat cgactggtct gaacaggaag ctgaggcagc cggaactggt | 900 |
| gccagtttcg accctgttat ctttcaacta accttgtcct tgctggcaat tcataccact | 960 |
| tacgatctgt tacaacaaac tatgattgat ttaggtagac acccagagta cattgaacca | 1020 |
| ctaagacaag aggtagtaca gctgttgaga gaagagggat ggaaaaagac cacattattc | 1080 |
| aagatgaagt tattagactc cgcgattaag gaaagtcaga gaatgaaacc tggttctata | 1140 |
| gtcacaatgc gtagatacgt tactgaggat atcacccttt catcaggtct tacattgaaa | 1200 |
| aagggaacaa gattgaacgt ggacaataga agattggatg atcctaagat ttacgataac | 1260 |
| ccagaagtct acaatccata cagattttac gatatgagat ccgaagcggg taaggaccat | 1320 |
| ggtgctcaat tagtatctac aggttcaaac cacatgggtt ttggtcatgg acaacattct | 1380 |
| tgtccaggca gattcttcgc tgcaaacgaa atcaaggttg cactatgtca tatcttagtg | 1440 |
| aaatacgact ggaagctctg tccagatact gaaactaagc cagacacaag aggcatgatt | 1500 |
| gctaagagtt ctccagttac tgatatcctt atcaaaagac gtgaaagcgt cgaacttgat | 1560 |
| ttggaagcaa tttag | 1575 |

SEQ ID NO: 233

| | |
|---|---|
| MSIFNMITSY AGSQLLPFYI AIFVFTLVPW AIRFSWLELR KGSVVPLANP PDSLFGTGKT | 60 |
| RRSFVKLSRE ILAKARSLFP NEPFRLITDW GEVLILPPDF ADEIRNDPRL SFSKAAMQDN | 120 |
| HAGIPGFETV ALVGREDQLI QKVARKQLTK HLSAVIEPLS RESTLAVSLN FGETTEWRAI | 180 |
| RLKPAILDII ARISSRIYLG DQLCRNEAWL KITKTYTINF YTASTNLRMF PRSIRPLAHW | 240 |
| FLPECRKLRQ ERKDAIGIIT PLIERRRELR RAAIAAGQPL PVFHDAIDWS EQEAEAAGTG | 300 |
| ASFDPVIFQL TLSLLAIHTT YDLLQQTMID LGRHPEYIEP LRQEVVQLLR EEGWKKTTLF | 360 |
| KMKLLDSAIK ESQRMKPGSI VIMRRYVTED ITLSSGLTLK KGTRLNVDNR RLDDPKIYDN | 420 |
| PEVYNPYRFY DMRSEAGKDH GAQLVSTGSN HMGFGHGQHS CPGRFFAANE IKVALCHILV | 480 |
| KYDWKLCPDT ETKPDTRGMI AKSSPVTDIL IKRRESVELD LEAI | 524 |

SEQ ID NO: 234

| | |
|---|---|
| atgagcatct tcaatatgat caccagctat gcgggtagcc aacttttgcc cttctacatc | 60 |
| gccatattcg tctttacttt agtcccatgg gcaatccgct tctcttggct agaattgcgc | 120 |
| aagggctcag tggtgccact tgcgaacccg cccgactcac tgttcggcac cggtaaaacc | 180 |
| aggaggagtt ttgtcaagct tagtagagaa attctcgcta aagcgaggag cttgttccct | 240 |
| aatgagccat ttcgcttgat tacggactgg ggtgaggttc tcattctccc cccagacttt | 300 |
| gcagatgaga ttagaaatga tccgagactg agcttctcca aggcggcgat gcaggataat | 360 |
| catgctggaa tacctggctt tgagactgtt gccctggtgg gtcgtgaaga ccaacttatt | 420 |
| cagaaggtgg cccgaaagca gttgaccaag catctttccg ctgtcataga gccactatct | 480 |
| agagagtcca ccctcgcagt gtcgctcaac tttggagaga caacagaatg gcgagcgata | 540 |
| cgcctcaagc ccgcaattct agacatcatc gcccgcatct cgtccagaat ctatctcggc | 600 |
| gaccaactat gccgcaacga agcttggctg aagatcacaa agacatacac caccaacttc | 660 |
| tacactgcat ctaccaacct ccgaatgttt cctcgatcga tccgtcctct cgcccactgg | 720 |
| ttcctccccg aatgcagaaa gcttcgacag gagcgcaagg atgcaatcgg tattattacg | 780 |
| ccactgattg agcgccgccg tgagcttcga agagctgcga tcgcagctgg tcagcctctg | 840 |
| cctgtgttcc acgacgctat tgactggtcg gaacaggagg cagaagctgc aggcacaggg | 900 |
| gcctcgtttg accccgtgat cttccagctt acgctctctc ttctggcaat tcatacgacg | 960 |
| tatgatctcc tccagcaaac gatgattgac cttggtcgcc acccagagta tatcgagcct | 1020 |
| cttagacagg aagttgttca acttcttcgt gaagaaggtt ggaagaaaac aacgcttttc | 1080 |
| aagatgaagc tccttgacag tgctatcaaa gagtctcagc gaatgaagcc tggaagcata | 1140 |
| gttaccatgc gtcgctacgt aaccgaagac atcaccctct ctagcggcct gaccctcaaa | 1200 |
| aaagggaccc gcctcaacgt tgacaacaga cgcctcgacg atcccaaaat ctacgataac | 1260 |
| cccgaggttt acaatcctta tcgcttctac gacatgcgct ccgaagccgg gaaagaccat | 1320 |
| ggggcacagc tagtatcaac tggctcaaac catatgggct tcggccacgg tcagcactca | 1380 |
| tgcccagggc gtttcttcgc tgcgaatgag atcaaagtag cgctatgcca catcttggtc | 1440 |
| aagtatgatt ggaagctgtg ccctgacacg gagaccaagc ctgataccag gggcatgatt | 1500 |
| gccaagtcca gccctgtcac ggacatcttg atcaagcgtc gggagtcagt tgagttggat | 1560 |
| ttggaagcaa tttga | 1575 |

SEQ ID NO: 235

| | |
|---|---|
| MSIFNMITSY AGSQLLPFYI AIFVFTLVPW AIRFSWLELR KGSVVPLANP PDSLFGTGKT | 60 |
| RRSFVKLSRE ILAKARSLFP NEPFRLITDW GEVLILPPDF ADEIRNDPRL SFSKAAMQDN | 120 |
| HAGIPGFETV ALVGREDQLI QKVARKQLTK HLSAVIEPLS RESTLAVSLN FGETTEWRAI | 180 |
| RLKPAILDII ARISSRIYLG DQLCRNEAWL KITKTYTINF YTASTNLRMF PRSIRPLAHW | 240 |
| FLPECRKLRQ ERKDAIGIIT PLIERRRELR RAAIAAGQPL PVFHDAIDWS EQEAEAAGTG | 300 |
| ASFDPVIFQL TLSLLAIHTT YDLLQQTMID LGRHPEYIEP LRQEVVQLLR EEGWKKTTLF | 360 |
| KMKLLDSAIK ESQRMKPGSI VIMRRYVTED ITLSSGLTLK KGTRLNVDNR RLDDPKIYDN | 420 |
| PEVYNPYRFY DMRSEAGKDH GAQLVSTGSN HMGFGHGQHS CPGRFFAANE IKVALCHILV | 480 |
| KYDWKLCPDT ETKPDTRGMI AKSSPVTDIL IKRRESVELD LEAI | 524 |

SEQ ID NO: 236

| | |
|---|---|
| atgaaacaca ttgatgtgat gaacttcata tcgaaaatat gctcctggtc taaggacagc | 60 |
| ccaggattcg tccttctgat ttcaattctg gtgatactcg gcagtgtcac cttcattccc | 120 |
| aagtgtggca gaagaagcgc ctttgatgct ttgcccattg tgaacaaacc aaagtttggt | 180 |
| cccattttct caatcattgc tcgatggaga tttattcacc aaagcaagaa gatattggaa | 240 |
| gagggacaga agtgctacag caaccgcccc tttcgcatat ggacagactg gggcgaagta | 300 |
| ctcatgttga caccggatta tgcgcacgaa atacgcaatg acccgcatct cagcttttct | 360 |
| ggagctgtga aaatcgacgg ccacgcggat ataccgggct tcgagactgt gaaactgatt | 420 |
| tcgcatccag acaacctgat tcagctagta gcaaggaagc aattaaccag acaccttgcg | 480 |
| gctgtgattc agcctctttc tagtgttaca gaggaagccc tcatcaagaa tttagggaaa | 540 |
| tcacaagaat ggtctgagat ttatctaaaa tatgctgttc tagatatcat tgcccgacta | 600 |
| tcctcgcgca tttacttcgg agaactactg taccagaacg aagaatggct ttccattgta | 660 |
| aaaaattatg ccactcactt cttcactgcc agctccgatc tacgcaaagt tccttgggcc | 720 |
| tttcgctcac tagtccattg gttcgtgccg tcctgccgag cgctaaggct tgagcgctac | 780 |
| aatgcgcgtc gtgtcttaga accggttatc agccagcgtc gtcaactgaa ggaagctgcc | 840 |
| aaaacggctg gaggtacacc gttacacttc gaggatgcca ttgaatgggc cgaagtagaa | 900 |
| gctcgagtga aaggaacaaa atatgatcca gtaattttcc aattgacgct ctcgcttctg | 960 |
| gcaatacaca caacatacga tctcctcgag atgtgtatga ttgatctcgc aaagcgcccc | 1020 |
| gactgtatcg aggaccttcg taaagaagtc attacagtac tccgcaagga tggctggacg | 1080 |
| aagaatgctc tgtacaacat gaagctgctc gactctgcaa taaaagagtc tcaacgcctc | 1140 |
| aaaccaggaa gtatcacatc aatgcgtcgc tacgctactt cagacgtaca actgcgcgac | 1200 |
| ggcgtagttc tcaaaaaggg caataggctg aatgttctta ccttgcaccg atccccagac | 1260 |
| ctattccctt caccggatac ctacgaccca tatcggttct acaacatacg cggacagcct | 1320 |
| gggaaagaga actgggcgca actagtatcg acatctgttg aacatatggg ctttggtcat | 1380 |
| ggggaacact cgtgccctgg acgattcttt gcggcaaacg aaattaaggt agcacttgcg | 1440 |
| catatcctcg tcaagtacga ctggaagctg tcagacgagg cgggcggttg tactgaggtc | 1500 |
| aagggcatgg tcgaaaaggc aggaagtaag gtcaagatac tggtgagaca aaggcaagac | 1560 |
| gtggagagcg tccttgatga ggcgtga | 1587 |

SEQ ID NO: 237

| | |
|---|---|
| MKHIDVMNFI SKICSWSKDS PGFVLLISIL VILGSVTFIP KCGRRSAFDA LPIVNKPKFG | 60 |
| PIFSIIARWR FIHQSKKILE EGQKCYSNRP FRIWTDWGEV LMLTPDYAHE IRNDPHLSFS | 120 |
| GAVKIDGHAD IPGFETVKLI SHPDNLIQLV ARKQLTRHLA AVIQPLSSVT EEALIKNLGK | 180 |
| SQEWSEIYLK YAVLDIIARL SSRIYFGELL YQNEEWLSIV KNYATHFFTA SSDLRKVPWA | 240 |
| FRSLVHWFVP SCRALRLERY NARRVLEPVI SQRRQLKEAA KTAGGTPLHF EDAIEWAEVE | 300 |
| ARVKGTKYDP VIFQLTLSLL AIHTTYDLLE MCMIDLAKRP DCIEDLRKEV ITVLRKDGWT | 360 |
| KNALYNMKLL DSAIKESQRL KPGSITSMRR YATSDVQLRD GVVLKKGNRL NVLTLHRSPD | 420 |
| LFPSPDTYDP YRFYNIRGQP GKENWAQLVS TSVEHMGFGH GEHSCPGRFF AANEIKVALA | 480 |
| HILVKYDWKL SDEAGGCTEV KGMVEKAGSK VKILVRQRQD VESVLDEA | 528 |

SEQ ID NO: 238

| | |
|---|---|
| ATGAGCGAAA CATACACGAC AGCAGAAGTT GGAAAGCATA AGGACGAGGC GAATGGCTTC | 60 |
| TGGTTGATAG TTGAGAATGA CGTTTACGAC GTCACGAAGT TTATTGACGA GCACCCTGGC | 120 |
| GGTGCCAAGA TTCTAAAAAG GTGGTCTGGA AAAAACGCAA CTAAGGCATT CTGGAAGTAT | 180 |
| CATAATGAAC ACGTACTTGC TAAATACGGT AAGGACCTTA AAATAGGCGC CGTTGGCGAG | 240 |
| AGCGCGAAAC TATGA | |

SEQ ID NO: 239

| | |
|---|---|
| MSETYTTAEV GKHKDEANGF WLIVENDVYD VTKFIDEHPG GAKILKRWSG KNATKAFWKY | 60 |
| HNEHVLAKYG KDLKIGAVGE SAKL | |

SEQ ID NO: 240
ATGTTTGCTAGGAGTGCTTTCAGAGCAGCACAACCCCTTAGAAGCGTTAGGAGGTATGCCACAGAAGCGGGTGGAGCGGGTGGTAGCA
ACGCTTTCCTGTACGCTGCGGGCGCAGCCGCCTTTGGAGGAGCAGGCTATTGGTATTTCAGCAAGGGTGGTGCTCCGAGCGCTGCGGC
TGCGGCGGCCGATGTGAAACAGGCCGTTGGTATCGAACCGAAAAAAGCATTCACGGGAGGCGATCAAGGTTTCGTTAGCTTGAAACTT
TCCGATGTGGAGTTGGTAAACCACAATACAAAACGTCTTAGATTCGAGCTACCCGAGCCCGACCAAGTTAGTGGATTGCATGTGGCTT
CAGCGATTTTGACGAAGTACAAAGGGCCGAATGACGAGAAGGCAACACTAAGGCCATATACGCCCATTTCTGACGAATCCGAAAAAGG
TTTTATAGACCTACTTGTAAAGAAGTACCCCGATGGCCCCATGAGTACGCACTTACACAATCTGGTACCAGGCCAACGTCTAGATATA
AAGGGTCCGCTTCCCAAGTACCCGTGGGAGGAGAATAAGCACGAACATATTGCGCTAATAGCGGGTGGTACCGGGATTACACCAATGT
ATCAGTTGGCGAGGGCGATATTTAACAATCCAAACGACAAGACAAAGGTGACACTGGTGTTTGGTAATGTTTCCGAACAGGACATTCT
GCTAAAAAAGGAGTTCGAGCACCTAGAAAACACGTTCCCTCAGAGGTTCCGTGCATTCTACGTTCTTGATAATCCGCCTAAGGAATGG
GTTGGTAACTCTGGTTATATAAGCAAAGAGCTACTGAAAACAGTTTTGCCTGAGCCTAAGAACGAGAATATTAAACTGTTCGTGTGCG
GCCCCCCGGGCTTAATGAACGCTATCTCAGGAAACAAGGTATCACCAAAAAACCAAGGAGAACTAACCGGCGCACTAAAGGAGCTAGG
GTATAAGGAGGATCAGGTCTATAAATTTTAA
SEQ ID NO: 241
MFARSAFRAAQPLRSVRRYATEAGGAGGSNAFLYAAGAAAFGGAGYWYFSKGGAPSAAAAAADVKQAVGIEPKKAFTGGDQGFVSLKL
SDVELVNHNTKRLRFELPEPDQVSGLHVASAILTKYKGPNDEKATLRPYTPISDESEKGFIDLLVKKYPDGPMSTHLHNLVEGQRLDI
KGPLPKYPWEENKHEHIALIAGGYGITPMYQLARAIFNNPNDKTKVTLVFGNVSEQDILLKKEFEHLENTFPQRFRAFYVLDNPPKEW
VGNSGYISKELLKTVLPEPKNENIKLFVCGPPGLMNAISGNKVSPKNQGELTGALKELGYKEDQVYKF

What is claimed is:

1. A recombinant host cell, comprising:
(a) a recombinant gene encoding a first cytochrome P450 (P450) polypeptide; and/or
(b) a recombinant gene encoding a 2-oxoglutarate-dependent dioxygenase (2-ODD) polypeptide and/or a second cytochrome P450 (P450) polypeptide;
wherein the recombinant host cell is capable of producing a gibberellin precursor and/or a gibberellin compound.

2. The recombinant host cell of claim 1, wherein the gene encoding the first P450 polypeptide encodes a kaurenoic acid oxidase (KAO) polypeptide or a cytochrome P450 monooxygenase-1 (P450-1) polypeptide.

3. The recombinant host cell of claim 1 or 2, wherein the gene encoding the first P450 polypeptide comprises:
(a) a gene encoding a kaurenoic acid oxidase (KAO1) polypeptide;
(b) a gene encoding a kaurenoic acid oxidase (KAO2) polypeptide;
(c) a gene encoding a kaurenoic acid oxidase (KAO3) polypeptide;
(d) a gene encoding a kaurenoic acid oxidase (KAO4) polypeptide;
(e) a gene encoding a kaurenoic acid oxidase (KAO5) polypeptide;
(f) a gene encoding a kaurenoic acid oxidase (KAO6) polypeptide;
(g) a gene encoding a kaurenoic acid oxidase (KAO9) polypeptide;
(h) a gene encoding a kaurenoic acid oxidase (KAO10) polypeptide;
(i) a gene encoding a kaurenoic acid oxidase (KAO11) polypeptide;
(j) a gene encoding a cytochrome P450 monooxygenase-2 (P450-2) polypeptide;
(k) a gene encoding a cytochrome P450 monooxygenase-3 (P450-3) polypeptide;
(l) a gene encoding a cytochrome P-450 BJ-1 (CYP112) polypeptide; and/or
(m) a gene encoding a gibberellin A13-oxidase (GA₁₃ₒₓ) polypeptide.

4. The recombinant host cell of claim 3, wherein:
(a) the KAO1 polypeptide comprises a KAO1 polypeptide having at least 50% sequence identity to the amino acid sequence set forth in SEQ ID NO:90;
(b) the KAO2 polypeptide comprises a KAO2 polypeptide having at least 50% sequence identity to the amino acid sequence set forth in SEQ ID NO:88;
(c) the KAO3 polypeptide comprises a KAO3 polypeptide having at least 50% sequence identity to the amino acid sequence set forth in SEQ ID NO:146;
(d) the KAO4 polypeptide comprises a KAO4 polypeptide having at least 50% sequence identity to the amino acid sequence set forth in SEQ ID NO:74;
(e) the KAO5 polypeptide comprises a KAO5 polypeptide having at least 60% sequence identity to the amino acid sequence set forth in SEQ ID NO:62;
(f) the KAO6 polypeptide comprises a KAO6 polypeptide having at least 60% sequence identity to the amino acid sequence set forth in SEQ ID NO:60;
(g) the KAO9 polypeptide comprises a KAO9 polypeptide having at least 70% sequence identity to the amino acid sequence set forth in SEQ ID NO:68;
(h) the KAO10 polypeptide comprises a KAO10 polypeptide having at least 70% sequence identity to the amino acid sequence set forth in SEQ ID NO:58;
(i) the KAO11 polypeptide comprises a KAO11 polypeptide having at least 65% sequence identity to the amino acid sequence set forth in SEQ ID NO:64;
(j) the P450-2 polypeptide comprises a P450-2 polypeptide having at least 50% sequence identity to the amino acid sequence set forth in SEQ ID NO:80;
(k) the P450-3 polypeptide comprises a P450-3 polypeptide having at least 50% sequence identity to the amino acid sequence set forth in SEQ ID NO:186;
(l) the CYP112 polypeptide comprises a CYP112 polypeptide having at least 70% sequence identity to the amino acid sequence set forth in SEQ ID NOs: 4, 6, 8, 10, 124, or 128; or
(m) the GA₁₃ₒₓ polypeptide comprises a GA₁₃ₒₓ polypeptide having at least 70% sequence identity to the amino acid sequence set forth in SEQ ID NO:98.

5. The recombinant host cell of claim 1, wherein the gene encoding the second P450 polypeptide comprises:
(a) a P450-2 polypeptide having at least 50% sequence identity to the amino acid sequence set forth in SEQ ID NO:80;
(b) a P450-2 polypeptide having at least 50% sequence identity to the amino acid sequence set forth in SEQ ID NO:233;
(c) a P450-2 polypeptide having at least 50% sequence identity to the amino acid sequence set forth in SEQ ID NO 235;
(d) a P450-2 polypeptide having at least 50% sequence identity to the amino acid sequence set forth in SEQ ID NO:237;
(e) a P450-2 polypeptide having at least 50% sequence identity to the amino acid sequence set forth in SEQ ID NO:18; or
(f) a CYP112 polypeptide having at least 70% sequence identity to the amino acid sequence set forth in SEQ ID NO:124.

6. The recombinant host cell of any one of claims 1-5 wherein the gene encoding the 2-ODD polypeptide comprises:
(a) a gene encoding a desaturase (DES) polypeptide;
(b) a gene encoding a gibberellin A7-oxidase (GA₇ₒₓ) polypeptide;
(c) a gene encoding a gibberellin A3-oxidase (GA₃ₒₓ) polypeptide; or
(d) a gene encoding a gibberellin A20-oxidase (GA₂₀ₒₓ) polypeptide.

7. The recombinant host cell of claim 6, wherein:
(a) the DES polypeptide comprises a DES polypeptide having at least 50% sequence identity to the amino acid sequence set forth in SEQ ID NO:26;
(b) the GA₇ₒₓ polypeptide comprises a GA₇ₒₓ polypeptide having 60% or greater sequence identity to the amino acid sequence set forth in SEQ ID NO:152;
(c) the GA₃ₒₓ polypeptide comprises a GA₃ₒₓ polypeptide having at least 50% sequence identity to the amino acid sequence set forth in SEQ ID NO:28, SEQ ID NO:30, SEQ ID NO:36, or SEQ ID NO:44; or
(d) the GA₂₀ₒₓ polypeptide comprises a GA₂₀ₒₓ polypeptide having at least 70% sequence identity to the amino acid sequence set forth in SEQ ID NO:40 or SEQ ID NO:42.

8. A recombinant host cell, comprising:
(a) a gene encoding a kaurenoic acid oxidase (KAO4) polypeptide having at least 50% sequence identity to the amino acid sequence set forth in SEQ ID NO:74;
(b) a gene encoding a desaturase (DES) polypeptide having at least 50% sequence identity to the amino acid sequence set forth in SEQ ID NO:26;
(c) a gene encoding a cytochrome P450 monooxygenase-2 (P450-2) polypeptide having at least 50% sequence identity to the amino acid sequence set forth in SEQ ID NO:80; and
(d) a gene encoding a cytochrome P450 monooxygenase-3 (P450-3) polypeptide having at least 50% sequence identity to the amino acid sequence set forth in SEQ ID NO:186;
wherein the recombinant host cell is capable of producing a gibberellin precursor and/or a gibberellin compound.

9. A recombinant host cell, comprising:
(a) a gene encoding a kaurenoic acid oxidase (KAO4) polypeptide having at least 50% sequence identity to the amino acid sequence set forth in SEQ ID NO:74;
(b) a gene encoding a gibberellin A20-oxidase (GA₂₀ₒₓ) polypeptide having at least 50% sequence identity to the amino acid sequence set forth in SEQ ID NO:42;
(c) a gene encoding a cytochrome P450 monooxygenase-3 (P450-3) polypeptide having at least 50% sequence identity to the amino acid sequence set forth in SEQ ID NO: 186; and
(d) a gene encoding a desaturase (DES) polypeptide having at least 50% sequence identity to the amino acid sequence set forth in SEQ ID NO:26;
wherein the recombinant host cell is capable of producing a gibberellin precursor and/or a gibberellin compound.

10. A recombinant host cell, comprising a gene encoding a kaurenoic acid oxidase (KAO) polypeptide having at least 60% sequence identity to the amino acid sequence set forth in SEQ ID NO:62, SEQ ID NO:60, or SEQ ID NO:152, at least 70% sequence identity to the amino acid sequence set forth in SEQ ID NO:58 or SEQ ID NO:68, at least 65% sequence identity to the amino acid sequence set forth in SEQ ID NO:64, or at least 50% sequence identity to the amino acid sequence set forth in SEQ ID NO:74;
wherein the recombinant host cell is capable of producing gibberellin precursor and/or a gibberellin compound.

11. The recombinant host cell of claim 10, further comprising:
(a) a gene encoding a gibberellin A20-oxidase (GA₂₀ₒₓ) polypeptide having at least 70% sequence identity to the amino acid sequence set forth in SEQ ID NO:40; and
(b) a gene encoding a gibberellin A13-oxidase (GA₁₃ₒₓ) polypeptide having at least 70% sequence identity to the amino acid sequence set forth in SEQ ID NO:98.

12. A recombinant host cell, comprising:
(a) a gene encoding a kaurenoic acid oxidase (KAO11) polypeptide having at least 65% sequence identity to the amino acid sequence set forth in SEQ ID NO:64; and
(b) a gene encoding a cytochrome P-450 BJ-1 (CYP112) polypeptide having at least 70% sequence identity to the amino acid sequence set forth in SEQ ID NO:124;
wherein the recombinant host cell is capable of producing a gibberellin precursor and/or a gibberellin compound.

13. A recombinant host cell, comprising:
(a) a gene encoding a kaurenoic acid oxidase (KAO4) polypeptide having at least 50% sequence identity to the amino acid sequence set forth in SEQ ID NO:74; and
(b) a gene encoding a cytochrome P-450 BJ-1 (CYP112) polypeptide having at least 70% sequence identity to the amino acid sequence set forth in SEQ ID NO:124;
wherein the recombinant host cell is capable of producing a gibberellin precursor and/or a gibberellin compound.

14. The recombinant host cell of any one of claims 1-13, further comprising:
(a) a gene encoding a polypeptide capable of synthesizing geranylgeranyl pyrophosphate (GGPP) from farnesyl diphosphate (FPP) and isopentenyl diphosphate (IPP);
(b) a gene encoding a polypeptide capable of synthesizing ent-copalyl diphosphate from GGPP;
(c) a gene encoding a polypeptide capable of synthesizing ent-kaurene from ent-copalyl pyrophosphate;
(d) a gene encoding a bifunctional polypeptide capable of synthesizing ent-copalyl diphosphate from GGPP and synthesizing ent-kaurene from ent-copalyl pyrophosphate;
(e) a gene encoding a polypeptide capable of synthesizing ent-kaurenoic acid from ent-kaurene;
(f) a gene encoding a cytochrome B5 polypeptide;
(g) a gene encoding a polypeptide capable of reducing cytochrome B5 polypeptide;
(h) a gene encoding a polypeptide capable of reducing cytochrome P450 complex;
(i) a gene encoding a ferredoxin polypeptide;
(j) a gene encoding a ferredoxin reductase polypeptide; and/or
(k) an alcohol dehydrogenase (ADH) polypeptide capable of reducing a gibberellin intermediate.

15. The recombinant host cell of claim 14, wherein:
(a) the polypeptide capable of synthesizing geranylgeranyl pyrophosphate (GGPP) from farnesyl diphosphate (FPP) and isopentenyl diphosphate (IPP) comprises a polypeptide having at least 70% sequence identity to the amino acid sequence set forth in SEQ ID NO:50, SEQ ID NO:134, or SEQ ID NO:178;
(b) the polypeptide capable of synthesizing ent-copalyl diphosphate from GGPP comprises a polypeptide having at least 70% sequence identity to the amino acid sequence set forth in SEQ ID NO:38, SEQ ID NO:102, SEQ ID NO:104, SEQ ID NO:106, SEQ ID NO:108, or SEQ ID NO:180;
(c) the polypeptide capable of synthesizing ent-kaurene from ent-copalyl pyrophosphate comprises a polypeptide having at least 70% sequence identity to the amino acid sequence set forth in SEQ ID NO:102 or SEQ ID NO:106;
(d) the bifunctional polypeptide capable of synthesizing ent-copalyl diphosphate from GGPP and synthesizing ent-kaurene from ent-copalyl pyrophosphate comprises a CDPS-KS polypeptide having at least 70% sequence identity to the amino acid sequence set forth in SEQ ID NO:104;
(e) the polypeptide capable of synthesizing ent-kaurenoic acid from ent-kaurene comprises a polypeptide having at least 70% sequence identity to the amino acid sequence set forth in SEQ ID NO:82, SEQ ID NO:164, SEQ ID NO:170, or SEQ ID NO:172;
(f) the cytochrome B5 polypeptide comprises a cytochrome B5 polypeptide having at least 60% sequence identity to the amino acid sequence set forth in SEQ ID NO:160 or SEQ ID NO:239;
(g) the cytochrome B5 reductase polypeptide comprises a cytochrome B5 reductase polypeptide having at least 80% sequence identity to the amino acid sequence set forth in SEQ ID NO:2 or SEQ ID NO:241;
(h) the polypeptide capable of reducing cytochrome P450 complex comprises a CPR reductase polypeptide having at least 50% sequence identity to the amino acid sequence set forth in SEQ ID NO:48, SEQ ID NO:100, SEQ ID NO:140, SEQ ID NO:158, SEQ ID NO:168, SEQ ID NO:192 or SEQ ID NO:194;
(i) the ferredoxin polypeptide comprises a ferredoxin polypeptide having at least 80% sequence identity to the amino acid sequence set forth in SEQ ID NO:148;
(j) the ferredoxin reductase polypeptide comprises a ferredoxin reductase polypeptide having at least 70% sequence identity to the amino acid sequence set forth in SEQ ID NO:150; and/or
(k) the ADH polypeptide comprises an ADH polypeptide having at least 70% sequence identity to the amino acid sequence set forth in SEQ ID NO:116.

16. The recombinant host cell of any one of claims 1-15, further comprising:
(a) a gene encoding an open reading frame (ORF) polypeptide;
(b) a gene encoding an aldehyde dehydrogenase (AIdDH) polypeptide;
(c) a gene encoding a myo-inositol transport protein ITR1 (smt) polypeptide;
(d) a gene encoding an endoplasmic reticulum (ER) membrane polypeptide; and/or
(e) a gene encoding a damage resistance protein 1 (DAP) polypeptide.

17. The recombinant host cell of claim 16, wherein:
(a) the ORF polypeptide comprises an ORF polypeptide having at least 50% sequence identity to the amino acid sequence set forth in SEQ ID NO:154 or SEQ ID NO:156;
(b) the AIdDH polypeptide comprises an AIdDH polypeptide having at least 60% sequence identity to the amino acid sequence set forth in SEQ ID NO:202;
(c) the smt polypeptide comprises an smt polypeptide having at least 90% sequence identity to the amino acid sequence set forth in SEQ ID NO:209;
(d) the ER membrane polypeptide comprises an inheritance of cortical ER protein 2 (ICE2) polypeptide having at least 60% sequence identity to the amino acid sequence set forth in SEQ ID NO:206; and/or
(e) the DAP polypeptide comprises a DAP polypeptide having at least 70% sequence identity to the amino acid sequence set forth in SEQ ID NO:213, SEQ ID NO:215, SEQ ID NO:217, or SEQ ID NO:224.

18. The recombinant host cell of any one of claims 1-17, wherein expression of the genes increases the portion of the gibberellin precursor and/or the gibberellin compound produced by the recombinant host cell by at least about 10%, 25%, 50%, 75%, 80%, 90%, 95%, 100% or more.

19. The recombinant host cell of any one of claims 1-18, wherein the gibberellin compound comprises GA₁, GA₃, GA₄, GA₅, GA₇, GA₉, GA₁₂, GA₁₃, GA₁₄, GA₁₅, GA₁₉, GA₂₀, GA₂₄, GA₂₅, GA₃₆, GA₃₇, GA₄₄, GA₅₃, or GA₁₁₀.

20. The recombinant host cell of any one of claims 1-19, wherein the recombinant host comprises a plant cell, a mammalian cell, an insect cell, a fungal cell, an algal cell or a bacterial cell.

21. A method of producing a gibberellin precursor and/or a gibberellin compound in a cell culture, comprising growing the recombinant host cell of any one of claims 1-20 in a cell culture, under conditions in which the genes are expressed;
wherein the gibberellin precursor and/or the gibberellin compound is produced by the recombinant host cell.

22. The method of claim 21, further comprising isolating the gibberellin precursor and/or the gibberellin compound from the cell culture.

23. The method of claim 22, wherein the isolating step comprises:
(a) contacting the cell culture comprising the gibberellin precursor and/or the gibberellin compound with:
(i) one or more adsorbent resins in order to bind at least a portion of the gibberellin precursor and/or the gibberellin compound to the resin, thereby isolating the gibberellin precursor and/or the gibberellin compound; or
(ii) one or more ion exchange or reversed-phase chromatography columns in order to bind at least a portion of the gibberellin precursor and/or the gibberellin compound in the column, thereby isolating the gibberellin precursor and/or the gibberellin compound; or
(b) crystallizing and/or extracting the gibberellin precursor and/or the gibberellin compound from the cell culture, thereby isolating the gibberellin precursor and/or the gibberellin compound; or
(c) separating the cell culture into a solid phase and a liquid phase, wherein the liquid phase comprises the gibberellin precursor and/or the gibberellin compound; and
(i) contacting the liquid phase with one or more adsorbent resins in order to bind at least a portion of the gibberellin precursor and/or the gibberellin compound to the resin, thereby isolating the gibberellin precursor and/or the gibberellin compound;
(ii) contacting the liquid phase with one or more ion exchange or reversed-phase chromatography columns in order to bind at least a portion of the gibberellin precursor and/or the gibberellin compound in the column, thereby isolating the gibberellin precursor and/or the gibberellin compound; or
(iii) crystallizing and/or extracting the gibberellin precursor and/or the gibberellin compound from the liquid phase, thereby isolating the gibberellin precursor and/or the gibberellin compound.

24. The method of any one of claims 21-23, further comprising recovering the gibberellin precursor and/or the gibberellin compound.

25. The method of any one of claims 21-23, further comprising:
(a) one or more steps of converting kaurenoic acid to GA₁₂ and GA₁₄ catalyzed by a first P450 polypeptide; and
(b) a step of converting GA₁₄ to GA₄ catalyzed by a second P450 polypeptide.

26. The method of claim 25, wherein:
(a) the first P450 polypeptide comprises:
(i) a KAO4 polypeptide having at least 50% sequence identity to the amino acid sequence set forth in SEQ ID NO:74;
(ii) a KAO1 polypeptide having at least 50% sequence identity to the amino acid sequence set for in SEQ ID NO:90; or
(iii) a KAO3 polypeptide having at least 50% sequence identity to the amino acid sequence set for in SEQ ID NO:146; and
(b) the second P450 polypeptide comprises:
(i) a P450-2 polypeptide having at least 50% sequence identity to the amino acid sequence set forth in SEQ ID NO:80;
(ii) a P450-2 polypeptide having at least 50% sequence identity to the amino acid sequence set forth in SEQ ID NO:233;
(iii) a P450-2 polypeptide having at least 50% sequence identity to the amino acid sequence set forth in SEQ ID NO 235;
(iv) a P450-2 polypeptide having at least 50% sequence identity to the amino acid sequence set forth in SEQ ID NO:237;
(v) a P450-2 polypeptide having at least 50% sequence identity to the amino acid sequence set forth in SEQ ID NO:18; or
(vi) a CYP112 polypeptide having at least 70% sequence identity to the amino acid sequence set forth in SEQ ID NO:124.

27. The method of claim 25 or 26, further comprising a step of converting GA₄ to GA₁ catalyzed by a third P450 polypeptide.

28. The method of claim 27, wherein the third P450 polypeptide comprises:
(a) a P450-3 polypeptide having at least 50% sequence identity to the amino acid sequence set forth in SEQ ID NO:186; or
(b) a GA₁₃ₒₓ₋₁ polypeptide having at least 50% sequence identity to the amino acid sequence set forth in SEQ ID NO:98.

29. The method of any one of claims 25-28, further comprising:
(a) a step of converting GA₄ to GA₇ catalyzed by a 2-ODD polypeptide; and
(b) a step of converting GA₇ to GA₃ catalyzed by a fourth P450 polypeptide.

30. The method of claim 29, wherein:
(a) the 2-ODD polypeptide comprises a DES polypeptide having at least 50% sequence identity to the amino acid sequence set forth in SEQ IN NO:26; and
(b) the fourth P450 polypeptide comprises:
(i) a P450-3 polypeptide having at least 50% sequence identity to the amino acid sequence set forth in SEQ ID NO:186; or
(ii) a GA₁₃ₒₓ₋₁ polypeptide having at least 50% sequence identity to the amino acid sequence set forth in SEQ ID NO:98.

31. The method of any one of claims 20-26, further comprising:
(a) one or more steps of converting kaurenoic acid to GA₁₂ and/or GA₁₄ catalyzed by a first P450 polypeptide; and
(b) a step of converting GA₁₄ to GA₄ catalyzed by a 2-ODD polypeptide.

32. The method of claim 31, wherein:
(a) the first P450 polypeptide comprises a KAO4 polypeptide having at least 50% sequence identity to the amino acid sequence set forth in SEQ ID NO:74; and
(b) the 2-ODD polypeptide comprises a GA₂₀ₒₓ polypeptide having at least 70% sequence identity to the amino acid sequence set forth in SEQ ID NO:40 or SEQ ID NO:42.

33. The method of claim 31 or 32, further comprising a step of converting GA₄ to GA₁ catalyzed by a second P450 polypeptide.

34. The method of claim 33, wherein the second P450 polypeptide comprises:
(a) a P450-3 polypeptide having at least 50% sequence identity to the amino acid sequence set forth in SEQ ID NO:186; or
(b) a GA₁₃ₒₓ₋₁ polypeptide having at least 50% sequence identity to the amino acid sequence set forth in SEQ ID NO:98.

35. The method of claim 31 or 32, further comprising:
(a) a step of converting GA₄ to GA₇ catalyzed by a second 2-ODD polypeptide; and
(b) a step of converting GA₇ to GA₃ catalyzed by a second P450 polypeptide.

36. The method of claim 35, wherein:
(a) the second 2-ODD polypeptide comprises a DES polypeptide having at least 50% sequence identity to the amino acid sequence set forth in SEQ ID NO:26; and
(b) the second P450 polypeptide comprises:
(i) a P450-3 polypeptide having at least 50% sequence identity to the amino acid sequence set forth in SEQ ID NO:186; or
(ii) a GA₁₃ₒₓ₋₁ polypeptide having at least 50% sequence identity to the amino acid sequence set forth in SEQ ID NO:98.

37. The method of any one of claims 21-36, wherein the recombinant host cell is grown in a fermentor at a temperature for a period of time, wherein the temperature and period of time facilitate the production of the gibberellin precursor and/or the gibberellin compound.

38. The method of any one of claims 21-37, wherein the gibberellin compound comprises GA₃ and its precursors, metabolites, or related compounds, including: GA₁, GA₄, GA₅, GA₇, GA₉, GA₁₂, GA₁₃, GA₁₄, GA₁₅, GA₁₉, GA₂₀, GA₂₄, GA₂₅, GA₃₆, GA₃₇, GA₄₄, GA₅₃, or GA₁₁₀.

39. The method of any one of claims 21-38, wherein the recombinant host comprises a plant cell, a mammalian cell, an insect cell, a fungal cell, an algal cell or a bacterial cell.

40. A cell culture, comprising the recombinant host cell of any one of claims 1-20, the cell culture further comprising:
(a) the gibberellin precursor and/or the gibberellin compound produced by the recombinant host cell;
(b) a carbon source; and
(c) supplemental nutrients comprising trace metals, vitamins, salts, YNB, a nitrogen source, and/or amino acids;
wherein one or more gibberellin precursors and/or the gibberellin compounds are present at a concentration of at least 100 mg/liter of the cell culture.

41. A cell lysate from the recombinant host cell of any one of claims 1-20 grown in the cell culture, comprising:
(a) the gibberellin precursor and/or the gibberellin compound produced by the recombinant host cell;
(b) a carbon source; and
(c) supplemental nutrients comprising trace metals, vitamins, salts, YNB, a nitrogen source, and/or amino acids;
wherein one or more gibberellin precursors and/or the gibberellin compounds are present at a concentration of at least 100 mg/liter of the cell culture.
